(19) United States
(12) Patent Application Publication
(10) Pub. No.: US 2003/0203890 A1
(43) Pub. Date: Oct. 30, 2003

(54) Method for treating nerve injury caused as a result of surgery

(76) Inventors: Joseph P. Steiner, Mount Airy, MD (US); Solomon Snyder, Baltimore, MD (US); Arthur L. Burnett, Baltimore, MD (US)

Correspondence Address: GUILFORD PHARMACEUTICALS C/O, FOLEY & LARDNER, 3000 K STREET, NW, WASHINGTON, DC, 20007-5143, US

(21) Appl. No.: 10/156,735

(22) Filed: May 29, 2002

Related U.S. Application Data
(60) Provisional application No. 60/293,544, filed on May 29, 2001.

(51) Int. Cl. A61K 31/554; A61K 31/553; A61K 31/55; A61K 31/551; A61K 31/54; A61K 31/537; A61K 31/4172; A61K 31/445

(52) U.S. Cl. 514/211.01; 514/217.11; 514/218; 514/227.5; 514/237.5; 514/255.01; 514/330; 514/365; 514/374; 514/385; 514/423

(57) ABSTRACT

The present invention relates generally to methods for treating or preventing nerve injury in a warm-blooded animal caused as a consequence of surgery by administering neurotrophic compounds described below. The invention relates more specifically to methods for treating or preventing nerve injury caused as a consequence of prostate surgery as well as erectile dysfunction.

### BACKGROUND OF THE INVENTION

The invention relates generally to methods for treating nerve injury caused as a consequence of surgery. The present invention relates more specifically to methods for treating nerve injury caused as a consequence of prostate surgery, or for methods of neuroprotection of penile innervation, by administering a neurotrophic compound to a patient in need thereof.

A. Neuroimmunophilins

The peptidyl-prolyl isomerases (“PPIases”) are a family of ubiquitous enzymes which catalyze the interconversion of cis and trans amide bond rotamers adjacent to proline residues in peptide substrates. See, for example, Galat, A., Eur. J. Biochem. (1993) 216:689-707 and Kay, J. E., Biochem. J. (1996) 314:361-385. The PPIases have been referred to as “immunophilins” because of their interaction with certain immunosuppressant drugs. Schreiber, S. L., Science (1991) 251:283-287; Rosen, M. K. and Schreiber, S. L., Angew. Chem. Intl. Ed. Engi. (1992) 31:384-400.

The PPIase, cyclophilin A, was found to be the intracellular protein target for the potent immunosuppressant drug cyclosporin A. Subsequently, the structurally unrelated macrolide immunosuppressant FK506 was discovered to bind to a different PPIase enzyme which was named FK506-binding protein, or FKBP. Rapamycin, another macrolide drug which is a structural analogue of FK506, also interacts with FKBP.

All three of these drugs bind to their respective immunophilins and inhibit the respective PPIase activities. However, inhibition of immunophilin enzymatic activity is not the cause of the observed immunosuppressive effects. Binding of the drugs to the immunophilins results in the formation of “activated complexes”, which interact with downstream proteins to inhibit proliferation of T-lymphocytes. Schreiber, supra; Rosen, et al., supra. In the case of FK506, binding to FKBP results in a drug-protein complex which is a potent inhibitor of the calcium-calmodulin-dependent protein phosphatase, calcineurin. Bierer, B. E., Mattila, P. S., Standaert, R. F., Herzenberg, L. A., Burakoff, S. J., Crabtree, G., Schreiber, S. L., Proc. Natl. Acad. Sci. USA (1990) 87:9231-9235; Liu, J., Farmer, J. D., Lane, W. S., Friedman, J., Weissman, I., Schreiber, S. L.; Cell (1991) 66:807-815.

Neither FK506 nor FKBP alone appreciably inhibits calcineurin's activity. Inhibiting calcineurin blocks the signaling pathway by which the activated T-cell receptor causes transcription of the gene for interleukin-2, inhibiting the immune response. Despite the structural dissimilarity between FK506 and cyclosporin A (and cyclophilin and FKBP), the cyclosporin A-cyclophilin complex also inhibits calcineurin, and thus cyclosporin A and FK506 have the same mechanism of action.

On the other hand, while rapamycin and FK506 have similar structures and bind to the same immunophilin (FKBP), rapamycin's mechanism of action is different from that of FK506. The complex of FKBP12 with rapamycin interacts with a protein called FRAP, or RAFT, and in so doing blocks the signal pathway leading from the IL-2 receptor on the surface of T-cells to promotion of entry into the cell cycle in the nucleus. Sabatini, D. M., Erdjument-Bromage, H., Lui, M.; Tempst, P., Snyder, S. H., Cell (1994) 78:35-43; Brown, E. J., Albers, M. W., Shin, T. B., Ichikawa, K., Keith, C. T., Lane, W. S., Schreiber, S. L. Nature (1994) 369:756-758; Brown, E. J., Beal, P. A., Keith, C. T., Chen, J., Shin, T. B., Schreiber, S. L., Nature (1995) 377:441-446.

Thus, all three drugs produce the same effect—suppression of T-cell proliferation—but do so by inhibiting distinct signal transduction pathways. The introduction of cyclosporin (“CsA”) marked a breakthrough in organ transplantation, and the drug became a major pharmaceutical product. The subsequent discovery of rapamycin (“Rapa”) and FK506 further fueled interest in the cellular basis of the actions of these drugs. The discovery of the interaction of the immunophilins with CsA, FK506 and Rapa led to research on the mechanistic basis of immunophilin-mediated immunosuppression.

### Immunophilins and the Nervous System

Because the initial interest in the immunophilins was largely driven by their role in the mechanism of action of the immunosuppressant drugs, most of the original studies of these proteins and their actions focused on the tissues of the immune system. In 1992, it was reported that levels of FKBP12 in the brain were 30 to 50 times higher than in the immune tissues. Steiner, J. P., Dawson, T. M., Fotuhi, M., Glatt, C. E., Snowman, A. M., Cohen, N., Snyder, S. H., Nature (1992) 358:584-587. This finding suggested a role for the immunophilins in the functioning of the nervous system. Both FKBP and cyclophilin were widely distributed in the brain and were found almost exclusively within neurons. The distribution of the immunophilins in the brain closely resembled that of calcineurin, suggesting a potential neurological link. Steiner, J. P., Dawson, T. M., Fotuhi, M., Glatt, C. E., Snowman, A. M., Cohen, N., Snyder, S. H., Nature (1992) 358:584-587; Dawson, T. M., Steiner, J. P., Lyons, W. E., Fotuhi, M., Blue, M., Snyder, S. H., Neuroscience (1994) 62:569-580.

Subsequent work demonstrated that the phosphorylation levels of several known calcineurin substrates were altered in the presence of FK506. Steiner, J. P., Dawson, T. M., Fotuhi, M., Glatt, C. E., Snowman, A. M., Cohen, N., Snyder, S. H., Nature (1992) 358:584-587. One of the proteins affected by FK506 treatment, GAP-43, mediates neuronal process elongation. Lyons, W. E., Steiner, J. P., Snyder, S. H., Dawson, T. M., J. Neurosci. (1995) 15:2985-2994. This research revealed that FKBP12 and GAP-43 were upregulated in damaged facial or sciatic nerves in rats. Also, FKBP12 was found in very high levels in the growth cones of neonatal neurons. FK506 was tested to determine whether or not it might have an effect on nerve growth or regeneration. In cell culture experiments with PC12 cells or sensory neurons from dorsal root ganglia, FK506 promoted process (neurite) extension with subnanomolar potency. Lyons, W. E., George, E. B., Dawson, T. M., Steiner, J. P., Snyder, S. H., Proc. Natl. Acad. Sci. USA (1994) 91:3191-3195. Gold et al. demonstrated that FK506 functioned as a neurotrophic agent in vivo. In rats with crushed sciatic nerves, FK506 accelerated nerve regeneration and functional recovery. Gold, B. G., Storm-Dickerson, T., Austin, D. R., Restorative Neurol. Neurosci., (1994) 6:287; Gold, B. G., Katoh, K., Storm-Dickerson, T. J, Neurosci. (1995) 15:7509-7516. See, also, Snyder, S. H., Sabatini, D. M., Nature Medicine (1995) 1:32-37 (regeneration of lesioned facial nerves in rats augmented by FK506).

Besides FK506, rapamycin and cyclosporin also produced potent neurotrophic effects in vitro in PC12 cells and chick sensory neurons. Steiner, J. P., Connolly, M. A., Valentine, H. L., Hamilton, G. S., Dawson, T. M., Hester, L., Snyder, S. H., Nature Medicine (1997) 3:421-428. As noted above, the mechanism for immunosuppression by rapamycin is different than that of FK506 or cyclosporin. The observation that rapamycin exerted neurotrophic effects similar to FK506 and cyclosporin suggested that the nerve regenerative effects of the compounds are mediated by a different mechanism than that by which they suppress T-cell proliferation.

Analogues of FK506, rapamycin, and cyclosporin which bind to their respective immunophilins, but are devoid of immunosuppressive activity, are known in the art. Thus, the FK506 analogue L-685,818 binds to FKBP but does not interact with calcineurin, and is therefore nonimmunosuppressive. Dumont, F. J., Staruch, M. J., Koprak, S. L., J. Exp. Med. (1992) 176:751-760.

Similarly, 6-methyl-alanyl cyclosporin A (6-[Me]-ala-CsA) binds to cyclophilin but likewise lacks the ability to inhibit calcineurin. The rapamycin analogue WAY-124,466 binds FKBP but does not interact with RAFT, and is likewise nonimmunosuppressive. Ocain, T. D., Longhi, D., Steffan, R. J., Caccese, R. G., Sehgal, S. N., Biochem. Biophys. Res. Commun. (1993) 192:1340-1346; Sigal, N. H., Dumont, F., Durette, P., Siekierka, J. J., Peterson, L., Rich, D., J. Exp. Med. (1991) 173:619-628. These nonimmunosuppressive compounds were shown to be potent neurotrophic agents in vitro, and one compound, L-685,818, was as effective as FK506 in promoting morphological and functional recovery following sciatic nerve crush in rats. Steiner, J. P., Connolly, M. A., Valentine, H. L., Hamilton, G. S., Dawson, T. M., Hester, L., Snyder, S. H., Nature Medicine (1997) 3:421-428. These results demonstrated that the neurotrophic properties of the immunosuppressant drugs could be functionally dissected from their immune system effects.

Published work by researchers studying the mechanism of action of FK506 and similar drugs had shown that the minimal FKBP-binding domain of FK506 (as formulated by Holt et al., BioMed. Chem. Lett. (1994) 4:315-320) possessed good affinity for FKBP. Hamilton et al. proposed that the neurotrophic effects of FK506 resided within the immunophilin binding domain, and synthesized a series of compounds which were shown to be highly effective in promoting neurite outgrowth from sensory neurons, often at picomolar concentrations. Hamilton, G. S., Huang, W., Connolly, M. A., Ross, D. T., Guo, H., Valentine, H. L., Suzdak, P. D., Steiner, J. P., BioMed. Chem. Lett. (1997). These compounds were shown to be effective in animal models of neurodegenerative disease.

### FKBP12 Inhibitors/Ligands

A number of researchers in the early 1990s explored the mechanism of immunosuppression by FK506, cyclosporin and rapamycin, and sought to design second-generation immunosuppressant agents that lacked the toxic side effects of the original drugs. A pivotal compound, 506BD (for “FK506 binding domain”—see Bierer, B. E., Somers, P. K., Wandless, T-J., Burakoff, S. J., Schreiber, S. L., Science (1990) 250:556-559), retained the portion of FK506 which binds FKBP12 in an intact form, while the portion of the macrocyclic ring of FK506 which extends beyond FKBP12 in the drug-protein complex was significantly altered. The finding that 506BD was a high-affinity ligand for, and inhibitor of, FK506, but did not suppress T-cell proliferation was the first demonstration that the immunosuppressant effects of FK506 were not simply caused by rotamase activity inhibition.

In addition to various macrocyclic analogues of FK506 and rapamycin, simplified compounds which represent the excised FKBP binding domain of these drugs were synthesized and evaluated. Non-macrocyclic compounds with the FKBP-binding domain of FK506 excised possess lower affinity for FKBP12 than the parent compounds. Such structures still possess nanomolar affinity for the protein. See, eg., Hamilton, G. S., Steiner, J. P., Curr. Pharm. Design (1997) 3:405-428; Teague, S. J., Stocks, M. J., BioMed. Chem. Lett., (1993) 3:1947-1950; Teague, S. J., Cooper, M. E., Donald, D. K., Furber, M., BioMed. Chem. Lett. (1994) 4:1581-1584.

Holt et al. published several studies of simple pipecolate FKBP12 inhibitors which possessed excellent affinity for FKBP12. In initial studies, replacement of the pyranose ring of FK506 mimetics demonstrated that simple alkyl groups such as cyclohexyl and dimethylpentyl worked well in this regard. Holt et al., BioMed. Chem. Lett. (1994) 4:315-320. Simple compounds possessed good affinity for FKBP12 (K₁ values of 250 and 25 nM, respectively). These structures demonstrated that these simple mimics of the binding domain of FK506 bound to the immunophilin in a manner nearly identical to that of the corresponding portion of FK506. Holt, D. A., Luengo, J. I., Yamashita, D. S., Oh, H. J., Konialian, A. L., Yen, H. K., Rozamus, L. W., Brandt, M., Bossard, M. J., Levy, M. A., Eggleston, D. S., Liang, J., Schultz, L. W.; Stout, T. J.; Clardy, I., J. Am. Chem. Soc. (1993) 115:9925-9938.

Armistead et al. also described several pipecolate FKBP12 inhibitors. X-ray structures of the complexes of these molecules with FKBP also demonstrated that the binding modes of these simple structures were related to that of FK506. Armistead, D. M., Badia, M. C., Deininger, D. D., Duffy, J. P., Saunders, J. O., Tung, R. D., Thomson, J. A.; DeCenzo, M. T.; Futer, O., Livingston, D. J., Murcko, M. A., Yamashita, M. M., Navia, M. A., Acta Cryst. (1995) D51:522-528.

As expected from the noted effector-domain model, FKBP12 ligands lacking an effector element were inactive as immunosuppressant agents, failing to suppress lymphocyte proliferation both in vitro and in vivo.

### Neuroprotective/Neuroregenerative Effects of FKBP12 Ligands

Steiner et al., U.S. Pat. No. 5,696,135 (issued Dec. 9, 1997) describe the neurotrophic actions of a large number of compounds such as those described above. Cultured chick sensory neurons were used as an in vitro assay to measure the ability of compounds to promote neurite outgrowth (fiber extension) in neurons. Compounds were also tested for their ability to bind to FKBP12 and inhibit its enzymatic (rotamase) activity. As the data demonstrate, many of these compounds were found to be extremely potent nerve growth agents, promoting fiber extension from cultured neurons with half-maximal effects seen in some cases at picomolar concentrations. The effects of these simple FKBP12 ligands on nervous tissue are comparable to, or in some cases more potent than, FK506 itself.

Some of the compounds were also shown to promote regrowth of damaged peripheral nerves in vivo. Steiner, J. P., Connolly, M. A., Valentine, H. L., Hamilton, G. S., Dawson, T. M., Hester, L., Snyder, S. H., Nature Medicine (1997) 3:421-428. In whole-animal experiments in which the sciatic nerves of rats were crushed with forceps and animals treated with these compounds subcutaneously, there was found significant regeneration of damaged nerves relative to control animals, resulting in both more axons in drug-treated animals and axons with a greater degree of myelination. Lesioning of the animals treated only with vehicle caused a significant decrease in axon number (50% decrease compared to controls) and degree of myelination (90% decrease compared to controls). Treatment with the FKBP12 ligands resulted in reduction in the decrease of axon number (25% and 5% reduction, respectively, compared to controls) and in the reduction of myelination levels (65% and 50% decrease compared to controls). Similar results were subsequently reported by Gold et al. Gold, B. G., Zeleney-Pooley, M., Wang, M. S., Chaturvedi, P.; Armistead, D. M., Exp. Neurobiol. (1997) 147:269-278.

Several of these compounds were shown to promote recovery of lesioned central dopaminergic neurons in an animal model of Parkinson's Disease. Hamilton, G. S., Huang, W., Connolly, M. A., Ross, D. T., Guo, H., Valentine, H. L., Suzdak, P. D., Steiner, J. P., BioMed. Chem. Lett. (1997). N-Methyl-4-phenyl-1,2,3,6-tetrahydropyridine (“MPTP”) is a neurotoxin which selectively destroys dopaminergic neurons. Gerlach, M., Riederer, P., Przuntek, H., Youdim, M. B., Eur. J. Pharmacol. (1991) 208:273-286. The nigral-striatal dopaminergic pathway in the brain is responsible for controlling motor movements.

Parkinson's Disease is a serious neurodegenerative disorder resulting from degeneration of this motor pathway. Lesioning of the nigral-striatal pathway in animals with MPTP has been utilized as an animal model of Parkinson's Disease. In mice treated with MPTP and vehicle, a substantial loss of 60-70% of functional dopaminergic terminals was observed as compared to non-lesioned animals. Lesioned animals receiving FKBP12 ligands concurrently with MPTP showed a striking recovery of TH-stained striatal dopaminergic terminals, as compared with controls, suggesting that FKBP12 ligands may possess potent neuroprotective and neuro-regenerative effects on both peripheral as well as central neurons.

Other compounds which have an affinity for FKBP12 may also possess neurotrophic activities similar to those described above. For example, one skilled in the art is referred to the following patents and patent applications for their teaching of neuroimmunophilin ligands, or neurotrophic compounds, which are lacking immunosuppressive activity, the contents of which are hereby incorporated by reference in their entirety:

Hamilton et al., U.S. Pat. No. 5,614,547 (Mar. 25, 1997);

Steiner et al., U.S. Pat. No. 5,696,135 (Dec. 9, 1997);

Hamilton et al., U.S. Pat. No. 5,721,256 (Feb. 24, 1998);

Hamilton et al., U.S. Pat. No. 5,786,378 (Jul. 28, 1998);

Hamilton et al., U.S. Pat. No. 5,795,908 (Aug. 18, 1998);

Steiner et al., U.S. Pat. No. 5,798,355 (Aug. 25, 1998);

Steiner et al., U.S. Pat. No. 5,801,187 (Sep. 1, 1998);

Li et al., U.S. Pat. No. 5,801,187 (Sep. 1, 1998);

Hamilton et al., U.S. Pat. No. 5,846,979 (Dec. 8, 1998);

Hamilton et al., U.S. Pat. No. 5,859,031 (Jan. 12, 1999);

Hamilton et al., U.S. Pat. No. 5,874,449 (Feb. 23, 1999);

Hamilton et al., U.S. Pat. No. 5,935,989 (Aug. 10, 1999);

Hamilton et al., U.S. Pat. No. 5,958,949 (Sep. 28, 1999);

Hamilton et al., U.S. Pat. No. 5,990,131 (Nov. 23, 1999);

Hamilton et al., U.S. Pat. No. 6,121,273 (Sep. 19, 2000);

Hamilton et al., U.S. Pat. No. 6,218,424 (Apr. 17, 2001).

These molecules are effective ligands for, and inhibitors of, FKBP12 and are also potent neurotrophic agents in vitro, promoting neurite outgrowth from cultured sensory neurons at nanomolar or subnanolar dosages.

Additionally, as noted, compounds which possess immunosuppressive activity, for example, FK506, CsA, Rapamycin, and WAY-124,466, among others, also may possess a significant level of neurotrophic activity. Thus, to the extent that such compounds additionally may possess activities, including neurotrophic activities, such compounds are intended to be included within the terms “neurotrophic compound” and “neuroimmunophilin ligand” as used herein. The following publications provide disclosures of compounds which presumably possess immunosuppressive activities, as well as possibly other activities, and are likewise intended to be included within the terms “neurotrophic compound” and “neuroimmunophilin ligand” as used herein, the contents of which are hereby incorporated by reference in their entirety:

Armistead et al., U.S. Pat. No. 5,192,773 (Mar. 9, 1993);

Armistead et al., U.S. Pat. No. 5,330,993 (Jul. 19, 1994);

Armistead et al., U.S. Pat. No. 5,516,797 (May 14, 1996);

Zelle et al., U.S. Pat. No. 5,543,423 (Aug. 6, 1996);

Armistead et al., U.S. Pat. No. 5,620,971 (Apr. 15, 1997);

Armistead et al., U.S. Pat. No. 5,622,970 (Apr. 22, 1997);

Armistead et al., U.S. Pat. No. 5,665,774 (Sep. 9, 1997);

Armistead et al., U.S. Pat. No. 5,717,092 (Feb. 10, 1998);

Armistead et al., U.S. Pat. No. 5,723,459 (Mar. 3, 1998);

Zelle, U.S. Pat. No. 5,726,184 (Mar. 10, 1998);

Zelle et al., U.S. Pat. No. 5,744,485 (Apr. 28, 1998);

Cottens et al., U.S. Pat. No. 6,200,985 (Mar. 13, 2001); and

Siegel et al., U.S. Pat. No. 6,204,245 (Mar. 20, 2001).

In this regard, it is to be noted that non-immunosuppressive compounds are particularly preferred in the methods of the present invention. It is not uncommon for a person who stays at a hospital following surgery to become infected with a nosocomial infection. These nosocomial infections often result in serious hardships for the person so infected. Accordingly, it is particularly desired to administer compounds which do not suppress the immune system in the present inventive methods to minimize the risk to the patient of receiving a nosocomial infection.

Additionally, the following publications provide disclosures of compounds which are likewise intended to be included within the terms “neurotrophic compound” and “neuroimmunophilin ligand” as used herein, the contents of which are hereby incorporated by reference in their entirety:

Zelle et al., U.S. Pat. No. 5,780,484 (Jul. 14, 1998);

Zelle et al., U.S. Pat. No. 5,811,434 (Sep. 22, 1998);

Zelle et al., U.S. Pat. No. 5,840,736 (Nov. 24, 1998);

Armistead, U.S. Pat. No. 6,037,370 (Mar. 14, 2000);

Vrudhula et al., U.S. Pat. No. 6,096,762 (Aug. 1, 2000);

Pikul et al., U.S. Pat. No. 6,121,258 (Sep. 19, 2000);

Almstead et al., U.S. Pat. No. 6,121,272 (Sep. 19, 2000);

Nagel et al., U.S. Pat. No. 6,121,280 (Sep. 19, 2000);

Armistead, U.S. Pat. No. 6,124,328 (Sep. 26, 2000);

Pikul et al., U.S. Pat. No. 6,150,370 (Nov. 21, 2000);

Zook et al., U.S. Pat. No. 6,153,757 (Nov. 28, 2000);

De et al., U.S. Pat. No. 6,166,005 (Dec. 26, 2000);

Wythes et al., U.S. Pat. No. 6,166,011 (Dec. 26, 2000);

Zelle et al., U.S. Pat. No. 6,172,086 (Jan. 9, 2001);

Thorwart et al., U.S. Pat. No. 6,207,672 (Mar. 27, 2001);

Dubowchik et al., U.S. Pat. No. 6,228,872 (May 8, 2001);

Barrish et al., U.S. Pat. No. 6,235,740 (May 22, 2001);

Duffy, PCT Publication No. 92/21313 (Dec. 10, 1992);

Armistead, PCT Publication No. 96/41609 (Dec. 27, 1996);

McCaffrey et al., PCT Publication No. 99/10340 (Mar. 4, 1999);

McClure et al., PCT Publication No. 00/09485 (Feb. 24, 2000);

McClure et al., PCT Publication No. 00/09492 (Feb. 24, 2000);

Bryans et al., PCT Publication No. 00/15611 (Mar. 23, 2000);

Dubowchik et al., PCT Publication No. 00/27811 (May 18, 2000);

Oliver, PCT Publication No. 00/40557 (Jul. 13, 2000) Brumby et al., PCT Publication No. 00/46181 (Aug. 10, 2000);

Brumby et al., PCT Publication No. 00/46193 (Aug. 10, 2000);

Brumby et al., PCT Publication No. 00/46222 (Aug. 10, 2000);

Mutel et al., PCT Publication No. 00/58285 (Oct. 5, 2000);

Watanabe et al., PCT Publication No. 00/58304 (Oct. 5, 2000);

Bedell et al., PCT Publication No. 00/69819 (Nov. 23, 2000);

Mitch et al., PCT Publication No. 00/75140 (Dec. 14, 2000);

Lauffer et al., PCT Publication No. 01/02358 (Jan. 11, 2001);

Lauffer et al., PCT Publication No. 01/02361 (Jan. 11, 2001);

Lauffer et al., PCT Publication No. 01/02362 (Jan. 11, 2001);

Lauffer et al., PCT Publication No. 01/02363 (Jan. 11, 2001);

Lauffer et al., PCT Publication No. 01/02368 (Jan. 11, 2001);

Lauffer et al., PCT Publication No. 01/02372 (Jan. 11, 2001);

Harbeson et al., PCT Publication No. 01/02376 (Jan. 11, 2001);

Lauffer et al., PCT Publication No. 01/02405 (Jan. 11, 2001);

Kanojia et al., PCT Publication No. 01/04116 (Jan. 18, 2001);

Mullican et al., PCT Publication No. 01/08685 (Feb. 8, 2001);

Mullican et al., PCT Publication No. 01/09097 (Feb. 8, 2001);

Degenhardt et al., PCT Publication No. 01/10839 (Feb. 15, 2001); and

Brumby et al., PCT Publication No. 01/12622 (Feb. 22, 2001).

The neuroregenerative and neuroprotective effects of FKBP12 ligands are not limited to dopaminergic neurons in the central nervous system. In rats treated with para-chloro-amphetamine (“PCA”), an agent which destroys neurons which release serotonin as a neurotransmitter, treatment with an FKBP ligand was reported to exert a protective effect. Steiner, J. P., Hamilton, G. S., Ross, D. T., Valentine, H. L., Guo, H., Connolly, M. A., Liang, S., Ramsey, C., Li, J. H., Huang, W., Howorth, P.; Soni, R., Fuller, M., Sauer, H., Nowotnick, A., Suzdak, P. D., Proc. Natl. Acad. Sci. USA (1997) 94:2019-2024. In rats lesioned with PCA, cortical density of serotonin fibers was reduced 90% relative to controls. Animals receiving the ligand showed a greater serotonin innervation in the cortex—serotonergic innervation in the somatosensory cortex was increased more than two-fold relative to lesioned, non-drug treated animals.

Similarly, such ligands have been shown to induce sprouting of residual cholinergic axons following partial transection of the fimbria formix in rats. Guo, H., Spicer, D. M., Howorth, P., Hamilton, G. S., Suzdak, P. D, Ross, D. T., Soc. Neurosci. Abstr. (1997) 677.12. The transection produced a 75-80% differentiation of the hippocampus. Subcutaneous administration of the FBKP12 ligand produced a four-fold sprouting of spared residual processes in the CA1, CA3 and dentate gyrus regions of the hippocampus, resulting in significant recovery of cholinergic innervation in all three regions as quantitated by choline acetyltransferase (ChAT) density.

In particular, certain ligands for FKBP 12, preferably those which are non-immunosuppressive, comprise a class of potent active neurotrophic compounds which have been referred to as “neuroimmunophilins” or “neuroimmunophilin ligands” with potential for therapeutic utility in the treatment or prevention of neurodegenerative diseases. Thus, in the context of the present invention, the terms “neurotrophic compound” and “neuroimmunophilin ligand” are meant to encompass those compounds which have been designated as neuroimmunophilins and which also may have, but are not required to have, binding affinity for an FKBP. The ultimate mechanism of action and whether or not such compounds also possess other activity such as, for example, immunosuppressive activity, is not determinative of whether the compound is a “neurotrophic compound” or a “neuroimmunophilin ligand” for purposes of the invention as long as the compound in question possesses the desired effect on nerve injuries caused as a consequence of surgery. Assays for determining “neurotrophic compounds” or “neuroimmunophilin ligands” are well known to those of ordinary skill in the art. Specific, non-limiting examples of well known assays include MPTP wherein MPTP lesioning of dopaminergic neurons in mice is used to determine the amount of neurite regrowth a compound provides as well as chick DRG wherein dorsal root ganglia dissected from chick embryos are treated with various compounds to effect neurite outgrowth.

Until the present invention, none of the prior work disclosed the use of the disclosed neurotrophic compounds in the treatment of nerve injury caused as a consequence of surgery and associated diseases. As described in more detail below, the present invention is directed to such uses.

B. Treating Nerve Injury Caused as a Result of Prostate Surgery

More males are afflicted with prostate cancer than any other malignancy. Advanced surgical techniques have been developed to effectively treat prostate cancer. Even with the use of these techniques, there remains a problem with the preservation of penile innervation following prostate surgery. This is because the cavernous nerves, which are NOS neurons, will die if bumped, contused, crushed, or compressed in any way, i.e. during surgery on the prostate. The amount of pressure placed on the cavernous nerve can be measured according to a pressure test, wherein when the nerve is squeezed, it dies. The pressure put on the nerve is measured in terms of mm of Mercury.

Accordingly, a substantial number of male patients lose erectile function following prostate surgery. This loss comes despite the fact that the cavernous nerves, the principal autonomic innervation of the penis, frequently remains intact following prostate surgery. Accordingly, many males afflicted with prostate cancer do not seek surgical treatment for fear of becoming impotent. In an attempt to alleviate this problem, many doctors are now attempting to use nerve sparing surgery to limit the collateral damage done to the cavernous nerve (2-3 cm long in humans, 1 cm long in rats) during prostate surgery.

Impotence is the consistent inability to achieve or sustain an erection of sufficient rigidity for sexual intercourse. It has recently been estimated that approximately 10 million American men are impotent (R. Shabsigh et al., “Evaluation of Erectile Impotence,” Urology, 32:83-90 (1988); W. L. Furlow, “Prevalence of Impotence in the United States,” Med. Aspects Hum. Sex. 19:13-6 (1985)). In 1985 in the United States, impotence accounted for more than several hundred thousand outpatient visits to physicians (National Center for Health Statistics, National Hospital Discharge Surbey, 1985, Bethesda, Md., Department of Health and Human Services, 1989 DHHS publication no. 87-1751). Depending on the nature and cause of the problem, treatments include psychosexual therapy, hormonal therapy, administration of vasodilators such as nitroglycerin and α-adrenergic blocking agents (“α-blockers”), oral administration of other pharmaceutical agents, vascular surgery, implanted penile prostheses, vacuum constriction devices and external aids such as penile splints to support the penis or penile constricting rings to alter the flow of blood through the penis.

A number of causes of impotence have been identified, including vasculogenic, neurogenic, endocrlnologic, and psychogenic. Vasculogenic impotence, which is caused by alterations in the flow of blood to and from the penis, is thought to be the most frequent organic cause of impotence. Common risk factors for vasculogenic impotence include hypertension, diabetes, cigarette smoking, pelvic trauma, and the like. Neurogenic impotence is associated with spinal-cord injury, multiple sclerosis, peripheral neuropathy caused by diabetes or alcoholism, and severance of the autonomic nerve supply to the penis consequent to prostate surgery. Erectile dysfunction is also associated with disturbances in endocrine function resulting in low circulating testosterone levels and elevated prolactin levels.

Penile erection requires (1) dilation of the arteries that regulate blood flow to the lacunae of the corpora cavernosum, (2) relaxation of trabecular smooth muscle, which facilitates engorgement of the penis with blood, and (3) compression of the venules by the expanding trabecular walls to decrease venous outflow.

Trabecular smooth muscle tone is controlled locally by adrenergic (constrictor), cholinergic (dilator) and nonadrenergic, noncholinergic (dilator) innervation, and by endothelium-derived vasoactive substances such as vasoactive intestinal polypeptide (VIP), prostanoids, endothelin, and nitric oxide. High sympathetic tone (noradrenergic) is implicated in erectile dysfunction, and, in some patients, the disorder can be successfully treated with noradrenergic receptor antagonists. See, Krane et al., New England Journal of Medicine 321:1648 (1989).

There is also evidence that dopaminergic mechanisms are involved in erectile dysfunction. For example, pharmacologic agents that elevate the level of brain dopamine or stimulate brain dopamine receptors increase sexual activity in animals (see, e.g., Gessa & Tagliamonte, Life Sciences 14:425 (1974); Da Prada et al., Brain Research 57:383 (1973)).

Administration of L-DOPA, a dopamine precursor, enhances sexual activity in male rats. L-DOPA has been used in the treatment of Parkinsonism and is know to act as an aphrodisiac in some patients (Gessa & Tagliamonte, supra; Hyppa et al., Acta Neurologic Scand. 46:223 (Supp. 43, 1970)). Specific dopamine agonists have been studied for their effects on erectile function. Apomorphine, (n-propyl) norapo-morphine, bromocryptine, amantidine, fenfluramine, L-DOPA, and various other pharmacological activators of central dopaminergic receptors have been found to increase episodes of penile erection in male rats (Benassi-Benelli et al., Arch. Int. Pharmacodyn. 242:241 (1979); Poggioli et al., Riv. di Farm. & Terap.9:213 (1978); Falaschi et al., Apomorphine and Other Dopaminomimetics, 1:117-121 (Gessa & Corsini, Eds., Raven Press, N.Y.)). In addition, U.S. Pat. No. 4,521,421 to Foreman relates to the oral or intravenous administration of quinoline compounds to treat sexual dysfunction in mammals, the entire contents of which are incorporated herein by reference.

The currently available dopamine agonists, with few exceptions, have found limited use in the treatment of erectile dysfunction because of their peripheral side effects. These effects include nausea and vomiting, postural hypotension, arrhythmias, tachycardia, dysphoria, psychosis, hallucinations, drowsiness, and dyskinesias (See e.g., Martindale The Extra Pharmacopoeia, 31^{st} Ed., pages 1151-1168).

Other pharmaceutical methods for treating erectile dysfunction have also proved to be problematic. For example, with Viagra.RTM., the most recently introduced oral drug therapy, not only have significant side effects been encountered, but interaction with other systemically administered medications has posed enormous risks and numerous fatalities have in fact been reported.

The invention described herein provides a means to avoid the above-mentioned problems encountered with the systemic administration of pharmacologically active agents to treat erectile dysfunction. Specifically, the invention relates to methods and formulations for effectively treating erectile dysfunction by administering a selected active agent.

The following documents are of interest insofar as they relate to the treatment of erectile dysfunction by delivering pharmacologically active agents to the penis, and are incorporated herein be reference in their entirety:

U.S. Pat. No. 4,127,118 to Latorre describes the injection of vasodilator drugs into the corpora cavernosa of the penis to dilate the arteries that supply blood to the erectile tissues, thereby inducing an erection;

U.S. Pat. No. 5,439,938 to Snyder et al. describes the administration of nitric oxide (NO) synthase inhibitors by direct injection of a drug into the corpora cavernosa, by topical drug administration, or transurethral drug administration, for inhibiting penile erection due to priapism and for treating urinary incontinence;

Virag et al., Angiology-Journal of Vascular Diseases (February 1984), pp. 79-87, Brindley, Brit. J. Psychiat. 143:332-337 (1983), and Stief et al., Urology XXXI:483-485 (1988) respectively describe the intracavernosal injection of papaverine (a smooth muscle relaxant), phenoxybenzamine or phentolamine α-receptor blockers), and a phentolamine-papaverine mixture to treat erectile dysfunction; and

PCT Publication No. WO 01/16021, U.S. Pat. No. 4,801,587 to Voss et al., and U.S. Pat. Nos. 5,242,391, 5,474,535, 5,686,093, and 5,773,020 to Place et al. relate to the treatment of erectile dysfunction by delivery of a vasoactive agent into the male urethra.

Regardless of the cause, there exists a need to prevent or treat nerve injury caused as a consequence of surgery. The present invention provides such a method.

### SUMMARY OF THE INVENTION

In particular, the present invention provides methods for treating or preventing nerve injury caused as a consequence of surgery comprising administering to a patient in need thereof a therapeutically effective amount of a neurotrophic compound. By way of example, the nerve injury may be caused as a consequence of prostate surgery. In particular, the nerve injury may be to the cavernous nerve. Accordingly, the present methods are also useful for the neuroprotection, pre-treatment, or prophylactic treatment of penile innervation following prostate surgery and for treating erectile dysfunction.

The present invention is based on the discovery that the penile cavernous nerve responds to a neurotrophic compound by preserving erectile function. Thus, a therapeutically effective amount of a neurotrophic compound may be administered to promote the protection of penile innervation from degeneration following prostate surgery as well as the preservation of erectile function.

According to the invention, a neurotrophic compound may be administered parenterally at a dose ranging from about 1 ng/kg/day to about 10 ng/kg/day, typically at a dose of about 1 μg/kg/day to about 10 μg/kg/day, and usually at a dose of about 5 mg/kg/day to about 20 mg/kg/day. It is also contemplated that, depending on the individual patient's needs and route of administration, the neurotrophic compound may be given at a lower frequency such as monthly, weekly or several times per week, rather than daily. It is further contemplated that the neurotrophic compound may be administered topically, for example in the form of a cream or lotion, orally, for example in the form of tablets or pills, parenterally, such as by subcutaneous or intramuscular injection, or directly into the penis. One skilled in the art will appreciate that with direct administration a smaller amount of the desired compound may be used.

It is further contemplated that the neurotrophic compound may be administered separately, sequentially, or simultaneously in combination or conjunction with an effective amount of a second therapeutic agent, such as neurotrophic growth factor, brain derived growth factor, glial derived growth factor, cilial neurotrophic factor, and neurotropin-3 or any other agent useful for the treatment of nerve regeneration.

The invention also provides for the use of a neurotrophic compound in the manufacture of a medicament or pharmaceutical composition for the treatment of nerve injury caused as a consequence of various surgeries. Such pharmaceutical compositions include topical, systemic, oral neurotrophic compound formulations, optionally in combination with an additional neurotrophic factor.

### BRIEF DESCRIPTION OF THE DRAWINGS

FIG. 1 shows the protective effect of the neurotrophic compound 153 on the right and left major pelvic ganglia as processed for nNOS immunoreactivity.

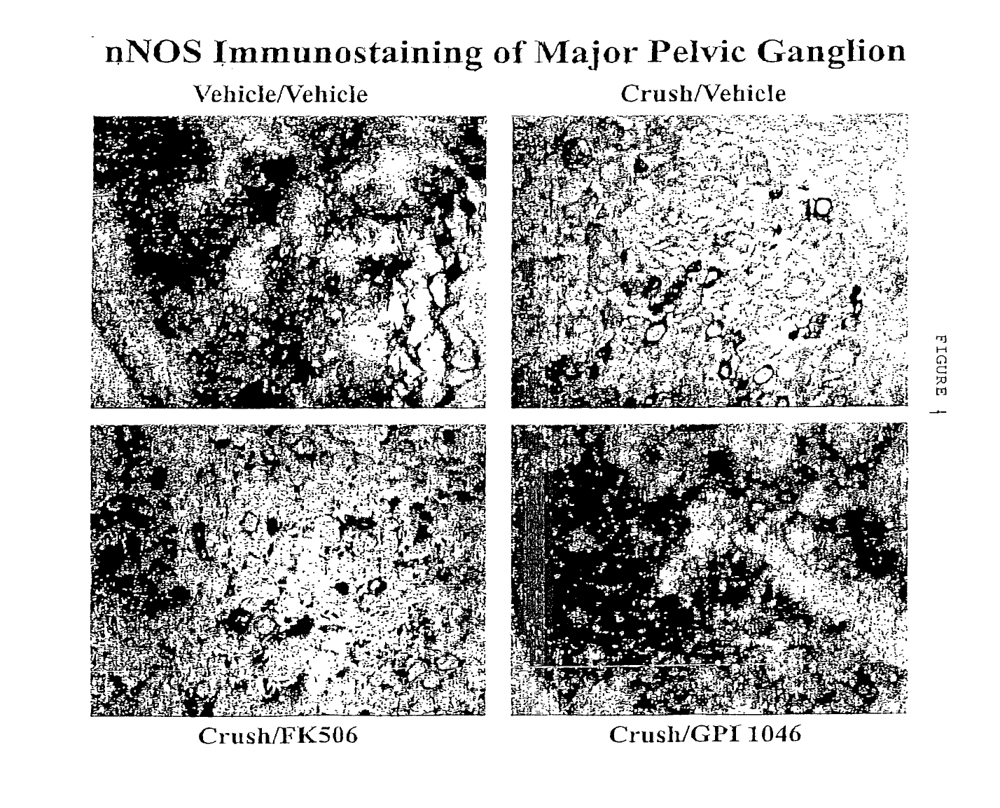

FIG. 2 shows the protective effect of the neurotrophic compound 153 on the right and left major pelvic ganglia as processed for Cresyl Violet staining.

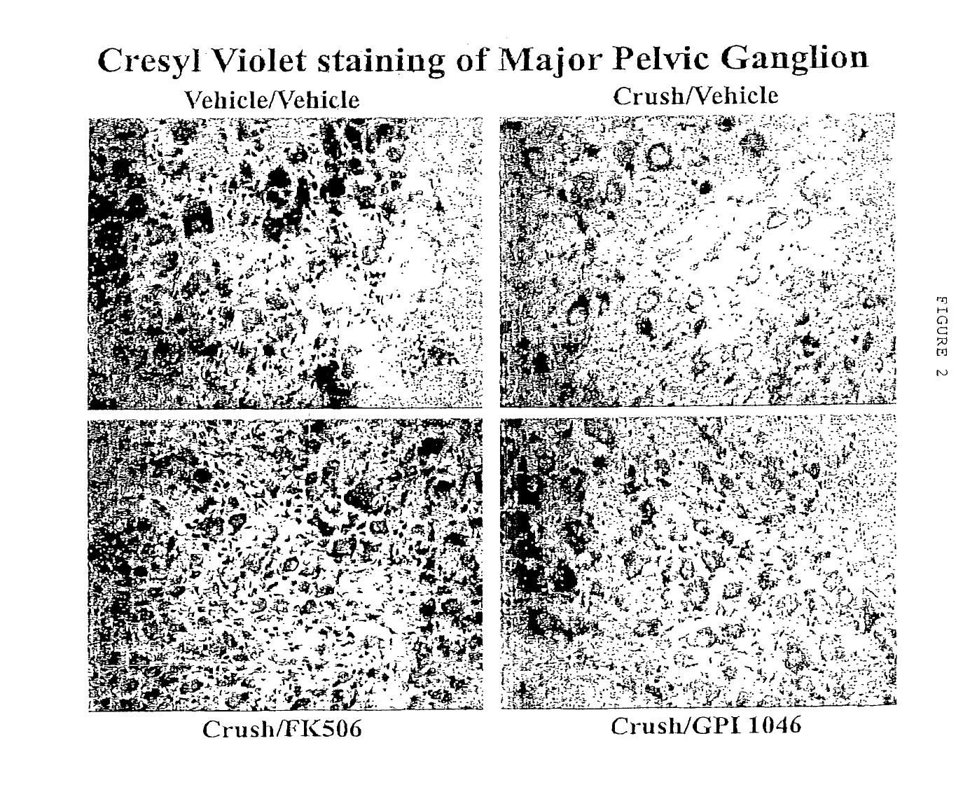

FIG. 3 shows a schematic of the human male urogenital system.

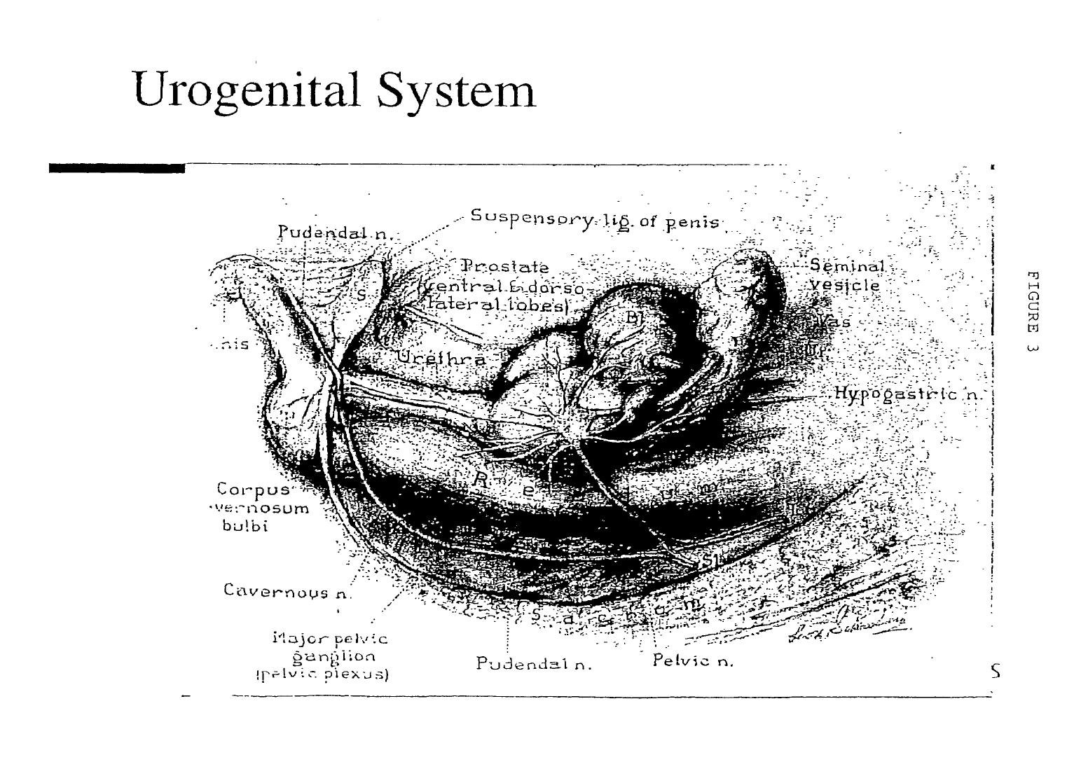

### DETAILED DESCRIPTION OF THE INVENTION

The present invention provides a method for treating or preventing nerve injury caused as a consequence of surgery by administering to a patient a therapeutically effective amount of a neurotrophic compound. According to one aspect of the invention, methods are provided for treating or preventing nerve injury caused as a consequence of prostate surgery by administering a therapeutically effective amount of a neurotrophic compound by means of a pharmaceutical composition.

The present invention is based on the discovery that a neurotrophic compound provides neuroprotection for penile innervation from degeneration following nerve crush injury in rats. Additionally, the present invention is based on the discovery that administration of a neurotrophic compound regenerates the cavernous nerve of the penis following cavernous nerve crush, preserving erectile dysfunction. It is contemplated that administration of exogenous neurotrophic compounds will protect the penile cavernous nerve from traumatic damage, for example damage caused by prostate surgery.

The present invention further provides methods for treating or preventing nerve injury caused as a consequence of surgeries other than prostate surgery. Several non-limiting examples of such surgeries include cardiac surgery, beating-heart surgery, thoracic surgery, bypass surgery, aortic valve replacement surgery, capsular shift procedures, ophthalmic surgery, lumbar surgery, knee surgery, arthroscopic surgery, neurosurgery, surgery to heal soft tissue in injured joints, pelvic surgery, radiation therapy, penile prosthetic implant surgery, tendon transfer surgery, surgery to remove a tumor other than a prostate tumor, carotid endarterectomy, vascular surgery, aortic surgery, orthopedic surgery, endovascular procedures, such as arterial catheterization (carotid, vertebral, aortic, cardia, renal, spinal, Adamkiewicz), renal surgery, kidney transplantation, spinal surgery, eye surgery, vertebral surgery, otologic surgery, spinal nerve ligation surgery, dental repair (root canal), neuropathogenic surgery, orthopedic surgery, rotator cuff surgery, surgery to repair a tendon rupture, endoscopic surgery, oral surgery, and any other surgery in which nearby nerves have the potential to become damaged.

According to the invention, the neurotrophic compound may be administered systemically at a dose ranging from about 1 to about 20 mg/kg/day. The neurotrophic compound may be administered directly into the area which has undergone a surgical procedure. In such cases, a smaller amount of neurotrophic compound may be administered. It is further contemplated that the neurotrophic compound may be administered with an effective amount of a second nerve growth agent, including neurotrophic growth factor, brain derived growth factor, glial derived growth factor, cilial neurotrophic factor, and neurotropin-3 as well as other neurotrophic factors or drugs used currently or in the future. A variety of pharmaceutical formulations and different delivery techniques are described in further detail below.

C. Neurotrophic Compound Pharmaceutical Compositions

Neurotrophic compound pharmaceutical compositions typically include a therapeutically effective amount of a neurotrophic compound described herein in admixture with one or more pharmaceutically and physiologically acceptable formulation materials. Suitable formulation materials include, but are not limited to, antioxidants, preservatives, coloring, flavoring and diluting agents, emulsifying agents, suspending agents, solvents, fillers, bulking agents, buffers, delivery vehicles, diluents, excipients and/or pharmaceutical adjuvants. For example, a suitable vehicle may be water for injection, physiological saline solution, or artificial perilymph, possibly supplemented with other materials common in compositions for parenteral administration. Neutral buffered saline or saline mixed with serum albumin are further exemplary vehicles.

The primary solvent in a vehicle may be either aqueous or non-aqueous in nature. In addition, the vehicle may contain other pharmaceutically-acceptable excipients for modifying, modulating or maintaining the pH, osmolarity, viscosity, clarity, color, sterility, stability, rate of dissolution, or odor of the formulation. Similarly, the vehicle may contain still other pharmaceutically-acceptable excipients for modifying or maintaining the rate of release of the therapeutic product(s), or for promoting the absorption or penetration of the therapeutic product(s) across the tympanic membrane. Such excipients are those substances usually and customarily employed to formulate dosages for middle-ear administration in either unit dose or multi-dose form.

Once the therapeutic composition has been formulated, it may be stored in sterile vials as a solution, suspension, gel, emulsion, solid, or dehydrated or lyophilized powder. Such formulations may be stored either in a ready to use form or in a form, e.g., lyophilized, requiring reconstitution prior to administration.

The optimal pharmaceutical formulations will be determined by one skilled in the art depending upon considerations such as the route of administration and desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the present therapeutic agents of the invention.

Other effective administration forms, such as slow-release formulations, inhalant mists, or orally active formulations are also envisioned. For example, in a sustained release formulation, the neurotrophic compound may be bound to or incorporated into particulate preparations of polymeric compounds (such as polylactic acid, polyglycolic acid, etc.) or liposomes. Hylauronic acid may also be used, and this may have the effect of promoting sustained duration in the circulation. Such therapeutic compositions are typically in the form of a pyrogen-free acceptable aqueous solution comprising the neurotrophic compound in a pharmaceutically acceptable vehicle. One preferred vehicle is sterile distilled water.

Certain formulations containing a neurotrophic compound may be administered orally. A neurotrophic compound which is administered in this fashion may be encapsulated and may be formulated with or without those carriers customarily used in the compounding of solid dosage forms. The capsule may be designed to release the active portion of the formulation at the point in the gastrointestinal tract when bioavailability is maximized and pre-systemic degradation is minimized. Additional excipients may be included to facilitate absorption of the neurotrophic compound. Diluents, flavorings, low melting point waxes, vegetable oils, lubricants, suspending agents, tablet disintegrating agents, and binders may also be employed.

The preparations of the present invention, particularly topical preparations, may include other components, for example acceptable preservatives, tonicity agents, cosolvents, complexing agents, buffering agents or other pH controlling agents, antimicrobials, antioxidants and surfactants, as are well known in the art. For example, suitable tonicity enhancing agents include alkali metal halides (preferably sodium or potassium chloride), mannitol, sorbitol and the like. Sufficient tonicity enhancing agent is advantageously added so that the formulation to be instilled into the ear is compatible with the osmolarity of the endo- and perilymph. Suitable preservatives include, but are not limited to, benzalkonium chloride, thimerosal, phenethyl alcohol, methylparaben, propylparaben, chlorhexidine, sorbic acid and the like. Hydrogen peroxide may also be used as preservative. Suitable cosolvents include, but are not limited to, glycerin, propylene glycol and polyethylene glycol. Suitable complexing agents include caffeine, polyvinyl-pyrrolidone, β-cyclodextrin or hydroxypropyl-β-cyclodextrin. The buffers can be conventional buffers such as borate, citrate, phosphate, bicarbonate, or tris-HCl.

The formulation components are present in a concentration and form that is acceptable for penile administration. For example, buffers are used to maintain the composition at physiological pH or at slightly lower pH, typically within a pH range of from about 5 to about 8.

Additional formulation components may include materials which prolong the residence in the penis of the administered therapeutic agent, particularly to maximize the topical contact and promote absorption of the therapeutic agent. Suitable materials may include polymers or gel forming materials which increase the viscosity of the penile preparation. The suitability of the formulations of the instant invention for controlled release (e.g., sustained and prolonged delivery) can be determined by various procedures known in the art. Yet another penile preparation may involve an effective quantity of neurotrophic compound in admixture with non-toxic penile treatment acceptable excipients. For example, the neurotrophic compound may be prepared in tablet form. Suitable excipients include, but are not limited to, inert diluents, such as calcium carbonate, sodium carbonate or bicarbonate, lactose, or calcium phosphate; or binding agents, such as starch, gelatin, or acacia.

### Administration/Delivery of Neurotrophic Compound

The neurotrophic compound may be administered parenterally via a subcutaneous, intramuscular, intravenous, transpulmonary, transdermal, intrathecal or intracerebral route. For the treatment of penile conditions, the neurotrophic compound may be administered orally, systemically, or directly into the penis by topical application, inserts, injection or implants. For example, slow-releasing implants containing the molecules embedded in a biodegradable polymer matrix can be used to deliver the neurotrophic compound. As noted, the neurotrophic compound may be administered to the penis in connection with one or more agents capable of promoting penetration or transport of the neurotrophic compound into the penis. The frequency of dosing will depend on the pharmacokinetic parameters of the neurotrophic compound as formulated, and the route of administration.

The specific dose may be calculated according to considerations of body weight, body surface area or organ size. Further refinement of the calculations necessary to determine the appropriate dosage for treatment involving each of the above mentioned formulations is routinely made by those of ordinary skill in the art and is within the ambit of tasks routinely performed, especially in light of the dosage information and assays disclosed herein. Appropriate dosages may be determined using established assays in conjunction with appropriate dose-response data.

The final dosage regimen involved in a method for treating the above-described conditions will be determined by the attending physician, considering various factors which modify the action of drugs, e.g., the age, condition, body weight, sex and diet of the patient, the severity of the condition, time of administration and other clinical factors familiar to one skilled in the art.

It is envisioned that the continuous administration or sustained delivery of neurotrophic compounds may be advantageous for a given condition. While continuous administration may be accomplished via a mechanical means, such as with an infusion pump, it is contemplated that other modes of continuous or near continuous administration may be practiced. For example, such administration may be by subcutaneous or muscular injections as well as oral pills.

Techniques for formulating a variety of other sustained- or controlled-delivery means, such as liposome carriers, bio-erodible particles or beads and depot injections, are also known to those skilled in the art.

The compounds described in Formulas I-LXXIV, below, possess asymmetric centers and thus can be produced as mixtures of stereoisomers or as individual R- and S-stereoisomers. The individual stereoisomers may be obtained by using an optically active starting material, by resolving a racemic or non-racemic mixture of an intermediate at some appropriate stage of the synthesis, or by resolving the compounds of Formulas I-LXXIV. It is understood that the compounds of Formulae I-LXXIV encompass individual stereoisomers as well as mixtures (racemic and non-racemic) of stereoisomers. Preferably, S-stereoisomers are used in the pharmaceutical compositions and methods of the present invention.

The term “carbocyclic”, as used herein, refers to an organic cyclic moiety in which the cyclic skeleton is comprised of only carbon atoms whereas the term “heterocyclic” refers to an organic cyclic moiety in which the cyclic skeleton contains one or more heteroatoms selected from nitrogen, oxygen, or sulfur and which may or may not include carbon atoms. Carbocyclic or heterocyclic includes within its scope a single ring system, multiple fused rings (for example, bi-or tricyclic ring systems) or multiple condensed ring systems. One skilled in the art, therefore, will appreciate that in the context of the present invention, a cyclic structure formed by A and B (or A′ and B′) as described herein may comprise bi- or tri-cyclic or multiply condensed ring systems.

“Heterocycle” or “heterocyclic”, as used herein, refers to a saturated, unsaturated or aromatic carbocyclic group having a single ring, multiple fused (for example, bi- or tri-cyclic ring systems) rings or multiple condensed rings, and having at least one hetero atom such as nitrogen, oxygen or sulfur within at least one of the rings. This term also includes “Heteroaryl” which refers to a heterocycle in which at least one ring is aromatic.

In the context of the invention, useful carbo- and heterocyclic rings include, for example and without limitation, phenyl, benzyl, naphthyl, indenyl, azulenyl, fluorenyl, anthracenyl, indolyl, isoindolyl, indolinyl, benzofuranyl, benzothiophenyl, indazolyl, benzimidazolyl, benzthiazolyl, tetrahydrofuranyl, tetrahydropyranyl, pyridyl, pyrrolyl, pyrrolidinyl, pyridinyl, pyrimidinyl, purinyl, quinolinyl, isoquinolinyl, tetrahydroquinolinyl, quinolizinyl, furyl, thiophenyl, imidazolyl, oxazolyl, benzoxazolyl, thiazolyl, isoxazolyl, isotriazolyl, oxadiazolyl, triazolyl, thiadiazolyl, pyridazinyl, pyrimidinyl, pyrazinyl, triazinyl, trithianyl, indolizinyl, pyrazolyl, pyrazolinyl, pyrazolidinyl, thienyl, tetrahydroisoquinolinyl, cinnolinyl, phthalazinyl, quinazolinyl, quinoxalinyl, naphthyridinyl, pteridinyl, carbazolyl, acridinyl, phenazinyl, phenothiazinyl, and phenoxazinyl.

“Aryl” or “aromatic” refers to an aromatic carbocyclic or heterocyclic group having a single ring, for example, a phenyl ring, multiple rings, for example, biphenyl, or multiple condensed rings in which at least one ring is aromatic, for example, naphthyl, 1,2,3,4,-tetrahydronaphthyl, anthryl, or phenanthryl, which can be unsubstituted or substituted. The substituents attached to a phenyl ring portion of an aryl moiety in the compounds of the invention may be configured in the ortho-, meta- or para- orientations, with the para-orientation being preferred.

Examples of typical aryl moieties included in the scope of the present invention may include, but are not limited to, the following:

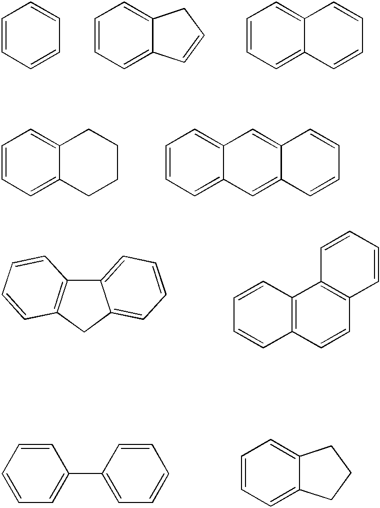

Examples of heterocyclic or heteroaryl moieties included in the scope of the present invention may include, but are not limited to, the following:

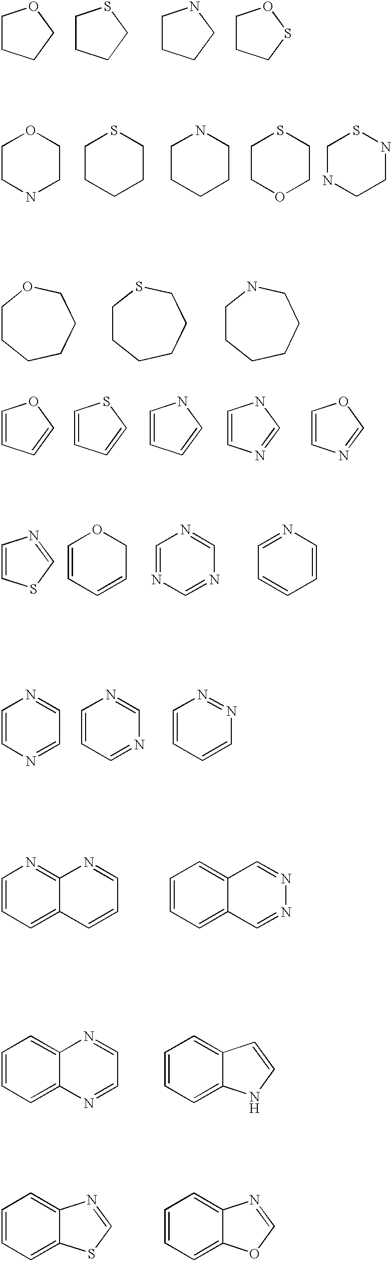

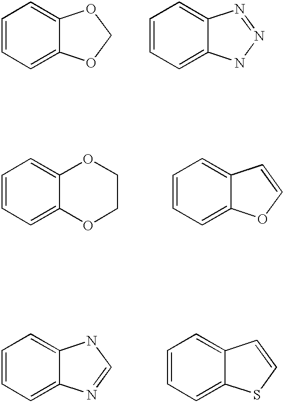

As one skilled in the art will appreciate such heterocyclic moieties may exist in several isomeric forms, all of which are to be encompassed by the present invention. For example, a 1,3,5-triazine moiety is isomeric to a 1,2,4-triazine group. Such positional isomers are to be considered within the scope of the present invention. Likewise, the heterocyclic or heteroaryl groups can be bonded to other moieties in the compounds of the invention. The point(s) of attachment to these other moieties is not to be construed as limiting on the scope of the invention. Thus, by way of example, a pyridyl moiety may be bound to other groups through the 2-, 3-, or 4-position of the pyridyl group. All such configurations are to be construed as within the scope of the present invention.

As used herein, “warm-blooded animal” includes a mammal, including a member of the human, equine, porcine, bovine, murine, canine or feline species. In the case of a human, the term “warm-blooded animal” may also be referred to as a “patient”. Further, as used herein, “a warm blooded animal in need thereof” refers to a warm-blooded animal having damaged nerves as a result of surgery. This term also refers to a warm blooded animal which has already suffered some degree of damaged nerves as a consequence of surgery because of genetic or environmental conditions to which the animal has been exposed or to which it has been predisposed. Environmental conditions can include the treatment with a therapeutic compound, such as an ototoxic substance, as well as other types of injury or insult.

“Pharmaceutically acceptable salt”, as used herein, refers to an organic or inorganic salt which is useful in the treatment of a warm-blooded animal in need thereof. Such salts can be acid or basic addition salts, depending on the nature of the neurotrophic agent compound to be used.

In the case of an acidic moiety in a neurotrophic agent of the invention, a salt may be formed by treatment of the neurotrophic agent with a basic compound, particularly an inorganic base. Preferred inorganic salts are those formed with alkali and alkaline earth metals such as lithium, sodium, potassium, barium and calcium. Preferred organic base salts include, for example, ammonium, dibenzylammonium, benzylammonium, 2-hydroxyethylammonium, bis(2-hydroxyethyl)ammonium, phenylethylbenzylamine, dibenzyl-ethylenediamine, and the like salts. Other salts of acidic moieties may include, for example, those salts formed with procaine, quinine and N-methylglucosamine, plus salts formed with basic amino acids such as glycine, ornithine, histidine, phenylglycine, lysine and arginine. An especially preferred salt is a sodium or potassium salt of a neurotrophic compound used in the invention.

With respect to basic moieties, a salt is formed by the treatment of the desired neurotrophic compound with an acidic compound, particularly an inorganic acid. Preferred inorganic salts of this type may include, for example, the hydrochloric, hydrobromic, hydroiodic, sulfuric, phosphoric or the like salts. Preferred organic salts of this type, may include, for example, salts formed with formic, acetic, succinic, citric, lactic, maleic, fumaric, palmitic, cholic, pamoic, mucic, d-glutamic, d-camphoric, glutaric, glycolic, phthalic, tartaric, lauric, stearic, salicyclic, methanesulfonic, benzenesulfonic, para-toluenesulfonic, sorbic, puric, benzoic, cinnamic and the like organic acids. An especially preferred salt of this type is a hydrochloride or sulfate salt of the desired neurotrophic compound. Also, the basic nitrogen-containing groups can be quarternized with such agents as: 1) lower alkyl halides, such as methyl, ethyl, propyl, and butyl chloride, bromides and iodides; 2) dialkyl sulfates like dimethyl, diethyl, dibutyl and diamyl sulfates; 3) long chain alkyls such as decyl, lauryl, myristyl and stearyl substituted with one or more halide such as chloride, bromide and iodide; and 4) aralkyl halides like benzyl and phenethyl bromide and others.

Also encompassed in the scope of the present invention are pharmaceutically acceptable esters of a carboxylic acid or hydroxyl containing group, including a metabolically labile ester or a prodrug form of a compound of Formula (I′). A metabolically labile ester is one which may produce, for example, an increase in blood levels and prolong the efficacy of the corresponding non-esterified form of the compound. A prodrug form is one which is not in an active form of the molecule as administered but which becomes therapeutically active after some in vivo activity or biotransformation, such as metabolism, for example, enzymatic or hydrolytic cleavage. Esters of a compound of Formula (I′), may include, for example, the methyl, ethyl, propyl, and butyl esters, as well as other suitable esters formed between an acidic moiety and a hydroxyl containing moiety. Metabolically labile esters, may include, for example, methoxymethyl, ethoxymethyl, iso-propoxymethyl, α-methoxyethyl, groups such as α-((C₁-C₄) alkyloxy) ethyl; for example, methoxyethyl, ethoxyethyl, propoxyethyl, iso-propoxyethyl, etc.; 2-oxo-1,3-dioxolen-4-ylmethyl groups, such as 5-methyl-2-oxo-1,3,dioxolen-4-ylmethyl, etc.; C₁-C₃ alkylthiomethyl groups, for example, methylthio-methyl, ethylthiomethyl, isopropylthio-methyl, etc.; acyloxymethyl groups, for example, pivaloyloxy-methyl, α-acetoxymethyl, etc.; ethoxycarbonyl-1-methyl; or α-acyloxy-α-substituted methyl groups, for example α-acetoxyethyl.

Further, the compounds of the invention may exist as crystalline solids which can be crystallized from common solvents such as ethanol, N,N-dimethyl-formamide, water, or the like. Thus, crystalline forms of the compounds of the invention may exist as solvates and/or hydrates of the parent compounds or their pharmaceutically acceptable salts. All of such forms likewise are to be construed as falling within the scope of the invention.

“Alkyl” means a branched or unbranched saturated hydrocarbon chain comprising a designated number of carbon atoms. For example, C₁-C₆ straight or branched alkyl hydrocarbon chain contains 1 to 6 carbon atoms, and includes but is not limited to substituents such as methyl, ethyl, propyl, iso-propyl, butyl, iso-butyl, tert-butyl, n-pentyl, n-hexyl, and the like.

“Alkenyl” means a branched or unbranched unsaturated hydrocarbon chain comprising a designated number of carbon atoms. For example, C₂-C₆ straight or branched alkenyl hydrocarbon chain contains 2 to 6 carbon atoms having at least one double bond, and includes but is not limited to substituents such as ethenyl, propenyl, iso-propenyl, butenyl, iso-butenyl, tert-butenyl, n-pentenyl, n-hexenyl, and the like.

“Alkoxy” means the group —OR wherein R is alkyl as herein defined. Preferably, R is a branched or unbranched saturated hydrocarbon chain containing 1 to 6 carbon atoms.

“Aryl, heteroaryl, carbocycle, or heterocycle” includes but is not limited to cyclic or fused cyclic ring moieties and includes a mono-, bi- or tricyclic, carbo- or heterocyclic ring, wherein the ring is either unsubstituted or substituted in one or more position(s) with hydroxy, carbonyl, amino, amido, cyano, isocyano, nitro, nitroso, nitrilo, isonitrilo, imino, azo, diazo, sulfonyl, sulfhydryl, sulfoxy, thio, thiocarbonyl, thiocyano, formanilido, thioformamido, sulfhydryl, halo, halo-(C₁-C₆)-alkyl, trifluoromethyl, (C₁-C₆)-alkoxy, (C₂-C₆)-alkenoxy, (C₁-C₆)-alkylaryloxy, aryloxy, aryl-(C₁-C₆)-alkyloxy, (C₁-C₆)-alkylamino, amino-(C₁-C₆)-alkyl, thio-(C₁-C₆)-alkyl, C₁-C₆-alkylthio, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl and carbocyclic and heterocyclic moieties; wherein the individual ring sizes are 5-8 members; wherein the heterocyclic ring contains 1-4 heteroatom(s) selected from the group consisting of O, N, or S; wherein aromatic or tertiary alkyl amines are optionally oxidized to a corresponding N-oxide.

Examples of preferred carbocyclic and heterocyclic moieties include, without limitation, phenyl, benzyl, naphthyl, indenyl, azulenyl, fluorenyl, anthracenyl, indolyl, isoindolyl, indolinyl, benzofuranyl, benzothiophenyl, indazolyl, benzimidazolyl, benzthiazolyl, tetrahydrofuranyl, tetrahydropyranyl, pyridyl, pyrrolyl, pyrrolidinyl, pyridinyl, pyrimidinyl, purinyl, quinolinyl, isoquinolinyl, tetrahydroquinolinyl, quinolizinyl, furyl, thiophenyl, imidazolyl, oxazolyl, benzoxazolyl, thiazolyl, isoxazolyl, isotriazolyl, oxadiazolyl, triazolyl, thiadiazolyl, pyridazinyl, pyrimidinyl, pyrazinyl, triazinyl, trithianyl, indolizinyl, pyrazolyl, pyrazolinyl, pyrazolidinyl, thienyl, tetrahydroisoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, and adamantyl.

“Halo” means at least one fluoro, chloro, bromo, or iodo moiety.

“Stereoisomers” are isomers that differ only in the way the atoms are arranged in space.

“Isomers” are different compounds that have the same molecular formula and includes cyclic isomers such as (iso)indole and other isomeric forms of cyclic moieties.

“Enantiomers” are a pair of stereoisomers that are non-superimposable mirror images of each other.

“Diastereoisomers” are stereoisomers which are not mirror images of each other.

“Racemic mixture” means a mixture containing equal parts of individual enantiomers. “Non-racemic mixture” is a mixture containing unequal parts of individual enantiomers or stereoisomers.

“Isosteres” are different compounds that have different molecular formulae but exhibit the same or similar properties. In particular, the term “carboxylic acid isostere” refers to compounds which mimic carboxylic acid stearically, electronically, and otherwise. Carboxylic acid isosteres possess chemical and physical similarities to carboxylic acid to produce a broadly similar biological property. In particular, these chemical and physical similarities are known to arise as a result of identical or similar valence electron configurations. For example, tetrazole is an isostere of carboxylic acid because it mimics the properties of carboxylic acid even though they both have very different molecular formulae. Prodrugs are not included among compounds which are carboxylic acid isosteres. Tetrazole is one of many possible isosteric replacements for carboxylic acid. Other carboxylic acid isosteres contemplated by the present invention include —COOH, —SO₃H, —SO₂HNR³, —PO₂ (R³)₂, —CN, PO₃(R³)₂, —OR³, —SR³, —NHCOR³, —N(R³)₂, —CON(R³)₂, —CONH(O)R³, —CONHNHSO₂R³′, —COHNSO₂R³, and —CONR³CN, wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carbocyclic and heterocyclic isosteres contemplated by this invention.

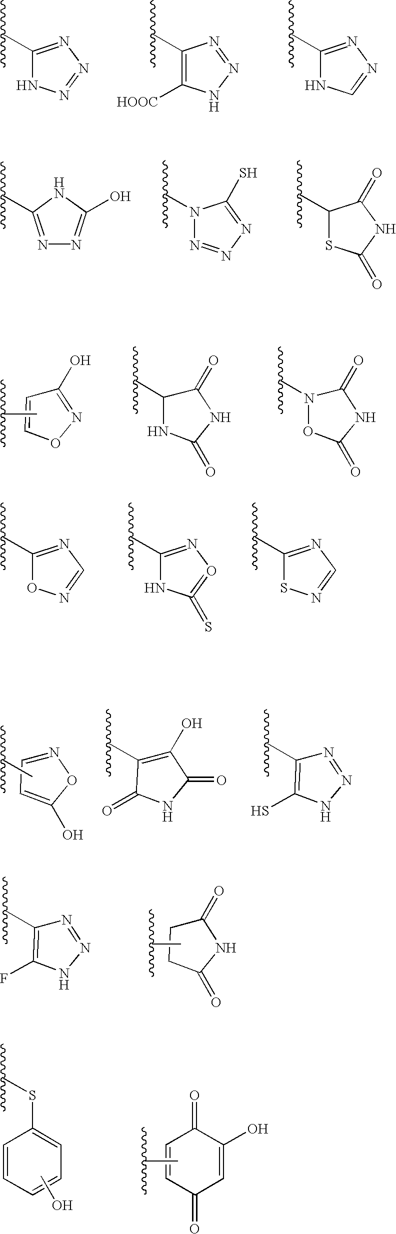

and —COOH, —SO₃H, —SO₂HNR³, —PO₂ (R³)₂, —CN, —PO₃(R³)₂, —OR³, —SR³, —NHCOR³, —N(R³)₂, —CON(R³ )₂, —CONH(O)R³, —CONHNHSO₂R³, —COHNSO₂R³, and —CONR³CN, wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl and where the atoms of said ring structure may be optionally substituted at one or more positions with R₁, as defined herein. The present invention contemplates that when chemical substituents are added to a carboxylic isostere then the inventive compound retains the properties of a carboxylic isostere.

The present invention contemplates that when a carboxylic isostere is optionally substituted with one or more moieties selected from R³, as defined herein, then the substitution cannot eliminate the carboxylic acid isosteric properties of the inventive compound. The present invention contemplates that the placement of one or more R³ substituents upon a carbocyclic or heterocyclic carboxylic acid isostere shall not be permitted at one or more atom(s) which maintain(s) or is/are integral to the carboxylic acid isosteric properties of the inventive compound, if such substituent(s) would destroy the carboxylic acid isosteric properties of the inventive compound.

Other carboxylic acid isosteres not specifically exemplified or described in this specification are also contemplated by the present invention.

Further, as used throughout the teaching of the invention, a designation of:

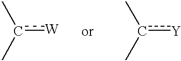

wherein W or Y is H₂, or similar designations, is meant to denote that two hydrogen atoms are attached to the noted carbon and that the bonds to each hydrogen are single bonds.

The term “prodrug” as used herein refers to an inactive precursor of a drug which is converted into its active form in the body by normal metabolic processes. In contrast, the isosteric compounds described herein are the active form of the drugs used in the present inventive methods. These compounds look, act, and feel like drugs, causing them to be directly administered to a person. Accordingly, the carboxylic acid isosteres described herein are used as pharmaceuticals in their own right and are not prodrugs which are administered to the body to be converted into an active form.

The terms “treating” or “preventing” as used herein relate to reducing, lessening, preventing, remedying, helping, redressing, correcting, pre-treating, prophylactically treating, re-balancing, regenerating, providing an essential element to, curing, precluding, obstructing, stopping, interrupting, intercepting, interclusing, hindering, impeding, retarding, restricting, restraining, inhibiting, or blocking nerve or neuronal injury, trauma, deterioration, debasement, waning, ebb, recession, retrogradation, decrease, degeneracy, degeneration, degradation, depravation, devolution, retrogression, impairment, inquination, injury, damage, loss, detriment, delaceration, ravage, declination, decay, dilapidation, erosion, blight, atrophy, collapse, destruction, or wreck caused as a consequence, effect, derivative, upshot, product, creation, or offspring of, resulting, arising, coming, or originating from, developing from, due to, or associated with surgery. A prophylactic treatment of nerve injury which will be caused as a consequence of surgery is particularly preferred in this regard. “Treating” or “preventing” also relate to encouraging, feeding, restoring, enhancing, ameliorating, or optimizing neuronal growth, regrowth, expansion, increase, enlargement, extension, augmentation, amplification, development, turgescence, turgidness, turgidity, swelling, or inflation following surgery.

The terms “immunosuppressive” and “non-immunosuppressive” as used herein refer to the ability or inability, respectively, of the compounds used in the present inventive methods to trigger an immune response when compared to a control such as FK506 or cyclosporin A. Assays for determining immunosuppression are well known to those of ordinary skill in the art. Specific non-limiting examples of well known assays include PMA and OKT3 assays wherein mitogens are used to stimulate proliferation of human peripheral blood lymphocytes (PBC). Compounds added to such assay systems are evaluated for their ability to inhibit such proliferation.

The neurotrophic compounds useful in the invention comprise a variety of structural families. As noted, the primary consideration is that the compounds possess the desired neurotrophic activity described herein. By way of description and not limitation, therefore, the following structural formulae are provided as exemplary of the neurotrophic compound compounds useful in the treatment of nerve injury caused as a consequence of prostate surgery:

In its broadest sense, the invention provides a method for the treatment of nerve injury caused as a consequence of prostate surgery which comprises administering to a warm-blooded animal a compound of formula (I′):

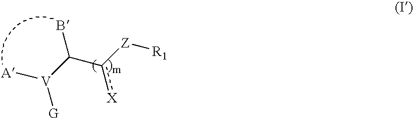

wherein

A′ is hydrogen, C₁ or C₂ alkyl, or benzyl;

B′ is C₁-C₄ straight or branched chain alkyl, benzyl or cyclohexylmethyl; or,

A′ and B′, taken together with the atoms to which they are attached, form a 5-7 membered saturated, unsaturated or aromatic heterocylic or carbocyclic ring which contains one or more additional O, C(R₁)₂, S(O)ₚ, N, NR₁, or NR₅ atoms;

V is CH, S, or N;

G is

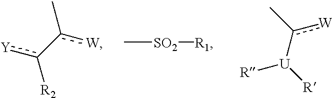

each R₁, independently, is hydrogen, C₁-C₉ straight or branched chain alkyl, or C₂-C₉ straight or branched chain alkenyl or alkynyl, C₃-C₉ cycloalkyl, C₅-C₇ cycloalkenyl, a carboxylic acid or carboxylic acid isostere, N(R₄)ₙ, Ar₁, Ar₄ or K-L wherein said alkyl, cycloalkyl, cycloalkenyl, alkynyl, alkenyl, Ar₁ or Ar₄ is optionally substituted with one or more substituent(s) independently selected from the group consisting of:

2-furyl, 2-thienyl, pyridyl, phenyl, C₃-C₆ cycloalkyl wherein said furyl thienyl, pyridyl, phenyl or cycloalkyl group optionally is substituted with C₁-C₄ alkoxy, (Ar₁)ₙ, halo, halo-C₁-C₆-alkyl, carbonyl, thiocarbonyl, C₁-C₆ thioester, cyano, imino, COOR₆ in which R₆ is C₁-C₉ straight or branched chain alkyl or alkenyl, hydroxy, nitro, trifluoromethyl, C₁-C₆ alkoxy, C₂-C₄ alkenyloxy, C₁-C₆ alkylaryloxy C₁-C₆ aryloxy, aryl-(C₁-C₆)-alkyloxy, phenoxy, benzyloxy, thio-(C₁-C₆)-alkyl, C₁-C₆-alkylthio, sulfhydryl, sulfonyl, amino, (C₁-C₆)-mono- or di-alkylamino, amino-(C₁-C₆)-alkyl, aminocarboxy, C₃-C₈ cycloalkyl, C₁-C₆ straight or branched chain alkyl, C₂-C₆ straight or branched chain alkenyl optionally substituted with (Ar₁), C₃-C₈ cycloalkyl, C₁-C₆ straight or branched chain alkyl, C₂-C₆ straight or branched chain alkenyl substituted with C₃-C₈ cycloalkyl, C₃-C₈ cycloalkyl, and Ar₂, and, wherein any carbon atom of an alkyl or alkenyl group may optionally replaced with O, NR₅, or S(O); or,

R₁ is a moiety of the formula:

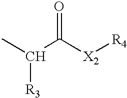

wherein:

R₃ is C₁-C₉ straight or branched chain alkyl which is optionally substituted with C₃-C₈ cycloalkyl or Ar₁;

X₂ is O or NR₆, wherein R₆ is selected from the group consisting of hydrogen, C₁-C₆ straight or branched chain alkyl, and C₂-C₆ straight or branched chain alkenyl;

R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl;

R₂ is C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl, or cycloalkenyl is optionally substituted with one or more substituents selected from the group consisting of C₁-C₆ straight or branched chain alkyl, C₂-C₆ straight or branched chain alkenyl, C₃-C₈ cycloalkyl, C₅-C₇ cycloalkenyl, (Ar₁)ₙ and hydroxy; or,

R₂ is either hydrogen or P; Y is either oxygen or CH—P, provided that if R₂ is hydrogen, then Y is CH—P, or if Y is oxygen then R₂ is P;

P is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar₅, or Ar₅

Ar₁ or Ar₂, independently, is an alicyclic or aromatic, mono-, bi- or tricyclic, carbo- or heterocyclic ring, wherein the ring is optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₃-C₈ cycloalkyl, C₅-C₇ cycloalkenyl, C₁-C₄ alkoxy, C₂-C₄ alkenyloxy, phenoxy, benzyloxy, and amino; wherein the individual ring contains 5-8 members; and wherein the heterocyclic ring contains 1-6 heteroatom(s) independently selected from the group consisting of O, N, and S, and, wherein any aromatic or tertiary alkylamine is optionally oxidized to a corresponding N-oxide;

m is 0 or 1

n is 1 or 2;

p is 0, 1, or 2;

t is 0, 1, 2, 3, or 4;

X is O, CH₂ or S;

W and Y, independently, are O, S, CH₂ or H₂;

Z is C(R₁)₂, O, S, a direct bond or NR₁; or, Z-R₁ is

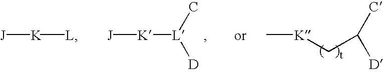

wherein:

C and D are, independently, hydrogen, Ar₄, Ar₁, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, Ar₁ and Ar₄; wherein said alkyl, alkenyl, cycloalkyl or cycloalkenyl is optionally substituted with C₁-C₆ alkyl, C₂-C₆ alkenyl, hydroxy, amino, halo, halo-(C₁-C₆)-alkyl, thiocarbonyl, C₁-C₆ ester, C₁-C₆ thioester, C₁-C₆ alkoxy, C₂-C₆ alkenoxy, cyano, nitro, imino, C₁-C₆ alkylamino, amino-(C₁-C₆)alkyl, sulfhydryl, thio-(C₁-C₆)alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NR₅, or (SO)ₚ;

C′ and D′ are independently hydrogen, Ar₅, C₁-C₆ straight or branched chain alkyl, or C₂-C₆ straight or branched chain alkenyl, wherein said alkyl or alkenyl is optionally substituted with C₅-C₇ cycloalkyl, C₅-C₇ cycloalkenyl, or Ar₅, wherein, one or two carbon atom(s) of said alkyl or alkenyl may be substituted with one or two heteroatom(s) independently selected from the group consisting of oxygen, sulfur, SO, and SO₂ in chemically reasonable substitution patterns, or

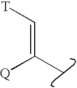

wherein

Q is hydrogen, C₁-C₆ straight or branched chain alkyl, or C₂-C₆ straight or branched chain alkenyl; and

T is Ar₅ or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl J is O, NR₁, S, or (CR₁)₂;

K is a direct bond, C₁-C₆ straight or branched chain alkyl, or C₂-C₆ straight or branched chain alkenyl; wherein said alkyl or alkenyl is optionally substituted with one or more substituenc(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar₃; wherein said alkyl, alkenyl, cycloalkyl, cycloalkenyl or Ar₃, is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl or Ar₃, is optionally replaced with O, NR′″, or S(O)ₚ;

K′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆) -alkyl, thiocarbonyl, C₁-C₆-ester, thio-C₁-C₆-ester, (C₁-C₆) -alkoxy, (C₂-C₆) -alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NR₅, S(O)ₚ;

K″ is C(R₁)₂, O, S, a direct bond or NR₁,

R′″ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar₃ group;

L is an aromatic amine or a tertiary amine oxidized to a corresponding N-oxide; said aromatic amine being selected from the group consisting of pyridyl, pyrimidyl, quinolinyl, and isoquinolinyl, said aromatic amine being optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; and wherein said tertiary amine is NRₓR_{y}R_{z}, wherein Rₓ, R_{y}, and R_{z} are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar₃; wherein said alkyl, alkenyl, cycloalkyl, cycloalkenyl, or Ar₃ is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar₃ is optionally replaced with O, NR′, S(O)ₚ;

L′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NR₅, S(O)ₚ

Ar₃ is selected from the group consisting of pyrrolidinyl, pyridyl, pyrimidyl, pyrazyl, pyridazyl, quinolinyl, and isoquinolinyl; or, Ar₄ is an alicyclic or aromatic, mono-, bi- or tricyclic, carbo- or heterocyclic ring, wherein the ring is optionally substituted with one or more substituent(s) independently selected from the group consisting of alkylamino, amido, amino, amino-(C₁-C₆)-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-(C₁-C₆)-alkyl, hydroxy, imino, isocyano, isonitrilo, nitrilo, nitro, nitroso, phenoxy, sulfhydryl, sulfonylsulfoxy, thio, thio-(C₁-C₆)-alkyl, thiccarbonyl, thiocyano, thio-C₁-C₆-ester, thioformamido, trifluoromethyl, and carboxylic and heterocyclic moieties; wherein the individual alicyclic or aromatic ring contains 5-8 members and wherein said heterocyclic ring contains 1-6 heteroatom(s) independently selected from the group consisting of O, N, and S; and wherein any aromatic or tertiary alkyl amine is optionally oxidized to a corresponding N-oxide;

Ar₅ is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar₅ optionally contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;

R₅ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar₄ or Ar₁ group;

U is either O or N, provided that:

when U is O, then R′ is a lone pair of electrons and R″ is selected from the group consisting of Ar₄, C₃-C₈ cycloalkyl, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; and

when U is N, then R′ and R″ are, independently, selected from the group consisting of hydrogen, Ar₄, C₃-C₁₀ cycloalkyl, a C₇-C₁₂ bi- or tri-cyclic carbocycle, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; or R′ and R″ are taken together to form a heterocyclic 5- or 6-membered ring selected from the group consisting of pyrrolidine, imidazolidine, pyrazolidine, piperidine, and piperazine; or,

a pharmaceutically acceptable salt, ester or solvate thereof.

Additionally, the invention provides a method for the treatment of nerve injury caused as a consequence of prostate surgery by administering a neurotrophic compound of Formula (I′) to a patient in need thereof.

Also provided are a compound of Formula (I′) for use in the preparation of a medicament for the treatment of nerve injury caused as a consequence of prostate surgery. Additionally, there is provided a compound of Formula (I′) for use in the preparation of a medicament for the treatment of erectile dysfunction. In this aspect of the invention, there are also provided a formulation comprising a compound of Formula (I′) for use in the preparation of a medicament for the treatment of nerve injury caused as a consequence of prostate surgery, as well as a formulation comprising a compound of Formula (I′) for use in the preparation of a medicament for the treatment penile cavernous nerve damage.

Additionally, there is provided a formulation adapted for use in the treatment of nerve injury caused as a consequence of prostate surgery which comprises a compound of Formula (I′) associated with a pharmaceutically acceptable carrier, diluent or excipient therefor, as well as a formulation adapted for use in the treatment of erectile dysfunction which comprises a compound of Formula (I′) associated with a pharmaceutically acceptable carrier, diluent or excipient therefor.

More specifically, the invention provides methods, uses, and formulations described above which comprise the use of any of the compounds described below,

I. Heterocyclic Thioesters and Ketones

### Formula I

In particular, the neurotrophic agent may be a compound of formula I:

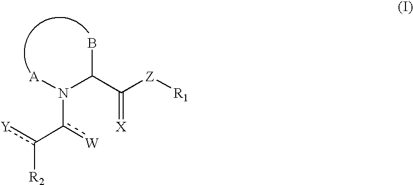

or a pharmaceutically acceptable salt, ester, or solvate thereof, wherein:

A and B, together with the nitrogen and carbon atoms to which they are respectively attached, form a 5-7 membered saturated or unsaturated heterocyclic ring containing one or more heteroatom(s) independently selected from the group consisting of O, S, SO, SO₂, N, NH, and NR₂;

X is either O or S;

Z is either S, CH₂, CHR₁ or CR₁R₃;

W and Y are independently O, S, CH₂ or H₂;

R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;

n is 1 or 2;

R₂ is either C₁-C₉ straight or branched chain alkyl, C₂-C₉ straight or branched chain alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxy; and

Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

### Formula II

The neurotrophic agent may also be a compound of formula II:

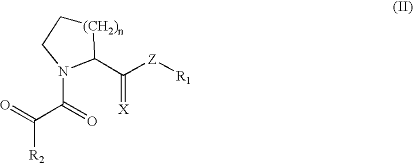

or a pharmaceutically acceptable salt, ester, or solvate thereof, wherein:

n is 1 or 2;

X is O or S;

Z is selected from the group consisting of S, CH₂, CHR₁, and CR₁R₃;

R₁ and R₃ are independently selected from the group consisting of C₁-C₅ straight or branched chain alkyl, C₂-C₅ straight or branched chain alkenyl, and Ar₁, wherein said alkyl, alkenyl or Ar₁ is unsubstituted or substituted with one or more substituent(s) independently selected from the group consisting of halo, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino, and Ar₁;

R₂ is selected from the group consisting of C₁-C₉ straight or branched chain alkyl, C₂-C₉ straight or branched chain alkenyl, C₃-C₈ cycloalkyl, C₅-C₇ cycloalkenyl, and Ar₁; and

Ar₁ is phenyl, benzyl, pyridyl, fluorenyl, thioindolyl or naphthyl, wherein said Ar₁ is unsubstituted or substituted with one or more substituent(s) independently selected from the group consisting of halo, trifluoromethyl, hydroxy, nitro, C₁-C₆ straight or branched chain alkyl, C₂-C₆ straight or branched chain alkenyl, C₁-C₄ alkoxy, C₂-C₄ alkenyloxy, phenoxy, benzyloxy, and amino.

Preferred compounds of formula II are presented in TABLE I.

TABLE I

| | |
|---|---|
| | (II) |
| 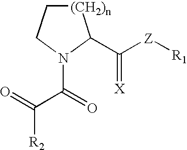 | |

| No | n | X | Z | R₁ | R₂ |
|---|---|---|---|---|---|
| 1 | 1 | O | CH₂ | 3-Phenylpropyl | 1,1-Dimethylpropyl |
| 2 | 1 | O | CH₂ | 3-(3-Pyridyl)propyl | 1,1-Dimethylpropyl |
| 3 | 1 | O | CH₂ | 3-Phenylpropyl | tert-Butyl |
| 4 | 1 | O | CH₂ | 3-(3-Pyridyl)propyl | tert-Buty1 |
| 5 | 1 | O | CH₂ | 3-(3-Pyridyl)propyl | Cyclohexyl |
| 6 | 1 | O | CH₂ | 3-(3-Pyridyl)propyl | Cyclopentyl |
| 7 | 1 | O | CH₂ | 3-(3-Pyridyl)propyl | Cycloheptyl |
| 8 | 1 | O | CH₂ | 2-(9-Fluorenyl)ethyl | 1,1-Dimethylpropyl |
| 9 | 1 | O | S | 2-Phenethyl | 1,1-Dimethylpropyl |
| 10 | 2 | O | S | 2-Phenethyl | 1,1-Dimethylpropyl |
| 11 | 1 | O | S | Methyl(2-thioindole) | 1,1-Dimethylpropyl |
| 12 | 1 | O | S | 2-Phenethyl | Cyclohexyl |
| 13 | 2 | O | S | 2-Phenethyl | tert-Butyl |
| 14 | 2 | O | S | 2-Phenethyl | Phenyl |
| 15 | 1 | O | CH₂ | 3-(4-Methoxyphenyl)propyl | 1,1-Dimethylpropyl |
| 16 | 2 | O | CH₂ | 4-(4-Methoxyphenyl)butyl | 1,1-Dimethylpropyl |
| 17 | 2 | O | CH₂ | 4-Phenylbutyl | 1,1-Dimethylpropyl |
| 18 | 2 | O | CH₂ | 4-Phenylburyl | Phenyl |
| 19 | 2 | O | CH₂ | 4-Phenylbutyl | Cyclohexyl |
| 20 | 1 | S | CH₂ | 3-Phenylpropyl | 1,1-Dimethylpropyl |
| 21 | 1 | S | S | 2-Phenethyl | 1,1-Dimethylpropyl |
| 22 | 2 | S | CH₂ | 3-Phenylpropyl | 1,1-Dimethylpropyl |
| 23 | 2 | S | S | 2-Phenethyl | 1,1-Dimethylpropyl |
| 24 | 2 | O | CHR₁ | 3-Phenylpropyl | 1,1-Dimethylpropyl |
| 25 | 2 | O | CHR₁ | 3-Phenylpropyl | Cyclohexyl |
| 26 | 2 | O | CHR₁ | 3-Phenylpropyl | Phenyl |
| 27 | 2 | O | CHR₁ | 3-Phenylpropyl | 3,4,5- |
| | | | | | Trimethoxyphenyl |
| 28 | 1 | O | S | 2-Phenethyl | Cyclopentyl |
| 29 | 2 | O | S | 3-Phenylpropyl | tert-Butyl |
| 30 | 1 | O | S | 3-Phenylpropyl | 1,1-Dimethylpropyl |
| 31 | 1 | O | S | 3-(3-Pyridyl)propyl | 1,1-Dimethylpropyl |
| 32 | 1 | O | S | 3-Phenylpropyl | Cyclohexyl |
| 33 | 1 | O | S | 4-Phenylbutyl | Cyclohexyl |
| 34 | 1 | O | S | 4-Phenylbutyl | 1,1-Dimethylpropyl |
| 35 | 1 | O | S | 3-(3-Pyridyl)propyl | Cyclohexyl |
| 36 | 1 | O | S | 3,3-Diphenylpropyl | 1,1-Dimethylpropyl |
| 37 | 1 | O | S | 3,3-Diphenylpropyl | Cyclohexyl |
| 38 | 1 | O | S | 3-(4-Methoxyphenyl)propyl | 1,1-Dimethylpropyl |
| 39 | 2 | O | S | 4-Phenylbutyl | tert-Butyl |
| 40 | 2 | O | S | 1,5-Diphenylpentyl | 1,1-Dimethylpropyl |
| 41 | 2 | O | S | 1,5-Diphenylpentyl | Phenyl |
| 42 | 2 | O | S | 3-(4-Methoxyphenyl)propyl | 1,1-Dimethylpropyl |
| 43 | 2 | O | S | 3-(4-Methoxyphenyl)propyl | Phenyl |
| 44 | 2 | O | S | 3-(1-Naphthyl)propyl | 1,1-Dimethylpropyl |
| 45 | 1 | O | S | 3,3-Di(4-fluoro)phenyl- | 1,1-Dimethylpropyl |
| | | | | propyl | |
| 46 | 1 | O | S | 4,4-Di(4- | 1,1-Dimethylpropyl |
| | | | | fluoro)phenylbutyl | |
| 47 | 1 | O | S | 3-(1-Naphthyl)propyl | 1,l-Dimethylpropyl |
| 48 | 1 | O | S | 2,2-Diphenylethyl | 1,1-Dimethylpropyl |
| 49 | 2 | O | S | 2,2-Diphenylethyl | 1,1-Dimethylpropyl |
| 50 | 2 | O | S | 3,3-Diphenylpropyl | 1,1-Dimethylpropyl |
| 51 | 1 | O | S | 3-(4- | 1,1-Dimethylpropyl |
| | | | | {Trifluoromethyl}phenyl)- | |
| | | | | propyl | |
| 52 | 1 | O | S | 3-(2-Naphthyl)propyl | 1,1-Dimethylpropyl |
| 53 | 2 | O | S | 3-(1-Naphthyl)propyl | 1,1-Dimethylpropyl |
| 54 | 1 | O | S | 3-(3-Chloro)phenylpropyl | 1,1-Dimethylpropyl |
| 55 | 1 | O | S | 3-(3- | 1,1-Dimethylpropyl |
| | | | | {Trifluoromethyl}phenyl)- | |
| | | | | propyl | |
| 56 | 1 | O | S | 3-(2-Biphenyl)propyl | 1,1-Dimethylpropyl |
| 57 | 1 | O | S | 3-(2-Fluorophenyl)propyl | 1,1-Dimethylpropyl |
| 58 | 1 | O | S | 3-(3-Fluorophenyl)propyl | 1,1-Dimethylpropyl |
| 59 | 2 | O | S | 4-Phenylbutyl | 1,1-Dimethylpropyl |
| 60 | 2 | O | S | 3-Phenylpropyl | 1,1-Dimethylpropyl |
| 61 | 1 | O | S | 3-(2-Chloro)phenylpropyl | 1,1-Dimethylpropyl |
| 62 | 2 | O | S | 3-(3-Chloro)phenylpropyl | 1,1-Dimethylpropyl |
| 63 | 2 | O | S | 3-(2-Fluoro)phenylpropyl | 1,1-Dimethylpropyl |
| 64 | 2 | O | S | 3-(3-Fluoro)phenylpropyl | 1,1-Dimethylpropyl |
| 65 | 1 | O | S | 3-(2,5- | 1,1-Dimethylpropyl |
| | | | | Dimethoxyphenyl)propyl | |
| 66 | 1 | O | CH₂ | 3-Phenylpropyl | Cyclohexyl |
| 67 | 1 | O | CH₂ | 3-Phenylethyl | tert-Butyl |
| 68 | 2 | O | CH₂ | 4-Phenylbutyl | Cyclohexyl |
| 69 | 2 | O | CHR₁ | 2-Phenylethyl | tert-Butyl |
| 70 | 1 | O | CH₂ | 3,3-Di(4- | 1,1-Dimethylpropyl |
| | | | | fluorophenyl)propyl | |
| 71 | 2 | O | CH₂ | 3-Phenylpropyl | 1,1-Dimethylpropyl |

Preferred compounds of TABLE I are named as follows:

1 (2S)-2-({1-Oxo-5-phenyl}-pentyl-1-(3,3-dimethyl-1,2-dioxopentyl)pyrrolidine

2 3,3-Dimethyl-1-[(2S)-2-(5-(3-pyridyl)pentanoyl)-1-pyrrolidine]-1,2-pentanedione

3 (2S)-2-({1-Oxo-4-phenyl}-butyl-1-(3,3-dimethyl-1,2-dioxobutyl)pyrrolidine

9 2-Phenyl-1-ethyl (2S)-1-(3,3-dimethyl-1,2-dioxopentyl)-2-pyrrolidinecarbothioate

10 2-Phenyl-1-ethyl 1-(3,3-dimethyl-1,2-dioxopentyl)-2-piperidinecarbothioate

11 (3-Thioindolyl)methyl (2S)-1-(3,3-dimethyl-1,2-dioxopentyl)-2-pyrrolidinecarbothioate

12 2-Phenyl-1-ethyl (2S)-1-(2-cyclohexyl-1,2-dioxoethyl)-2-pyrrolidinecarbothioate

14 2-Phenyl-1-ethyl 1-(2-phenyl-1,2-dioxoethyl)-2-piperidinecarbothioate

28 2-Phenyl-1-ethyl (2S)-1-(1-cyclopentyl-1,2-dioxoethyl)-2-pyrrolidinecarbothioate

29 3-Phenyl-1-propyl 1-(3,3-dimethyl-1,2-dioxobutyl)-2-piperidinecarbothioate

30 3-Phenyl-1-propyl (2S)-1-(3,3-dimethyl-1,2-dioxopentyl)-2-pyrrolidir.ecarbothioate

31 3-(3-Pyridyl)-1-propyl (2S)-1-(3,3-dimethyl-1,2-dioxopentyl)-2-pyrrolidinecarbothioate

32 3-Phenyl-1-propyl (2S)-1-(2-cyclohexyl-1,2-dioxoethyl)-2-pyrrolidinecarbothioate

33 4-Phenyl-1-butyl (2S)-1-(2-cyclohexyl-1,2-dioxoethyl)-2-pyrrolidinecarbothioate

34 4-Phenyl-1-butyl (2s)-1-(3,3-dimethyl-1,2-dioxopentyl)-2-pyrrolidinecarbothioate

35 3-(3-Pyridyl)-1-propyl (2S)-1-(2-cyclohexyl-1,2-dioxoethyl)-2-pyrrolidinecarbothioate

36 3,3-Diphenyl-1-propyl (2S)-1-(3,3-dimethyl-1,2-dioxopentyl)-2-pyrrolidinecarbothioate

37 3,3-Diphenyl-1-propyl (2S)-1-(2-cyclohexyl-1,2-dioxoethyl)-2-pyrrolidinecarbothioate

38 3-(para-Methoxyphenyl)-1-propyl (2S)-1-(3,3-dimethyl-1,2-dioxopentyl)-2-pyrrolidine-carbothioate

39 4-Phenyl-1-butyl 1-(1,2-dioxo-3,3-dimethylbutyl)-2-piperidinecarbothioate

40 1,5-Diphenyl-3-pentyl 1-(3,3-dimethyl-1,2-dioxopentyl)-2-piperidinecarbothioate-

41 1,5-Diphenyl-3-mercaptopentyl 1-(3-phenyl-1,2-dioxoethyl)-2-piperidinecarbothioate

42 3-(para-Methoxyphenyl)-1-propyl 1-(1,2-dioxo-3,3-dimethylpentyl)piperidine-2-carbothioate

43 3-(para-Methoxyphenyl)-1-propyl 1-(2-phenyl-1,2-dioxoethyl)piperidine-2-carbothioate

44 3-(1-Naphthyl)-1-propyl 1-(3,3-dimethyl-1,2-dioxopentyl)piperidine-2-carbothioate

45 3,3-Di(para-fluoro)phenyl-1-propyl (2S)-1-(3,3-dimethyl-1,2-dioxopentyl)-2-pyrrolidine-carbothioate

46 4,4-Di(para-fluorophenyl)butyl 1-(3,3-dimethyl-2-oxopentanoyl)-2-pyrrolidinecarbothioate

47 3-(1-Naphthyl)propyl (2S)-1-(3,3-dimethyl-2-oxopentanoyl)-2-pyrrolidinecarbothioate

48 2,2-Diphenylethyl (2S)-1-(3,3-dimethyl-2-oxopentanoyl)tetrahydro-1H-2-pyrrolidine-carbothioate

49 2,2-Diphenylethyl (2S)-1-(3,3-dimethyl-2-oxopentanoyl)-2-piperidinecarbothioate

50 3,3-Diphenylpropyl 1-(3,3-dimethyl-2-oxopentanoyl)-2-piperidinecarbothioate

51 3-[4-(Trifluoromethyl)phenyl]propyl (2S)-1-(3,3-dimethyl-2-oxopentanoyl)-2-pyrrolidine-carbothioate

52 3-(2-Naphthyl)propyl (2S)-1-(3,3-dimethyl-2-oxopentanoyl)-2-pyrrolidinecarbothioate

53 3-(2-Naphthyl)propyl (2R,S)-1-(3,3-dimethyl-2-oxopentanoyl)-2-piperidinecarbothioate

54 3-(3-Chlorophenyl)propyl (2S)-1-(3,3-dimethyl-2-oxopentanoyl)-2-pyrrolidinecarbothioate

55 3-[3-(Trifluoromethyl)phenyl]propyl (2S)-1-(3,3-dimethyl-2-oxopentanoyl)-2-pyrrolidine-carbothioate

56 3-(1-Biphenyl)propyl (2S)-1-(3,3-dimethyl-2-oxopentanoyl)-2-pyrrolidinecarbothioate

57 3-(2-Fluorophenyl)propyl (2S)-1-(3,3-dimethyl-2-oxopentanoyl)-2-pyrrolidinecarbothioate

58 3-(3-Fluorophenyl)propyl (2S)-1-(3,3-dimethyl-2-oxopentanoyl)-2-pyrrolidinecarbothioate

59 4-Phenylbutyl 1-(3,3-dimethyl-2-oxopentanoyl)-2-piperidinecarbothioate

60 3-Phenylpropyl 1-(3,3-dimethyl-2-oxopentanoyl)-2-piperidinecarbothioate

61 3-(2-Chlorophenyl)propyl (2S)-1-(3,3-dimethyl-2-oxopentanoyl)-2-pyrrolidinecarbothioate

62 3-(2-Chlorophenyl)propyl 1-(3,3-dimethyl-2-oxopentanoyl)-2-piperidinecarbothioate

63 3-(2-Fluorophenyl)propyl 1-(3,3-dimethyl-2-bxopentanoyl)-2-piperidinecarbothioate

64 3-(3-Fluorophenyl)propyl 1-(3,3-dimethyl-2-oxopentanoyl)-2-piperidinecarbothioate

65 3-(3,4-Dimethoxyphenyl)propyl (2S)-1-(3,3-dimethyl-2-oxopentanoyl)-2-pyrrolidinecarbothioate

66 (2S)-2-({1-Oxo-4-phenyl}-butyl-1-(2-Cyclohexyl-1,2-dioxoethyl)pyrrolidine

67 2-({1-Oxo-4-phenyl}-butyl-1-(3,3-dimethyl-1,2-dioxobutyl)pyrrolidine

68 2-({1-Oxo-6-phenyl}-hexyl-1-(2-Cyclohexyl-1,2-dioxoethyl)piperidine

69 2-({1-Oxo-[2-12′-phenyl}ethyl]-4-phenyl}-butyl-1-(3,3-dimethyl-1,2-dioxobutyl)piperidine

70 1-{(2S)-2-[5,5-di(4-Fluorophenyl)pentanoyl]-2-pyrrolidine}-3,3-dimethyl-1,2-pentanedione

71 3,3-Dimethyl-1-[2-(4-phenylpentanoyl)piperidino]-1,2-pentanedione

### Formula III

Furthermore, the neurotrophic agent may be a compound of formula III:

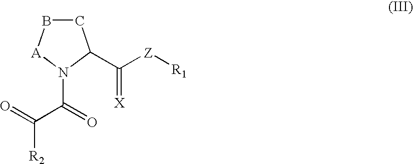

or a pharmaceutically acceptable salt, ester, or solvate thereof, wherein:

A, B, and C are independently CH₂, O, S, SO, SO₂, NH or NR₂;

X is O or S;

Z is S, CH₂, CHR₁ or CR₁R₃;

R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;

n is 1 or 2;

R₂ is either C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and

Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

Preferred compounds of formula III are presented in TABLE II:

TABLE II

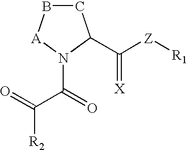

| No. | A | B | C | X | Z | R₁ | R₂ |
|---|---|---|---|---|---|---|---|
| 72 | CH₂ | S | CH₂ | O | S | 2-phenethyl | 1,1-dimethylpropyl |
| 73 | CH₂ | S | CH₂ | O | CH₂ | 3-phenylpropyl | 1,1-dimethylpropyl |
| 74 | CH₂ | CH₂ | NH | O | S | 2-phenethyl | 1,1-dimethylpropyl |
| 75 | CH₂ | S | CH₂ | S | S | 2-phenethyl | 1,1-dimethylpropyl |

### Formula IV

Alternatively, the neurotrophic agent may be a compound of formula IV:

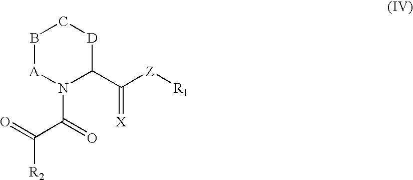

or a pharmaceutically acceptable salt, ester, or solvate thereof, wherein:

A, B, C and D are independently CH₂, O, S, SO, SO₂, NH or NR₂;

X is O or S;

Z is S, CH₂, CHR₁ or CR₁R₃;

R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;

n is 1 or 2;

R₂ is either C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is either unsubstituted or substituted with one or more substituent(s) independently selected from the group consisting of C₃-C₈ cycloalkyl, C₁-C₄ straight or branched chain alkyl, C₂-C₄ straight or branched chain alkenyl, and hydroxyl; and

Ar₁ and Ar₂ are independently an alicyclic or aromatic, mono-, bi- or tricyclic, carbo- or heterocyclic ring, wherein said ring is either unsubstituted or substituted with one or more substituent(s) independently selected from the group consisting of halo, hydroxyl, nitro, trifluoro-methyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

Preferred compounds of formula IV are presented in TABLE III.

TABLE III

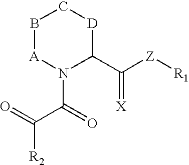

| No. | A | B | C | D | X | Z | R₁ | R₂ |
|---|---|---|---|---|---|---|---|---|
| 76 | CH₂ | CH₂ | O | CH₂ | O | CH₂ | 3-phenylpropyl | 1,1-dimethylpropyl |
| 77 | CH₂ | CH₂ | O | CH₂ | O | S | 2-phenethyl | 1,1-dimethylpropyl |
| 78 | CH₂ | CH₂ | S | CH₂ | O | CH₂ | 3-phenylpropyl | 1,1-dimethylpropyl |
| 79 | CH₂ | CH₂ | S | CH₂ | O | S | 2-phenethyl | 1,1-dimethylpropyl |

### Formula V

The neurotrophic agent may further be a compound of formula V:

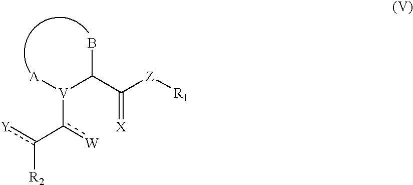

or a pharmaceutically acceptable salt, ester, or solvate thereof, wherein:

V is CH, N, or S;

A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR₄;

R₄ is either C₁-C₉ straight or branched chain alkyl, C₂-C₉ straight or branched chain alkenyl, C₃-C₉ cycloalkyl, C₅-C₇ cycloalkenyl, or Ar₃, wherein R₄ is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;

Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S; and

R₁, R₂, W, X, Y, and Z are as defined in Formula I above.

II. Heterocyclic Esters and Amides

### Formula VI

Additionally, the neurotrophic agent may be a compound of formula VI:

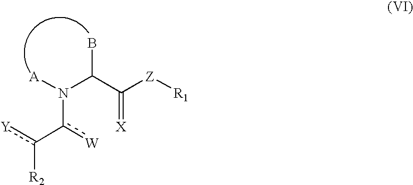

or a pharmaceutically acceptable salt, ester, or solvate thereof, wherein:

A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₁;

X is O or S;

Z is O, NH or NR₁;

W and Y are independently O, S, CH₂ or H₂;

R₁ is C₁-C₆ straight or branched chain alkyl or C₂-C₆ straight or branched chain alkenyl, which is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;

n is 1 or 2;

R₂ is either C₁-C₉ straight or branched chain alkyl, C₂-C₉ straight or branched chain or alkenyl, C₃-C₈ cycloalkyl, C₅-C₇ cycloalkenyl, or Ar₁, wherein said alkyl, alkenyl, cycloalkyl or cycloalkenyl is either unsubstituted or substituted with one or more substituent(s) independently selected from the group consisting of C₁-C₄ straight or branched chain alkyl, C₂-C₄ straight or branched chain alkenyl, and hydroxyl; and

Ar₁ and Ar₂ are independently an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

Suitable carbo- and heterocyclic rings include without limitation naphthyl, indolyl, furyl, thiazolyl, thienyl, pyridyl, quinolinyl, isoquinolinyl, fluorenyl and phenyl.

### Formula VII

The neurotrophic agent may also be a compound of formula VII:

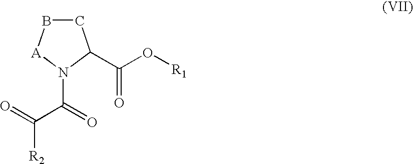

or a pharmaceutically acceptable salt, ester, or solvate thereof, wherein:

A, B and C are independently CH₂, O, S, SO, SO₂, NH or NR_{1;}

R₁ is C₁-C₅ straight or branched chain alkyl or C₂-C₅ straight or branched chain alkenyl, which is substituted with one or more substituent(s) independently selected from the group consisting of (Ar₁)ₙ and C₁-C₆ straight or branched chain alkyl or C₂-C₆ straight or branched chain alkenyl substituted with (Ar₁)ₙ;

n is 1 or 2;

R₂ is either C₁-C₉ straight or branched chain alkyl, C₂-C₉ straight or branched chain alkenyl, C₃-C₈ cycloalkyl, C₅-C₇ cycloalkenyl, or Ar₁; and

Ar₁ is an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

A preferred compound of formula VII is:

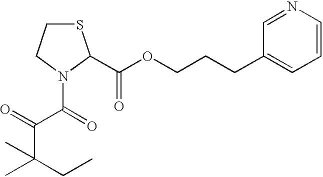

In a particularly preferred embodiment of formula VII compounds:

A is CH₂;

B is CH₂ or S;

C is CH₂ or NH;

R₁ is selected from the group consisting of 3-phenylpropyl and 3-(3-pyridyl)propyl; and

R₂ is selected from the group consisting of 1,1-dimethylpropyl, cyclohexyl, and tert-butyl.

Specific examples of this embodiment are presented on TABLE IV:

TABLE IV

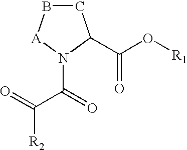

| No. | A | B | C | R₁ | R₂ |
|---|---|---|---|---|---|
| 80 | CH₂ | S | CH₂ | 3-phenylpropyl | 1,1-dimethylpropyl |
| 81 | CH₂ | S | CH₂ | 3-(3-pyridyl)propyl | 1,1-dimethylpropyl |
| 82 | CH₂ | S | CH₂ | 3-phenylpropyl | cyclohexyl |
| 83 | CH₂ | S | CH₂ | 3-phenylpropyl | tert-butyl |
| 84 | CH₂ | CH₂ | NH | 3-phenylpropyl | 1,1-dimethylpropyl |
| 85 | CH₂ | CH₂ | NH | 3-phenylpropyl | cyclohexyl |
| 86 | CH₂ | CH₂ | NH | 3-phenylpropyl | tert-butyl |

### Formula VIII

In a further embodiment of this invention, the neurotrophic agent may be a compound of formula VIII:

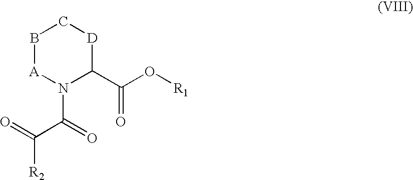

or a pharmaceutically acceptable salt, ester, or solvate thereof, wherein:

A, B, C and D are independently CH₂, O, S, SO, SO₂, NH or NR₁;

R₁ is C₁-C₅ straight or branched chain alkyl or C₂-C₅ straight or branched chain alkenyl, which is substituted with one or more substituent(s) independently selected from the group consisting of (Ar₁)ₙ, and C₁-C₆ straight or branched chain alkyl or C₂-C₆ straight or branched chain alkenyl substituted with (Ar₁)ₙ;

n is 1 or 2;

R₂ is either C₁-C₉ straight or branched chain alkyl, C₂-C₉ straight or branched chain alkenyl, C₃-C₈ cycloalkyl, C₅-C₇ cycloalkenyl, or Ar₁; and

Ar₁ is an alicyclic or aromatic, mono-, bi- or tricyclic, carbo- or heterocyclic ring, wherein the ring is either unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; wherein the individual ring size is 5-8 members; and wherein the heterocyclic ring contains 1-6 heteroatom(s) independently selected from the group consisting of O, N, and S.

In a particularly preferred embodiment of formula VIII compounds:

A is CH₂;

B is CH₂;

C is S, O or NH;

D is CH₂;

R₁ is selected from the group consisting of 3-phenylpropyl and (3,4,5-trimethoxy)phenylpropyl; and

R₂ is selected from the group consisting of 1,1-dimethylpropyl, cyclohexyl, tert-butyl, phenyl, and trimethoxyphenyl.

Specific examples of this embodiment are presented in TABLE V.

TABLE V

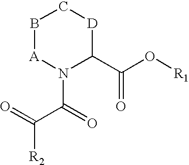

| No. | A | B | C | D | R₁ | R₂ |
|---|---|---|---|---|---|---|
| 87 | CH₂ | CH₂ | S | CH₂ | 3-phenylpropyl | 1,1-dimethylpropyl |
| 88 | CH₂ | CH₂ | O | CH₂ | 3-phenylpropyl | 1,1-dimethylpropyl |
| 89 | CH₂ | CH₂ | S | CH₂ | 3-phenylpropyl | cyclohexyl |
| 90 | CH₂ | CH₂ | O | CH₂ | 3-phenylpropyl | cyclohexyl |
| 91 | CH₂ | CH₂ | S | CH₂ | 3-phenylpropyl | phenyl |
| 92 | CH₂ | CH₂ | O | CH₂ | 3-phenylpropyl | phenyl |
| 93 | CH₂ | CH₂ | NH | CH₂ | 3-phenylpropyl | 1,1-dimethylpropyl |
| 94 | CH₂ | CH₂ | NH | CH₂ | 3-phenylpropyl | phenyl |

### Formula IX

Additionally, the neurotrophic agent may be a compound of formula IX:

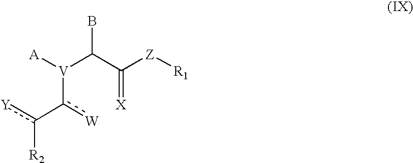

or a pharmaceutically acceptable salt, ester, or solvate thereof, wherein:

V is CH, N, or S;

A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR;

R is either C₁-C₉ straight or branched chain alkyl, C₂-C₉ straight or branched chain alkenyl, C₃-C₉ cycloalkyl, C₅-C₇ cycloalkenyl, or Ar₃, wherein R is is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;

Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S; and

R₁, R₂, W, X, Y, and Z are as defined in Formula VI above.

III. N-Oxides of Heterocyclic Esters, Amides, Thio-Esters and Ketones

### Formula X

The neurotrophic agent may further be a compound of formula X:

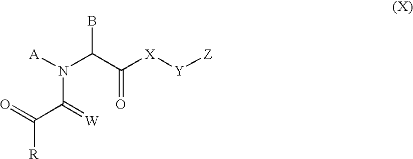

or a pharmaceutically acceptable salt, ester, or solvate thereof, wherein:

A and B, together with the nitrogen and carbon atoms to which they are respectively attached, form a 5-7 membered saturated or unsaturated heterocyclic ring containing one or more heteroatom(s) independently selected from the group consisting of CH, CH₂, O, S, SO, SO₂, N, NH, and NR₁;

W is O, S, CH₂, or H₂;

R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₂;

Ar₁ and Ar₂ are independently selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;

X is O, NH, NR₁, S, CH, CR₁, or CR₁R₃;

Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is an aromatic amine or a tertiary amine oxidized to a corresponding N-oxide;

said aromatic amine is selected from the group consisting of pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

said tertiary amine is NR₄R₅R₆, wherein R₄, R₅, and R₆ are independently selected from the group consisting of C₁-C₆ straight or branched chain alkyl or C₂-C₆ straight or branched chain alkenyl optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;

Ar is selected from the group consisting of pyrrolidinyl, pyridyl, pyrimidyl, pyrazyl, pyridazyl, quinolinyl, and isoquinolinyl; and

R₁ and R₃ are independently hydrogen, C₁-C₄ straight or branched chain alkyl, C₃-C₄ straight or branched chain alkenyl or alkynyl, or Y-Z.

### Formula XI

Moreover, the neurotrophic agent may be a compound of formula XI:

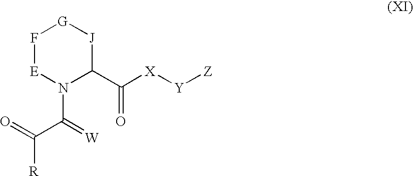

or a pharmaceutically acceptable salt, ester, or solvate thereof, wherein:

E, F, G and J are independently CH₂, O, S, SO, SO₂, NH or NR₁;

W is O, S, CH₂, or H₂;

R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;

Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl, and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;

X is O, NH, NR₁, S, CH, CR₁, or CR₁R₃;

Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is an aromatic amine or a tertiary amine oxidized to a corresponding N-oxide;

said aromatic amine is pyridyl, pyrimidyl, quinolinyl, and isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;

Ar is selected from the group consisting of pyrrolidinyl, pyridyl, pyrimidyl, pyrazyl, pyridazyl, quinolinyl, and isoquinolinyl; and

R₁ and R₃ are independently hydrogen, C₁-C₄ straight or branched chain alkyl, C₃-C₄ straight or branched chain alkenyl or alkynyl, or Y-Z.

### Formula XII

Furthermore, the neurotrophic agent may be a compound of formula XII:

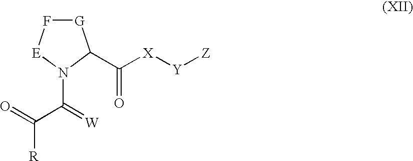

or a pharmaceutically acceptable salt, ester, or solvate thereof, wherein:

E, F, and G are independently CH₂, O, S, SO, SO₂, NH or NR₁;

W is O, S, CH₂, or H₂;

R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;

Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, triflucromethyl, C₁-C₆ straight or branched chain alkyl, C₂-C₆ straight or branched chain alkenyl, C₂-C₄ alkenyloxy, phenoxy, benzyloxy, and amino;

X is O, NH, NR₁, S, CH, CR₁, or CR₁R₃;

Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, aikenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is an aromatic amine or a tertiary amine oxidized to a corresponding N-oxide;

said aromatic amine is pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;

Ar is selected from the group consisting of pyrrolidinyl, pyridyl, pyrimidyl, pyrazyl, pyridazyl, quinolinyl, and isoquinolinyl; and

R₁ and R₃ are independently hydrogen, C₁-C₄ straight or branched chain alkyl, C₃-C₄ straight or branched chain alkenyl or alkynyl, or Y-Z.

### Formula XIII

The neurotrophic agent may also be a compound of formula XIII:

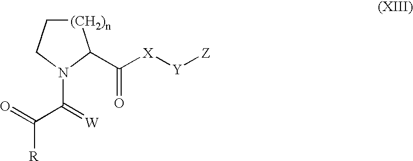

or a pharmaceutically acceptable salt, ester, or solvate thereof, wherein:

n is 1, 2, or 3, forming a 5-7 member heterocyclic ring;

W is O, S, CH₂, or H₂;

R is C₁-C₆ straight or branched chain alkyl, C₂-C₆ straight or branched chain alkenyl, C₃-C₈ cycloalkyl, C₅-C₇ cycloalkenyl, or Ar₁, which is optionally substituted with one or more substituent(s) independently selected from the group consisting of C₁-C₄ alkyl, C₂-C₄ alkenyl, hydroxy, C₃-C₈ cycloalkyl, C₅-C₇ cycloalkenyl, and Ar₁;

Ar₁ is selected from the group consisting of 1-napthyl, 2-napthyl, 1-indolyl, 2-indolyl, 2-furyl, 3-furyl, 2-thienyl, 3-thienyl, 2-pyridyl, 3-pyridyl, 4-pyridyl and phenyl, having one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₂-C₄ alkenyloxy, phenoxy, benzyloxy, and amino;

X is O, NH, NR₁, S, CH, CR₁, or CR₁R₃;

Y is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is an aromatic amine or a tertiary amine oxidized to a corresponding N-oxide;

said aromatic amine is pyridyl, pyrimidyl, quinolinyl, or isoquinolinyl, which is either unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

said tertiary amine is NR₄R₅R₆, wherein R₄, R₅, and R₆ are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, and Ar; wherein said alkyl, alkenyl, cycloalkyl, cycloalkenyl, or Ar is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar is optionally replaced with O, NH, NR₁, S, SO, or SO₂;

Ar is selected from the group consisting of pyrrolidinyl, pyridyl, pyrimidyl, pyrazyl, pyridazyl, quinolinyl, and isoquinolinyl; and

R₁ and R₃, independently, are hydrogen, C₁-C₄ straight or branched chain alkyl, C₃-C₄ straiaht or branched chain alkenyl or alkynyl, or Y-Z.

Examples of the compounds of formula XIII when W is O are presented in TABLE VI:

TABLE VI

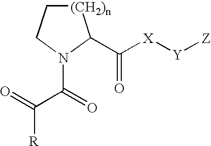

| No. | N | X | Y | Z | R |
|---|---|---|---|---|---|
| 95 | 1 | O | (CH₂)₃ | 3-Pyridyl N-oxide | 1,1-dimethylpropyl |
| 96 | 1 | O | (CH₂)₃ | 2-Pyridyl N-oxide | 1,1-dimethylpropyl |
| 97 | 1 | O | (CH₂)₃ | 4-Pyridyl N-oxide | 1,1-dimethylpropyl |
| 98 | 1 | O | (CH₂)₃ | 2-Quinolyl N-oxide | 1,1-dimethylpropyl |
| 99 | 1 | O | (CH₂)₃ | 3-Quinolyl N-oxide | 1,1-dimethylpropyl |
| 100 | 1 | O | (CH₂)₃ | 4-Quinolyl N-oxide | 1,1-dimethylpropyl |

Preferred compounds of formula XIII may be selected from the group consisting of:

3-(2-Pyridyl)-1-propyl(2S) -1(1,1-Dimethyl-1,2-dioxopentyl)-2-pyrrolidinecarboxylate, N-oxide;

3-(3-Pyridyl)-1-propyl(2S)-1-(1,1-Dimethyl-1,2-dioxopentyl)-2-pyrrolidinecarboxylate, N-oxide;

3-(4-Pyridyl)-1-propyl(2S)-1-(1,1-Dimethyl-1,2-dioxopentyl)-2-pyrrolidinecarboxylate, N-oxide;

3-(2-Quinolyl)-1-propyl(2S)-1-(1,1-Dimethyl-1,2-dioxopentyl)-2-pyrrolidinecarboxylate, N-oxide;

3-(3-Quinolyl)-1-propyl(2S)-1-(1,1-Dimethyl-1,2-dioxopentyl)-2-pyrrolidinecarboxylate, N-oxide;

3-(4-Quinolyl)-1-propyl(2S)-1-(1,1-Dimethyl-1,2-dioxopentyl)-2-pyrrolidinecarboxylate, N-oxide; and

pharmaceutically acceptable salts, esters, and solvates thereof.

### Formula XIV

Additionally, the neurotrophic agent may be a compound of formula XIV:

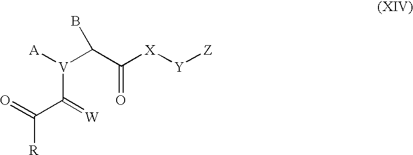

or a pharmaceutically acceptable salt, ester, or solvate thereof, wherein:

V is CH, N, or S;

A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR₇;

R₇ is either C₁-C₉ straight or branched chain alkyl, C₂-C₉ straight or branched chain alkenyl, C₃-C₉ cycloalkyl, C₅-C₇ cycloalkenyl, or Ar₃, wherein R₇ is either unsubstituted or substituted with one or more substituent(s) independently selected from the group consisting of halo, halo-C₁-C₆-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-C₁-C₆-alkyl, C₁-C₆-alkylthio, sulfhydryl, amino, C₁-C₆-alkylamino, amino-C₁-C₆-alkyl, aminocarboxyl, and Ar₄;

Ar₃ and Ar₄ are independently an alicyclic or aromatic, mono-, bi- or tricyclic, carbo- or heterocyclic ring; wherein the individual ring size is 5-8 members; wherein said heterocyclic ring contains 1-6 heteroatom(s) independentiy selected from the group consisting of O, N, and S; and

R, W, X, Y, and Z are as defined in Formula X above.

IV. N-Linked Ureas and Carbamates of Heterocyclic Thioesters

The neurotrophic agent may further be a compound of formula XV:

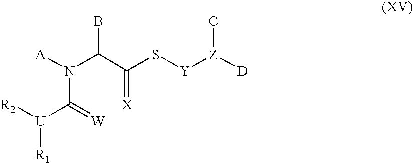

or a pharmaceutically acceptable salt, ester, or solvate thereof, wherein:

A and B, together with the nitrogen and carbon atoms to which they are respectively attached, form a 5-7 membered saturated or unsaturated heterocyclic ring containing, in addition to the nitrogen atom, one or more additional heteroatom(s) independently selected from the group consisting of O, S, SO, SO₂, N, NH, and NR₃;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₃ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;

W is O or S; and

U is either O or N, provided that:

when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and

when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

In a preferred embodiment of formula XV, Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

### Formula XVI

Moreover, the neurotrophic agent may be a compound of formula XVI:

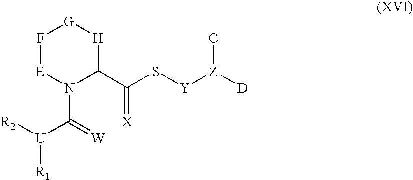

or a pharmaceutically acceptable salt, ester, or solvate thereof, wherein:

E, F, G and J are independently CH₂, O, S, SO, SO₂, NH, or NR₃;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicycllc or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-CB cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thiol-C₁-C₆ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;

W is O or S; and

U is either O or N, provided that:

when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and

when U is N, then R₁ and R₂ are, independently, selected from the group consisting of hydrogen, Ar, C₃-C₁₀cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered ring selected from the group consisting of pyrrolidine, imidazolidine, pyrazolidine, piperidine, and piperazine.

In a preferred embodiment of formula XVI, Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

### Formula XVII

The neurotrophic agent may also be a compound of formula XVII:

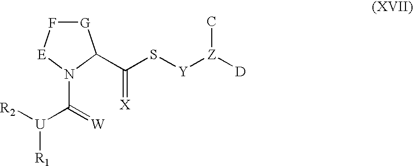

or a pharmaceutically acceptable salt, ester, or solvate thereof, wherein:

E, F, and G are independently CH₂, O, S, SO, SO₂, NH, and NR₃;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, -sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;

W is O or S; and

U is either O or N, provided that:

when U is 0, then R₁ is a lone pair of electrons and R₂ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; and

when U is N, then R₁ and R₂ are, independently, selected from the group consisting of hydrogen, Ar, C₃-C₈ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cyclcalkyl; or R₁ and R₂ are taken together to form a heterocyclic 5 or 6 membered ring selected from the group consisting of pyrrolidine, imidazolidine, pyrazolidine, piperidine, and piperazine.

In a preferred embodiment of formula XVII, Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

### Formula XVIII

The neurotrophic agent may further be a compound of formula XVIII:

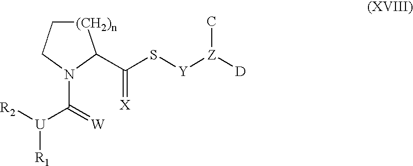

or a pharmaceutically acceptable salt, ester, or solvate thereof, wherein:

n is 1, 2 or 3;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₃ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s) independently selected from the group consisting of C₁-C₆-alkylamino, amido, amino, amino-C₁-C₆-alkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, C₁-C₆-ester, formanilido, halo, halo-C₁-C₆-alkyl, hydroxy, imino, isocyano, isonitrilo, nitrilo, nitro, nitroso, phenoxy, sulfhydryl, sulfonylsulfoxy, thio, thio-C₁-C₆-alkyl, thiocarbonyl, thiocyano, thio-C₁-C₆-ester, thioformamido, trifluoromethyl, and carboxylic and heterocyclic moieties, including alicyclic and aromatic structures; wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂;

W is O or S; and

U is either O or N, provided that:

when U is O, then R₁ is a lone pair of electrons and R₂ is selected from the group consisting of Ar, C₃-C₈ cycloalkyl, C₁-C₆ straight or branched chain alkyl, and C₂-C₆ straight or branched chain or alkenyl, wherein said alkyl or alkenyl is optionally substituted with one or more substituent(s) independently selected from the group consisting of Ar and C₃-C₈ cycloalkyl; and

when U is N, then R₁ and R₂ are, independently, selected from the group consisting of hydrogen, Ar, C₃-C₁₀ cycloalkyl, C₇-C₁₂ bi- or tri-cyclic carbocycle, C₁-C₆ straight or branched chain alkyl, and C₂-C₆ straight or branched chain alkenyl, wherein said alkyl or alkenyl is optionally substituted with one or more substituent(s) independently selected from the group consisting of Ar and C₃-C₈ cycloalkyl; or R₁ and R₂ are taken together to form a heterocyclic 5 or 6 membered is ring selected from the group consisting of pyrrolidine, imidazolidine, pyrazolidine, piperidine, and piperazine.

In a preferred embodiment of formula XVIII, Ar is selected from the group consisting of phenyl, benzyl, naphthyl, pyrrolyl, pyrrolidinyl, pyridinyl, pyrimidinyl, purinyl, quinolinyl, isoquinolinyl, furyl, thiophenyl, imidazolyl, oxazolyl, thiazolyl, pyrazolyl, and thienyl.

Exemplary compounds in which U is N and X is Q of formula XVIII are presented in TABLE VII.

TABLE VII

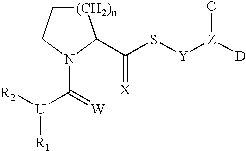

| No. | n | W | Y | Z | C | O | R₁ | R₂ |
|---|---|---|---|---|---|---|---|---|
| 101 | 1 | O | (CH₂)₂ | CH | 3-Pyridyl | H | H | 2-Methylbutyl |
| 102 | 1 | O | (CH₂)₂ | CH | 3-Pyridyl | H | H | 1,1-dimethylpropyl |
| 103 | 1 | O | (CH₂)₂ | CH | 4-Methoxyphenyl | H | H | 1,1-dimethylpropyl |
| 104 | 1 | O | CH₂ | CH | Phenyl | H | H | 1,1-dimethylpropyl |
| 105 | 1 | S | (CH₂)₂ | CH | 4-Methoxyphenyl | H | H | Cyclohexyl |
| 106 | 1 | O | (CH₂)₂ | CH | 3-Pyridyl | H | H | Cyclohexyl |
| 107 | 1 | S | (CH₂)₂ | CH | 3-Pyridyl | H | H | Cyclohexyl |
| 108 | 1 | S | (CH₂)₂ | CH | 3-Pyridyl | H | H | 1-Adamantyl |
| 109 | 1 | S | (CH₂)₂ | CH | 3-Pyridyl | H | H | 1,1-dimethylpropyl |
| 110 | 1 | O | (CH₂)₂ | CH | Phenyl | Phenyl | H | 1,1-dimethylpropyl |
| 111 | 2 | O | (CH₂)₂ | CH | Phenyl | H | H | 1,1-dimethylpropyl |
| 112 | 2 | O | (CH₂)₂ | CH | Phenyl | H | H | Phenyl |
| 113 | 2 | O | Direct bond | CH | 2-Phenylethyl | 2-Phenylethyl | H | Phenyl |
| 114 | 2 | O | Direct bond | CH | 2-Phenylethyl | 2-Phenylethyl | H | Cyclohexyl |
| 115 | 2 | S | Direct bond | CH | 2-Phenylethyl | 2-Phenylethyl | H | Cyclohexyl |
| 116 | 2 | O | (CH₂)₂ | CH | 4-Methoxyphenyl | H | H | Cyclohexyl |

The most preferred compounds of formula XVIII are selected from the group consisting of:

3-(3-Pyridyl)-1-propyl-2S-1-[(2-methylbutyl) carbamoyl]pyrrolidine-2-carboxylate;

3-(3-Pyridyl)-1-propyl-2S-1-[(1′,1′-Dimethylpropyl) carbamoyl]pyrrolidine-2-carboxylate;

3-(3-Pyridyl)-1-propyl-2S-1-[(cyclohexyl) thiocarbamoyl]pyrrolidine-2-carboxylate; and

pharmaceutically acceptable salts, esters, and solvates thereof.

### Formula XIX

Additionally, the neurotrophic agent may be a compound of formula XIX:

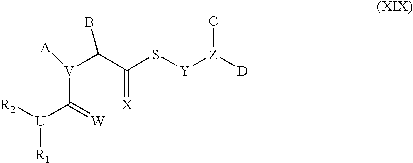

or a pharmaceutically acceptable salt, ester, or solvate thereof, wherein:

V is CH, N, or S;

Y is a direct bond, C₁-C₆ straight or branched-chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₃ is selected from the group consisting of hydrogen, C₁-C₆ straight or branched chain alkyl, C₃-C₆ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein said heterocyclic ring contains 1-6 heteroatom(s) independently selected from the group consisting of O, N, and S; and wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₃, S, SO, or SO₂; and

A, B, R₁, R₂, U, W, and X are as otherwise defined in formula XV.

V. N-Linked Sulfonamides of Heterocyclic Thioesters

### Formula XX

The neurotrophic agent may further be a compound of formula XX:

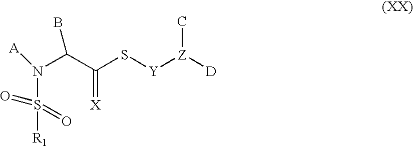

a pharmaceutically acceptable salt, ester, or solvate thereof, wherein:

A and B, together with the nitrogen and carbon atoms to which they are respectively attached, form a 5-7 membered saturated or unsaturated heterocyclic ring containing, in addition to the nitrogen atom, one or more heteroatom(s) independently selected from the group consisting of O, S, SO, SO₂, N, NH, and NR₂;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₃, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and

R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, and sulfonyl, wherein any carbon atom of said alkyl or alkenyl is optionally replaced with O, NH, NR₂, S, SO, or SO₂.

In a preferred embodiment of formula XX, Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

In another preferred embodiment of formula XX, A and B, together with the nitrogen and carbon atoms to which they are respectfully attached, form a 6 membered saturated or unsaturated heterocyclic ring; and R₂ is C₄-C₇ branched chain alkyl, C₄-C₇ cycloalkyl, phenyl, or 3,4,5-trimethoxyphenyl.

In the most preferred embodiment of formula XX, the compound is selected from the group consisting of:

3-(para-Methoxyphenyl)-1-propylmercaptyl(2S)-N-(benzenesulfonyl)pyrrolidine-2-carboxylate;

3-(para-Methoxyphenyl)-1-propylmercaptyl(2S)-N-(α-toluenesulfonyl)pyrrolidine-2-carboxylate;

3-(para-Methoxyphenyl)-1-propylmercaptyl(2S)-N-(α-toluenesulfonyl)pyrrolidine-2-carboxylate;

1,5-Diphenyl-3-pentylmercaptyl N-(paratoluenesulfonyl)pipecolate; and

pharmaceutically acceptable salts, esters, and solvates thereof.

### Formula XXI

Moreover, the neurotrophic agent may be a compound of formula XXI:

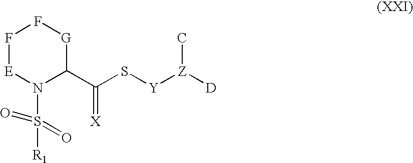

or a pharmaceutically acceptable salt, ester, or solvate thereof, wherein:

E, F, G and J are independently CH₂, O, S, SO, SO₂, NH or NR₂;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any carbon atom of said alkyl or alkenyl is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, sulfonyl, or oxygen to form a carbonyl, or wherein any atom of said alkyl or alkenyl is optionally replaced with O, NH, NR₂, S, SO, or SO₂;

Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;

C and D are independently hydrogen, Ar, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₆-alkyl, C₂-C₆ alkenyl, hydroxy, amino, halo, halo-C₁-C₆-alkyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C₆-alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NH, NR₂, S, SO, or SO₂; and

R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-C₁-C₆-alkyl, hydroxy, trifluoromethyl, C₁-C₆ straight or branched chain alkyl, C₂-C₆ straight or branched chain alkenyl, carbonyl, thiocarbonyl, C₁-C₆-ester, thio-C₁-C₆-ester, C₁-C₆-alkoxy, C₂-C₆-alkenoxy, cyano, nitro, imino, C₁-C₆-alkylamino, amino-C₁-C₆-alkyl, sulfhydryl, thio-C₁-C6-alkyl, and sulfonyl, wherein any carbon atom of said alkyl or alkenyl is optionally replaced with O, NH, NR₂, S, SO, or SO₂.

In a preferred embodiment of formula XXI, Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

### Formula XXII

The neurotrophic agent may also be a compound of formula XXII:

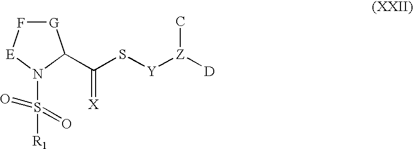

or a pharmaceutically acceptable salt, ester, or solvate thereof, wherein:

E, F, and G are independently CH₂, O, S, SO, SO₂, NH or NR₂;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any atom of said alkyl or alkenyl is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

C and D are independently hydrogen, Ar, C₁-C₆ straight or branched chain alkyl, or C₂-C₆ straight or branched chain alkenyl, wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₄ alkyl, C₂-C₄ alkenyl, or hydroxy; wherein any carbon atom of said alkyl or alkenyl is optionally substituted in one or more position(s) with oxygen to form a carbonyl, or wherein any carbon atom of said alkyl or alkenyl is optionally replaced with O, NH, NR₂, S, SO, or SO₂; and

R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-(C₁-C₆)-alkyl, hydroxy, trifluoromethyl, C₁-C₆ straight or branched chain alkyl, C₂-C₆ straight or branched chain alkenyl, carbonyl, thiocarbonyl, (C₁-C₆) -ester, thio- (C₁-C₆) -ester, (C₁-C₆)-alkoxy, (C₂-C₆) -alkenoxy, cyano, nitro, imino, (C₁-C₆)-alkylamino, amino-(C₁-C₆) -alkyl, sulfhydryl, thio-(C₁-C₆)-alkyl, and sulfonyl, wherein any carbon atom of said alkyl or alkenyl is optionally replaced with O, NH, NR₂, S, SO, or SO₂.

In a preferred embodiment of formula XXII, Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

### Formula XXIII

Additionally, the neurotrophic agent may be a compound of formula XXIII:

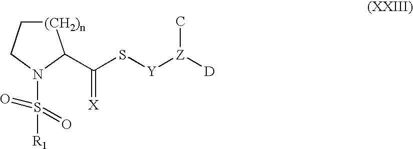

or a pharmaceutically acceptable salt, ester, or solvate thereof, wherein:

n is 1, 2 or 3;

X is either O or S;

Y is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any carbon atom of said alkyl or alkenyl is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Z is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, halo-(C₁-C₆)-alkyl, thiocarbonyl, (C₁-C₆)-ester, thio-(C₁-C₆)-ester, (C₁-C₆)-alkoxy, (C₂-C₆)-alkenoxy, cyano, nitro, imino, (C₁-C₆)-alkylamino, amino-(C₁-C₆)-alkyl, sulfhydryl, thio-(C₁-C₆)-alkyl, sulfonyl, or oxygen to form a carbonyl, or wherein any atom of said alkyl or alkenyl is optionally replaced with O, NH, NR₂, S, SO, or SO₂;

R₂ is selected from the group consisting of hydrogen, C₁-C₄ straight or branched chain alkyl, C₃-C₄ straight or branched chain alkenyl or alkynyl, and C₁-C₄ bridging alkyl wherein a bridge is formed between the nitrogen and a carbon atom of said alkyl or alkenyl chain containing said heteroatom to form a ring, wherein said ring is optionally fused to an Ar group;

Ar is an alicyclic or aromatic, mono-, bi- or tricyclic, carbo- or heterocyclic ring, wherein the ring is either unsubstituted or substituted with one or more substituent(s); wherein the individual ring size is 5-8 members; wherein the heterocyclic ring contains 1-6 heteroatom(s) independently selected from the group consisting of O, N, and S; wherein any aromatic or tertiary alkyl amine is optionally oxidized to a corresponding N-oxide;

C and D are independently hydrogen, Ar, C₁-C₆ straight or branched chain alkyl, or C₂-C₆ straight or branched chain alkenyl, wherein said alkyl or alkenyl is optionally substituted with one or more substituent(s) independently selected from the group consisting of C₃-C₈ cycloalkyl, C₅-C₇ cycloalkenyl, hydroxy, carbonyl oxygen, and Ar; wherein said alkyl, alkenyl, cycloalkyl or cycloalkenyl is optionally substituted with C₁-C₄ alkyl, C₂-C₄ alkenyl, or hydroxy; wherein any carbon atom of said alkyl or alkenyl is optionally substituted in one or more position(s) with oxygen to form a carbonyl, or wherein any carbon atom of said alkyl or alkenyl is optionally replaced with O, NH, NR₂, S, SO, or SO₂; and

R₁ is selected from the group consisting of Ar, C₃-C₈ cycloalkyl, C₁-C₆ straight or branched chain alkyl, and C₂-C₆ straight or branched chain alkenyl, wherein said alkyl or alkenyl is optionally substituted with one or more substituent(s) independently selected from the group consisting of Ar, C₃-C₈ cycloalkyl, amino, halo, halo-(C₁-C₆)-alkyl, hydroxy, trifluoromethyl, C₁-C₆ straight or branched chain alkyl, C₂-C₆ straight or branched chain alkenyl, carbonyl, thiocarbonyl, (C₁-C₆)-ester, thio-(C₁-C₆) -ester, (C₁-C₆) -alkoxy, (C₂-C₆) -alkenoxy, cyano, nicro, imino, (C₁-C₆)-alkylamino, amino-(C₁-C₆)-alkyl, sulfhydryl, thio-(C₁-C₆)-alkyl, and sulfonyl, wherein any carbon atom of said alkyl or alkenyl is optionally replaced with O, NH, NR₃, S, SO, or SO₂.

In a preferred embodiment of formula XXIII, Ar is selected from the group consisting of phenyl, benzyl, naphthyl, indolyl, pyridyl, pyrrolyl, pyrrolidinyl, pyridinyl, pyrimidinyl, purinyl, quinolinyl, isoquinolinyl, furyl, fluorenyl, thiophenyl, imidazolyl, oxazolyl, thiazolyl, pyrazolyl, and thienyl.

Exemplary compounds of formula XXIII are presented in TABLE VIII:

TABLE VIII

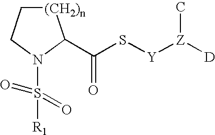

| No. | n | Y | Z | C | D | R₁ |
|---|---|---|---|---|---|---|
| 117 | 1 | CH₂ | CH | Phenyl | H | Phenyl |
| 118 | 1 | CH₂ | CH | Phenyl | H | α- |
| | | | | | | Methylphenyl |
| 119 | 1 | CH₂ | CH | Phenyl | H | 4- |
| | | | | | | Methylphenyl |
| 120 | 1 | (CH₂)₂ | CH | ρ-Methoxyphenyl | H | Phenyl |
| 121 | 1 | (CH₂)₂ | CH | ρ-Methoxyphenyl | H | α- |
| | | | | | | Methylphenyl |
| 122 | 1 | (CH₂)₂ | CH | ρ-Methoxyphenyl | H | 4- |
| | | | | | | Methylphenyl |
| 123 | 1 | (CH₂)₂ | CH | Phenyl | Phenyl | Phenyl |
| 124 | 1 | (CH₂)₂ | CH | Phenyl | Phenyl | |
| | | | | | | Methylphenyl |
| 125 | 1 | (CH₂)₂ | CH | Phenyl | Phenyl | 4- |
| | | | | | | Methylphenyl |
| 126 | 2 | (CH₂)₃ | CH | Phenyl | H | Phenyl |
| 127 | 2 | (CH₂)₃ | CH | Phenyl | H | α- |
| | | | | | | Methylphenyl |
| 128 | 2 | (CH₂)₃ | CH | Phenyl | H | 4- |
| | | | | | | Methylphenyl |
| 129 | 2 | (CH₂)₃ | CH | Phenyl | H | 3,4,5- |
| | | | | | | trimethoxy- |
| | | | | | | phenyl |
| 130 | 2 | (CH₂)₃ | CH | Phenyl | H | Cyclohexyl |
| 131 | 2 | Direct | CH | 3-Phenylpropyl | 3- | Phenyl |
| | | bond | | | Phenyl- | |
| | | | | | propyl | |
| 132 | 2 | Direct | CH | 3-Phenylpropyl | 3- | α- |
| | | bond | | | Phenyl- | Methylphenyl |
| | | | | | propyl | |
| 133 | 2 | Direct | CH | 3-Phenylpropyl | 3- | 4- |
| | | bond | | | Phenyl- | Methylphenyl |
| | | | | | propyl | |
| 134 | 2 | Direct | CH | 3-Phenylethyl | 3- | 4- |
| | | | | | Phenyl- | |
| | | | | | ethyl | |
| | | bond | | | | Methylphenyl |
| 135 | 2 | Direct | CH | 3-(4- | 3- | 4- |
| | | bond | | Methoxyphenyl)- | Phenyl- | Methylphenyl |
| | | | | propyl | propyl | |
| 136 | 2 | Direct | CH | 3-(2- | 3- | 4- |
| | | bond | | Pyridyl)propyl | Phenyl- | Methylphenyl |
| | | | | | propyl | |

The most preferred compounds of formula XXIII are selected from the group consisting of:

3-(para-Methoxyphenyl)-1-propylmercaptyl(2S)-N-(benzenesulfonyl)pyrrolidine-2-carboxylate;

3-(para-Methoxyphenyl)-1-propylmercaptyl(2S)-N-(α-toluenesulfonyl)pyrrolidine-2-carboxylate;

3-(para-Methoxyphenyl)-1-propylmercaptyl(2S)-N-(α-toluenesulfonyl)pyrrolidine-2-carboxylate;

1,5-Diphenyl-3-pentylmercaptyl N-(paratoluenesulfonyl)pipecolate; and

pharmaceutically acceptable salts, esters, and vates thereof.

### Formula XXIV

Moreover, the neurotrophic agent may be a compound of formula XXIV:

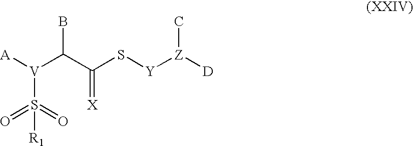

or a pharmaceutically acceptable salt, ester, or solvate thereof, wherein:

V is CH, N, or S;

A, B, C, D, R₁, X, Y, and Z are as defined in formula XX above.

VI. Pyrrolidine Derivatives

### Formula XXV

The neurotrophic agent may also be a compound of formula XXV:

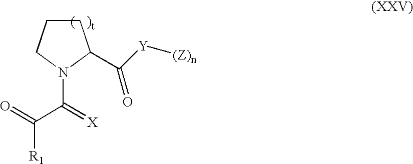

or a pharmaceutically acceptable salt, ester, or solvate thereof, wherein:

R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;

Ar₁ and Ar₂ are independently selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₆ alkenyloxy, phenoxy, benzyloxy, and amino;

X is O, S, CH₂ or H₂;

Y is O or NR₂, wherein R₂ is a direct bond to a Z, hydrogen or C₁-C₆ alkyl; and

each Z, independently, is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment

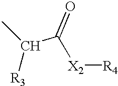

wherein:

R₃ is C₁-C₉ straight or branched chain alkyl which is unsubstituted or substituted with C₃-C₈ cycloalkyl or Ar₁;

X₂ is O or NR₅, wherein R₅ is selected from the group consisting of hydrogen, C₁-C₆ straight or branched chain alkyl, and C₂-C₆ straight or branched chain alkenyl;

R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl;

n is 1 or 2, and;

t is 1, 2 or 3.

In a preferred embodiment of formula XXV, Z and R₁ are lipophilic.

In a more preferred embodiment of formula XXV, the compound is selected from the group consisting of:

3-phenyl-1-propyl (2S)-1-(3,3-dimethyl-1,2-dioxopentyl)-2-pyrrolidinecarboxylate;

3-phenyl-1-prop-2-(E)-enyl (2S)-1-(3,3-dimethyl-1,2-dioxopentyl)-2-pyrrolidinecarboxylate;

3-(3,4,5-trimethoxyphenyl)-1-propyl (2S)-1-(3,3-dimethyl-1,2-dioxopentyl)-2-pyrrolidine-carboxylate;

3-(3,4,5-trimethoxyphenyl)-1-prop-2-(E)-enyl (2S)-1-(3,3-dimethyl-1,2-dioxopentyl)-2-pyrrolidinecarboxylate;

3-(4,5-dichlorophenyl)-1-propyl (2S)-1-(3,3-dimethyl-1,2-dioxopentyl)-2-pyrrolidinecarboxylate;

3-(4,5-dichlorophenyl)-1-prop-2-(E)-enyl (2S)-1-(3,3-dimethyl-1,2-dioxopentyl)-2-pyrrolidine-carboxylate;

3-(4,5-methylenedioxyphenyl)-1-propyl (2S)-1-(3,3-dimethyl-1,2-dioxopentyl)-2-pyrrolidine-carboxylate;

3-(4,5-methylenedioxyphenyl)-1-prop-2-(E)-enyl (2S)-1-(3,3-dimethyl-1,2-dioxopentyl)-2-pyrrolidinecarboxylate;

3-cyclohexyl-1-propyl (2S)-1-(3,3-dimethyl-1,2-dioxopentyl)-2-pyrrolidinecarboxylate;

3-cyclohexyl-1-prop-2-(E)-enyl (2S)-1-(3,3-dimethyl-1,2-dioxopentyl)-2-pyrrolidinecarboxylate;

(1R)-1,3-diphenyl-1-propyl (2S)-1-(3,3-dimethyl-1,2-dioxopentyl)-2-pyrrolidinecarboxylate;

(1R)-1,3-diphenyl-1-prop-2-(E)-enyl (2S)-1-(3,3-dimethyl-1,2-dioxopentyl)-2-pyrrolidine-carboxylate;

(1R)-1-cyclohexyl-3-phenyl-1-propyl (2S)-1-(3,3-dimethyl-1,2-dioxopentyl)-2-pyrrolidine-carboxylate;

(1R)-1-cyclohexyl-3-phenyl-1-prop-2-(E)-enyl (2S)-1-(3,3-dimethyl-1,2-dioxopentyl)-2-pyrrolidinecarboxylate;

(1R)-1-(4,5-dichlorophenyl)-3-phenyl-1-propyl (2S)-1-(3,3-dimethyl-1,2-dioxopentyl)-2-pyrrolidine-carboxylate;

3-phenyl-1-propyl (2S)-1-(1,2-dioxo-2-cyclohexyl)ethyl-2-pyrrolidinecarboxylate;

3-phenyl-1-propyl (2S)-1-(1,2-dioxo-4-cyclohexyl)butyl-2-pyrrolidinecarboxylate;

3-phenyl-1-propyl (2S)-1-(1,2-dioxo-2-[2-furanyl])ethyl-2-pyrrolidinecarboxylate;

3-phenyl-1-propyl (2S)-1-(1,2-dioxo-2-[2-thienyl])ethyl-2-pyrrolidinecarboxylate;

3-phenyl-1-propyl (2S)-1-(1,2-dioxo-2-[2-thiazolyl])ethyl-2-pyrrolidinecarboxylate;

3-phenyl-1-propyl (2S)-1-(1,2-dioxo-2-phenyl)ethyl-2-pyrrolidinecarboxylate;

1,7-diphenyl-4-heptyl (2S)-1-(3,3-dimethyl-1,2-dioxopentyl)-2-pyrrolidinecarboxylate;

3-phenyl-1-propyl (2S)-1-(3,3-dimethyl-1,2-dioxo-4-hydroxybutyl)-2-pyrrolidinecarboxylate;

3-phenyl-1-propyl (2S)-1-(3,3-dimethyl-1,2-dioxopentyl)-2-pyrrolidinecarboxamide;

1-[1-(3,3-dimethyl-1,2-dioxopentyl)-L-proline]-L-phenylalanine ethyl ester;

1-[1-(3,3-dimethyl-1,2-dioxopentyl)-L-proline]-L-leucine ethyl ester;

1-[1-(3,3-dimethyl-1,2-dioxopentyl)-L-proline]1-L-phenylglycine ethyl ester;

1-[1-(3,3-dimethyl-1,2-dioxopentyl)-L-proline]-L-phenylalanine phenyl ester;

1-[1-(3,3-dimethyl-1,2-dioxopentyl)-L-prolinel-L-phenylalanine benzyl ester;

1-[1-(3,3-dimethyl-1,2-dioxopentyl)-L-proline]-L-isoleucine ethyl ester; and

pharmaceutically acceptable salts, esters, and solvates thereof.

### Formula XXVI

Additionally, the neurotrophic agent may be a compound of formula XXVI:

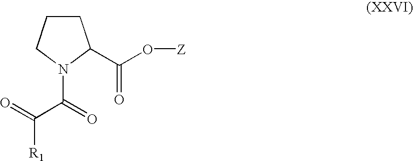

or a pharmaceutically acceptable salt, ester, or solvate thereof, wherein:

R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₈ cycloalkyl, C₅-C₇ cycloalkenyl, hydroxy, and Ar₂;

Ar₁ and Ar₂ are independently selected from the group consisting of 1-napthyl, 2-napthyl, 2-indolyl, 3-indolyl, 2-furyl, 3-furyl, 2-thienyl, 3-thienyl, 2-pyridyl, 3-pyridyl, 4-pyridyl and phenyl, wherein said Ar₁ is unsubstituted or substituted with one or more substituent(s) independently selected from the group consisting of hydrogen, halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino;

Z is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment

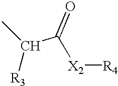

wherein:

R₃ is C₁-C₉ straight or branched chain alkyl which is unsubstituted or substituted with C₃-C₈ cycloalkyl or Ar₁;

X₂ is O or NR₅, wherein R₅ is selected from the group consisting of hydrogen, C₁-C₆ straight or branched chain alkyl, and C₂-C₆ straight or branched chain alkenyl; and

R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl.

In a preferred embodiment of formula XXVI, R₁ is selected from the group consisting of C₁-C₉ straight or branched chain alkyl, 2-cyclohexyl, 4-cyclohexyl, 2-furanyl, 2-thienyl, 2-thiazolyl, and 4-hydroxybutyl.

In another preferred embodiment of formula XXVI, Z and R₁ are lipophilic.

### Formula XXVII

Furthermore, the neurotrophic agent may be a compound of formula XXVII:

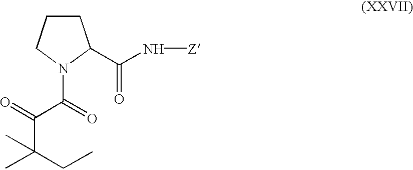

or a pharmaceutically acceptable salt, ester, or solvate thereof, wherein:

Z′ is the fragment

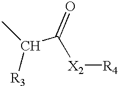

wherein:

R₃ is C₁-C₉ straight or branched chain alkyl or unsubstituted Ar₁, wherein said alkyl is unsubstituted or substituted with C₃-C₈ cycloalkyl or Ar₁;

X₂ is O or NR₅, wherein R₅ is selected from the group consisting of hydrogen, C₁-C₆ straight or branched chain alkyl, and C₂-C₆ straight or branched chain alkenyl;

R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl; and

Ar₁ is as defined in formula XXVI.

In a preferred embodiment of formula XXVII, Z′ is lipophilic.

### Formula XXVIII

The neurotrophic agent may also be a compound of formula XXVIII:

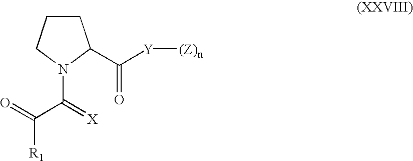

wherein:

R₁ is C₁-C₆ straight or branched chain alkyl, C₂-C₆ straight or branched chain alkenyl, C₃-C₆ cycloalkyl or Ar₁, wherein said alkyl or alkenyl is unsubstituted or substituted with C₃-C₆ cycloalkyl or Ar₂;

Ar₁ and Ar₂ are independently selected from the group consisting of 2-furyl, 2-thienyl, and phenyl;

X is selected from the group consisting of oxygen and sulfur;

Y is oxygen or NR₂, wherein R₂ is a direct bond to a Z, hydrogen or C₁-C₆ alkyl;

Z is hydrogen, C₁-C₆ straight or branched chain alkyl, or C₂-C₆ straight or branched chain alkenyl, wherein said Z is substituted with one or more substituent(s) independently selected from the group consisting of 2-furyl, 2-thienyl, C₃-C₆ cycloalkyl, pyridyl, and phenyl, each having one or more substituent(s) independently selected from the group consisting of hydrogen and C₁-C₄ alkoxy; and n is 1 or 2.

In a preferred embodiment of formula XXVIII, Z and R₁ are lipophilic.

In another preferred embodiment of formula XXVIII, the compound is selected from the group consisting of:

3-(2,5-dimethoxyphenyl)-1-propyl (2S)-1-(3,3-dimethyl-1,2-dioxopentyl)-2-pyrrolidinecarboxylate;

3-(2,5-dimethoxyphenyl)-1-prop-2-(E)-enyl (2S)-1-(3,3-dimethyl-1,2-dioxopentyl)-2-pyrrolidine-carboxylate;

2-(3,4,5-trimethoxyphenyl)-1-ethyl (2S)-1-(3,3-dimethyl-1,2-dioxopentyl)-2-pyrrolidinecarboxylate;

3-(3-pyridyl)-1-propyl (2S)-1-(3,3-dimethyl-1,2-dioxopentyl)-2-pyrrolidinecarboxylate;

3-(2-pyridyl)-1-propyl (2S)-1-(3,3-dimethyl-1,2-dioxopentyl)-2-pyrrolidinecarboxylate;

3-(4-pyridyl)-1-propyl (2S)-1-(3,3-dimethyl-1,2-dioxopentyl)-2-pyrrolidinecarboxylate;

3-phenyl-1-propyl (2S)-1-(2-tert-butyl-1,2-dioxoethyl)-2-pyrrolidinecarboxylate;

3-phenyl-1-propyl (2S)-1-(2-cyclohexylethyl-1,2-dioxoethyl)-2-pyrrolidinecarboxylate;

3-(3-pyridyl)-1-propyl (2S)-1-(2-cyclohexylethyl-1,2-dioxoethyl)-2-pyrrolidine-carboxylate;

3-(3-pyridyl)-1-propyl (2S)-1-(2-tert-butyl-1,2-dioxoethyl)-2-pyrrolidinecarboxylate;

3,3-diphenyl-1-propyl (2S)-1-(3,3-dimethyl-1,2-dioxopentyl)-2-pyrrolidinecarboxylate;

3-(3-pyridyl)-1-propyl (2S)-1-(2-cyclohexyl-1,2-dioxoethyl)-2-pyrrolidinecarboxylate;

3-(3-pyridyl)-1-propyl (2S)-N-([2-thienyl]glyoxyl)pyrrolidinecarboxylate;

3,3-diphenyl-1-propyl (2S)-1-(3,3-dimethyl-1,2-dioxobutyl)-2-pyrrolidinecarboxylate;

3,3-diphenyl-1-propyl (2S)-1-cyclohexylglyoxyl-2-pyrrolidinecarboxylate;

3,3-diphenyl-1-propyl (2S)-1-(2-thienyl)glyoxyl-2-pyrrolidinecarboxylate; and

pharmaceutically acceptable salts, esters, and solvates thereof.

In a more preferred embodiment of formula XXVIII, the compound is selected from the group consisting of:

3-(3-pyridyl)-1-propyl (2S)-1-(3,3-dimethyl-1,2-dioxopentyl)-2-pyrrolidinecarboxylate;

3-(2-pyridyl)-1-propyl (2S)-1-(3,3-dimethyl-1,2-dioxopentyl)-2-pyrrolidinecarboxylate;

3-(3-pyridyl)-1-propyl (2S)-1-(2-cyclohexyl-1,2-dioxoethyl)-2-pyrrolidinecarboxylate; and pharmaceutically acceptable salts, esters, and solvates thereof.

In the most preferred embodiment of formula XXVIII, the compound is 3-(3-pyridyl)-1-propyl (2S)-1-(3,3-dimethyl-1,2-dioxopentyl)-2-pyrrolidine-carboxylate, and pharmaceutically acceptable salts, esters, and solvates thereof.

### Formula XXIX

Additionally, the neurotrophic agent may be a compound of formula XXIX:

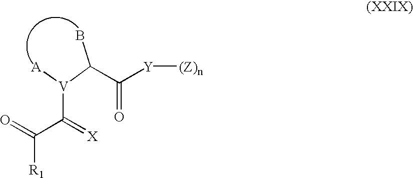

or a pharmaceutically acceptable salt, ester, or solvate thereof, wherein:

V is CH, N, or S;

A and B, together with V and the carbon atom to which they are respectively attached, form a 5-7 membered saturated or unsaturated heterocyclic ring containing, in addition to V, one or more heteroatom(s) independently selected from the group consisting of O, S, SO, SO₂, N, NH, and NR;

R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo-(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, alkylthio, sulfhydryl, amino, (C₁-C₆)-alkylamino, amino-(C₁-C₆)-alkyl, aminocarboxyl, and Ar₂;

R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituents independently selected from the group consisting of C₁-C₆ alkyl, C₂-C₆ alkenyl, C₃-C₉ cycloalkyl, C₅-C₇ cycloalkenyl, hydroxy, and Ar₂;

Ar₁ and Ar₂ are independently an alicyclic or aromatic, mono-, bi- or tricyclic, carbo- or heterocyclic ring, wherein the ring is either unsubstituted or substituted with one or more substituent(s); wherein the individual ring size is 5-8 members; wherein said heterocyclic ring contains 1-6 heteroatom(s) independently selected from the group consisting of O, N, and S;

X is O, S, CH₂ or H₂;

Y is O or NR₂, wherein R₂ is a direct bond to a Z, hydrogen or C₁-C₆ alkyl; and

Z is C₁-C₆ straight or branched chain alkyl, or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is the fragment

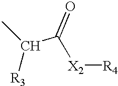

wherein:

R₃ is C₁-C₉ straight or branched chain alkyl which is unsubstituted or substituted with C₃-C₈ cycloalkyl or Ar₁;

X₂ is O or NR₅, wherein R₅ is selected from the group consisting of hydrogen, C₁-C₆ straight or branched chain alkyl, and C₂-C₆ straight or branched chain alkenyl; and

R₄ is selected from the group consisting of phenyl, benzyl, C₁-C₅ straight or branched chain alkyl, C₂-C₅ straight or branched chain alkenyl, C₁-C₅ straight or branched chain alkyl substituted with phenyl, and C₂-C₅ straight or branched chain alkenyl substituted with phenyl; and,

n is 1 or 2.

Other compounds which are neurotrophic agents within the scope of the present invention are those compounds which may possess immunosuppressive, non-immunosuppressive or other activities as long as they also are useful for the treatment of nerve injury caused as a consequence of prostate surgery. For example, such compounds may include, but are not limited to those below:

### Compound 167

Ocain et al., Biochemical and Biophysical Research Communications (1993) 3:192, incorporated herein by reference, discloses an exemplary pipecolic acid derivative represented by Formula XXX. This compound is prepared by reacting 4-phenyl-1,2,4-triazoline-3,5-dione with rapamycin.

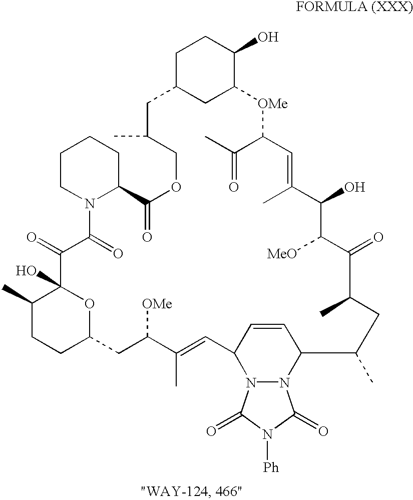

### Compound 168

Chakraborty et al., Chemistry and Biology (1995) 2:157-161, incorporated herein by reference, discloses an exemplary pipecolic acid derivative represented by Formula XXXI.

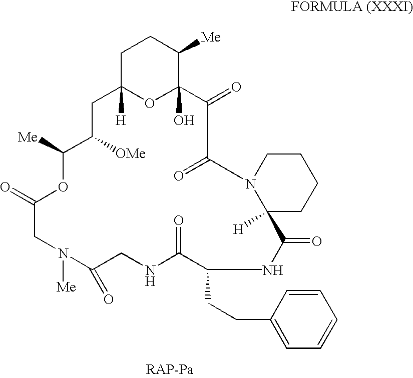

### Compounds 169-171

Ikeda et al., J. Am. Chem. Soc. (1994) 116:4143-4144, incorporated herein by reference, discloses exemplary pipecolic acid derivatives represented by Formula XXXII and Table XII.

TABLE XII

| | |
|---|---|
| | Formula (XXXII) |
| 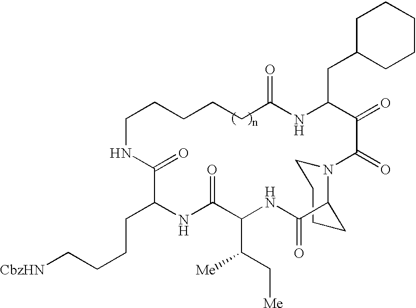 | |

| Compound | Structure |
|---|---|
| 169 | n = 1 |
| 170 | n = 2 |
| 171 | n = 3 |

### Compounds 172-175

Wang et al., Bioorganic & Medicinal Chemistry Letters (1994) 4:1161-1166, 9, incorporated herein by reference, discloses exemplary pipecolic acid derivatives represented by Formula XXXIII and Table XIII.

TABLE XIII

| | |
|---|---|
| | FORMULA (XXXIII) |
| 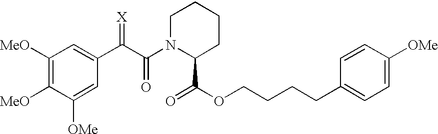 | |

| Compound | Structure |
|---|---|
| 172 | X = H, H |
| 173 | X = CH₂ |
| 174 | X = H, CH₃ |
| 175 | X = O |

### Compound 176

Birkenshaw et al., Bioorganic & Medicinal Chemistry Letters (1994) 4(21):2501-2506, incorporated herein by reference, discloses an exemplary pipecolic acid derivative represented by Formula XXXIV:

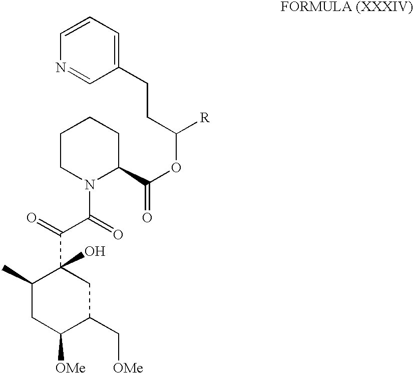

### Compounds 177-187

Holt e al., J. Am. Chem. Soc.(1993) 115:9925-9938, incorporated herein by reference, discloses exemplary pipecolic acid derivatives represented by Formula XXXV and Tables XIV and XV.

TABLE XV

| | |
|---|---|
| | FORMULA (XXXV) |
| 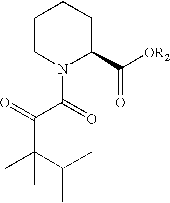 | |

| Compound | R₂ |
|---|---|
| 177 | 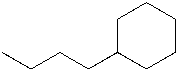 |
| 178 | 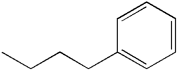 |
| 179 | 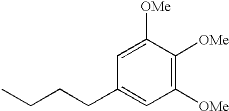 |
| 180 | 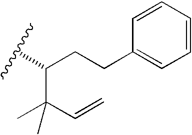 |
| 181 | 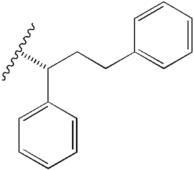 |
| 182 | 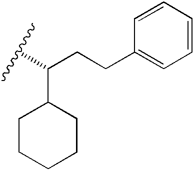 |
| 183 | 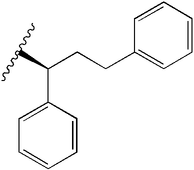 |
| 184 | 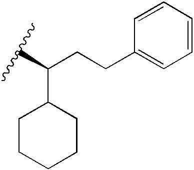 |

TABLE XV

| Compound | Structure |
|---|---|
| 185 | 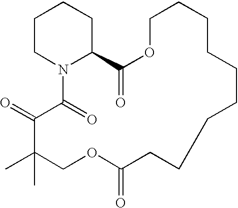 |
| 186 | 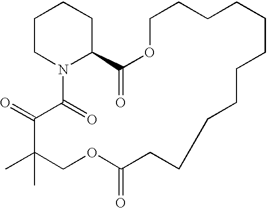 |
| 187 | 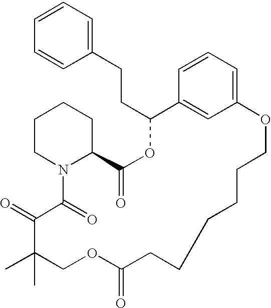 |

### Compounds 188-196

Caffery et al., Bioorganic & Medicinal Chemistry Letters (1994) 4(21):2507-2510, incorporated herein by reference, discloses exemplary pipecolic acid derivatives represented by Formulas XXXVI-XXXVIII and Tables XVI-XVIII.

TABLE XVI

| | |
|---|---|
| | FORMULA XXXVI |
| 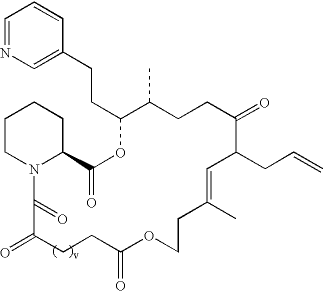 | |

| Compound | Structure |
|---|---|
| 188 | y = 1 |
| 189 | y = 2 |
| 190 | y = 3 |

TABLE XVII

| | |
|---|---|
| | FORMULA XXXVII |
| 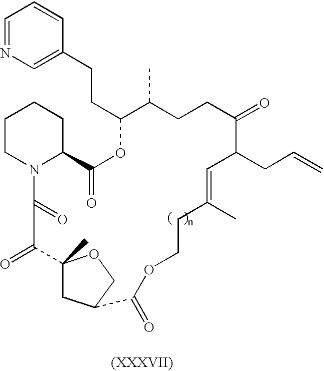 | |

| Compound | Structure |
|---|---|
| 191 | n = 1 |
| 192 | n = 2 |
| 193 | n = 3 |

TABLE XVIII

| | |
|---|---|
| | FORMULA XXXVIII |
| 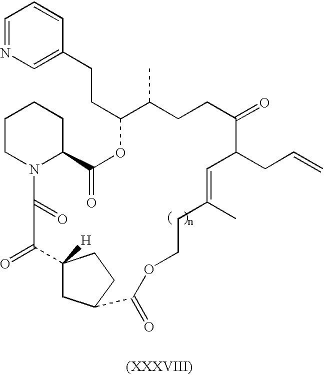 | |

| Compound | Structure |
|---|---|
| 194 | n = 1 |
| 195 | n = 2 |
| 196 | n = 3 |

### Compound 197

Teague et al., Bioorganic & Medicinal Chemistry Letters (1993) 3(10):1947-1950, incorporated herein by reference, discloses an exemplary pipecolic acid derivative represented by Formula XXXIX.

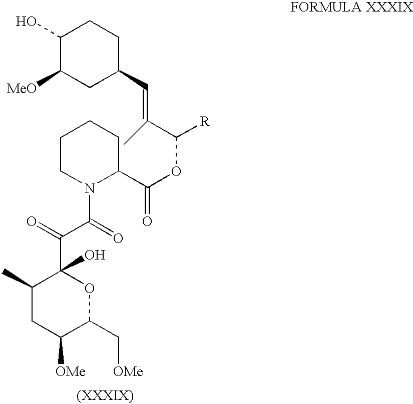

### Compounds 198-200

Yamashita et al., Bioorganic & Medicinal Chemistry Letters (1994) 4(2):325-328, incorporated herein by reference, discloses exemplary pipecolic acid derivatives represented by Formula XL and Table XIX.

TABLE XIX

| | |
|---|---|
| | FORMULA XL |
| 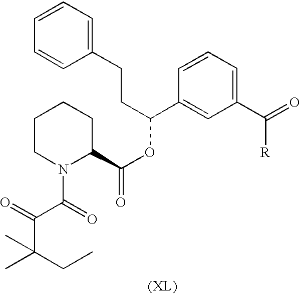 | |

| Compound | Structure |
|---|---|
| 198 | R = phenyl |
| 199 | R = N(allyl)₂ |
| 200 | 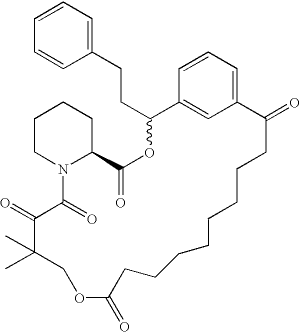 |

### Compounds 201-221

Holt et al., Bioorganic & Medicinal Chemistry Letters(1994) 4(2):315-320, incorporated herein by reference, discloses exemplary pipecolic acid derivatives represented by Formula XLI and Tables XX-XXII.

TABLE XX

| | |
|---|---|
| | FORMULA XLI |
| 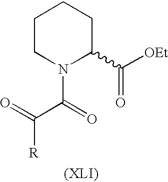 | |

| Compound No. | R |
|---|---|
| 201 | 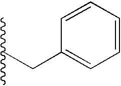 |
| 202 | 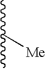 |
| 203 | 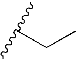 |
| 204 | 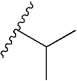 |
| 205 | 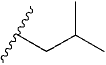 |
| 206 | 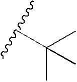 |
| 207 | 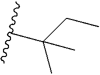 |
| 208 | 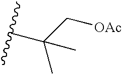 |
| 209 | 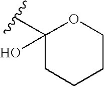 |
| 210 | 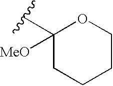 |
| 211 | 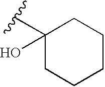 |
| 212 | 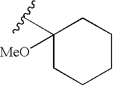 |
| 213 | 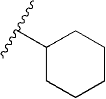 |
| 214 | 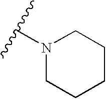 |
| 215 | 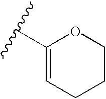 |
| 216 | 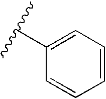 |

TABLE XXI

| Com- | |
|---|---|
| pound No. | Structure |
| 217 | 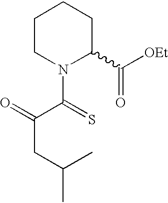 |
| 218 | 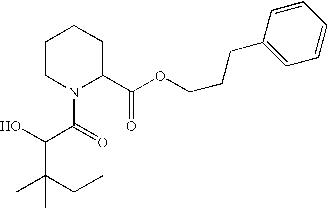 |
| 219 | 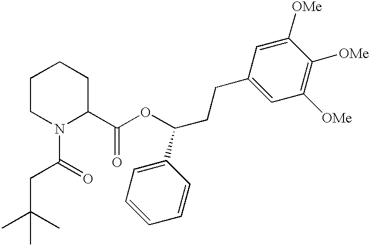 |

TABLE XXII

| Com- | |
|---|---|
| pound No. | Structure |
| 220 | 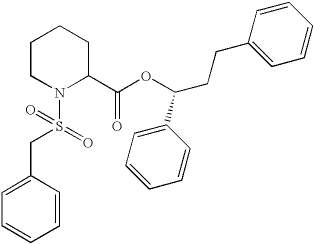 |
| 221 | 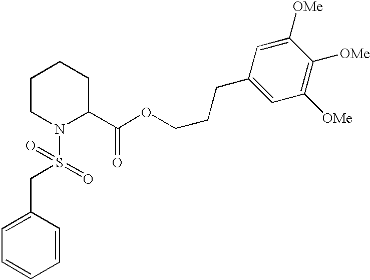 |

### Compounds 222-234

Holt et al., Bioorganic & Medicinal Chemistry Letters (1993) 3(10):1977-1980, incorporated herein by reference, discloses exemplary pipecolic acid derivatives represented by Formulas XLII and XLIII and Tables XXIII-XXV.

TABLE XXIII

| | |
|---|---|
| | FORMULA XLII |
| 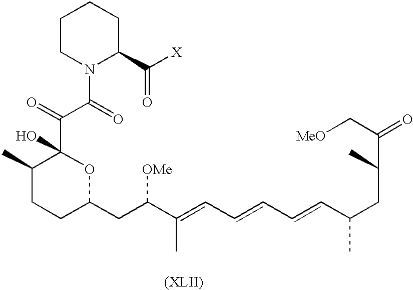 | |

| Compound | Structure |
|---|---|
| 222 | X = OH |
| 223 | X = OMe |
| 224 | X = O-iso-Pr |
| 225 | X = OBn |
| 226 | X = OCH(Me)Ph |
| 227 | X = OCH₂CHCHPh |
| 228 | X = OCH₂CH₂CH₂(3,4-OMe₂)Ph |
| 229 | X = NHBn |
| 230 | X = NHCH₂CH₂CH₂Ph |

TABLE XXIV

| | |
|---|---|
| | FORMULA XLIII |
| 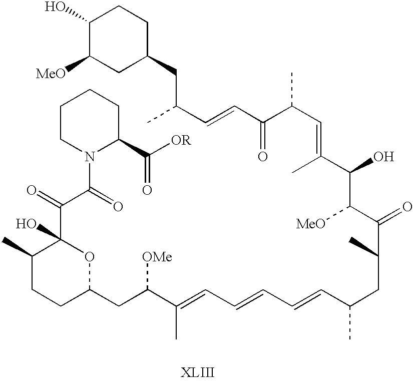 | |

| Compound | Structure |
|---|---|
| 231 | R = Me |
| 232 | R = Bn |

TABLE XXV

| Compound | Structure |
|---|---|
| 233 | 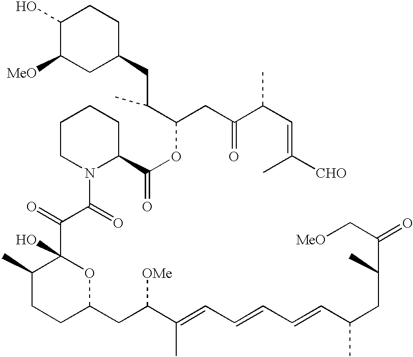 |
| 234 | 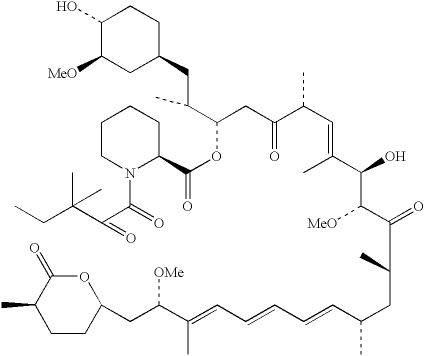 |

### Compounds 235-249

Hauske et al., J. Med. Chem. (1992) 35:4284-4296, incorporated herein by reference, discloses exemplary pipecolic acid derivatives represented by Formulas XLIV-XLVII and Tables XXVI-XXIX.

TABLE XXVI

| | |
|---|---|
| | FORMULA XLIV |
| 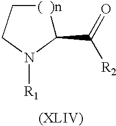 | |

| Compound | Structure | |
|---|---|---|
| 235 | n = | 2 |
| | R₁ = | 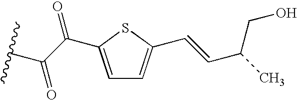 |
| | R₂ = | Phe-O-tert-butyl |
| 236 | n = | 2 |
| | R₁ = | 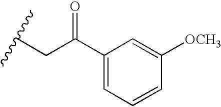 |
| | R₂ = | Phe-O-tert-butyl |

TABLE XXVII

| | |
|---|---|
| | FORMULA XLV |
| 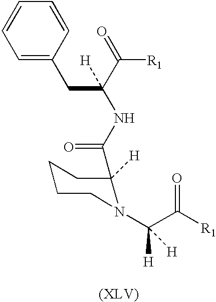 | |

| Compound | Structure |
|---|---|
| 237 | R₁ = m-OCH₃Ph |
| | R₃ = Val-O-tert-butyl |
| 236 | R₁ = m-OCH₃Ph |
| | R₃ = Leu-O-tert-butyl |
| 239 | R₁ = m-OCH₃Ph |
| | R₃ = Ileu-O-tert-butyl |
| 240 | R₁ = m-OCH₃Ph |
| | R₃ = hexahydro-Phe-O-tert-butyl |
| 241 | R₁ = m-OCH₃Ph |
| | R₃ = allylalanine-O-tert-butyl |
| 242 | R₁ = β-naphthyl |
| | R₃ = Val-O-tert-butyl |

TABLE XXVIII

| | |
|---|---|
| | FORMULA XLVI |
| 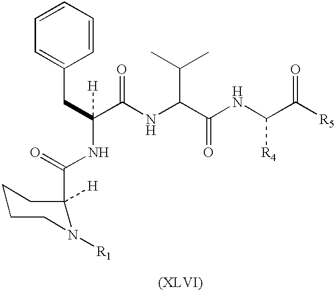 | |

| Compound | Structure |
|---|---|
| 243 | R₁ = CH₂(CO)-m-OCH₃Ph |
| | R₄ = CH₂Ph |
| | R₅ = OCH₃ |
| 244 | R₁ = CH₂(CO)-β-naphthyl |
| | R₄ = CH₂Ph |
| | R₅ = OCH₃ |

TABLE XXIX

| | |
|---|---|
| | FORMULA XLVII |
| 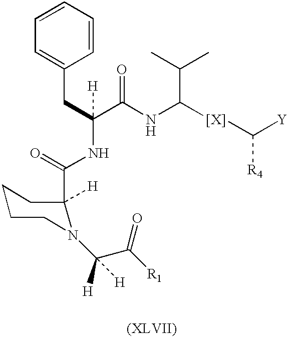 | |

| Compound | Structure |
|---|---|
| 245 | R₁ = m-OCH₃Ph |
| | X = trans-CH═CH— |
| | R₄ = H |
| | Y = OC(O)Ph |
| 246 | R₁ = m-OCH₃Ph |
| | X = trans-CH═CH |
| | R₄ = H |
| | Y = OC(O)CF₃ |
| 247 | R₁ = m-OCH₃Ph |
| | X = trans-CH═CH— |
| | R₄ = - |
| | Y = - |
| 248 | R₁ = m-OCH₃Ph |
| | X = trans-CH═CH— |
| | R₄ = H |
| | Y = OCH₂CH═CH₂ |
| 249 | R₁ = m-OCH₃Ph |
| | X = C═O |
| | R₄ = H |
| | Y = Ph |

### Compound 250

Teague et al., Bioorganic & Med. Chem. Letters (1994) 4(13):1581-1584, incorporated herein by reference, discloses an exemplary pipecolic acid derivative represented by Formula XLVIII.

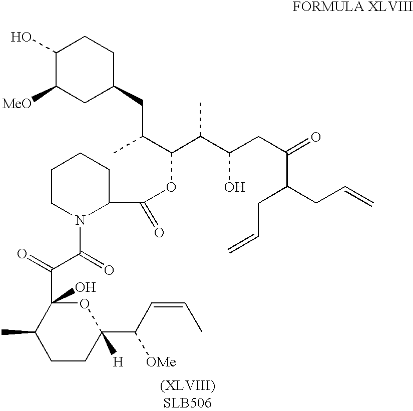

### Compounds 251-254

Stocks et al., Bioorganic & Med. Chem. Letters (1994) 4(12):1457-1460, incorporated herein by reference, discloses exemplary pipecolic acid derivatives represented by Formula XLIX and Tables XXX and XXXI.

TABLE XXX

| Compound No. | Structure |
|---|---|
| 251 | 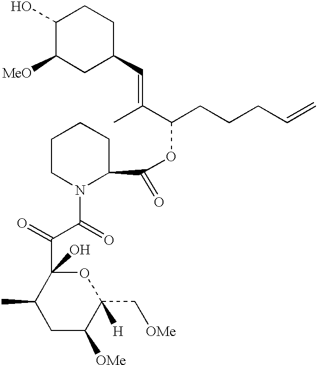 |

| | |
|---|---|
| | FORMULA XLIX |
| 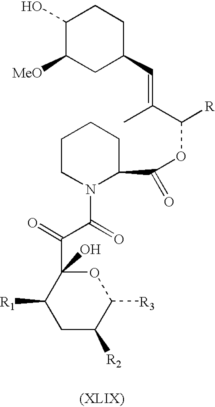 | |

TABLE XXXI

| Compound | Structure |
|---|---|
| 252 | R₁ = H |
| | R₂ = OMe |
| | R₃ = CH₂Ome |
| 253 | R₁ = H |
| | R₂ = H |
| | R₃ = H |
| 254 | R₁ = Me |

R₂=H R₃H

### Compounds 255-276

Additional exemplary pipecolic acid derivatives are represented by Formulas L-LIV and Tables XXXII-XXXVI.

TABLE XXXII

| | |
|---|---|
| | FORMULA L |
| 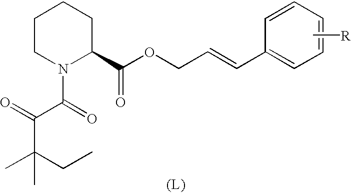 | |

| Compound | Structure |
|---|---|
| 255 | R = 3,4-dichloro |
| 256 | R = 3,4,5-trimethoxy |
| 257 | R = H |
| 258 | R = 3-(2,5-Dimethoxy)phenylpropyl |
| 259 | R = 3-(3,4-Methylenedioxy)phenylpropyl |

TABLE XXXIII

| | |
|---|---|
| | FORMULA LI |
| 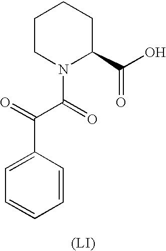 | |

| Compound | Structure |
|---|---|
| 260 | R = 4-(ρ-Methoxy)butyl |
| 261 | R = 3-Phenylpropyl |
| 262 | R = 3-(3-Pyridyl)propyl |

TABLE XXXIV

| | |
|---|---|
| | FORMULA LII |
| 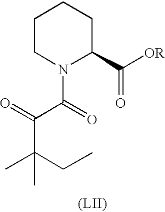 | |

| Compound | Structure |
|---|---|
| 263 | R = 3-(3-Pyridyl)propyl |
| 264 | R = 1,7-Diphenyl-4-heptyl |
| 265 | R = 4-(4-Methoxy)butyl |
| 266 | R = 1-Phenyl-6-(4-methoxyphenyl)-4-hexyl |
| 267 | R = 3-(2,5-Dimethoxy)phenylpropyl |
| 268 | R = 3-(3,4-Methylenedioxy)phenylpropyl |
| 269 | R = 1,5-Diphenylpentyl |

TABLE XXXV

| | |
|---|---|
| | FORMULA LIII |
| 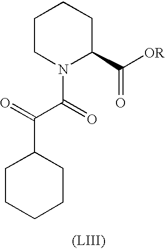 | |

| Compound | Structure |
|---|---|
| 270 | R = 4-(4-Methoxy)butyl |
| 271 | R = 3-Cyclohexylpropyl |
| 272 | R = 3-Phenylpropyl |

TABLE XXXVI

| | |
|---|---|
| | FORMULA LIV |
| 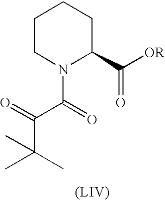 | |

| Compound | Structure |
|---|---|
| 273 | R = 3-Cyclohexylpropyl |
| 274 | R = 3-Phenylpropyl |
| 275 | R = 4-(4-Methoxy)butyl |
| 276 | R = 1,7-Diphenyl-4-heptyl |

The names of some of the compounds identified above are provided below in Table XXXVII.

TABLE XXXVII

| Compound | Name of Species |
|---|---|
| 172 | 4-(4-methoxyphenyl)butyl (2S)-1-[2-(3,4,5- |
| | trimethoxyphenyl)acetyl]hexahydro-2- |
| | pyridinecarboxylate |
| 173 | 4-(4-methoxyphenyl)butyl (2S)-1-[2-(3,4,5- |
| | trimethoxyphenyl)acryloyl]hexahydro-2- |
| | pyridinecarboxylate |
| 174 | 4-(4-methoxyphenyl)butyl (2S)-1-[2-(3,4,5- |
| | trimethoxyphenyl)propanoyl]hexahydro-2- |
| | pyridinecarboxylate |
| 175 | 4-(4-methoxyphenyl)butyl (2S)-1-[2-oxo-2- |
| | (3,4,5-trimethoxyphenyl)acetyl]hexahydro-2- |
| | pyridinecarboxylate |
| 177 | 3-cyclohexylpropyl (2S)-1-(3,3-dimethyl-2- |
| | oxopentanoyl)hexahydro-2-pyridinecarboxylate |
| 178 | 3-phenylpropyl (2S)-1-(3,3-dimethyl-2- |
| | oxopentanoyl)hexahydro-2-pyridinecarboxylate |
| 179 | 3-(3,4,5-trimethoxyphenyl)propyl (2S)-1- |
| | (3,3-dimethyl-2-oxopentanoyl)hexahydro-2- |
| | pyridinecarboxylate |
| 180 | (1R)-2,2-dimethyl-1-phenethyl-3-butenyl |
| | (2S)-1-(3,3-dimethyl-2-oxopentanoyl) |
| | hexahydro-2-pyridinecarboxylate |
| 181 | (1R)-1,3-diphenylpropyl (2S)-1-(3,3- |
| | dimethyl-2-oxopentanoyl)hexahydro-2- |
| | pyridinecarboxylate |
| 182 | (1R)-1-cyclohexyl-3-phenylpropyl (2S)-1- |
| | (3,3-dimethyl-2-oxopentanoyl)hexahydro-2- |
| | pyridinecarboxylate |
| 183 | (1S)-1,3-diphenylpropyl (2S)-1-(3,3- |
| | dimethyl-2-oxopentanoyl)hexahydro-2- |
| | pyridinecarboxylate |
| 184 | (1S)-1-cyclohexyl-3-phenylpropyl (2S)-1- |
| | (3,3-dimethyl-2-oxopentanoyl)hexahydro-2- |
| | pyridinecarboxylate |
| 185 | (22aS)-15,15-dimethylperhydropyrido[2,1- |
| | c] [1,9,4,]dioxazacyclononadecine-1,12,16,17- |
| | tetraone |
| 186 | (24aS)-17,17-dimethylperhydropyrido[2,1- |
| | c] [1,9,4]dioxazacyclohenicosine-1,14,18,19- |
| | tetraone |
| 201 | ethyl 1-(2-oxo-3phenylpropanoyl)-2- |
| | piperidinecarboxylate |
| 202 | ethyl 1-pyruvoyl-2-piperidinecarboxylate |
| 203 | ethyl 1-(2-oxobutanoyl)-2-piperidine- |
| | carboxylate |
| 204 | ethyl 1-(3-methyl-2-oxobutanoyl)-2- |
| | piperidinecarboxylate |
| 205 | ethyl 1-(4-methyl-2-oxopentanoyl)-2- |
| | piperidinecarboxylate |
| 206 | ethyl 1-(3,3-dimethyl-2-oxobutanoyl)-2- |
| | piperidinecarboxylate |
| 207 | ethyl 1-(3,3-dimethyl-2-oxopentanoyl)-2- |
| | piperidinecarboxylate |
| 208 | 4-[2-(ethyloxycarbonyl)piperidino]-2,2- |
| | dimethyl-3,4-dioxobutyl acetate |
| 209 | ethyl□1-[2-(2-hydroxytetrahydro-2H-2- |
| | pyranyl)-2-oxoacetyl]-2- |
| | piperidinecarboxylate |
| 210 | ethyl□1-[2-(2-methoxytetrahydro-2H-2- |
| | pyranyl)-2-oxoacetyl]-2- |
| | piperidinecarboxylate |
| 211 | ethyl 1-[2-(1-hydroxycyclohexyl)-2- |
| | oxoacetyl]-2-piperidinecarboxylate |
| 212 | ethyl 1-[2-(1-methoxycyclohexyl)-2- |
| | oxoacetyl]-2-piperidinecarboxylate |
| 213 | ethyl 1-(2-cyclohexyl-2-oxoacetyl)-2- |
| | piperidinecarboxylate |
| 214 | ethyl 1-(2-oxo-2-piperidinoacetyl)-2- |
| | piperidinecarboxylate |
| 215 | ethyl 1-[2-(3,4-dihydro-2H-6-pyranyl)-2- |
| | oxoacetyl)-2-piperidinecarboxylate |
| 216 | ethyl 1-(2-oxo-2-phenylacetyl)-2- |
| | piperidinecarboxylate |
| 217 | ethyl 1-(4-methyl-2-oxo-1-thioxopentyl)-2- |
| | piperidinecarboxylate |
| 218 | 3-phenylpropyl 1-(2-hydroxy-3,3-dimethyl- |
| | pentanoyl)-2-piperidinecarboxylate |
| 219 | (1R)-1-phenyl-3-(3,4,5-trimethoxy- |
| | phenyl)propyl 1-(3,3-dimethylbutanoyl)-2- |
| | piperidinecarboxylate |
| 220 | (1R)-1,3-diphenylpropyl 1-(benzylsulfonyl)- |
| | 2-piperidinecarboxylate |
| 221 | 3-(3,4,5-trimethoxyphenyl)propyl 1- |
| | (benzylsulfonyl)-2-piperidinecarboxylate |
| 222 | 1-(2-[(2R,3R,6S)-6-[(2S,3E,5E,7E,9S,11R)- |
| | 2,13-dimethoxy-3,9,11-trimethyl-12-oxo- |
| | 3,5,7-tridecatrienyl]-2-hydroxy-3- |
| | methyltetrahydro-2H-2-pyranyl)-2-oxoacetyl)- |
| | 2-piperidinecarboxylic acid |
| 223 | methyl 1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyl-tetrahydro-2H-2-pyranyl)-2- |
| | oxoacetyl)-2-piperidinecarboxylate |
| 224 | isopropyl 1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyl-tetrahydro-2H-2-pyranyl)-2- |
| | oxoacetyl)-2-piperidinecarboxylate |
| 225 | benzyl 1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyl-tetrahydro-2H-2-pyranyl)-2- |
| | oxoacetyl)-2-piperidinecarboxylate |
| 226 | 1-phenylethyl 1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyl-tetrahydro-2H-2-pyranyl)-2- |
| | oxoacetyl)-2-piperidinecarboxylate |
| 227 | (Z)-3-phenyl-2-propenyl 1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyltetrahydro-2H-2-pyranyl)-2- |
| | oxoacetyl)-2-piperidinecarboxylate |
| 228 | 3-(3,4-dimethoxyphenyl)propyl 1-(2- |
| | [(2R,3R,6S)-6-[(2S,3E,5E,7E,9S,11R)-2,13- |
| | dimethoxy-3,9,11-trimethyl-12-oxo-3,5,7- |
| | tridecatrienyl]-2-hydroxy-3-methyl- |
| | tetrahydro-2H-2-pyranyl)-2-oxoacetyl)-2- |
| | piperidinecarboxylate |
| 229 | N2-benzyl-1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyl-tetrahydro-2H-2-pyranyl)-2- |
| | oxoacetyl)-2-piperidinecarboxylate |
| 230 | N2-(3-phenylpropyl)-1-(2-[(2R,3R,6S)-6- |
| | [(2S,3E,5E,7E,9S,11R)-2,13-dimethoxy-3,9,11- |
| | trimethyl-12-oxo-3,5,7-tridecatrienyl]-2- |
| | hydroxy-3-methyltetrahydro-2H-2-pyranyl)-2- |
| | oxoacetyl)-2-piperidinecarboxylate |
| 231 | (E)-3-(3,4-dichlorophenyl)-2-propenyl 1- |
| | (3,3-dimethyl-2-oxopentanoyl)-2-piperidine- |
| | carboxylate |
| 232 | (E)-3-(3,4,5-trimethoxyphenyl)-2-propenyl 1- |
| | (3,3-dimethyl-2-oxopentanoyl)-2-piperidine- |
| | carboxylate |
| 233 | (E)-3-phenyl-2-propenyl 1-(3,3-dimethyl-2- |
| | oxo-pentanoyl)-2-piperidinecarboxylate |
| 234 | (E)-3-((3-(2,5-dimethoxy)-phenylpropyl)- |
| | phenyl)-2-propenyl 1-(3,3-dimethyl-2- |
| | oxopentanoyl)-2-piperidinecarboxylate |
| 235 | (E)-3-(1,3-benzodioxol-5-yl)-2-propenyl 1- |
| | (3,3-dimethyl-2-oxopentanoyl)-2-piperidine- |
| | carboxylate |
| 236 | 4-(4-methoxyphenyl)butyl 1-(2-oxo-2- |
| | phenylacetyl)-2-piperidinecarboxylate |
| 237 | 3-phenylpropyl 1-(2-oxo-2-phenylacetyl)-2- |
| | piperidinecarboxylate |
| 238 | 3-(3-pyridyl)propyl 1-(2-oxo-2- |
| | phenylacetyl)-2-piperidinecarboxylate |
| 239 | 3-(3-pyridyl)propyl 1-(3,3-dimethyl-2- |
| | oxopentanoyl)-2-piperidinecarboxylate |
| 240 | 4-phenyl-1-(3-phenylpropyl)butyl 1-(3,3- |
| | dimethyl-2-oxopentanoyl)-2-piperidine- |
| | carboxylate |
| 241 | 4-(4-methoxyphenyl)butyl 1-(3,3-dimethyl-2- |
| | oxopentanoyl)-2-piperidinecarboxylate |
| 242 | 1-(4-methoxyphenethyl)-4-phenylbutyl 1-(3,3- |
| | dimethyl-2-oxopentanoyl)-2-piperidine- |
| | carboxylate |
| 243 | 3-(2,5-dimethoxyphenyl)propyl 1-(3,3- |
| | dimethyl-2-oxopentanoyl)-2- |
| | piperidinecarboxylate |
| 244 | 3-(1,3-benzodioxol-5-yl)propyl 1-(3,3- |
| | dimethyl-2-oxopentanoyl)-2-piperidine- |
| | carboxylate |
| 245 | 1-phenethyl-3-phenylpropyl 1-(3,3-dimethyl- |
| | 2-oxopentanoyl)-2-piperidinecarboxylate |
| 246 | 4-(4-methoxyphenyl)butyl 1-(2-cyclohexyl-2- |
| | oxoacetyl)-2-piperidinecarboxylate |
| 247 | 3-cyclohexylpropyl 1-(2-cyclohexyl-2- |
| | oxoacetyl)-2-piperidinecarboxylate |
| 248 | 3-phenylpropyl 1-(2-cyclohexyl-2-oxoacetyl)- |
| | 2-piperidinecarboxylate |
| 249 | 3-cyclohexylpropyl 1-(3,3-dimethyl-2- |
| | oxobutanoyl)-2-piperidinecarboxylate |
| 250 | 3-phenylpropyl 1-(3,3-dimethyl-2- |
| | oxobutanoyl)-2-piperidinecarboxylate |
| 251 | 4-(4-methoxyphenyl)butyl 1-(3,3-dimethyl-2- |
| | oxobutanoyl)-2-piperidinecarboxylate |
| 252 | 4-phenyl-1-(3-phenylpropyl)butyl 1-(3,3- |
| | dimethyl-2-oxobutanoyl)-2-piperidine- |
| | carboxylate |

In yet a further embodiment, there is provided a method for the treatment of nerve injury caused as a consequence of prostate surgery which comprises administering to a patient a compound of formula LV:

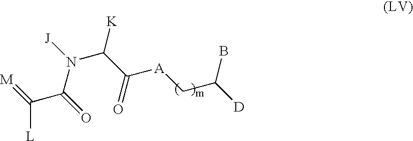

or a pharmaceutically acceptable salt, ester, or solvate thereof, wherein:

m is 0-3;

A is CH₂, O, NH, or N—(C₁-C₄ alkyl);

B and D are independently hydrogen, Ar, C₅-C₇ cycloalkyl substituted C₁-C₆ straight or branched chain alkyl or C₂-C₆ straight or branched chain alkenyl, C₅-C₇ cycloalkenyl substituted C₁-C₆ straight or branched chain alkyl or C₂-C₆ straight or branched chain alkenyl, or Ar substituted C₁-C₆ straight or branched chain alkyl or C₂-C₆ straight or branched chain alkenyl, wherein in each case, one or two carbon atom(s) of said alkyl or alkenyl may be substituted with one or two heteroatom(s) independently selected from the group consisting of oxygen, sulfur, SO, and SO₂ in chemically reasonable substitution patterns, or

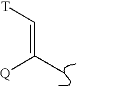

wherein

Q is hydrogen, C₁-C₆ straight or branched chain alkyl, or C₂-C₆ straight or branched chain alkenyl; and

T is Ar or C₅-C₇ cycloalkyl substituted at positions 3 and 4 with substituents independently selected from the group consisting of hydrogen, hydroxy, O—(C₁-C₄ alkyl), O—(C₂-C₄ alkenyl), and carbonyl;

Ar is selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar contains 1-3 substituent(s) independently selected from the group consisting of hydrogen, halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl;

L is either hydrogen or U; M is either oxygen or CH—U, provided that if L is hydrogen, then M is CH—U, or if M is oxygen then L is U;

U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar, or Ar;.

J is hydrogen, CO or C2 alkyl, or benzyl; K is C₁-C₄ straight or branched chain alkyl, benzyl or cyclohexylmethyl; or J and K are taken together to form a 5-7 membered heterocyclic ring which is substituted with oxygen, sulfur, SO, or SO₂. Representative species of Formula LV are presented in Table XXXVIII:

TABLE XXXVIII

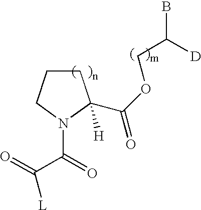

| Cpd. | n | m | B | D | L |
|---|---|---|---|---|---|
| 253 | 2 | O | 3-Phenylpropyl | 3-(3-Pyridyl)propyl | Phenyl |
| 254 | 2 | O | 3-Phenylpropyl | 3-(2-Pyridyl)propyl | Phenyl |
| 255 | 2 | O | 3-Phenylpropyl | 2-(4-Methoxyphenyl)ethyl | Phenyl |
| 256 | 2 | O | 3-Phenylpropyl | 3-Phenylpropyl | Phenyl |
| 257 | 2 | O | 3-Phenylpropyl | 3-Phenylpropyl | 3,4,5- |
| | | | | | Trimethoxyphenyl |
| 258 | 2 | O | 3-Phenylpropyl | 2-(3-Pyridyl)propyl | 3,4,5- |
| | | | | | Trimethoxyphenyl |
| 259 | 2 | O | 3-Phenylpropyl | 3-(2-Pyridyl)propyl | 3,4,5- |
| | | | | | Trimethoxyphenyl |
| 260 | 2 | O | 3-Phenylpropyl | 3-(4-Methoxyphenyl)propyl | 3,4,5- |
| | | | | | Trimethoxyphenyl |
| 261 | 2 | O | 3-Phenylpropyl | 3-(3-Pyridyl)propyl | 3-iso-propoxyphenyl |

### Formula (LVI)

U.S. Pat. No. 5,330,993, incorporated herein by reference, discloses an exemplary pipecolic acid derivative of Formula LVI:

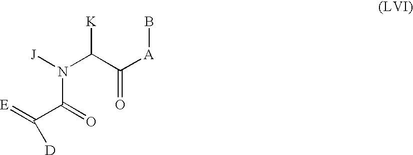

or a pharmaceutically acceptable salt, ester, or solvate thereof, wherein:

A is O, NH, or N—(C₁-C₄ alkyl);

B is hydrogen, CHL-Ar, C₁-C₆ straight or branched chain alkyl, C₂-C₆ straight or branched chain alkenyl, C₅-C₇ cycloalkyl, C₅-C₇ cyclcalkenyl, Ar substituted C₁-C₆ alkyl or C₂-C₆ alkenyl, or

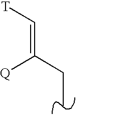

wherein

L and Q are independently hydrogen, C₁-C₆ straight or branched chain alkyl, or C₂-C₆ straight or branched chain alkenyl; and

T is Ar or C₅-C₇ cyclohexyl substituted at positions 3 and 4 with substituents independently selected from the group consisting of hydrogen, hydroxy, O—(C₁-C₄ alkyl), O—(C₂-C₄ alkenyl), and carbonyl;

Ar is selected from the group consisting of 1-napthyl, 2-napthyl, 2-furyl, 3-furyl, 2-thienyl, 2-pyridyl, 3-pyridyl, 4-pyridyl and phenyl having 1-3 substituent(s) independently selected from the group consisting of hydrogen, halo, hydroxy, nitro, CF₃, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, and phenyl.

D is hydrogen or U; E is oxygen or CH—U, provided that if D is hydrogen, then E is CH—U, or if E is oxygen, then D is U;

U is hydrogen, O—(C₁-C₄ straight or branched chain alkyl), O—(C₂-C₄ straight or branched chain alkenyl), C₁-C₆ straight or branched chain alkyl, C₂-C₆ straight or branched chain alkenyl, C₅-C₇-cycloalkyl, C₅-C₇ cycloalkenyl substituted with C₁-C₄ straight or branched chain alkyl or C₂-C₄ straight or branched chain alkenyl, 2-indolyl, 3-indolyl, (C₁-C₄ alkyl or C₂-C₄ alkenyl)-Ar, or Ar;

J is hydrogen, C₁ or C₂ alkyl, or benzyl; K is C₁-C₄ straight or branched chain alkyl, benzyl or cyclohexylethyl; or J and K are taken together to form a 5-7 membered heterocyclic ring which is substituted with oxygen, sulfur, SO, or SO₂.

### Formula LVII

A preferred pipecolic acid derivative is a compound of Formula LVII:

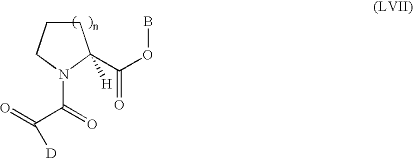

or a pharmaceutically acceptable salt, ester, or solvate thereof, wherein:

n is 2;

D is phenyl, methoxy, 2-furyl, or 3,4,5-trimethoxyphenyl; and

B is benzyl, 3-phenylpropyl, 4-(4-methoxyphenyl)butyl, 4-phenylbutyl, phenethyl, 3-cyclohexylpropyl, 4-cyclohexylbutyl, 3-cyclopentylpropyl, 4-cyclohexylbutyl, 3-phenoxybenzyl, 3-(3-indolyl)propyl, or 4-(4-methoxyphenyl)butyl;

provided that:

when D is phenyl, then B is benzyl, 3-phenylpropyl, 4-(4-methoxyphenyl)butyl, 4-phenylbutyl, phenethyl, ore 4-cyclohexylbutyl;

when D is methoxy, B is benzyl, 4-cyclohexylbutyl, 3-cyclohexylpropyl, or 3-cyclopentylpropyl;

when D is 2-furyl, then B is benzyl; and

when D is 3,4,5-trimethoxyphenyl, then B is 4-cyclohexylbutyl, 3-phenoxybenzyl, 4-phenylbutyl, 3-(3-indolyl)propyl, or 4-(4-methoxyphenyl)butyl.

Representative species of Formula LVII are presented in Table XXXIX.

TABLE XXXIX

| Cpd. | B | D | n |
|---|---|---|---|
| 262 | Benzyl | Phenyl | 2 |
| 263 | 3-Phenylpropyl | Phenyl | 2 |
| 264 | 4-(4-Methoxyphenyl)butyl | Phenyl | 2 |
| 265 | 4-Phenylbutyl | Phenyl | 2 |
| 266 | Phenethyl | Phenyl | 2 |
| 267 | 4-Cyclohexylbutyl | Phenyl | 2 |
| 268 | Benzyl | Methoxy | 2 |
| 269 | 4-Cyclohexylbutyl | Methoxy | 2 |
| 269 | 3-Cyclohexylpropyl | Methoxy | 2 |
| 270 | 3-Cyclopentylpropyl | Methoxy | 2 |
| 271 | Benzyl | 2-Furyl | 2 |
| 272 | 4-Cyclohexylbutyl | 3,4,5-Trimethoxyphenyl | 2 |
| 273 | 3-Phenoxybenzyl | 3,4,5-Trimethoxyphenyl | 2 |
| 274 | 4-Phenylbutyl | 3,4,5-Trimethoxyphenyl | 2 |
| 275 | 3-(3-Indolyl)propyl | 3,4,5-Trimethoxyohenyl | 2 |
| 276 | 4-(4-Methoxyphenyl)butyl | 3,4,5-Trimethoxyphenyl | 2 |

### Formula LVIII

The pipecolic acid derivative may also be a compound of formula LVIII:

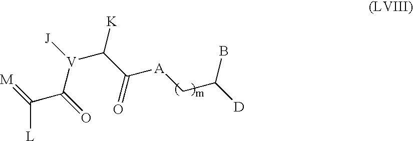

or a pharmaceutically acceptable salt, ester, or solvate thereof, wherein:

V is CH, N, or S;

J and K, taken together with V and the carbon atom to which they are respectively attached, form a 5-7 membered saturated or unsaturated heterocyclic ring containing, in addition to V, one or more heteroatom(s) selected from the group consisting of O, S, SO, SO₂, N, NH, and NR;

R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, (C₁-C₆)-alkylthio, sulfhydryl, amino, (C₁-C₆)-alkylamino, amino-(C₁-C₆)-alkyl, aminocarboxyl, and Ar₂;

Ar₁ and Ar₂ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S;

A, B, D, L, M, and m are as defined in Formula LV, above.

In an additional embodiment of the invention, there is provided a method for the treatment of nerve injury caused as a consequence of prostate surgery which comprises administering to a warm-blooded animal a compound of the following formulae:

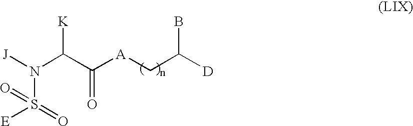

or a pharmaceutically acceptable salt, ester or solvate thereof, wherein:

A is CH₂, O, NH, or N—(C₁-C₄ alkyl);

B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or

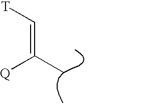

wherein

Q is hydrogen, C₁-C₆ straight or branched chain alkyl, or C₂-C₆ straight or branched chain alkenyl; and

T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl; provided that both B and D are not hydrogen;

Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;

E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar;

J is hydrogen, C₁ or C₂ alkyl, or benzyl; K is C₁-C₄ straight or branched chain alkyl, benzyl, or cyclohexylmethyl; or J and K are taken together to form a 5-7 membered heterocyclic ring which is substituted with O, S, SO, or SO₂;

n is 0 to 3; and

the stereochemistry at carbon positions 1 and 2 is R or S.

### Formula LX

In a preferred embodiment of Formula I, J and K are taken together and the small molecule sulfonamide is a compound of Formula LX:

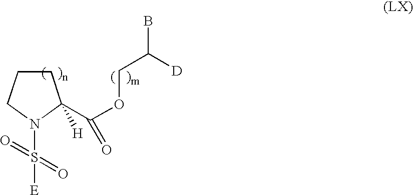

or a pharmaceutically acceptable salt thereof, wherein:

n is 1 or 2; and

m is 0 or 1.

In a more preferred embodiment, B is selected from the group consisting of hydrogen, benzyl, 2-phenylethyl, and 3-phenylpropyl;

D is selected from the group consisting of phenyl, 3-phenylpropyl, 3-phenoxyphenyl, and 4-phenoxyphenyl; and

E is selected from the group consisting of phenyl, 4-methylphenyl, 4-methoxyphenyl, 2-thienyl, 2,4,6-triisopropylphenyl, 4-fluorophenyl, 3-methoxyphenyl, 2-methoxyphenyl, 3,5-dimethoxyphenyl, 3,4,5-trimethoxyphenyl, methyl, 1-naphthyl, 8-quinolyl, 1-(5-N,N-dimethylamino)-naphthyl, 4-iodophenyl, 2,4,6-trimethylphenyl, benzyl, 4-nitrophenyl, 2-nitrophenyl, 4-chlorophenyl, and E-styrenyl.

### Formula LXI

Another exemplary small molecule sulfonamide is a compound of Formula LXI:

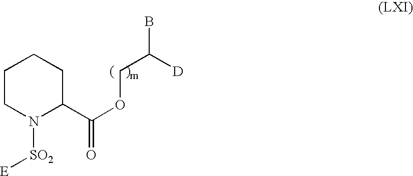

or a pharmaceutically acceptable salt thereof, wherein:

B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl or Ar, and wherein one or two carbon atom(s) of said alkyl or alkenyl may be substituted with one or two heteroatom(s) independently selected from the group consisting of O, S, SO, and SO₂ in chemically reasonable substitution patterns, or

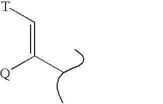

wherein

Q is hydrogen, C₁-C₆ straight or branched chain alkyl, or C₂-C₆ straight or branched chain alkenyl; and

T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl; provided that both B and D are not hydrogen;

Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;

E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar; and

m is 0 to 3.

A further exemplary small molecule sulfonamide is a compound of Formula (LXII):

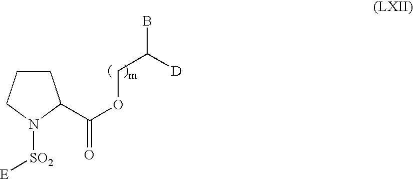

or a pharmaceutically acceptable salt thereof, wherein:

B and D are independently Ar, hydrogen, C₁-C₆ straight or branched chain alkyl, or C₂-C₆ straight or branched chain alkenyl, wherein said alkyl or alkenyl is unsubstituted or substituted with C₅-C₇ cycloalkyl, C₅-C₇ cycloalkenyl, or Ar, and wherein one or two carbon atom(s) of said alkyl or alkenyl may be substituted with one or two heteroatom(s) independently selected from the group consisting of O, S, SO, and SO₂ in chemically reasonable substitution patterns, or

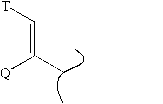

wherein

Q is hydrogen, C₁-C₆ straight or branched chain alkyl, or C₂-C₆ straight or branched chain alkenyl; and

T is Ar or C₅-C₇ cycloalkyl substituted at positions 3 and 4 with one or more substituent(s) independently selected from the group consisting of hydrogen, hydroxy, O—(C₁-C₄ alkyl), O—(C₂-C₄ alkenyl), and carbonyl; provided that both B and D are not hydrogen;

Ar is selected from the group consisting of phenyl, 1-napthyl, 2-naphthyl, 2-furyl, 3-furyl, 2-thienyl, 3-thienyl, 2-pyridyl, 3-pyridyl, 4-pyridyl, monocyclic and bicyclic heterocyclic ring systems with individual ring sizes being 5 or 6 which contain in either or both rings a total of 1-4 heteroatoms independently selected from the group consisting of O, N, and S; wherein Ar contains 1-3 substituent(s) independently selected from the group consisting of hydrogen, halo, hydroxy, nitro, trifluoromethyl, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, 1,2-methylenedioxy, amino, carboxyl, and phenyl;

E is C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₂-C₄ alkyl or C₂-C₄ alkenyl)-Ar, or Ar; and

m is 0 to 3.

A further exemplary small molecule sulfonamide is a compound of Formula LXIII:

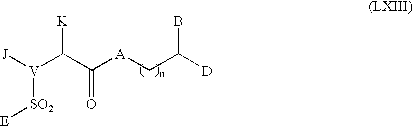

or a pharmaceutically acceptable salt, ester, or solvate thereof, wherein:

V is CH, N, or S;

J and K, taken together with V and the carbon atom to which they are respectively attached, form a 5-7 membered saturated or unsaturated heterocyclic ring containing, in addition to V, one or more heteroatom(s) selected from the group consisting of O, S, SO, SO₂, N, NH, and NR;

R is either C₁-C₉ straight or branched chain alkyl, C₂-C₉ straight or branched chain alkenyl, C₃-C₉ cycloalkyl, C₅-C₇ cycloalkenyl, or Ar₁, wherein R is either unsubstituted of substituted with one or more substituent(s) independently selected from the group consisting of halo, halo(C₁-C₆)-alkyl, carbonyl, carboxy, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, thio-(C₁-C₆)-alkyl, (C₁-C₆)-alkylthio, sulfhydryl, amino, (C₁-C₆)-alkylamino, amino-(C₁-C₆)-alkyl, aminocarboxyl, and Ar₂;

Ar₁ and Ar₂ are independently an alicyclic or aromatic, mono-, bi- or tricyclic, carbo- or heterocyclic ring; wherein the individual ring size is 5-8 members; wherein said heterocyclic ring contains 1-6 heteroatom(s) independently selected from the group consisting of O, N, and S;

A, B, D, E, and n are as defined in Formula I above.

Representative species of Formulas LIX-LXIII are presented in Table XL.

TABLE XL

| Cpd. | Structure and name |
|---|---|
| 278 | 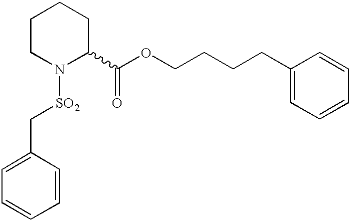 |
| | 4-phenyl-1-butyl-1-(benzylsulfonyl)-(2R,S)-2- |
| | pipecolinate |
| 279 | 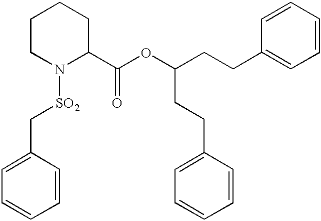 |
| | 1,5-diphenyl-3-pentyl-N-(a-toluenesulfonyl)- |
| | pipecolate |
| 280 | 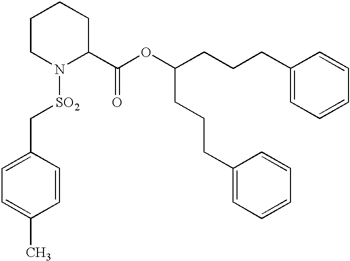 |
| | 1,7-diphenyl-4-heptyl-N-(para-toluene- |
| | sulfonyl)pipecolate |
| 281 | 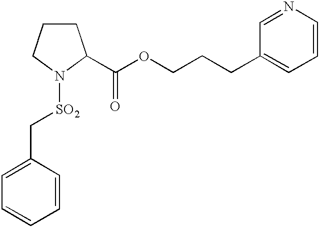 |
| | 3-(3-pyridyl)-1-propyl-(2S)-N-(a- |
| | toluenesulfonyl)-pyrrolidine-2-carboxylate |
| 282 | 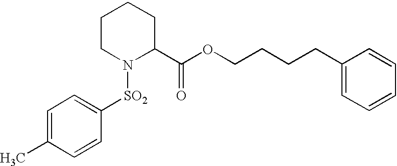 |
| | 4-phenyl-1-butyl-N-(para- |
| | toluenesulfonyl)pipecolate |
| 283 | 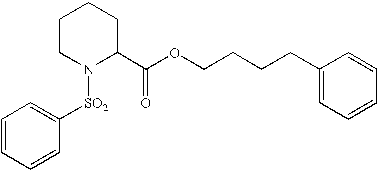 |
| | 4-phenyl-1-butyl-N-(benzenesulfonyl)- |
| | pipecolate |
| 284 | 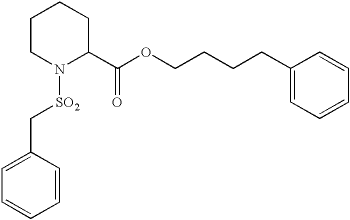 |
| | 4-phenyl-1-butyl-N-(a-toluenesulfonyl)- |
| | pipecolate |

VII. Carboxylic Acid Isosteres as Neurotrophic Compounds

Another especially preferred embodiment of the invention is a compound of formula (LXIV):

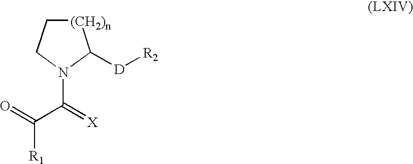

in which:

n is 1-3;

X is either O or S;

R₁ is selected from the group consisting of C₁-C₉ straight or branched chain alkyl, C₂-C₉ straight or branched chain alkenyl, aryl, heteroaryl, carbocycle, or heterocycle;

D is a bond, or a C₁-C₁₀ straight or branched chain alkyl, C₂-C₁₀alkenyl or C₂-C₁₀ alkynyl; and

R₂ is a carboxylic acid or a carboxylic acid isostere; or a pharmaceutically acceptable salt, ester, or solvate thereof.

Preferred embodiments of this invention are where R₂ is a carbocycle or heterocycle containing any combination of CH₂, O, S, or N in any chemically stable oxidation state, where any of the atoms of said ring structure are optionally substituted in one or more positions with R³.

Especially preferred embodiments of this invention are where R₂ is selected from the group below:

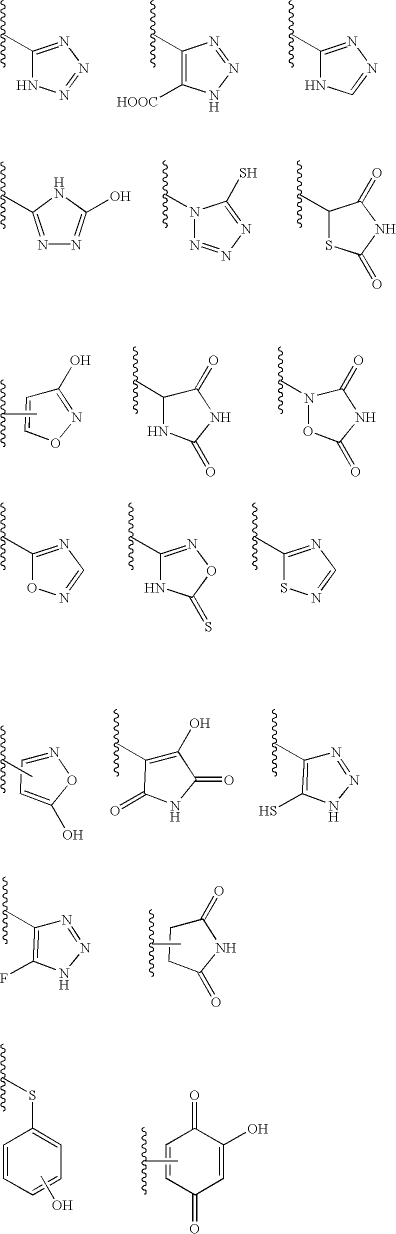

where the atoms of said ring structure may be optionally substituted at one or more positions with R³.

Another preferred embodiment of this invention is where R₂ is selected from the group consisting of —COOH, —SO₃H, —SO₂HNR³, —PO₂(R³)₂, —CN, —PO₃(R³)₂, —OR³, —SR3, —NHCOR³, —N(R³)₂, —CON(R³)₂, —CONH(O)R³, —CONHNHSO₂R³, —COHNSO₂R³, and —CONR³CN wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl.

Preferred embodiments of this invention are: (2S)-1-(1,2-dioxo-3,3-dimethylpentyl)-2-hydroxymethyl pyrrolidine; (2S)-1-(1,2-dioxo-3,3-dimethylpentyl)-2-pyrrolidinetetrazole; (2S)-1-(1,2-dioxo-3,3-dimethylpentyl)-2-pyrrolidinecarbonitrile; and (2S)-1-(1,2-dioxo-3,3-dimethylpentyl)-2-aminocarbonyl piperidine.

A compound of the present invention, especially formula LXIV, wherein n is 1, X is O, D is a bond, R₁ is 1,1 dimethylpropyl, and R₂ is —CN, is named (2S)-1-(1,2-dioxo-3,3-dimethylpentyl)-2-pyrrolidine-carbonitrile.

Specific embodiments of the inventive compounds are presented in Tables XLI, XLII, and XLIII. The present invention contemplates employing the compounds of Tables XLI, XLII, XLIII, and XLIV, below.

TABLE XLI

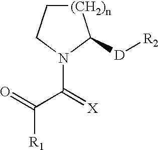

| No. | X | n | R₁ |
|---|---|---|---|
| 285 | O | 1 | 3,4,5-trimethylphenyl |
| 286 | O | 2 | 3,4,5-trimethylphenyl |
| 287 | O | 1 | tert-butyl |
| 287 | O | 3 | tert-butyl |
| 288 | O | 1 | cyclopentyl |
| 289 | O | 2 | cyc lopentyl |
| 290 | O | 3 | cyclopentyl |
| 291 | O | 1 | cyclohexyl |
| 292 | O | 2 | cyclohexyl |
| 293 | O | 3 | cyclohexyl |
| 294 | O | 1 | cycloheptyl |
| 295 | O | 2 | cycloheptyl |
| 296 | O | 3 | cycloheptyl |
| 297 | O | 1 | 2-thienyl |
| 298 | O | 2 | 2-thienyl |
| 299 | O | 3 | 2-thienyl |
| 300 | O | 1 | 2-furyl |
| 301 | O | 2 | 2-furyl |
| 302 | O | 3 | 2-furyl |
| 303 | O | 3 | phenyl |
| 304 | O | 1 | 1,1-dimethylpentyl |
| 305 | O | 2 | 1,1-dimethylhexyl |
| 306 | O | 3 | ethyl |
| 307 | | | |

when D is a bond and R2 is COOH

TABLE XLII

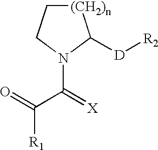

| No. | X | n | R₁ | D | R₂ |
|---|---|---|---|---|---|
| 308 | S | 1 | 1,1-dimethyl propyl | CH₂ | COOH |
| 309 | S | 1 | 1,1-dimethyl propyl | bond | COOH |
| 310 | O | 1 | 1,1-dimethyl propyl | CH₂ | CH |
| 311 | O | 1 | 1,1-dimethyl propyl | bond | SO₃H |
| 312 | O | 1 | 1,1-dimethyl propyl | CH₂ | CN |
| 313 | O | 1 | 1,1-dimethyl propyl | bond | CN |
| 314 | O | 1 | 1,1-dimethyl propyl | bond | tetrazolyl |
| 315 | S | 1 | Phenyl | (CH₂)₂ | COOH |
| 316 | S | 1 | Phenyl | (CH₂)₃ | COOH |
| 317 | S | 2 | Phenyl | CH₂ | COOH |
| 318 | O | 1 | 1,1-dimethyl propyl | bond | CONH₂ |
| 319 | O | 2 | 1,1-dimethyl propyl | bond | CONH₂ |
| 320 | S | 2 | 2-furyl | bond | PO₃H₂ |
| 321 | O | 2 | Propyl | (CH₂)₂ | COOH |
| 322 | O | 1 | Propyl | (CH₂)₂ | COOH |
| 323 | O | 1 | tert-butyl | (CH₂)₄ | COOH |
| 324 | O | 1 | Methyl | (CH₂)₅ | COOH |
| 325 | O | 2 | Phenyl | (CH₂)₆ | COOH |
| 326 | O | 2 | 3,4,5-trimethoxy- | CH₂ | COOH |
| | | | phenyl | | |
| 327 | O | 2 | 3,4,5-trimethoxy- | CH₂ | tetrazolyl |
| | | | phenyl | | |

TABLE XLIII

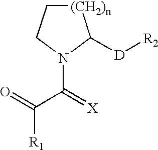

| No. | n | X | D | R₂ | R₁ |
|---|---|---|---|---|---|
| 328 | 1 | S | Bond | COOH | Phenyl |
| 329 | 1 | O | Bond | COOH | a-MethylBenzyl |
| 330 | 2 | O | Bond | COOH | 4-MethylBenzyl |
| 331 | 1 | O | Bond | Tetrazole | Benzyl |
| 332 | 1 | O | Bond | SO₃H | a-MethylBenzyl |
| 333 | 1 | O | CH₂ | COOH | 4-MethylBenzyl |
| 334 | 1 | O | Bond | SO₂HNMe | Benzyl |
| 335 | 1 | O | Bond | CN | a-MethylBenzyl |
| 336 | 1 | O | Bond | PO₃H₂ | 4-MethylBenzyl |
| 337 | 2 | O | Bond | COOH | Benzyl |
| 338 | 2 | O | Bond | COOH | a-MethylBenzyl |
| 339 | 2 | O | Bond | COOH | 4-MethylBenzyl |
| 340 | 2 | S | Bond | COOH | 3,4,5- |
| | | | | | trimethoxyphenyl |
| 341 | 2 | O | Bond | COOH | Cyclohexyl |
| 342 | 2 | O | Bond | PO₂Het | i-propyl |
| 343 | 2 | O | Bond | PO₃HPropyl | ethyl |
| 344 | 2 | O | Bond | PO₃(Et)₂ | Methyl |
| 345 | 2 | O | Bond | Ome | tert-butyl |
| 346 | 1 | O | Bond | Oet | n-pentyl |
| 347 | 2 | O | Bond | Opropyl | n-hexyl |
| 348 | 1 | O | Bond | Obutyl | Cyclohexyl |
| 349 | 1 | O | Bond | Opentyl | cyclopentyl |
| 350 | 1 | O | Bond | Ohexyl | n-heptyl |
| 351 | 1 | O | Bond | Sme | n-octyl |
| 352 | 1 | O | Bond | Set | n-nonyl |
| 353 | 2 | O | Bond | Spropyl | 2-indolyl |
| 354 | 2 | O | Bond | Sbutyl | 2-furyl |
| 355 | 2 | O | Bond | NHCOMe | 2-thiazolyl |
| 356 | 2 | O | Bond | NHCOEt | 2-thienyl |
| 357 | 1 | O | CH₂ | N(Me)₂ | 2-pyridyl |
| 358 | 1 | O | (CH₂)₂ | N(Me)Et | 1,1- |
| | | | | | dimethylpropyl |
| 359 | 1 | O | (CH₂)₃ | CON(Me)₂ | 1,1- |
| | | | | | dimethylpropyl |
| 360 | 1 | O | (CH₂)₄ | CONHMe | 1,1- |
| | | | | | dimethylpropyl |
| 361 | 1 | O | (CH₂)₅ | CONHEt | 1,1-dimethylpropyl |
| 362 | 1 | O | (CH₂)₆ | CONHPropyl | 1,1-dimethylpropyl |
| 363 | 1 | O | Bond | CONH(O)Me | Benzyl |
| 364 | 1 | O | Bond | CONH(O)Et | a-Methylphenyl |
| 365 | 1 | O | Bond | CONH(O)Propyl | 4-Methylphenyl |
| 366 | 1 | O | (CH₂)₂ | COOH | Benzyl |
| 367 | 1 | O | Bond | COOH | a-Methylphenyl |
| 368 | 1 | O | Bond | COOH | 4-Methylphenyl |
| 369 | 1 | O | CH₂ | COOH | 1,1-dimethylpropyl |
| 370 | 1 | O | (CH₂)₂ | COOH | 1,1-dimethylbutyl |
| 371 | 1 | O | (CH₂)₂ | COOH | 1, 1-dimethylpentyl |
| 372 | 1 | O | (CH₂)₄ | COOH | 1,1-dimethylhexyl |
| 373 | 1 | O | (CH₂)₅ | COOH | 1,1-dimethylethyl |
| 374 | 1 | O | (CH₂)₆ | COOH | iso-propyl |
| 375 | 1 | O | (CH₂)₇ | COOH | tert-butyl |
| 376 | 1 | O | (CH₂)₆ | COOH | 1, 1-dimethylpropyl |
| 377 | 1 | O | (CH₂)₉ | COOH | benzyl |
| 378 | 1 | O | (CH₂)₁₀ | COOH | 1,1-dimethylpropyl |
| 379 | 1 | O | C₂H₂ | COOH | cyclohexylmethyl |
| 380 | 1 | O | 2-OH, | COOH | 1,1-dimethylpropyl |
| | | | Et | | |
| 381 | 1 | O | 2-but- | COOH | 1,1-dimethylpropyl |
| | | | ylene | | |
| 382 | 1 | S | i-Pro | COOH | 1,1-dimethylpropyl |
| 383 | 2 | S | t-Bu | COOH | phenyl |
| 384 | 2 | O | 2-NO₂- | COOH | 1,1-dimethylpropyl |
| | | | hexyl | | |
| 385 | 1 | O | (CH₂)₂ | CN | 1,1-dimethylpropyl |
| 386 | 1 | O | (CH₂)₃ | CN | 1,1-dimethylpropyl |
| 387 | 3 | O | Bond | CONHNHSO₂Me | Benzyl |
| 388 | 3 | O | Bond | CONHNHSO₂Et | a-Methylphenyl |
| 389 | 3 | O | Bond | CONHSO₂Me | 4-Methylphenyl |
| 390 | 1 | O | Bond | CONHNHSO₂Et | Phenyl |
| 391 | 2 | O | Bond | CON(Me)CN | a-Methylphenyl |
| 392 | 1 | O | Bond | CON(Et)CN | 4-Methylphenyl |
| 393 | 1 | O | (CH₂)₂ | COOH | methyl |
| 394 | 1 | O | (CH₂)₃ | COOH | ethyl |
| 395 | 1 | O | (CH₂)₄ | COOH | n-propyl |
| 396 | 1 | O | (CH₂)₅ | COOH | t-butyl |
| 397 | 1 | O | (CH₂)₆ | COOH | Pentyl |
| 398 | 1 | O | (CH₂)₇ | COOH | Hexyl |
| 399 | 1 | O | (CH₂)₈ | COOH | Heptyl |
| 400 | 1 | O | (CH₂)₉ | COOH | Octyl |
| 401 | 1 | O | C₂H₂ | COOH | Cyclohexyl |
| 402 | 2 | O | bond | 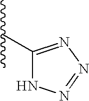 | 1,1-dimethylpropyl |
| 403 | 1 | O | bond | 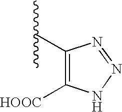 | 1,1-dimethylpropyl |
| 404 | 1 | O | bond | 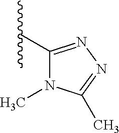 | 1,1-dimethylpropyl |
| 405 | 1 | O | bond | 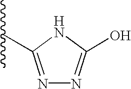 | 1,1-dimethylpropyl |
| 406 | 1 | O | bond | 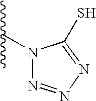 | 1,1-dimethylpropyl |
| 407 | 1 | O | bond | 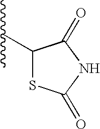 | 1,1-dimethylpropyl |
| 408 | 1 | O | bond | 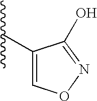 | 1,1-dimethylpropyl |
| 409 | 1 | O | bond | 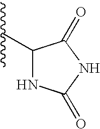 | 1,1-dimethylpropyl |
| 410 | 1 | O | bond | 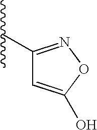 | 1,1-dimethylpropyl |
| 411 | 1 | O | bond | 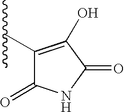 | 1,1-dimethylpropyl |
| 412 | 1 | O | bond | 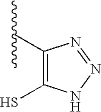 | 1,1-dimethylpropyl |
| 413 | 1 | O | bond | 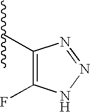 | 1,1-dimethylpropyl |
| 414 | 1 | O | bond | 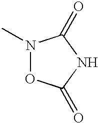 | 1,1-dimethylpropyl |
| 415 | 1 | O | bond | 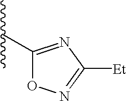 | 1,1-dimethylpropyl |
| 416 | 1 | O | bond | 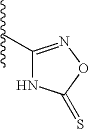 | 1,1-dimethylpropyl |
| 417 | 1 | O | bond | 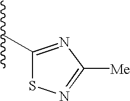 | 1,1-dimethylpropyl |
| 418 | 1 | O | bond | 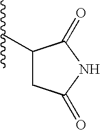 | 1,1-dimethylpropyl |
| 419 | 1 | O | bond | 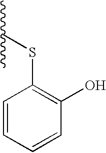 | 1,1-dimethylpropyl |
| 420 | 1 | O | bond | 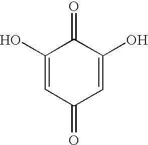 | 1,1-dimethylpropyl |
| 421 | 1 | O | bond | COOH | 1,1-dimethylpropyl |
| 422 | 2 | O | bond | COOH | 1,1-dimethylpropyl |

TABLE XLIV

| Com- | |
|---|---|
| pound | Compound |
| No. | Structure |
| 423 | 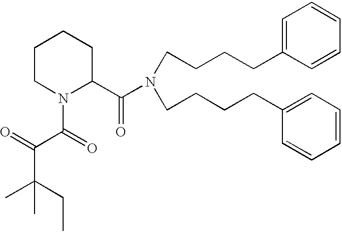 |
| 424 | 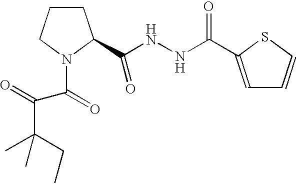 |
| 425 | 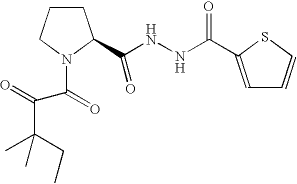 |
| 426 | 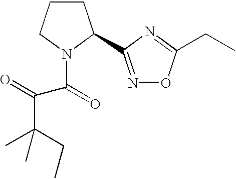 |
| 427 | 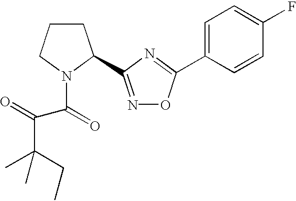 |
| 428 | 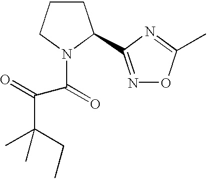 |
| 429 | 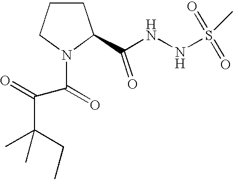 |
| 430 | 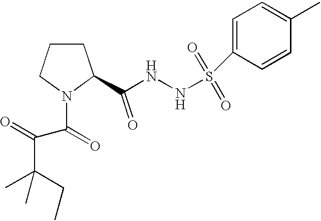 |
| 431 | 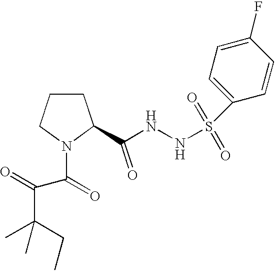 |
| 432 | 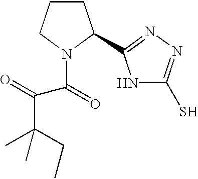 |
| 433 | 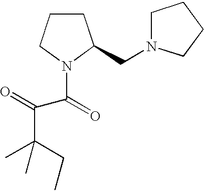 |
| 434 | 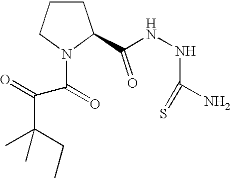 |
| 435 | 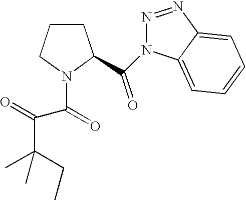 |
| 436 | 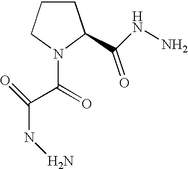 |
| 437 | 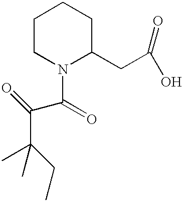 |
| 438 | 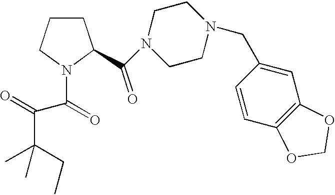 |
| 439 | 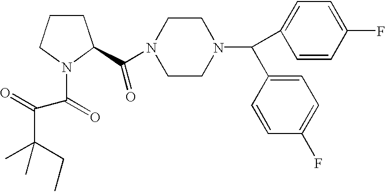 |

Another preferred embodiment of this aspect of the invention is the use for the treatment of nerve injury caused as a consequence of prostate surgery of a compound of the formula (LXV):

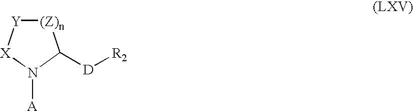

in which

X, Y, and Z are independently selected from the group consisting of C, O, S, or N, provided that X, Y, and Z are not all C;

n is 1-3;

A is selected from the group consisting of L₁, L₂, L₃, or L₄, in which

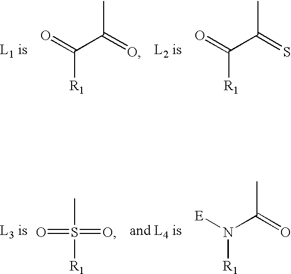

and R₁ and E, independently, are selected from the group consisting of hydrogen, C₁-C₉ straight or branched chain alkyl, C₂-C₉ straight or branched chain alkenyl, aryl, heteroaryl, carbocycle, and heterocycle;

R₂ is carboxylic acid or a carboxylic acid isostere; wherein said alkyl, alkenyl, alkynyl, aryl, heteroaryl, carbocycle, heterocycle, or carboxylic acid isostere is optionally substituted with one or more substituents selected from R³, where

R³ is hydrogen, hydroxy, halo, halo(C₁-C₆)-alkyl, thiocarbonyl, (C₁-C₆)-alkoxy, (C₂-C₆)-alkenoxy, (C₁-C₆)— alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)-alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl;

or a pharmaceutically acceptable salt, ester, or solvate thereof.

Preferred embodiments of this embodiment of the invention are those in which R₂ is a carbocycle or heterocycle containing any combination of CH₂, O S, or N in any chemically stable oxidation state, where any of the atoms of said ring structure are optionally substituted in one or more positions with R³.

Especially preferred embodiments of this aspect of the invention are the use of those compounds in which R₂ is selected from the group below:

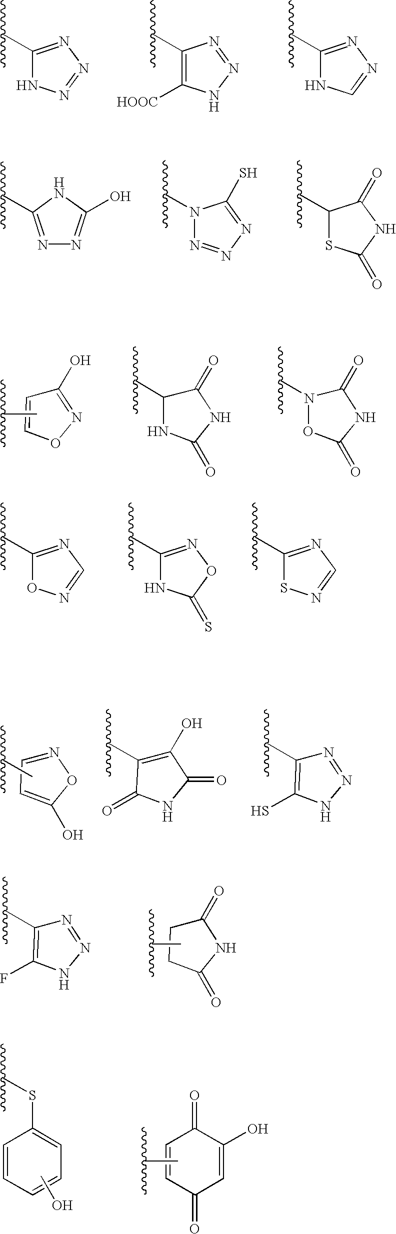

where the atoms of said ring structure may be optionally substituted at one or more positions with R³.

Another preferred embodiment of this invention is where R₂ is selected from the group consisting of —COOH, —SO₃H, —SO₂HNR³, —PO₂ (R³)₂ , —CN, —PO₃(R³)₂, —OR³, —SR³, —NHCOR³, —N(R³)₂, —CON(R³)₂, —CONH(O)R³, —CONHNHSO₂R³, —COHNSO₂R³, and —CONR³CN.

Preferred embodiments of this embodiment are the neurotrophic compounds (2S)-1-(phenylmethyl) carbamoyl-2-hydroxymethyl (4-thiazolidine), (2S)-1-(1,1-dimethyl propyl)carbamoyl-2-(4-thiazolidine)tetrazole and (2S)-1-(phenylmethyl) carbamoyl-2-(4-thiazolidine) carbonitrile.

The following structures are non-limiting examples of preferred carbocyclic and heterocyclic isosteres contemplated by this aspect of the invention:

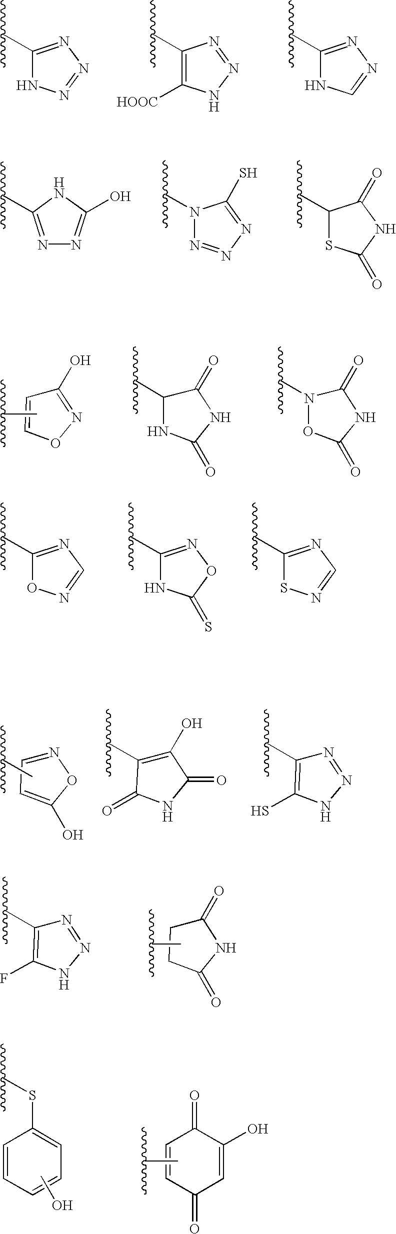

in which the atoms of said ring structure may be optionally substituted at one or more positions with R³ wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl. The present invention contemplates that when chemical substituents are added to a carboxylic isostere then the compound retains the properties of a carboxylic isostere. Particularly, the present invention contemplates that when a carboxylic isostere is optionally substituted with one or more moieties selected from R³, then the substitution cannot eliminate the carboxylic acid isosteric properties of the compound. The present invention contemplates that the placement of one or more R³ substituents upon a carbocyclic or heterocyclic carboxylic acid isostere shall not be at an atom(s) which maintains or is integral to the carboxylic acid isosteric properties of the inventive compound if such a substituent(s) would destroy the carboxylic acid isosteric properties of the inventive compound.

Other carboxylic acid isosteres not specifically exemplified or described in this specification are also contemplated by the present invention.

A compound for use in the present invention, especially formula LXV, wherein n is 1, X is O, D is a bond, R₁ is 1,1 dimethylpropyl, and R₂ is —CN, is named (2S)-1-(1,2-dioxo-3,3-dimethylpentyl)-2-pyrrolidinecarbonitrile.

Specific embodiments of the inventive compounds are presented in Tables XLV, XLVI, and XLVII. The present invention contemplates employing the compounds of Tables XLV, XLVI, and XLVII, below, for use in compositions and methods of the invention.

TABLE XLV

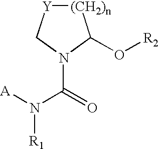

| No. | n | D | R₂ | A | Y | R₁ |
|---|---|---|---|---|---|---|
| 440 | 1 | bond | COOH | H | S | Benzyl |
| 441 | 1 | bond | COOH | H | S | a-MethylBenzyl |
| 442 | 1 | bond | COOH | H | S | 4-MethylBenzyl |
| 443 | 1 | bond | Tetrazole | H | S | Benzyl |
| 444 | 1 | bond | SO₃H | H | O | a-MethylBenzyl |
| 445 | 1 | CH₂ | COOH | H | O | 4-MethylBenzyl |
| 446 | 1 | bond | SO₂HNMe | H | O | Benzyl |
| 447 | 1 | bond | CN | H | N | a-MethylBenzyl |
| 448 | 1 | bond | PO₃H₂ | H | N | 4-MethylBenzyl |
| 449 | 2 | bond | COOH | H | N | Benzyl |
| 450 | 2 | bond | COOH | H | S | a-MethylBenzyl |
| 451 | 2 | bond | COOH | H | S | 4-MethylBenzyl |
| 452 | 2 | bond | COOH | H | S | 3,4,5-trimethoxy-phenyl |
| 453 | 2 | bond | COOH | H | S | Cyclohexyl |
| 454 | 2 | bond | PO₂HEt | H | O | a-propyl |
| 455 | 2 | bond | PO₃HHPropyl | H | O | ethyl |
| 456 | 2 | bond | PO₃(Et)₂ | H | N | Methyl |
| 457 | 2 | bond | Ome | H | S | tert-butyl |
| 458 | 2 | bond | Oet | H | S | n-pentyl |
| 459 | 2 | bond | OPropyl | H | S | n-hexyl |
| 460 | 1 | bond | OButyl | H | O | Cyclohexyl |
| 461 | 1 | bond | OPentyl | H | N | cyclopentyl |
| 462 | 1 | bond | OHexyl | H | S | n-heptyl |
| 463 | 1 | bond | Sme | H | S | n-octyl |
| 464 | 1 | bond | Set | H | O | n-nonyl |
| 465 | 2 | bond | SPropyl | H | N | 2-indolyl |
| 466 | 2 | bond | SButyl | H | O | 2-furyl |
| 467 | 2 | bond | NHCOMe | H | S | 2-thiazolyl |
| 463 | 2 | bond | NHCOEt | H | S | 2-thienyl |
| 469 | 1 | CH₂ | N(Me)₂ | H | N | 2-pyridyl |
| 470 | 1 | (CH₂)₂ | N(Me)Et | H | S | 1,1-dimethylpropyl |
| 471 | 1 | (CH₂)₃ | CON(Me)₂ | H | O | 1,1-dimethylpropyl |
| 472 | 1 | (CH₂)₄ | CONHMe | H | N | 1,1-dimethylpropyl |
| 473 | 1 | (CH₂)₅ | CONHEt | H | S | 1,1-dimethylpropyl |
| 474 | 1 | (CH₂)₆ | CONHPropyl | H | S | 1,1-dimethylpropyl |

TABLE XLVI

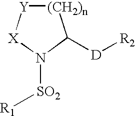

| No. | n | D | R₂ | Y | R₁ |
|---|---|---|---|---|---|
| 475 | | bond | CONH(O)Me | S | Benzyl |
| 476 | | bond | CONH(O)Et | S | a-Methylphenyl |
| 477 | 1 | bond | CONH(O)Propyl | S | 4-Methylphenyl |
| 478 | 2 | bond | COOH | S | Benzyl |
| 479 | 2 | bond | COOH | O | a-Methylphenyl |
| 480 | 2 | bond | COOH | O | 4-Methylphenyl |
| 481 | 1 | CH₂ | COOH | N | benzyl |
| 482 | 1 | (CH₂)₂ | COOH | N | benzyl |
| 483 | 1 | (CH₂)₃ | COOH | N | benzyl |
| 484 | 1 | (CH₂)₄ | COOH | S | benzyl |
| 485 | 1 | (CH₂)₅ | COOH | S | benzyl |
| 486 | 1 | (CH₂)₆ | COOH | S | benzyl |
| 487 | 1 | (CH₂)₉ | COOH | S | benzyl |
| 488 | 1 | (CH₂)₈ | COOH | O | benzyl |
| 489 | 1 | (CH₂)₉ | COOH | O | benzyl |
| 490 | 1 | (CH₂)₁₀ | COOH | O | benzyl |
| 491 | 1 | C₂H₂ | COOH | N | benzyl |
| 492 | 1 | 2-OH, Et | COOH | N | benzyl |
| 493 | 1 | 2butylene | COOH | S | benzyl |
| 494 | 1 | i-Pro | COOH | S | benzyl |
| 495 | 1 | tert-Bu | COOH | S | benzyl |
| 496 | 1 | 2-nitro | COOH | S | benzyl |
| | | | | | Hexyl |
| 497 | 3 | (CH₂)₂ | CN | S | benzyl |
| 499 | 1 | (CH₂)₂ | CN | S | benzyl |
| 499 | 3 | bond | CONHNHSO₂Me | N | Benzyl |
| 500 | 3 | bond | CONHNHSO₂Et | N | a-Methylphenyl |
| 501 | 3 | bond | CONHSO₂Me | N | 4-Methylphenyl |
| 502 | 2 | bond | CONHNHSO₂Et | N | Phenyl |
| 503 | 2 | bond | CON(Me)CN | O | a-Methylphenyl |
| 504 | 2 | bond | CON(Et)CN | O | 4-Methylphenyl |
| 505 | 1 | (CH₂)₂ | COOH | O | methyl |
| 506 | 1 | (CH₂)₂ | COOH | O | ethyl |
| 507 | 1 | (CH₂)₄ | COOH | N | n-propyl |
| 508 | 1 | (CH₂)₅ | COOH | N | t-butyl |
| 509 | 1 | (CH₂)₆ | COOH | N | Pentyl |
| 510 | 1 | (CH₂)₂ | COOH | S | Hexyl |
| 511 | 1 | (CH₂)₃ | COOH | S | Heptyl |
| 512 | 1 | (CH₂)₉ | COOH | S | Octyl |
| 513 | 1 | (CH₂)₁₀ | COOH | S | Nonyl |
| 514 | 1 | C₂H₂ | COOH | S | Cyclohexyl |

TABLE XLVII

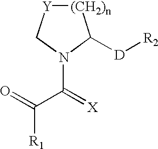

| No. | n | X | D | R₂ | Y | R₁ |
|---|---|---|---|---|---|---|
| 515 | 1 | O | bond | 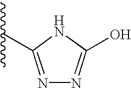 | O | 1,1-dimethylpropyl |
| 516 | 1 | O | bond | 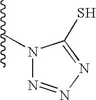 | S | 1,1-dimethylpropyl |
| 517 | 1 | O | bond | 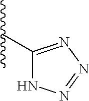 | S | 1,1-dimethylpropyl |
| 518 | 1 | O | bond | 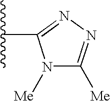 | O | 1,1-dimethylpropyl |
| 519 | 1 | O | bond | 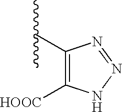 | N | 1,1-dimethylpropyl |
| 520 | 1 | O | bond | 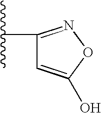 | S | 1,1-dimethylpropyl |
| 521 | 1 | O | bond | 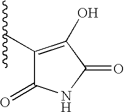 | N | 1,1-dimethylpropyl |
| 522 | 1 | O | bond | 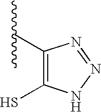 | N | 1,1-dimethylpropyl |
| 523 | 1 | O | bond | 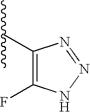 | S | 1,1-dimethylpropyl |
| 524 | 1 | O | bond | 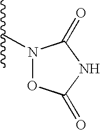 | O | 1,1-dimethylpropyl |
| 525 | 1 | O | bond | 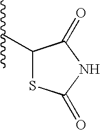 | S | 1,1-dimethylpropyl |
| 526 | 1 | O | bond | 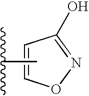 | S | 1,1-dimethylpropyl |
| 527 | 1 | O | bond | 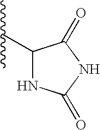 | O | 1,1-dimethylpropyl |
| 528 | 1 | O | bond | 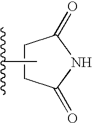 | S | 1,1-dimethylpropyl |
| 529 | 1 | O | bond | 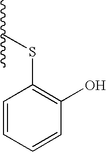 | O | 1,1-dimethylpropyl |
| 530 | 1 | O | bond | 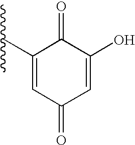 | S | 1,1-dimethylpropyl |
| 531 | 1 | O | bond | 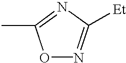 | N | 1,1-dimethylpropyl |
| 532 | 1 | O | bond | 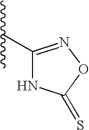 | O | 1,1-dimethylpropyl |
| 533 | 1 | O | bond | 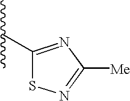 | S | 1,1-dimethylpropyl |

Compounds 534-627 are also exemplified for use in the present invention, and are defined as where Y is located at the 3-position of the heterocyclic ring for compounds 440-533, and n, A, D, Y, X, R₁, and R₂ remain the same as defined for compounds 440-533 in Tables XLV, XLVI, and XLVII.

Exemplary compound 628 is defined where S is located at the 3-position of the heterocvclic ring (3-thiazolidine), n is 1, R₁ is 1,1-dimethylpropyl, D is a bond, R₂ is COOH.

Exemplary compound 629 is defined where O is located at the 2-position of the heterocyclic ring (2-oxopentanoyl), n is 1, R₁ is 1,1-dimethylpropyl, D is a bond, R₂ is COOH (i.e. 3-(3,3-dimethyl-2-oxopentanoyl)-1,3-oxazolidine-4-carboxylic acid).

The present invention also contemplates other ring locations for the heteroatoms O, N, and S in neurotrophic heterocyclic compounds. Also contemplated by the present invention are neurotrophic heterocycles containing 3 or more heteroatoms chosen independently from O, N, and S.

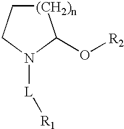

| No. | n | D | R₂ | L | R₁ |
|---|---|---|---|---|---|
| 630 | 1 | CH₂ | OH | 1,2-dioxoethyl | benzyl |
| 631 | 1 | bond | —CN | 1,2-dioxoethyl | 1,1-dimethylpropyl |
| 632 | 1 | bond | tetrazole | 1,2-dioxoethyl | 1,1-dimethylpropyl |
| 633 | 2 | bond | CONH₂ | 1,2-dioxoethyl | 1,1-dimethylpropyl |
| 634 | 1 | bond | COOH | 1,2-dioxoethyl | 1,1-dimethylpropyl |
| 635 | 2 | bond | COOH | 1,2-dioxoethyl | 1,1-dimethylpropyl |

In another embodiment of the invention, there is provided a compound for the treatment of nerve injury caused as a consequence of prostate surgery of formula (LXVI):

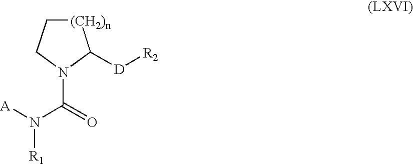

in which:

n is 1-3;

R₁ and A are independently selected from the group consisting of hydrogen, C₁-C₉ straight or branched chain alkyl, C₂-C₉ straight or branched chain alkenyl, aryl, heteroaryl, carbocycle, and heterocycle;

D is a bond, or a C₁-C₁₀ straight or branched chain alkyl, C₂-C₁₀ alkenyl or C₂-C₁₀ alkynyl;

R₂ is carboxylic acid or a carboxylic acid isostere; wherein said alkyl, alkenyl, alkynyl, aryl, heteroaryl, carbocycle, heterocycle, or carboxylic acid isostere is optionally substituted with one or more substituents selected from R³, where

R³ is hydrogen, hydroxy, halo, halo(C₁-C₆)-alkyl, thiocarbonyl, (C₁-C₆)-alkoxy, (C₂-C₆)-alkenoxy, (C₁-C₆)— alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)-alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl;

or a pharmaceutically acceptable salt, ester, or solvate thereof.

A preferred compound for use in this embodiment of this invention is (2S)-1-(cyclohexyl)carbamoyl-2-pyrrolidinecarboxylic acid.

Other preferred compounds for use in this embodiment of this invention are those in which R₂ is a carbocycle or heterocycle containing any combination of CH₂, O, S, or N in any chemically stable oxidation state, where any of the atoms of said ring structure are optionally substituted in one or more positions with R³.

Especially preferred embodiments of this aspect of the invention are those in which R₂ is selected from the group below:

(See figures on next page)

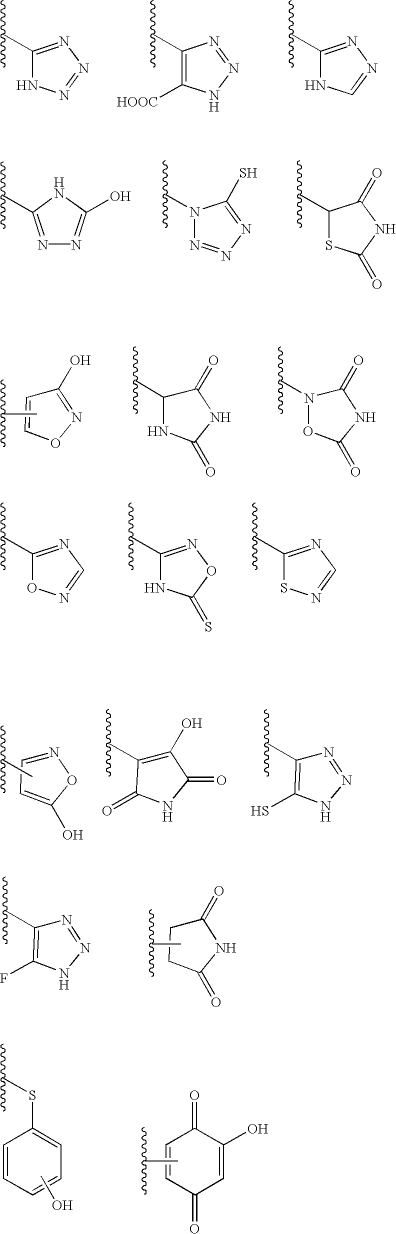

where the atoms of said ring structure may be optionally substituted at one or more positions with R³.

Another preferred embodiment of this invention is where R₂ is selected from the group consisting of —COOH, —SO₃H, —SO₂HNR³, —PO₂(R³)₂, CN, —PO₃(R³,)₂, —OR³, —SR³, —NHCOR³, —N(R³)₂, —CON(R³)₂, —CONH(O)R³, —CONHNHSO₂R³, —COHNSO₂R³, and —CONR³CN.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —COOH, —SO₃H, —SO₂HNR³, —PO₂(R³)₂, —CN, —PO₃ (R³)₂, —OR³, —SR³, —NHCOR³, —N(R³)₂, —CON(R³)₂, —CONH(O)R³, —CONHNHSO₂R³, —COHNSO₂R³, and —CONR³CN wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulthydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl.

In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carbocyclic and heterocyclic isosteres contemplated by this aspect of the invention.

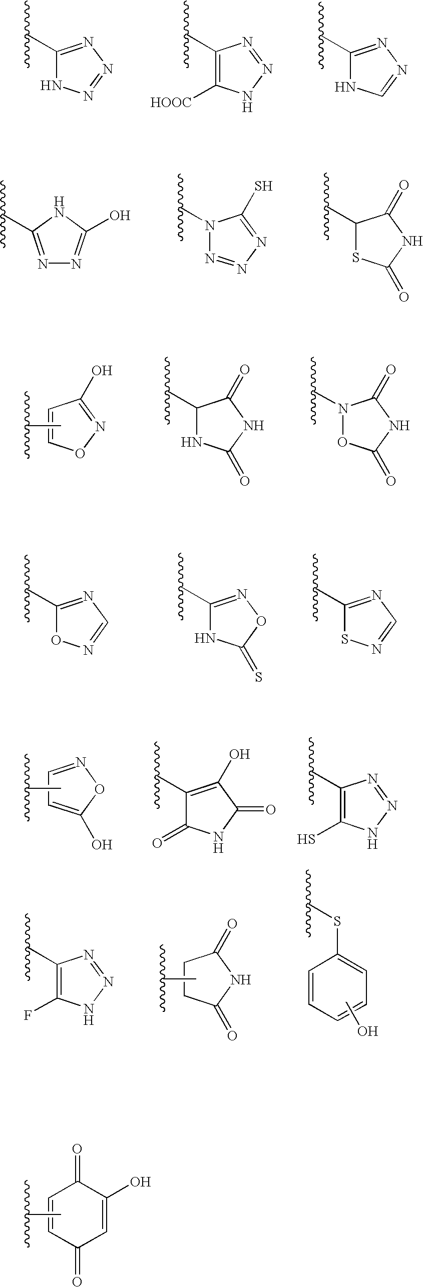

where the atoms of said ring structure may be optionally substituted at one or more positions with R³ wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle, and CO₂R⁴ where R⁴ is hydrogen or C₁-C₉ straight or branched chain alkyl or alkenyl. The present invention contemplates that when chemical substituents are added to a carboxylic isostere then the inventive compound retains the properties of a carboxylic isostere.

The present invention contemplates that when a carboxylic isostere is optionally substituted with one or more moieties selected from R³, then the substitution cannot eliminate the carboxylic acid isosteric properties of the inventive compound. The present invention contemplates that the placement of one or more R³ substituents upon a carbocyclic or heterocyclic carboxylic acid isostere shall not be permitted at one or more atom(s) which maintain(s) or is/are integral to the carboxylic acid isosteric properties of the inventive compound, if such substituent(s) would destroy the carboxylic acid isosteric properties of the inventive compound.

A compound of the present invention, especially formula LXVI, wherein n is 1, X is O, D is a bond, R₁ is 1,1,dimethylpropyl, and R₂ is —CN, is named (2S)-1-(1,2-dioxo-3,3-dimethylpentyl)-2-pyrrolidinecarbonitrile.

Specific embodiments of the inventive compounds are presented in Table XLVIII. The present invention contemplates employing the compounds of Table XLVIII, below, for use in compositions and methods of the invention.

TABLE XLVIII

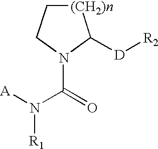

| No. | n | D | R₂ | A | R₁ |
|---|---|---|---|---|---|
| 636 | 1 | bond | COOH | H | cyclohexyl |
| 637 | 1 | bond | COOH | H | a-MethylBenzyl |
| 638 | 1 | bond | COOH | H | 4-MethylBenzyl |
| 639 | 1 | bond | Tetrazole | H | Benzyl |
| 640 | 1 | bond | SO₂H | H | a-MethylBenzyl |
| 641 | 1 | CH₂ | COOH | H | 4-MethylBenzyl |
| 642 | 1 | bond | SO₂HNMe | H | Benzyl |
| 643 | 1 | bond | CN | H | a-MethylBenzyl |
| 644 | 1 | bond | PO₃H₂ | H | 4-MethylBenzyl |
| 645 | 2 | bond | COOH | H | Benzyl |
| 646 | 2 | bond | COOH | H | a-MethylBenzyl |
| 647 | 2 | bond | COOH | H | 2-butyl |
| 648 | 2 | bond | COOH | H | 2-butyl |
| 649 | 2 | bond | COOH | H | Cyclohexyl |
| 650 | 2 | bond | PO₂Het | H | i-propyl |
| 651 | 2 | bond | PO₃HPropyl | H | ethyl |
| 652 | 2 | bond | PO₃(Et)₂ | H | Methyl |
| 653 | 2 | bond | Ome | H | tert-butyl |
| 654 | 2 | bond | Oet | H | n-pentyl |
| 655 | 2 | bond | Opropyl | H | n-hexyl |
| 656 | 1 | bond | Obutyl | H | Cyclohexyl |
| 657 | 1 | bond | Opentyl | H | cyclopentyl |
| 658 | 1 | bond | Ohexyl | H | heptyl |
| 659 | 1 | bond | Sme | H | n-octyl |
| 660 | 1 | bond | Set | H | n-hexyl |
| 661 | 2 | bond | Spropyl | H | n-hexyl |
| 662 | 2 | bond | Sbutyl | H | n-hexyl |
| 663 | 2 | bond | NHCOMe | H | n-hexyl |
| 664 | 2 | bond | NHCOEt | H | 2-thienyl |
| 665 | 1 | CH₂ | N(Me)₂ | H | adamantyl |
| 666 | 1 | (CH₂)₂ | N(Me)Et | H | adamantyl |
| 667 | 1 | (CH₂)₃ | CON(Me)₂ | H | adamantyl |
| 668 | 1 | (CH₂)₄ | CONHMe | H | adamantyl |
| 669 | 1 | (CH₂)₅ | CONHEt | H | adamantyl |
| 670 | 1 | (CH₂)₆ | CONHPropyl | H | adamantyl |
| 671 | 1 | bond | CONH(O)Me | H | Benzyl |
| 672 | 1 | bond | CONH(O)Et | H | α-methylphenyl |
| 673 | 1 | bond | CONH(O)Propyl | H | 4-Methylphenyl |
| 674 | 2 | bond | COOH | H | Benzyl |
| 675 | 2 | bond | COOH | H | α-Methylphenyl |
| 676 | 2 | bond | COOH | H | 4-Methylphenyl |
| 677 | 1 | CH₂ | COOH | Me | cyclohexyl |
| 678 | 1 | (CH₂)₂ | COOH | Et | cyclohexyl |
| 679 | 1 | (CH₂)₃ | COOH | Prop | cyclohexyl |
| 680 | 1 | (CH₂)₄ | COOH | But | cyclohexyl |
| 681 | 1 | (CH₂)₅ | COOH | H | cyclohexyl |
| 682 | 1 | (CH₂)₆ | COOH | H | cyclohexyl |
| 683 | 1 | (CH₂)₇ | COOH | H | cyclohexyl |
| 684 | 1 | (CH₂)₄ | COOH | H | cyclohexyl |
| 685 | 1 | (CH₂)₉ | COOH | H | cyclohexyl |
| 686 | 1 | (CH₂)₁₀ | COOH | H | cyclohexyl |
| 687 | 1 | C₂H₂ | COOH | H | cyclohexyl |
| 688 | 1 | 2-OH, Et | COOH | H | cyclohexyl |
| 689 | 1 | 2-butylene- | COOH | H | cyclohexyl |
| 690 | 1 | i-Pro | COOH | H | cyclohexyl |
| 691 | 1 | tert-Bu | COOH | H | cyclohexyl |
| 692 | 1 | 2-nitro Hexyl | COOH | H | cyclohexyl |
| 693 | 3 | (CH₂)₂ | CN | H | cyclohexyl |
| 694 | 1 | (CH₂)₃ | CN | H | cyclohexyl |
| 695 | 3 | bond | CONHNHSO₂Me | H | Benzyl |
| 696 | 3 | bond | CONHNHSO₂Et | H | α-Methylphenyl |
| 697 | 3 | bond | CONHSO₂Me | H | 4-Methylphenyl |
| 698 | 2 | bond | CONHNHSO₂Et | H | Phenyl |
| 699 | 2 | bond | CON(Me)CN | H | α-Methylphenyl |
| 700 | 2 | bond | CON(Et)CN | H | 4-Methylphenyl |
| 701 | 1 | (CH₂)₂ | COOH | H | methyl |
| 702 | 1 | (CH₂)₃ | COOH | H | ethyl |
| 703 | 1 | (CH₂)₄ | COOH | H | n-propyl |
| 704 | 1 | (CH₂)₅ | COOH | H | t-butyl |
| 705 | 1 | (CH₂)₆ | COOH | H | Bentyl |
| 706 | 1 | (CH₂)₇ | COOH | H | Hexyl |
| 707 | 1 | (CH₂)₄ | COOH | H | Heptyl |
| 708 | 1 | (CH₂)₉ | COOH | H | Octyl |
| 709 | 1 | (CH₂)₁₀ | COOH | H | Nonyl |
| 710 | 1 | C₂H₂ | COOH | H | Cyclohexyl |
| 711 | 1 | bond | 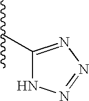 | H | cyclohexyl |
| 712 | 1 | bond | 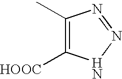 | H | cyclohexyl |
| 713 | 1 | bond | 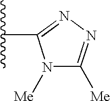 | H | cyclohexyl |
| 714 | 1 | bond | 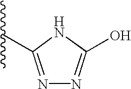 | H | cyclohexyl |
| 715 | 1 | bond | 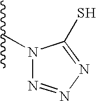 | H | cyclohexyl |
| 716 | 1 | bond | 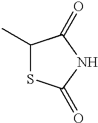 | H | cyclohexyl |
| 717 | 1 | bond | 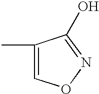 | H | cyclohexyl |
| 718 | 1 | bond | 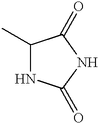 | H | cyclohexyl |
| 719 | 1 | bond | 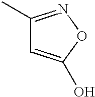 | H | cyclohexyl |
| 720 | 1 | bond | 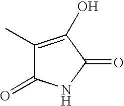 | H | cyclohexyl |
| 721 | 1 | bond | 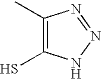 | H | cyclohexyl |
| 722 | 1 | bond | 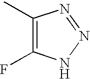 | H | cyclohexyl |
| 723 | 1 | bond | 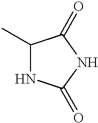 | H | cyclohexyl |
| 724 | 1 | bond | 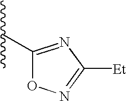 | H | cyclohexyl |
| 725 | 1 | bond | 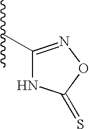 | H | cyclohexyl |
| 726 | 1 | bond | 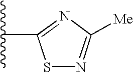 | H | cyclohexyl |
| 727 | 1 | bond | 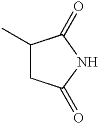 | H | cyclohexyl |
| 728 | 1 | bond | 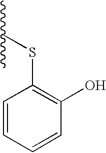 | H | cyclohexyl |
| 729 | 1 | bond | 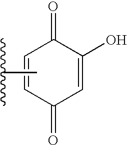 | H | cyclohexyl |

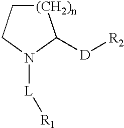

| No. | n | D | R₂ | L | R₁ |
|---|---|---|---|---|---|
| 730 | 1 | CH₂ | OH | 1,2-dioxoethyl | benzyl |
| 731 | 1 | bond | —CN | 1,2-dioxoethyl | 1,1-dimethylpropyl |
| 732 | 1 | bond | tetrazole | 1,2-dioxoethyl | 1,1-dimethylpropyl |
| 733 | 2 | bond | CONH₂ | 1,2-dioxoethyl | 1,1-dimethylpropyl |
| 734 | 1 | bond | COOH | 1,2-dioxoethyl | 1,1-dimethylpropyl |
| 735 | 2 | bond | COOH | 1,2-dioxoethyl | 1,1-dimethylpropyl |

Another preferred embodiment of the invention is the use for the treatment of nerve injury caused as a consequence of prostate surgery with a compound of the formula (LXVII):

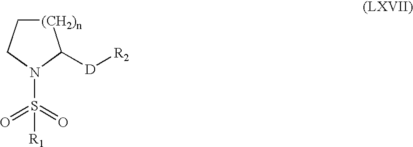

in which:

n is 1-3;

R₁ is selected from the group consisting of hydrogen, C₁-C₉ straight or branched chain alkyl, C₂-C₉ straight or branched chain alkenyl, aryl, heteroaryl, carbocycle, or heterocycle;

D is a bond, or a C₁-C₁₀ straight or branched chain alkyl, C₂-C₁₀ alkenyl or C₂-C₁₀alkynyl; R₂ is a carboxylic acid or a carboxylic acid isostere;

wherein said alkyl, alkenyl, alkynyl, aryl, heteroaryl, carbocycle, heterocycle, or carboxylic acid isostere is optionally substituted with one or more substituents selected from R³, where R³ is hydrogen, hydroxy, halo, halo-(C₁-C₆)-alkoxy, thiocarbonyl, (C₁-C₆)-alkoxy, (C₂-C₆)-alkenyloxy, (C₁-C₆)-alkylaryloxy, aryloxy, aryl-(C₁-C₆)-alkyloxy, cyano, nitro, imino, (C₁-C₆)-alkylamino, amino-(C₁-C₆)-alkyl, sulfhydryl, thio-(C₁-C₆)alkyl, (C₁-C₆)-alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl;

or a pharmaceutically acceptable salt, ester or solvate thereof.

A preferred embodiment of this invention is the use of a compound in which R₂ is a carbocycle or heterocycle containing any combination of CH₂, O, S, or N in any chemically stable oxidation state, where any of the atoms of said ring structure are optionally substituted in one or more positions with R³.

Especially preferred embodiments of this aspect of the invention are the use of those compounds in which R₂ is selected from the group below:

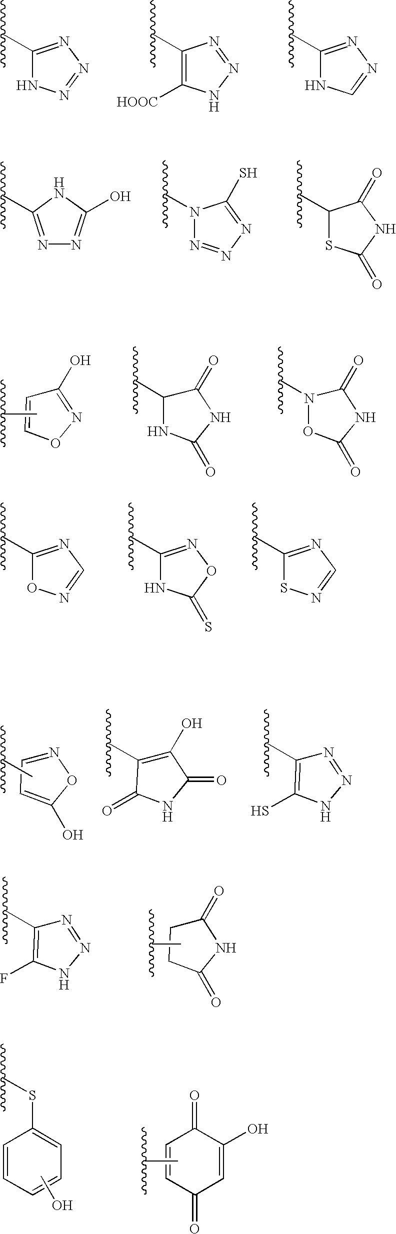

in which the atoms of said ring structure may be optionally substituted at one or more positions with R³.

Another preferred embodiment of this invention is where R₂ is selected from the group consisting of —COOH, —SO₃H, —SO₂HNR³, —PO₂(R³)₂, —CN, —PO₃(R³)₂, —OR³, —SR³, —NHCOR³, —N(R³)₂, —CON(R³)₂, —CONH(O)R³, —CONHNHSO₂R³, —COHNSO₂R³, and —CONR³CN.

Preferred embodiments of this invention are the following compounds: (2S)-1-(phenylmethyl)sulfonyl-2-hydroxymethylpyrrolidLne; (2S)-1-(phenylmethyl)-sulfonyl-2-pyrrolidinetetrazole; (2S)-1-(phenyl-methyl)-sulfonyl-2-pyrrolidine carbonitrile; and compounds 719-821.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —COOH, —SO₃H, —SO₂HNR³, —PO₂ (R³)₂, —CN, —O₃(R³)₂, —OR³, —SR³, —NHCOR³, —N(R³)₂, —CON(R³)₂, —CONH(O)R³, —CONHNHSO₂R³, —COHNSO₂R³, and —CONR³CN, wherein R³ is hydrogen, hydroxy, halo, halo-C₁-C₆-alkyl, thiocarbonyl, C₁-C₆-alkoxy, C₂-C₆-alkenoxy, C₁-C₆-alkylaryloxy, aryloxy, aryl-C₁-C₆-alkyloxy, cyano, nitro, imino, C₁-C₆-alkylamino, amino-C₁-C₆-alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycler and CO₂R⁴ where R⁴ is hydrogen or C₁-C₉ straight or branched chain alkyl or alkenyl.

In addition, carboxylic acid isosteres can include 5-7 membered carbocycles or heterocycles containing any combination of CH₂, O S, or N in any chemically stable oxidation state, where any of the atoms of said ring structure are optionally substituted in one or more positions. The following structures are non-limiting examples of preferred carbocyclic and heterocyclic isosteres contemplated by this aspect of the invention.

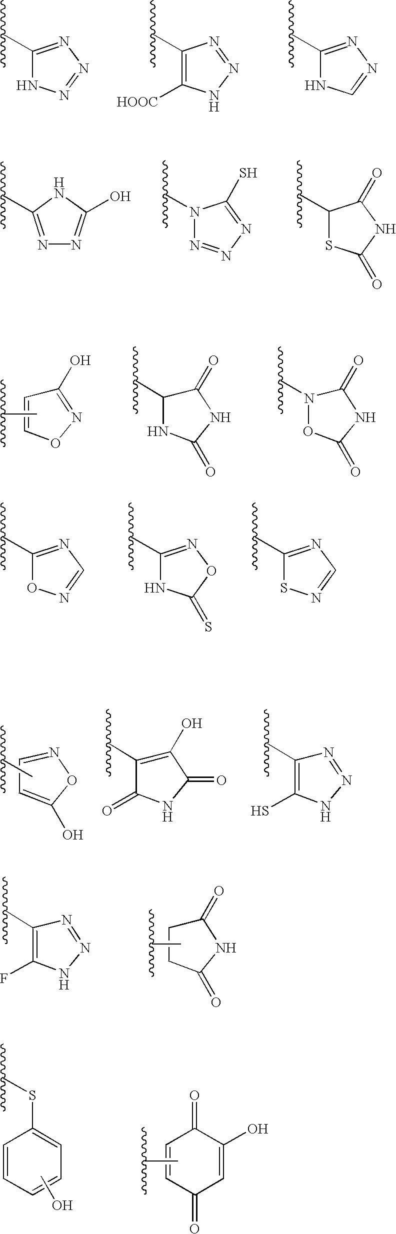

where the atoms of said ring structure may be optionally substituted at one or more positions with R³. The present invention contemplates that when chemical substituents are added to a carboxylic isostere then the inventive compound retains the properties of a carboxylic isostere. The present invention contemplates that when a carboxylic isostere is optionally substituted with one or more moieties selected from R³, then the substitution can not eliminate the carboxylic acid isosteric properties of the inventive compound. The present invention contemplates that the placement of one or more R³ substituents upon a carbocyclic or heterocyclic carboxylic acid isostere shall not be at an atom(s) which maintains or is integral to the carboxylic acid isosteric properties of the inventive compound if such a substituent(s) would destroy the carboxylic acid isosteric properties of the inventive compound.

Other carboxylic acid isosteres not specifically exemplified or described in this specification are also contemplated by the present invention.

A compound of the present invention, especially formula LXVII, wherein n is 1, D is a bond, R₁ is phenylmethyl, and R₂ is —CN, is named (2S)-1-(phenylmethyl) sulfonyl-2-pyrrolidine carbonitrile.

Specific embodiments of the inventive compounds are presented in Table XLIX. The present invention contemplates employing the compounds of Table XLVIX, below, for use in compositions and methods of the invention.

TABLE XLVIX

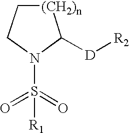

| No. | n | D | R₂ | R₁ |
|---|---|---|---|---|
| 736 | 1 | bond | COOH | Benzyl |
| 737 | 1 | bond | COOH | a-MethylBenzyl |
| 738 | 1 | bond | COOH | 4-MethylBenzyl |
| 739 | 1 | bond | Tetrazole | Benzyl |
| 740 | 1 | bond | SO₃H | a-MethylBenzyl |
| 741 | 1 | CH₂ | COOH | 4-MethylBenzyl |
| 742 | 1 | bond | SO₂HNMe | Benzyl |
| 743 | 1 | bond | CN | a-MethylBenzyl |
| 744 | 1 | bond | PO₃H₂ | 4-MethylBenzyl |
| 745 | 2 | bond | COOH | Benzyl |
| 746 | 2 | bond | COOH | a-MethylBenzyl |
| 747 | 2 | bond | COOH | 4-MethylBenzyl |
| 748 | 2 | bond | COOH | 3,4,5-trimethoxy- |
| | | | | phenyl |
| 749 | 2 | bond | COOH | Cyclohexyl |
| 750 | 2 | bond | PO₂HEt | 1-propyl |
| 751 | 2 | bond | PO₃HPropyl | ethyl |
| 752 | 2 | bond | PO₃(Et)₂ | Methyl |
| 753 | 2 | bond | OMe | tert-butyl |
| 754 | 2 | bond | OEt | n-pentyl |
| 755 | 2 | bond | OPropyl | n-hexyl |
| 756 | 1 | bond | OButyl | Cyclohexyl |
| 757 | 1 | bond | OPentyl | cyclopentyl |
| 758 | 1 | bond | OHexyl | n-heptyl |
| 759 | 1 | bond | SMe | n-octyl |
| 760 | 1 | bond | SEt | n-nonyl |
| 761 | 2 | bond | SPropyl | 2-indolyl |
| 762 | 2 | bond | SButyl | 2-furyl |
| 763 | 2 | bond | NHCOMe | 2-thiazolyl |
| 764 | 2 | bond | NHCOEt | 2-thienyl |
| 765 | 1 | CH₂ | N(Me)₂ | 2-pyridyl |
| 766 | 1 | (CH₂)₂ | N(Me)Et | benzyl |
| 767 | 1 | (CH₂)₃ | CON(Me)₂ | benzyl |
| 768 | 1 | (CH₂)₄ | CONHMe | benzyl |
| 769 | 1 | (CH₂)₅ | CONHEt | benzyl |
| 770 | 1 | (CH₂)₆ | CONHPropyl | 1,1-dimethylpropyl |
| 771 | 1 | bond | CONH(O)Me | Benzyl |
| 772 | 1 | bond | CONH(O)Et | a-Methylphenyl |
| 773 | 1 | bond | CONN(O)Propyl | 4-Methylphenyl |
| 774 | 2 | bond | COOH | Benzyl |
| 775 | 2 | bond | COOH | a-Methylphenyl |
| 776 | 2 | bond | COOH | 4-Methylphenyl |
| 777 | 1 | CH₂ | COOH | benzvl |
| 778 | 1 | (CH₂)₂ | COOH | benzyl |
| 779 | 1 | (CH₂)₃ | COOH | benzyl |
| 780 | 1 | (CH₂)₄ | COOH | benzyl |
| 781 | 1 | (CH₂)₅ | COOH | benzyl |
| 782 | 1 | (CH₂)₆ | COOH | benzyl |
| 783 | 1 | (CH₂)₉ | COOH | benzyl |
| 784 | 1 | (CH₂)₃ | COOH | benzyl |
| 785 | 1 | (CH₂)₉ | COOH | benzyl |
| 786 | 1 | (CH₂)₁₀ | COOH | benzyl |
| 787 | 1 | C₂H₂ | COOH | benzyl |
| 788 | 1 | 2-hydroxyethyl | COOH | benzyl |
| 789 | 1 | 2-butylene | COOH | benzyl |
| 790 | 1 | i-Propyl | COOH | benzyl |
| 791 | 1 | Tert-Butyl | COOH | benzyl |
| 792 | 1 | 2-nitrohexyl | COOH | benzyl |
| 793 | 3 | (CH₂)₂ | CN | benzyl |
| 794 | 1 | (CH₂)₂ | CN | benzyl |
| 795 | 3 | bond | CONHNHSO₂Me | Benzyl |
| 796 | 3 | bond | CONHNHSO₂Et | a-Methylphenyl |
| 797 | 3 | bond | CONHSO₂Me | 4-Methylphenyl |
| 798 | 2 | bond | CONHNHSO₂Et | Phenyl |
| 799 | 2 | bond | CON(Me)CN | a-Methylphenyl |
| 800 | 2 | bond | CON(Et)CN | 4-Methylphenyl |
| 801 | 1 | (CH₂)₂ | COOH | methyl |
| 802 | 1 | (CH₂)₃ | COOH | ethyl |
| 803 | 1 | (CH₂)₄ | COOH | n-propyl |
| 804 | 1 | (CH₂)₅ | COOH | t-butyl |
| 805 | 1 | (CH₂)₆ | COOH | Pentyl |
| 806 | 1 | (CH₂)₇ | COOH | Hexyl |
| 807 | 1 | (CH₂)₈ | COOH | Heptyl |
| 808 | 1 | (CH₂)₉ | COOH | Octyl |
| 809 | 1 | (CH₂)₁₀ | COOH | Nonyl |
| 810 | 1 | C₂H₂ | COOH | Cyclohexyl |
| 811 | 1 | bond | 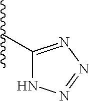 | benzyl |
| 812 | 1 | bond | 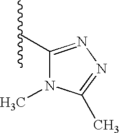 | benzyl |
| 813 | 1 | bond | 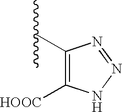 | benzyl |
| 814 | 1 | bond | 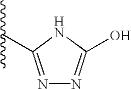 | benzyl |
| 815 | 1 | bond | 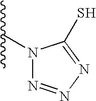 | benzyl |
| 816 | 1 | bond | 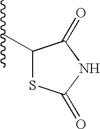 | benzyl |
| 817 | 1 | bond | 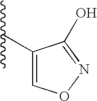 | benzyl |
| 818 | 1 | bond | 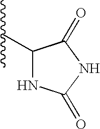 | benzyl |
| 819 | 1 | bond | 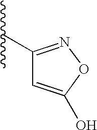 | benzyl |
| 820 | 1 | bond | 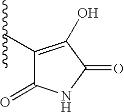 | benzyl |
| 821 | 1 | bond | 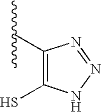 | benzyl |
| 822 | 1 | bond | 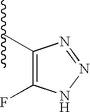 | benzyl |
| 823 | 1 | bond | 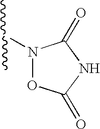 | benzyl |
| 824 | 1 | bond | 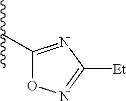 | benzyl |
| 825 | 1 | bond | 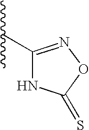 | benzyl |
| 826 | 1 | bond | 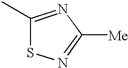 | benzyl |
| 827 | 1 | bond | 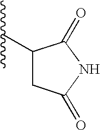 | benzyl |
| 828 | 1 | bond | 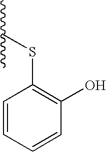 | benzyl |
| 829 | 1 | bond | 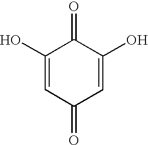 | benzyl |
| 830 | 1 | bond | CH₂OH | benzyl |
| 831 | 1 | bond | CONH₂ | benzyl |
| 832 | 1 | bond | CN | benzyl |

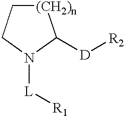

| No. | n | D | R₂ | L | R₁ |
|---|---|---|---|---|---|
| 833 | 1 | CH₂ | OH | 1,2-dioxoethyl | benzyl |
| 834 | 1 | bond | —CN | 1,2-dioxoethyl | 1,1-dimethylpropyl |
| 835 | 1 | bond | tetrazole | 1,2-dioxoethyl | 1,1-dimethylpropyl |
| 836 | 2 | bond | CONH₂ | 1,2-dioxoethyl | 1,1-dimethylpropyl |
| 837 | 1 | bond | COOH | 1,2-dioxoethyl | 1,1-dimethylpropyl |
| 838 | 2 | bond | COOH | 1,2-dioxoethyl | 1,1-dimethylpropyl |

VII. Aza Derivative Compounds

Another preferred embodiment of the invention is the use for the treatment of nerve injury caused as a consequence of prostate surgery with a compound of the formula (LXVIII):

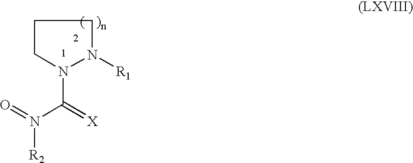

or a pharmaceutically acceptable salt, ester or solvate thereof, wherein:

n is 1-3;

R₁ is selected from the group consisting of —CR₃, —COOR₃, —COR₃, —COOH, —SO₃H, —SO₂HNR₃, —PO₂(R₃)₂, —CN, —PO₃ (R₃)₂, —OR₃, —SR₃, —NHCOR₃, —N(R₃)₂, —CON(R₃)₂, —CONH(O)R₃, —CONHNHSO₂R₃, —COHNSO₂R₃, —CONR₃CN,

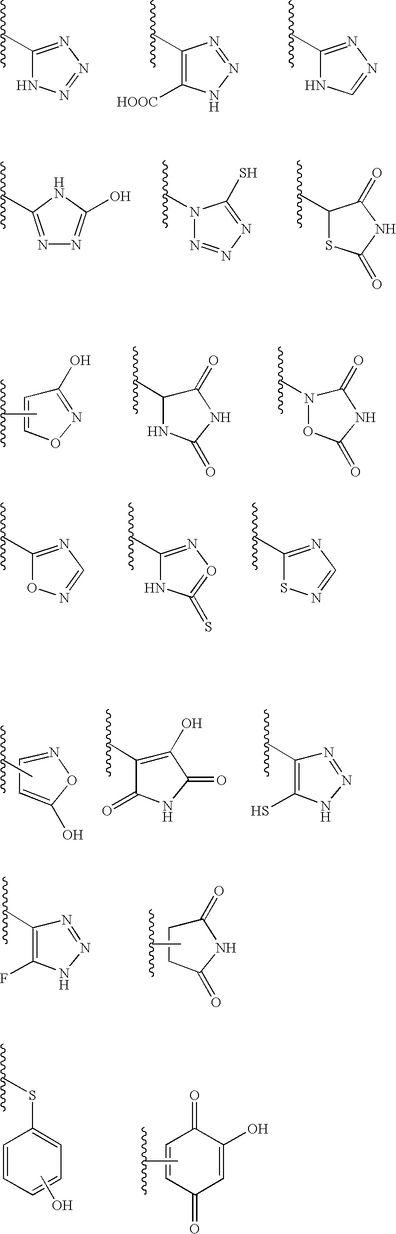

wherein said R₁ group is either unsubstituted or additionally substituted with R₃;

R₂ is selected from the group consisting of hydrogen, C₁-C₉ straight or branched chain alkyl, C₂-C₉ straight or branched chain alkenyl, C₂-C₉ straight or branched chain alkynyl, aryl, heteroaryl, carbocycle, or heterocycle, wherein said alkyl, alkenyl, alkynyl, aryl, heteroaryl, carbocycle, or heterocycle is unsubstituted on substituted with one or more substituents selected from R₃;

R₃ is selected from the group consisting of hydrogen, C₁-C₉ alkyl, C₂-C₉ straight or branched chain alkenyl, C₂-C₉ straight or branched chain alkynyl, C₁-C₉ alkoxy, C₂-C₉ alkenyloxy, aryloxy, phenoxy, benzyloxy, hydroxy, carboxy, C₁-C₉ thioalkyl, C₂-C₉ thioalkenyl, C₁-C₉ alkylamino, C₂-C₉ alkenylamino, cyano, nitro, imino, sulfonyl, thiocarbonyl, sulfhydryl, halo, haloalkyl, trifluoromethyl, aryl, heteroaryl, carbocycle, and heterocycle,

wherein said alkyl, alkenyl, alkynyl, alkoxy, alkenyloxy, aryloxy, thioalkyl, thioalkenyl, alkylamino, alkenylamino, aryl, heteroaryl, carbocycle, or heterocycle group is optionally substituted with a hydroxy, carboxy, carbonyl, cyano, nitro, imino, sulfonyl, thiocarbonyl, sulfhydryl, halo, haloalkyl, trifluoromethyl, aryl, heteroaryl, carbocycle, or heterocycle group; and X is O or S.

Specific embodiments of the inventive compounds are presented in Table L. The present invention contemplates employing the compounds of Table L, below, for use in compositions and methods of the invention.

TABLE L

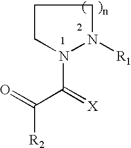

| No | N | X | R₁ | R₂ |
|---|---|---|---|---|
| 839 | 1 | O | 5-Phenylpentanoyl | 1,1-Dimethylpropyl |
| 840 | 1 | O | 3-Phenylpropanoyl | 1,1-Dimethylpropyl |
| 841 | 1 | O | 5-(3-Pyridyl)pent-4-ynoyl | 1,1-Dimethylpropyl |
| 842 | 1 | O | 5-(Cyano)pent-4-ynoyl | 1,1-Dimethylpropyl |
| 842 | 1 | O | 4-Phenylbutanoyl | 1,1-Dimethylpropyl |
| 844 | 1 | O | 6-Phenylhexanoyl | 1,1-Dimethylpropyl |
| 845 | 1 | O | 5-(3-Pyridyl)pentanoyl | 1,1-Dimethylpropyl |
| 846 | 1 | O | 3-Phenylpropyl ester | 1,1-Dimethylpropyl |
| 847 | 1 | O | 3-(3-Pyridyl)propyl ester | 1,1-Dimethylpropyl |
| 848 | 1 | O | 4-Phenylbutyl ester | 1,1-Dimethylpropyl |
| 849 | 1 | O | 2-Phenylethyl ester | 1,1-Dimethylpropyl |
| 850 | 2 | O | 6-Phenylhexanoyl | 1,1-Dimethylpropyl |
| 851 | 2 | O | 6-(3-Pyridyl)hexanoyl | 1,1-Dimethylpropyl |
| 852 | 2 | O | 3-Phenylpropyl ester | 1,1-Dimethylpropyl |
| 853 | 2 | O | 4-Phenylbutyl ester | 1,1-Dimethylpropyl |
| 854 | 2 | O | 5-Phenylpentyl ester | 1,1-Dimethylpropyl |
| 855 | 2 | O | 4-(3-Pyridyl)butyl ester | 1,1-Dimethylpropyl |
| 856 | 2 | O | 5-Phenylpentanoyl | 1,1-Dimethylpropyl |
| 857 | 1 | O | COOH | 3,4,5-trimethylphenyl |
| 858 | 2 | O | COOH | 3,4,5-trimethylphenyl |
| 859 | 1 | O | COOH | tert-butyl |
| 860 | 3 | O | COOH | tert-butyl |
| 861 | 1 | O | COOH | cyclopentyl |
| 862 | 2 | O | COOH | cyclopentyl |
| 863 | 3 | O | COOH | cyclopentyl |
| 864 | 1 | O | COOH | cyclohexyl |
| 865 | 2 | O | COOH | cyclohexyl |
| 866 | 3 | O | COOH | cyclohexyl |
| 867 | 1 | O | COOH | cycloheptyl |
| 868 | 2 | O | COOH | cycloheptyl |
| 869 | 3 | O | COOH | cycloheptyl |
| 870 | 1 | O | COOH | 2-thienyl |
| 871 | 2 | O | COOH | 2-thienyl |
| 872 | 3 | O | COOH | 2-thienyl |
| 873 | 1 | O | COOH | 2-furyl |
| 874 | 2 | O | COOH | 3-furyl |
| 875 | 3 | O | COOH | 4-furyl |
| 876 | 3 | O | COOH | phenyl |
| 877 | 1 | O | COOH | 1,1-dimethylpentyl |
| 878 | 2 | O | COOH | 1,1-dimethylhexyl |
| 879 | 3 | O | COOH | ethyl |
| 880 | 1 | O | SO₃H | 1,1-dimethylpropyl |
| 881 | 1 | O | CN | 1,1-dimethylpropyl |
| 882 | 1 | O | Tetrazole | 1,1-dimethylpropyl |
| 883 | 1 | O | CONH₂ | 1,1-dimethylpropyl |
| 884 | 2 | O | CONH₂ | 1,1-dimethylpropyl |
| 885 | 1 | O | COOH | α-methylbenzyl |
| 886 | 2 | O | COOH | 4-methylbenzyl |
| 887 | 1 | O | Tetrazole | benzyl |
| 888 | 1 | O | SO₃H | α-methylbenzyl |
| 889 | 1 | O | SO₂HNMe | benzyl |
| 890 | 1 | O | CN | α-methylbenzyl |
| 891 | 1 | O | PO₃H₂ | 4-methylbenzyl |
| 892 | 2 | O | COOH | benzyl |
| 893 | 2 | O | COOH | α-methylbenzyl |
| 894 | 2 | O | COOH | 4-methylbenzyl |
| 895 | 2 | O | COOH | cyclohexyl |
| 896 | 2 | O | PO₂Het | i-propyl |
| 897 | 2 | O | PO₃Hpropyl | ethyl |
| 898 | 2 | O | PO₃(Et)₂ | methyl |
| 899 | 2 | O | methyl ester | tert-butyl |
| 900 | 1 | O | ethyl ester | n-pentyl |
| 901 | 2 | O | propyl ester | n-hexyl |
| 902 | 1 | O | butyl ester | cyclohexyl |
| 903 | 1 | O | pentyl ester | cyclopentyl |
| 904 | 1 | O | hexyl ester | n-heptyl |
| 905 | 1 | O | S-Me | n-octyl- |
| 906 | 1 | O | S-Et | n-nonyl |
| 907 | 2 | O | S-propyl | 2-indolyl |
| 908 | 2 | O | S-butyl | 2-furyl |
| 909 | 2 | O | NHCOMe | 2-thiazolyl |
| 910 | 2 | O | NHCOEt | 2-thienyl |
| 911 | 1 | O | CONH(O)Me | benzyl |
| 912 | 1 | O | CONH(O)Et | α-methylphenyl |
| 913 | 1 | O | CONH(O)propyl | 4-methylphenyl |
| 914 | 3 | O | CONHNHSO₂Me | benzyl |
| 915 | 3 | O | CONHNHSO₂Et | α-methylphenyl |
| 916 | 3 | O | CONHSO₂Me | 4-methylphenyl |
| 917 | 1 | O | CONHNHSO₂Et | phenyl |
| 913 | 2 | O | CON(Me)CN | α-methylphenyl |
| 919 | 1 | O | CON(Et)CN | 4-methylphenyl |
| 920 | 1 | O | COOH | 1,1-dimethylpropyl |
| 921 | 2 | O | COOH | 1,1-dimethylpropyl |
| 922 | 2 | O | 5-(3-pyridyl)pentyl ester | 1,1-dimethylpropyl |
| 923 | 1 | O | 4-(3-pyridyl)-3-butynyl ester | 1,1-dimethylpropyl |
| 924 | 1 | O | 3-butynyl ester | 1,1-dimethylpropyl |
| 925 | 1 | O | 5-phenylpentyl ester | 1,1-dimethylpropyl |
| 926 | 1 | O | 4-(3-pyridyl)butyl ester | 1,1-dimethylpropyl |
| 927 | 1 | O | 3-phenylpropyl ester | 1,1-dimethylpentyl |
| 928 | 1 | O | 3-(3-pyridyl)propyl ester | 1,1-dimethylpentyl |
| 929 | 1 | O | 4-phenylbutyl ester | 1,1-dimethylpentyl |
| 930 | 1 | O | 2-phenylethyl ester | 1,1-dimethylpropyl |
| 931 | 1 | O | 2-phenylethanoyl | 1,1-dimethylpropyl |
| 932 | 2 | O | 5-(3-pyridyl)pentanoyl | 1,1-dimethylpropyl |
| 933 | 2 | O | 4-phenylbutanoyl | 1,1-dimethylpropyl |
| 934 | 1 | O | 4-(3-pyridyl)butanoyl | 1,1-dimethylpropyl |
| 935 | 2 | S | 2-phenylethyl ester | 1,1-dimethylpropyl |
| 936 | 2 | S | 3-phenylpropyl ester | 1,1-dimethylpropyl |
| 937 | 1 | S | 3-phenylpropyl ester | 1,1-dimethylpropyl |
| 938 | 1 | S | 2-phenethylester | 1,1-dimethylpropyl |
| 939 | 1 | S | COOH | 1,1-dimethylpropyl |
| 940 | 2 | S | PO₃H₂ | 2-turyl |
| 941 | 1 | S | COOH | phenyl |
| 942 | 2 | S | COOH | 3,4,5-trimethoxyphenyl |

Particularly preferred embodiments of the compounds found in Table L are selected from the group consisting of:

3-phenyl-1-propyl 1-(3,3-dimethyl-1,2-dioxopentyl)-2-dazinecarboxylate,

4-phenyl-1-n-butyl 1-(3,3-dimethyl-1,2-dioxopentyl)-2-dazinecarboxylate,

5-phenyl-1-n-pentyl 1-(3,3-dimethyl-1,2-dioxopentyl)-2-pyridazinecarboxylate,

4-(3-pyridyl)-1-n-butyl 1-(3,3-dimethyl-1,2-dioxopentyl)-2-pyridazinecarboxylate,

3-phenyl-1-propyl 1-(3,3-dimethyl-1,2-dioxopentyl)-2-pyrazinecarboxylate,

3-(3-pyridyl)-1-propyl 1-(3,3-dimethyl-1,2-dioxopentyl)-2-pyrazinecarboxylate,

4-phenyl-1-n-butyl 1-(3,3-dimethyl-1,2-dioxopentyl)-2-pyrazinecarboxylate,

2-phenyl-1-ethyl 1-(3,3-dimethyl-1,2-dioxopentyl)-2-pyrazinecarboxylate,

2-[(4-phenylbutyl)carbonyl]-1-(3,3-dimethyl-1,2-dioxopentyl)pyridazine,

2-[(2-phenylethyl)carbonyl]-1-(3,3-dimethyl-1,2-dioxopentyl)pyridazine,

2-[(5-phenylpentyl)carbonyl]-1-(3,3-dimethyl-1,2-dioxopentyl)piperazine,

2-[(5-(3-pyridyl)pentyl)carbonyl]-1-(3,3-dimethyl-1,2-dioxopentyl)piperazine,

2-[(4-phenylbutyl)carbonyl]-1-(3,3-dimethyl-1,2-dioxopentyl)piperazine,

2-[(3-phenylpropyl)carbonyl]-1-(3,3-dimethyl-1,2-dioxopentyl)pyridazine,

2-[(5-phenylpentyl)carbonyl]-1-(3,3-dimethyl-1,2-dioxopentyl)pyridazine, and

2-[((4-(3-pyridyl)butyl)carbonyl]-1-(3,3-dimethyl-1,2-dioxopentyl)pyridazine.

Another preferred embodiment of the invention is the use for the treatment of nerve injury caused as a consequence of prostate surgery with a compound of the formula (LXIX):

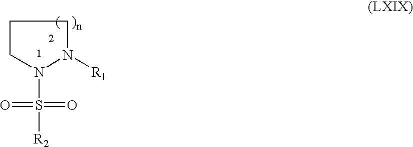

or a pharmaceutically acceptable salt, ester or solvate thereof, wherein:

n is 1-3;

R₁ is selected from the group consisting of —CR₃, —COOR₃, —COR₃, —COOH, —SO₃H, —SO₂HNR₃, —PO₂(R₃)₂, —CN, —PO₃ (R₃)₂, —OR₃, —SR₃, —NHCOR₃, —N(R₃)₂, —CON(R₃)₂, —CONH(O)R₃, —CONHNHSO₂R₃, —COHNSO₂R₃, —CONR₃CN,

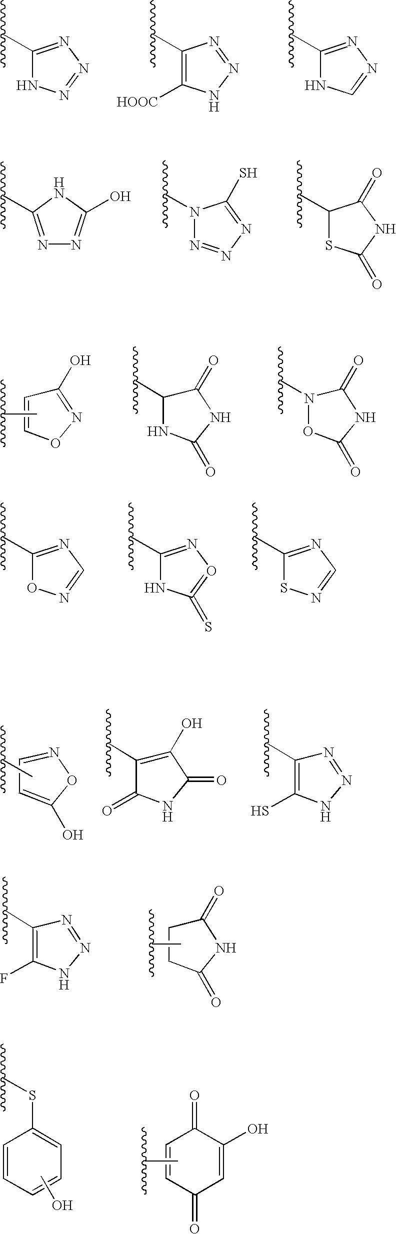

wherein said R₁ group is either unsubstituted or additionally substituted with R₃;

R₂ is selected from the group consisting of hydrogen, C₁-C₉ straight or branched chain alkyl, C₂-C₉ straight or branched chain alkenyl, C₂-C₉ straight or branched chain alkynyl, aryl, heteroaryl, carbocycle, or heterocycle, wherein said alkyl, alkenyl, alkynyl, aryl, heteroaryl, carbocycle, or heterocycle is unsubstituted or substituted with one or more substituents selected from R₃; and

R₃ is selected from the group consisting of hydrogen, C₁-C₉ alkyl, C₂-C₉ straight or branched chain alkenyl, C₂-C₉ straight or branched chain alkynyl, C₁-C₉ alkoxy, C₂-C₉ alkenyloxy, aryloxy, phenoxy, benzyloxy, hydroxy, carboxy, C₁-C₉ thioalkyl, C₂-C₉ thioalkenyl, C₁-C₉ alkylamino, C₂-C₉ alkenylamino, cyano, nitro, imino, sulfonyl, thiocarbonyl, sulfhydryl, halo, haloalkyl, trifluoromethyl, aryl, heteroaryl, carbocycle, and heterocycle,

wherein said alkyl, alkenyl, alkynyl, alkoxy, alkenyloxy, aryloxy, thioalkyl, thioalkenyl, alkylamino, alkenylamino, aryl, heteroaryl, carbocycle, or heterocycle group is optionally substituted with a hydroxy, carboxy, carbonyl, cyano, nitro, imino, sulfonyl, thiocarbonyl, sulfhydryl, halo, haloalkyl, trifluoromethyl, aryl, heteroaryl, carbocycle, or heterocycle group.

Specific embodiments of the inventive compounds are presented in Table LI. The present invention contemplates employing the compounds of Table LI, below, for use in compositions and methods of the invention.

TABLE LI

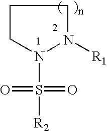

| No. | n | R₁ | R₂ |
|---|---|---|---|
| 943 | 1 | 3-Phenylpropyl ester | benzyl |
| 944 | 2 | 4-Phenylbutyl ester | benzyl |
| 945 | 1 | 5-Phenylpentanoyl | benzyl |
| 946 | 1 | COOH | benzyl |
| 947 | 1 | COOH | α-methylbenzyl |
| 948 | 1 | COOH | 4-methylbenzyl |
| 949 | 1 | tetrazole | benzyl |
| 950 | 1 | SO₃H | α-methylbenzyl |
| 951 | 1 | SO₂HNMe | benzyl |
| 952 | 1 | CN | α-methylbenzyl |
| 953 | 1 | PO₃H₂ | 4-methylbenzyl |
| 954 | 2 | COOH | benzyl |
| 955 | 2 | COOH | α-methylbenzyl |
| 956 | 2 | COOH | 4-methylbenzyl |
| 957 | 2 | COOH | 3,4,5-trimethoxyphenyl |
| 958 | 2 | COOH | cyclohexyl |
| 959 | 2 | PO₂HEt | i-propyl |
| 960 | 2 | PO₃HPropyl | ethyl |
| 961 | 2 | PO₃(Et)₂ | methyl |
| 962 | 2 | methyl ester | tert-butyl |
| 963 | 2 | ethyl ester | n-pentyl |
| 964 | 2 | propyl ester | n-hexyl |
| 965 | 1 | butyl ester | cyclohexyl |
| 966 | 1 | pentyl ester | cyclopentyl |
| 967 | 1 | hexyl ester | n-heptyl |
| 968 | 1 | S-Me | n-octyl |
| 969 | 1 | S-Et | n-nonyl |
| 970 | 2 | S-propyl | 2-indolyl |
| 971 | 2 | S-butyl | 2-furyl |
| 972 | 2 | NHCOMe | 2-thiazolyl |
| 973 | 2 | NHCOEt | 2-thienyl |
| 974 | 1 | CONH(O)Me | benzyl |
| 975 | 1 | CONH(O)Et | α-methylphenyl |
| 976 | 1 | CONH(O)propyl | 4-methylphenyl |
| 977 | 2 | COOH | benzyl |
| 978 | 2 | COOH | α-methylphenyl |
| 979 | 2 | COOH | 4-methylphenyl |
| 980 | 3 | CONHNHSO₂Me | benzyl |
| 981 | 3 | CONHNHSO₂Et | α-methylphenyl |
| 982 | 3 | CONHSO₂Me | 4-methylphenyl |
| 983 | 2 | CONHNHSO₂Et | phenyl |
| 984 | 2 | CON(Me)CN | α-methylphenyl |
| 985 | 2 | CON(Et)CN | 4-methylphenyl |

Particularly preferred embodiments of the compounds in Table LI are selected from the group consisting of:

4-phenyl-1-n-butyl 1-(phenylmethyl)sulfonyl-2-pyridazinecarboxylate, and

3-phenyl-1-propyl 1-(phenylmethyl)sulfonyl-2-pyrazinecarboxylate.

Another preferred embodiment of the invention is the use for the treatment of nerve injury caused as a consequence of prostate surgery with a compound of the formula (LXX):

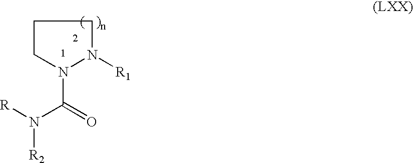

or a pharmaceutically acceptable salt, ester or solvate thereof, wherein:

n is 1-3;

R₁ is selected from the group consisting of —CR₃, —COOR₃, —COR₃, —COOH, —SO₃H, —SO₂HNR₃, —PO₂ (R₃)₂, —CN, —PO₃ (R₃)₂, —OR₃, —SR₃, —NHCOR₃, —N(R₃)₂, —CON(R₃)₂, —CONH(O)R₃, —CONHNHSO₂R₃, —COHNSO₂R₃, —CONR₃CN,

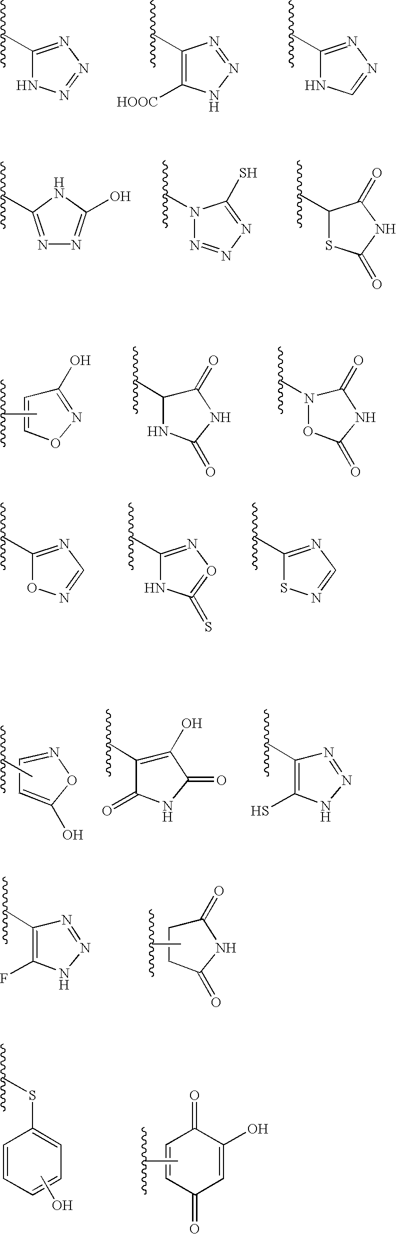

wherein said R₁ group is either unsubstituted or additionally substituted with R₃;

R and R₂ are independently C₁-C₉ alkyl, C₂-C₉ alkenyl, aryl, heteroaryl, carbocycle, or heterocycle, wherein said alkyl, alkenyl, aryl, heteroaryl, carbocycle, or heterocycle is unsubstituted or substituted with one or more substituent(s) selected from R₃; and

R₃ is selected from the group consisting of hydrogen, C₁-C₉ alkyl, C₂-C₉ straight or branched chain alkenyl, C₂-C₉ straight or branched chain alkynyl, C₁-C₉ alkoxy, C₂-C₉ alkenyloxy, aryloxy, phenoxy, benzyloxy, hydroxy, carboxy, C₁-C₉ thioalkyl, C₂-C₉ thioalkenyl, C₁-C₉ alkylamino, C₂-C₉ alkenylamino, cyano, nitro, imino, sulfonyl, thiocarbonyl, sulfhydryl, halo, haloalkyl, trifluoromethyl, aryl, heteroaryl, carbocycle, and heterocycle,

wherein said alkyl, alkenyl, alkynyl, alkoxy, alkenyloxy, aryloxy, thioalkyl, thioalkenyl, alkylamino, alkenylamino, aryl, heteroaryl, carbocycle, or heterocycle group is optionally substituted with a hydroxy, carboxy, carbonyl, cyano, nitro, imino, sulfonyl, thiocarbonyl, sulfhydryl, halo, haloalkyl, trifluoromethyl, aryl, heteroaryl, carbocycle, or heterocycle group.

Specific embodiments of the inventive compounds are presented in Table LII. The present invention contemplates employing the compounds of Table LII, below, for use in compositions and methods of the invention.

TABLE LII

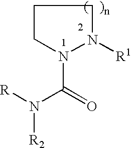

| No. | n | R₁ | R₄ | R₅ |
|---|---|---|---|---|
| 986 | 1 | 5-Phenylpentanoyl | cyclohexyl | cyclohexyl |
| 987 | 1 | COCH | cyclohexyl | methyl |
| 988 | 1 | COOH | cyclohexyl | ethyl |
| 989 | 1 | COOH | cyclohexyl | propyl |
| 990 | 1 | COOH | cyclohexyl | butyl |

Another preferred embodiment of the invention is the use for the treatment of nerve injury caused as a consequence of prostate surgery with a compound of the formula (LXXI):

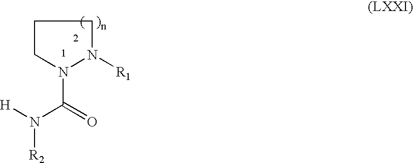

or a pharmaceutically acceptable salt, ester or solvate thereof, wherein:

n is 1-3;

R₁ is selected from the group consisting of —CR₃, —COOR₃, —COR₃, —COOH, —SO₃H, —SO₂HNR₃, —PO₂ (R₃)₂, —CN, —PO₃ (R₃)₂, —OR₃, —SR₃, —NHCOR₃, —N(R₃)₂, —CON(R³)₂, —CONH(O)R₃, —CONHNHSO₂R₃, —COHNSO₂R₃, —CONR₃CN,

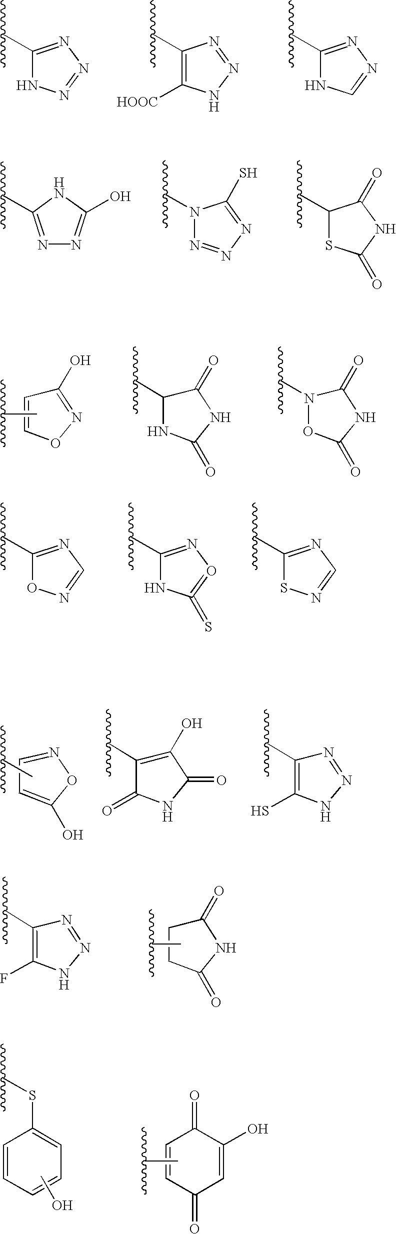

wherein said R₁ group is either unsubstituted or additionally substituted with R₃; and

R₂ is C₁-C₉ alkyl, C₂-C₉ alkenyl, aryl, heteroaryl, carbocycle, or heterocycle, wherein said alkyl, alkenyl, aryl, heteroaryl, carbocycle, or heterocycle is substituted with one or more substituent(s) selected from R₃; and

R₃ is selected from the group consisting of hydrogen, C₁-C₉ alkyl, C₂-C₉ straight or branched chain alkenyl, C₂-C₉ straight or branched chain alkynyl, C₁-C₉ alkoxy, C₂-C₉ alkenyloxy, aryloxy, phenoxy, benzyloxy, hydroxy, carboxy, C₁-C₉ thioalkyl, C₂-C₉ thioalkenyl, C₁-C₉ alkylamino, C₂-C₉ alkenylamino, cyano, nitro, imino, sulfonyl, thiocarbonyl, sulfhydryl, halo, haloalkyl, trifluoromethyl, aryl, heteroaryl, carbocycle, and heterocycle,

wherein said alkyl, alkenyl, alkynyl, alkoxy, alkenyloxy, aryloxy, thioalkyl, thioalkenyl, alkylamino, alkenylamino, aryl, heteroaryl, carbocycle, or heterocycle group is optionally substituted with a hydroxy, carboxy, carbonyl, cyano, nitro, imino, sulfonyl, thiocarbonyl, sulfhydryl, halo, haloalkyl, trifluoromethyl, aryl, heteroaryl, carbocycle, or heterocycle group.

Specific embodiments of the inventive compounds are presented in Table LIII. The present invention contemplates employing the compounds of Table LIII, below, for use in compositions and methods of the invention.

TABLE LIII

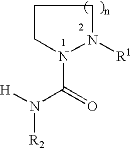

| No. | n | R₁ | R₂ |
|---|---|---|---|
| 991 | 1 | 3-phenylpropyl ester | cyclohexyl |
| 992 | 2 | 4-phenylbutyl ester | Cyclohexyl |
| 993 | 1 | 5-phenylpentanoyl | Cyclohexyl |
| 994 | 1 | COOH | Cyclohexyl |
| 995 | 1 | COOH | α-methylbenzyl |
| 996 | 1 | COOH | 4-methylbenzyl |
| 997 | 1 | tetrazole | benzyl |
| 998 | 1 | SO₃H | α-methylbenzyl |
| 999 | 1 | SO₂HNMe | benzyl |
| 1000 | 1 | CN | α-methylbenzyl |
| 1001 | 1 | PO₃H₂ | 4-methylbenzyl |
| 1002 | 2 | COOH | benzyl |
| 1003 | 2 | COOH | α-methylbenzyl |
| 1004 | 2 | COOH | 2-butyl |
| 1005 | 2 | COOH | cyclohexyl |
| 1006 | 2 | PO₂HEt | 1-propyl |
| 1007 | 2 | PO₃HPropyl | ethyl |
| 1008 | 2 | PO₃(Et)₂ | methyl |
| 1009 | 2 | Methyl ester | tert-butyl |
| 1010 | 2 | Ethyl ester | n-pentyl |
| 1011 | 2 | propyl ester | n-hexyl |
| 1012 | 1 | butyl ester | cyclohexyl |
| 1013 | 1 | pentyl ester | cyclopentyl |
| 1014 | 1 | hexyl ester | heptyl |
| 1015 | 1 | SMe | n-octyl |
| 1016 | 1 | SEt | n-hexyl |
| 1017 | 2 | S-propyl | n-hexyl |
| 1018 | 2 | S-butyl | n-hexyl |
| 1019 | 2 | NHCOMe | n-hexyl |
| 1020 | 2 | NHCOEt | 2-thienyl |
| 1021 | 1 | CONH(O)Me | benzyl |
| 1022 | 1 | CONH(O)Et | α-methylphenyl |
| 1023 | 1 | CONH(O)propyl | 4-methylphenyl |
| 1024 | 2 | COOH | benzyl |
| 1025 | 2 | COOH | α-methylphenyl |
| 1026 | 2 | COOH | 4-methylphenyl |
| 1027 | 3 | CONHNHSO₂Me | benzyl |
| 1028 | 3 | CONHNHSO₂Et | α-methylphenyl |
| 1029 | 3 | CONHSO₂Me | 4-methylphenyl |
| 1030 | 2 | CONHNHSO₂Et | phenyl |
| 1031 | 2 | CON(Me)CN | α-methylphenyl |
| 1032 | 2 | CON(Et)CN | 4-methylphenyl |
| 1033 | 1 | 3-phenylpropyl ester | cyclohexyl |

Particularly preferred embodiments of the compounds in Table LIII are selected from the group consisting of:

4-phenyl-1-n-butyl 1-(cyclohexyl)carbamoyl-2-pyridazinecarboxylate, and

3-phenyl-1-propyl 1-(cyclohexyl)carbamoyl-2-pyrazinecarboxylate.

IX. Hydantoin Compounds

Another preferred embodiment of the invention is the use for the treatment of nerve injury caused as a consequence of prostate surgery with a compound of the formula (LXXII):

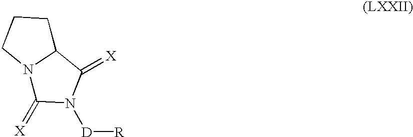

where

each X independently is O, S, or NR₂;

R₂ is selected from the group consisting of cyano, nitro, hydrogen, C₁-C₄ alkyl, hydroxy, and C₁-C₄ alkoxy;

D is a direct bond or C₁-C₈ alkyl or alkenyl;

R is hydrogen, or an alicyclic or aromatic, mono-, bi- or tricyclic, carbo- or heterocyclic ring;

wherein R is optionally substituted with one substituent selected from the group consisting of hydrogen, halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenyl, phenoxy, benzyloxy, and amino;

or a pharmaceutically acceptable salt, ester, or solvate thereof.

Specific embodiments of the inventive compounds are presented in Table LIV. The present invention contemplates employing the compounds of Table LIV, below, for use in compositions and methods of the invention.

TABLE LIV

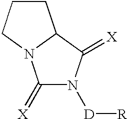

| No. | X₁ | X₂ | D | R |
|---|---|---|---|---|
| 1034 | O | O | bond | Naphthyl |
| 1035 | O | O | bond | 2-(Phenyl)phenyl |
| 1036 | O | O | bond | 4-Trifluoromethylphenyl |
| 1037 | S | O | methyl | Phenyl |
| 1038 | O | O | hexyl | Hydrogen |
| 1039 | O | O | bond | 2-(Ethyl)phenyl |
| 1040 | S | O | propyl | Phenyl |
| 1041 | S | O | ethyl | Phenyl |
| 1042 | O | O | heptyl | Hydrogen |
| 1043 | O | O | octyl | Hydrogen |
| 1044 | S | O | pentyl | 3-Pyridyl |
| 1045 | O | O | propyl | Phenyl |
| 1046 | O | O | bond | 3-(Hydroxy)phenyl |
| 1047 | O | O | bond | 4-(tert-butyl)phenyl |
| 1048 | O | O | bond | 2-(Prop-2-enyl)phenyl |
| 1049 | O | O | bond | 3-(Ethoxy)phenyl |
| 1050 | S | O | bond | Cyclopentyl |
| 1051 | S | O | bond | Quinolinyl |
| 1052 | O | O | hexyl | Phenyl |
| 1053 | O | O | ethyl | Phenyl |
| 1054 | O | O | bond | Cyclopentyl |
| 1055 | S | S | bond | 2-thienyl |
| 1056 | O | S | bond | 2-thienyl |
| 1057 | O | O | bond | 2-oxazolyl |
| 1058 | S | O | bond | 2-furyl |
| 1059 | O | NH | bond | 3-furyl |
| 1060 | O | NH | hexyl | 4-furyl |
| 1061 | O | S | bond | Adamantyl |
| 1062 | S | N—CN | bond | Carbazole |
| 1063 | O | N—NO₂ | bond | Isoquinoline |
| 1064 | NH | NH | methyl | 3-Pyridinyl |
| 1065 | O | NCH₃ | hexyl | Hydrogen |
| 1066 | NOH | O | bond | 2-Thiazolyl |
| 1067 | NOCH₃ | S | bond | 4-(tert-butyl)phenyl |
| 1063 | O | S | bond | Cyclohexyl |
| 1069 | O | O | bond | Phenyl |
| 1070 | S | O | bond | Phenyl |

Particularly preferred embodiments of the compounds in Table LIV are selected from the group consisting of:

(7aS)-2-(1-Naphthyl)perhydropyrrolo(1,2-c]imidazole-1,3-dione,

(7aS)-2-(2′-Phenyl)phenylperhydropyrrolo[1,2-c]imidazole-1,3-dione,

(7aS)-2-(4-(Trifluoromethyl)phenyl)perhydropyrrolo [1,2-c]imidazole-1,3-dione,

2-benzyl-3-thioxo-2,5,6,7,7a-pentahydro-2-azapyrrolizin-1-one,

2-hexyl-2,5,6,7,7a-pentahydro-2-azapyrrolizine-1,3-dione,

2-(2-ethyl)phenyl-2,5,6,7,7a-pentahydro-2-azapyrrolizin-1,3-dione,

2-(3-phenylpropyl)-3-thioxo-2,5,6,7,7a-pentahydro-2-azapyrrolizin-1-one,

2-(2-phenylethyl)-3-thioxo-2,5,6,7,7a-pentahydro-2-azapyrrolizin-1-one,

(7aS)-2-Cyclohexyl-3-thioxoperhydropyrrolo [1,2-c]imidazole-1-one,

2-Phenyl-2,5,6,7,7a-pentahydro-2-azapyrrolizine-1,3-dione, and

2-phenyl-3-thioxo-2,5,6,7,7a-pentahydro-2-azapyrrolizin-1-one.

Another preferred embodiment of the invention is the use for the treatment of nerve injury caused as a consequence of prostate surgery with a compound of the formula (LXXIII):

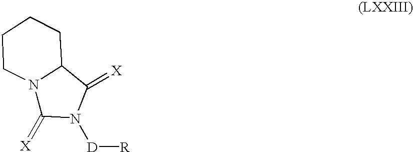

where

each X independently is O, S, or NR₂;

R₂ is selected from the group consisting of cyano, nitro, hydrogen, C₁-C₄ alkyl, hydroxy, and C₁-C₄ alkoxy;

D is a direct bond or C₁-C₈ alkyl or alkenyl;

R is hydrogen, or an alicyclic or aromatic, mono, bi- or tricyclic, carbo- or heterocyclic ring;

wherein R is optionally substituted with one substituent selected from the group consisting of hydrogen, halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenyl, phenoxy, benzyloxy, and amino;

or a pharmaceutically acceptable salt, ester, or solvate thereof.

Specific embodiments of the inventive compounds are presented in Table LV. The present invention contemplates employing the compounds of Table LV, below, for use in compositions and methods of the invention.

TABLE LV

| No. | X₁ | X₂ | D | R |
|---|---|---|---|---|
| 1071 | O | O | methyl | Phenyl |
| 1072 | S | O | methyl | Phenyl |
| 1073 | S | O | ethyl | Phenyl |
| 1074 | O | O | heptyl | Hydrogen |
| 1075 | O | O | octyl | Hydrogen |
| 1076 | S | O | propyl | Phenyl |
| 1077 | O | O | hexyl | Hydrogen |
| 1078 | O | O | bond | Cyclohexyl |
| 1079 | O | O | ethyl | Phenyl |
| 1080 | 5 | O | heptyl | Hydrogen |
| 1081 | O | O | octyl | Hydrogen |
| 1082 | S | O | pentyl | 3-Pyridyl |
| 1083 | O | O | propyl | Phenyl |
| 1084 | O | O | bond | 3-(Phenoxy)phenyl |
| 1085 | O | O | bond | 4-(tert-butyl)phenyl |
| 1086 | O | O | bond | 2-(Prop-2-enyl)phenyl |
| 1087 | O | O | bond | 3-(Ethoxy)phenyl |
| 1088 | S | O | bond | Cyclopentyl |
| 1089 | S | O | bond | Quinolinyl |
| 1090 | O | O | hexyl | Phenyl |
| 1091 | O | O | ethyl | Phenyl |
| 1092 | O | O | bond | Cyclopentyl |
| 1093 | S | S | bond | 2-thienyl |
| 1094 | O | S | bond | 2-thienyl |
| 1095 | O | NH | bond | 2-oxazolyl |
| 1096 | S | O | bond | 2-furyl |
| 1097 | O | O | bond | 3-furyl |
| 1098 | S | NH | hexyl | 4-furyl |
| 1099 | O | N—CN | bond | Adamantyl |
| 1100 | 5 | N—NO₂ | bond | Carbazole |
| 1101 | O | S | bond | Adamantyl |
| 1102 | S | NC₃H₇ | bond | 2-Pyrazolyl |
| 1103 | NOH | O | hexyl | Hydrogen |
| 1104 | NOCH₃ | O | bond | Cyclopentyl |
| 1105 | O | O | bond | Phenyl |
| 1106 | S | O | bond | Phenyl |
| 1107 | O | O | butyl | Hydrogen |

Particularly preferred embodiments of the compounds in Table LV are selected from the group consisting of:

2-Benzyl-2,5,6,7,8,8a-hexahydro-2-azaindolizine-1,3dione,

2-benzyl-3-thioxo-2,5,6,7,8,8a-hexahydro-2-azaindolizin-1-one,

2-(2-phenylethyl)-3-thioxo-2,5,6,7,8,8a-hexahydro-2-azaindolizin-1-one,

2-Heptyl-2,5,6,7,8,8a-hexahydro-2-azaindolizine-1,3-dione,

2-Octyl-2,5,6,7,8,8a-hexahydro-2-azaindolizine-1,3-dione,

2-(3-phenylpropyl)-3-thioxo-2,5,6,7,8,8a-hexahydro-2-azaindolizin-1-one,

2-hexyl-2,5,6,7,8,8a-hexahydro-2-azaindolizine-1,3-dione,

2-Cyclohexyl-2,5,6,7,8,8a-hexahydro-2-azaindolizine-1,3-dione,

2-phenyl-2,5,6,7,8,8a-hexahydro-2-azaindolizine-1,3-dione,

2-phenyl-3-thioxo-2,5,6,7,8,8a-hexahydro-2-azaindolizin-1-one, and

2-butyl-2,5,6,7,8,8a-hexahydro-2-azaindolizine-1,3-dione.

X. Bridged Ring Compounds

Another preferred embodiment of the invention is the use for the treatment of nerve injury caused as a consequence of prostate surgery with a compound of the formula (LXXIV):

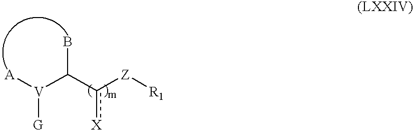

or a pharmaceutically acceptable salt, ester, or solvate thereof, wherein:

A and B, taken together with the atoms to which they are attached, form a saturated, unsaturated, or aromatic heterocylic or carbocyclic bridged ring moiety which contains one or more O, C(R₁)₂, S(O)ₚ, N, NR₁, or NR₅ atoms;

V is CH, S, or N;

X is O, CH₂ or S;

m is 0 or 1;

G is

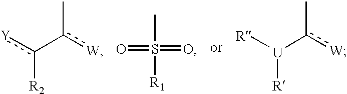

R₁ is independently hydrogen, C₁-C₉ straight or branched chain alkyl, or C₂-C₉ straight or branched chain alkenyl or alkynyl, C₃-C₉ cycloalkyl, C₅-C₇ cycloalkenyl, a carboxylic acid or carboxylic acid isostere, N(R₄)ₙ, Ar₁, Ar₄, a bridged ring moiety, or K-L, wherein said alkyl, cycloalkyl, cycloalkenyl, alkynyl, alkenyl, Ar₁, Ar₄, or bridged ring moiety, is optionally substituted with one or more substituent(s) independently selected from the group consisting of:

2-furyl, 2-thienyl, pyridyl, phenyl, C₃-C₆ cycloalkyl wherein said furyl, thienyl, pyridyl, phenyl or cycloalkyl group optionally is substituted with C₁-C₄ alkoxy, (Ar₁)ₙ, halo, halo-C₁-C₆-alkyl, carbonyl, thiocarbonyl, C₁-C₆ thioester, cyano, imino, COOR₆ in which R₆ is independently C₁-C₉ straight or branched chain alkyl or alkenyl, hydroxy, nitro, trifluoromethyl, C₁-C₆ alkoxy, C₂-C₄ alkenyloxy, C₁-C₆ alkylaryloxy C₁-C₆ aryloxy, aryl-(C₁-C₆)-alkyloxy, phenoxy, benzyloxy, thio-(C₁-C₆)-alkyl, C₁-C₆-alkylthio, sulfhydryl, sulfonyl, amino, (C₁-C₆)-mono- or di-alkylamino, amino-(C₁-C₆)-alkyl, aminocarboxy, C₃-C₈ cycloalkyl, C₁-C₆ straight or branched chain alkyl, C₂-C₆ straight or branched chain alkenyl optionally substituted with (Ar₁)ₙ, C₃-C₈ cycloalkyl, C₁-C₆ straight or branched chain alkyl, C₂-C₆ straight or branched chain alkenyl substituted with C₃-C₈ cycloalkyl, C₃-C₈ cycloalkyl, and Ar₂, and, wherein any carbon atom of an alkyl or alkenyl group may optionally replaced with O, NR₅, or S(O)ₚ;

Ar₁ or Ar₂, independently, is an alicyclic or aromatic, mono-, bi- or tricyclic, carbo- or heterocyclic ring, wherein the ring is optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₃-C₈ cycloalkyl, C₅-C₇ cycloalkenyl, C₁-C₄ alkoxy, C₂-C₄ alkenyloxy, phenoxy, benzyloxy, and amino; wherein the individual ring contains 5-8 members; and wherein the heterocyclic ring contains 1-6 heteroatom(s) independently selected from the group consisting of O, N, and S, and, wherein any aromatic or tertiary alkylamine is optionally oxidized to a corresponding N-oxide;

or, R₁ is independently a moiety of the formula:

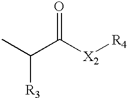

wherein:

R₃ is independently C₁-C₉ straight or branched chain alkyl which is optionally substituted with C₃-C₈ cycloalkyl, a bridged ring moiety, or Ar₁;

X₂ is O or NR₆, wherein R₆ is independently selected from the group consisting of hydrogen, C₁-C₆ straight or branched chain alkyl, and C₂-C₆ straight or branched chain alkenyl;

R₄ is independently selected from the group consisting of phenyl, benzyl, C₁-C₅ straight or branched chain alkyl, C₂-C₅ straight or branched chain alkenyl, C₁-C₅ straight or branched chain alkyl substituted with phenyl, C₂-C₅ straight or branched chain alkenyl substituted with phenyl, and a bridged ring moiety;

R₂ is independently C₁-C₉ straight or branched chain alkyl, C₂-C₉ straight or branched chain alkenyl, C₃-C₈ cycloalkyl, C₅-C₇ cycloalkenyl, a bridged ring moiety, or Ar₁, wherein said alkyl, alkenyl, cycloalkyl, cycloalkenyl, or bridged ring moiety, is optionally substituted with one or more substituents selected from the group consisting of C₁-C₆ straight or branched chain alkyl, C₂-C₆ straight or branched chain alkenyl, C₃-C₈ cycloalkyl, C₅-C₇ cycloalkenyl, (Ar₁)ₙ and hydroxy; or,

R₂ is independently either hydrogen or P;

Y is either oxygen or CH—P, provided that if R₂ is hydrogen, then Y is CH—P, or if Y is oxygen then R₂ is P;

P is hydrogen, O—(C₁-C₄ straight or branched chain alkyl), O—(C₂-C₄ straight or branched chain alkenyl) C₁-C₆ straight or branched chain alkyl, C₂-C₆ straight or branched chain alkenyl, C₅-C₇ cycloalkyl, C₅-C₇ cycloalkenyl substituted with C₁-C₄ straight or branched chain alkyl or C₂-C₄ straight or branched chain alkenyl, (C₁-C₄ alkyl or C₂-C₄ alkenyl)-Ar₅, or Ar₅;

U is either O or N, provided that:

when U is O, then R′ is a lone pair of electrons and R″ is selected from the group consisting of Ar₄, a bridged ring moiety, C₃-C₈ cycloalkyl, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; and

when U is N, then R′ and R″ are, independently, selected from the group consisting of hydrogen, Ar₄, a bridged ring moiety, C₃-C₁₀ cycloalkyl, a C₇-C₁₂ bi- or tri-cyclic carbocycle, C₁-C₉ straight or branched chain alkyl, and C₂-C₉ straight or branched chain alkenyl, wherein said alkyl or alkenyl is optionally substituted with one or more substituent(s) independently selected from the group consisting of Ar₄ and C₃-C₈ cycloalkyl; or R′ and R″ are taken together to form a heterocyclic 5- or 6-membered ring selected from the group consisting of pyrrolidine, imidazolidine, pyrazolidine, piperidine, and piperazine.

W and Y, independently, are O, S, CH₂ or H₂;

Z is C(R₁)₂, O, S, a direct bond or NR₁; or,

Z-R₁ is independently

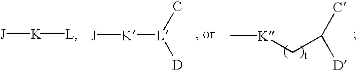

wherein:

C and D are, independently, hydrogen, a bridged ring moiety, Ar₄, Ar₁, C₁-C₆ straight or branched chain alkyl, or C₂-C₆ straight or branched chain alkenyl; wherein said alkyl or alkenyl is optionally substituted with one or more substituent(s) independently selected from the group consisting of C₃-C₃ cycloalkyl, C₅-C₇ cycloalkenyl, hydroxy, carbonyl oxygen, Ar₁ and Ar₄; wherein said alkyl, alkenyl, cycloalkyl or cycloalkenyl is optionally substituted with C₁-C6 alkyl, C₂-C₆ alkenyl, hydroxy, amino, halo, haloalkyl, thiocarbonyl, C₁-C₆ ester, C₁-C₆ thioester, C₁-C₆ alkoxy, C₁-C₆ alkenoxy, cyano, nitro, imino, C₁-C₆ alkylamino, amino-(C₁-C₆)alkyl, sulfhydryl, thio-(C₁-C₆)alkyl, or sulfonyl; wherein any carbon atom of said alkyl or alkenyl is optionally substituted in one or more position(s) with oxygen to form a carbonyl; or wherein any carbon atom of said alkyl or alkenyl is optionally replaced with O, NR₅, or (SO)ₚ;

C′ and D′ are independently hydrogen, a bridged ring moiety, Ar₅, C₁-C₆ straight or branched chain alkyl, or C₂-C₆ straight or branched chain alkenyl, wherein said alkyl or alkenyl is optionally substituted with C₅-C₇ cycloalkyl, C₅-C₇ cycloalkenyl, or Ar₅, wherein, one or two carbon atom(s) of said alkyl or alkenyl may be substituted with one or two heteroatom(s) independently selected from the group consisting of oxygen, sulfur, SO, and SO₂ in chemically reasonable substitution patterns, or

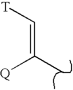

wherein

Q is hydrogen, C₁-C₆ straight or branched chain alkyl, or C₂-C₆ straignt or branched chain alkenyl; and

T is Ar₅ or C₅-C₇ cycloalkyl substituted at oositions 3 and 4 with substituents independently selected from the group consisting of hydrogen, hydroxy, O—(C₁-C₄ alkyl), O—(C₂-C₄ alkenyl), and carbonyl,

J is O, NR₁, S, or (CR₁)₂;

K is a direct bond, C₁-C₆ straight or branched chain alkyl, or C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, a bridged ring moiety, hydroxy, carbonyl oxygen, and Ar₃; wherein said alkyl, alkenyl, cycloalkyl, cycloalkenyl or Ar₃, is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl or Ar₃, is optionally replaced with O, NR′″, or S(O)ₚ,

wherein R′″ is selected from the group consisting of hydrogen, C₁-C₄ straight or branched chain alkyl, C₃-C₄ straight or branched chain alkenyl or alkynyl, a bridged ring moiety, and C₁-C₄ bridging alkyl wherein a bridge is formed between the nitrogen and a carbon atom of said alkyl or alkenyl chain containing said heteroatom to form a ring, wherein said ring is optionally fused to an Ar₃ group;

K′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, haloalkyl, thiocarbonyl, ester, thioester, alkoxy, alkenoxy, cyano, nitro, imino, alkylamino, aminoalkyl, sulfhydryl, thioalkyl, sulfonyl, or oxygen to form a carbonyl, or wherein any carbon atom of said alkyl or alkenyl is optionally replaced with O, NR₅, S(O)ₚ;

K″ is C(R₁)₂, O, S, a direct bond or NR₁;

L is an aromatic amine or a tertiary amine oxidized to a corresponding N-oxide; said aromatic amine being selected from the group consisting of pyridyl, pyrimidyl, quinolinyl, and isoquinolinyl, said aromatic amine being optionally substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy, and amino; and wherein said tertiary amine is NRₓR_{y}R_{z}, wherein Rₓ, R_{y}, and R_{z} are independently selected from the group consisting of C₁-C₆ straight or branched chain alkyl and C₂-C₆ straight or branched chain alkenyl; wherein said alkyl or alkenyl is optionally substituted with one or more substituent(s) independently selected from the group consisting of C₁-C₆ straight or branched chain alkyl, C₂-C₆ straight or branched chain alkenyl, C₃-C₈ cycloalkyl, C₅-C₇ cycloalkenyl, hydroxy, carbonyl oxygen, a bridged ring moiety, and Ar₃; wherein said alkyl, alkenyl, cycloalkyl, cycloalkenyl, or Ar₃ is optionally substituted with C₁-C₄ alkyl, C₂-C₄ alkenyl, hydroxy, or carbonyl oxygen; wherein any carbon atom of said alkyl, alkenyl, cycloalkyl, cycloalkenyl, or Ar₃ is optionally replaced with O, NR′, S(O)ₚ;

L′ is a direct bond, C₁-C₆ straight or branched chain alkyl, or C₂-C₆ straight or branched chain alkenyl, wherein any carbon atom of said alkyl or alkenyl is optionally substituted in one or more position(s) with amino, halo, haloalkyl, thiocarbonyl, ester, thioester, alkoxy, alkenoxy, cyano, nitro, imino, alkylamino, aminoalkyl, sulfhydryl, thioalkyl, sulfonyl, or oxygen to form a carbonyl, or wherein any carbon atom of said alkyl or alkenyl is optionally replaced with O, NR₅, S(O)ₚ;

n is 1 or 2;

p is 0, 1, or 2;

t is 0, 1, 2, 3, or 4;

Ar₃ is independently selected from the group consisting of pyrrolidinyl, pyridyl, pyrimidyl, pyrazyl, pyridazyl, quinolinyl, and isoquinolinyl;

Ar₄ is independently an alicyclic or aromatic, mono-, bi- or tricyclic, carbo- or heterocyclic ring, wherein the ring is optionally substituted with one or more substituent(s) independently selected from the group consisting of alkylamino, amido, amino, aminoalkyl, azo, benzyloxy, C₁-C₉ straight or branched chain alkyl, C₁-C₉ alkoxy, C₂-C₉ alkenyloxy, C₂-C₉ straight or branched chain alkenyl, C₃-C₈ cycloalkyl, C₅-C₇ cycloalkenyl, carbonyl, carboxy, cyano, diazo, ester, formanilido, halo, haloalkyl, hydroxy, imino, isocyano, isonitrilo, nitrilo, nitro, nitroso, phenoxy, sulfhydryl, sulfonylsulfoxy, thio, thioalkyl, thiocarbonyl, thiocyano, thioester, thioformamido, trifluoromethyl, and carboxylic and heterocyclic moieties; wherein the individual alicyclic or aromatic ring contains 5-8 members and wherein said heterocyclic ring contains 1-6 heteroatom(s) independently selected from the group consisting of O, N, and S; and wherein any aromatic or tertiary alkyl amine is optionally oxidized to a corresponding N-oxide;

Ar₅ is independently selected from the group consisting of 1-napthyl, 2-napthyl, 2-furyl, 3-furyl, 2-thienyl, 3-thienyl, 2-pyridyl, 3-pyridyl, 4-pyridyl and phenyl, monocyclic and bicyclic heterocyclic ring systems with individual ring sizes being 5 or 6 which contain in either or both rings a total of 1-4 heteroatom(s) independently selected from the group consisting of oxygen, nitrogen and sulfur; wherein Ar₅ optionally contains 1-3 substituent(s) independently selected from the group consisting of hydrogen,. halo, hydroxy, hydroxymethyl, nitro, CF₃, trifluoromethoxy, C₁-C₆ straight or branched chain alkyl, C₂-C₆ straight or branched chain alkenyl, O—(C₁-C₄ straight or branched chain alkyl), O—(C₂-C₄ straight or branched chain alkenyl), O-benzyl, O-phenyl, amino, 1,2-methylenedioxy, carbonyl, and phenyl; and

R₅ is independently selected from the group consisting of hydrogen, C₁-C₆ straight or branched chain alkyl, C₃-C₆ straight or branched chain alkenyl or alkynyl, a bridged ring moiety, and C₁-C₄ bridging alkyl wherein a bridge is formed between the nitrogen and a carbon atom of said alkyl or alkenyl chain containing said heteroatom to form a ring, wherein said ring is optionally fused to an Ar₄ or Ar₁ group;

R₆ is hydrogen, hydroxy, halo, haloalkyl, thiocarbonyl, alkoxy, alkenoxy, alkylaryloxy, aryloxy, arylalkyloxy, cyano, nitro, imino, alkylamino, aminoalkyl, sulfhydryl, thioalkyl, alkylthio, sulfonyl, C₁-C₆ straight or branched chain alkyl, C₂-C₆ straight or branched chain alkenyl or alkynyl, aryl, heteroaryl, carbocycle, heterocycle, or CO₂R₇ where R₇ is hydrogen or C₁-C₉ straight or branched chain alkyl or alkenyl;

R₈ is halo, haloalkyl, aminoalkyl, thioalkyl, C₂-C₆ straight or branched chain alkenyl or alkynyl, carbocycle, or heterocycle;

R₉ is independently hydrogen, halo, haloalkyl, thiocarbonyl, alkoxy, alkenoxy, alkylaryloxy, aryloxy, arylalkyloxy, cyano, nitro, imino, alkylamino, aminoalkyl, sulfhydryl, thioalkyl, alkylthio, sulfonyl, C₁-C₆ straight or branched chain alkyl, C₂-C₆ straight or branched chain alkenyl or alkynyl, aryl, heteroaryl, carbocycle, heterocycle, or CO₂R⁴ where R⁴ is hydrogen or C₁-C₉ straight or branched chain alkyl or alkenyl; and

R₁₀ is C₁-C₆ straight or branched chain alkyl, C₂-C₆ straight or branched chain alkenyl or alkynyl, aryl, heteroaryl, carbocycle, or heterocycle.

### Synthesis of Neurotrophic Compounds

The compounds for use in the methods and compositions of the invention may be readily prepared by standard techniques of organic chemistry, utilizing the general synthetic pathways depicted below.

In the preparation of the compounds of the invention, one skilled in the art will understand that one may need to protect or block various reactive functionalities on the starting compounds or intermediates while a desired reaction is carried out on other portions of the molecule. After the desired reactions are complete, or at any desired time, normally such protecting groups will be removed by, for example, hydrolytic or hydrogenolytic means. Such protection and deprotection steps are conventional in organic chemistry. One skilled in the art is referred to “Protective Groups in Organic Chemistry,” McOmie, ed., Plenum Press, New York, N.Y.; and “Protective Groups in Organic Synthesis,” Greene, ed., John Wiley & Sons, New York, N.Y. (1981) for the teaching of protective groups which may be useful in the preparation of compounds of the present invention.

The product and intermediates may be isolated or purified using one or more standard purification techniques, including, for example, one or more of simple solvent evaporation, recrystallization, distillation, sublimation, filtration, chromatography, including thin-layer chromatography, HPLC (e.g. reverse phase HPLC), column chromatography, flash chromatography, radial chromatography, trituration, and the like.

As described by Scheme I, cyclic amino acids 1 protected by suitable blocking groups P on the amino acid nitrogen may be reacted with thiols RSH to generate thioesters 2. After removal of the protecting group, the free amine 3 may be reacted with a variety of isocyanates or isothiocyanates to provide the final ureas or thioureas, respectively.

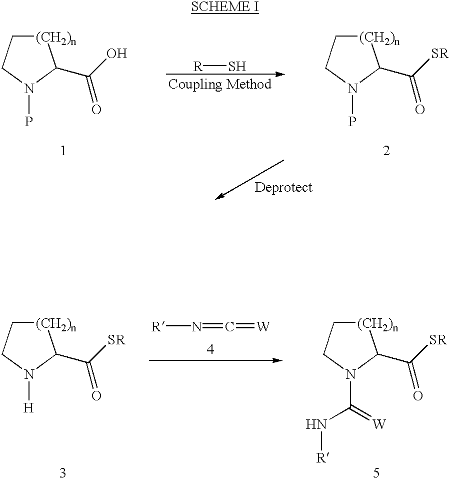

Isocyanates (R′NCO) or isothiocyanates (R′NCS) 4 may be conveniently prepared from the corresponding readily available amines by reaction with phosgene or thiophosgene, as depicted in Scheme II.

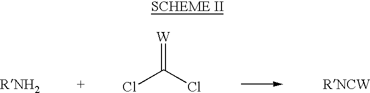

Thiols R—SH may be conveniently prepared from the corresponding readily available alcohols or halides via a two step replacement of halide by sulfur, as described in Scheme III. Halides may be reacted with thiourea, and the corresponding alkyl thiouronium salts hydrolyzed to provide thiols RSH. If alcohols are used as the starting materials, they may be first converted to the corresponding halides by standard methods.

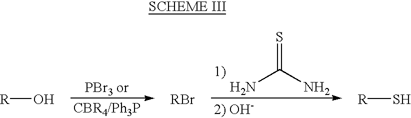

The compounds of formulas XX to XXIV may be readily prepared by standard techniques of organic chemistry, utilizing the general synthetic pathway depicted below. As described by Scheme IV, cyclic amino acids 1 protected by suitable blocking groups P on the amino acid nitrogen may be reacted with thiols RSH to generate thioesters 2. After removal of the protecting group, the free amine 3 may be reacted with various sulfonyl chlorides 4 to provide final products 5 in good to excellent yield.

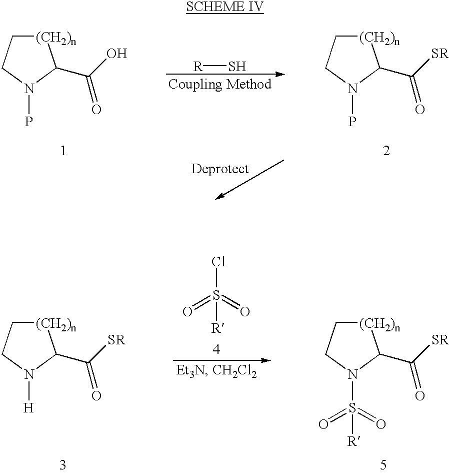

Thiols R—SH may be conveniently prepared from the corresponding readily available alcohols or halides via a two step replacement of halogen by sulfur, as described in Scheme V. Halides may be reacted with thiourea, and the corresponding alkyl thiouronium salts hydrolyzed to provide thiols RSH. If alcohols are used as the starting materials, they may be first converted to the corresponding halides by standard methods.

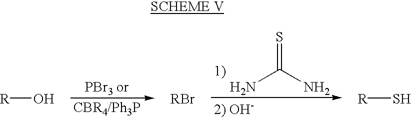

The compounds of formulas XXV to XXIX may be prepared by a variety of synthetic sequences that utilize established chemical transformations. The general pathway to the present compounds is described in Scheme VI. N-glyoxylproline derivatives may be prepared by reacting L-proline methyl ester with methyl oxalyl chloride as shown in Scheme VI. The resulting oxamates may be reacted with a variety of carbon nucleophiles to obtain intermediates compounds. These intermediates are then reacted with a variety of alcohols, amides, or protected amino acid residues to obtain the propyl esters and amides of the invention.

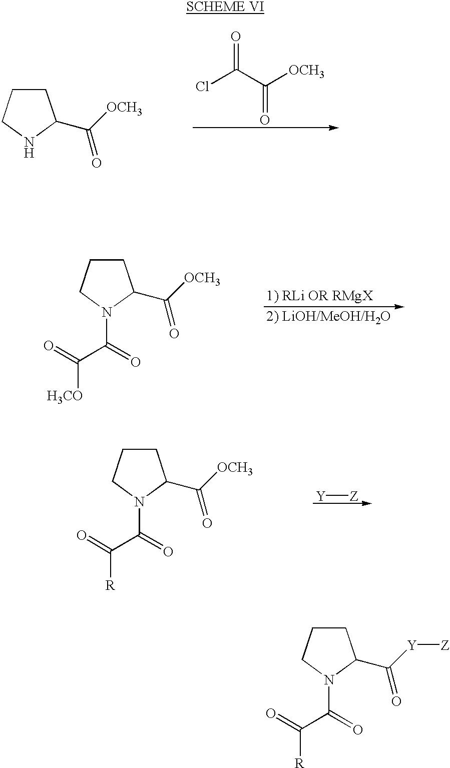

The substituted alcohols may be prepared by a number of methods known to those skilled in the art of organic synthesis. As described in Scheme VII, alkyl or aryl aldehydes may be homologated to phenyl propanols by reaction with methyl(triphenyl-phosphoranylidene)acetate to provide a variety of trans-cinnamates; these latter compounds may be reduced to the saturated alcohols by reaction with excess lithium aluminum hydride, or sequentially by reduction of the double bond by catalytic hydrogenation and reduction of the saturated ester by appropriate reducing agents. Alternatively, the transcinnamates may be reduced to (E)-allylic alcohols by the use of diisobutylaluminum hydride.

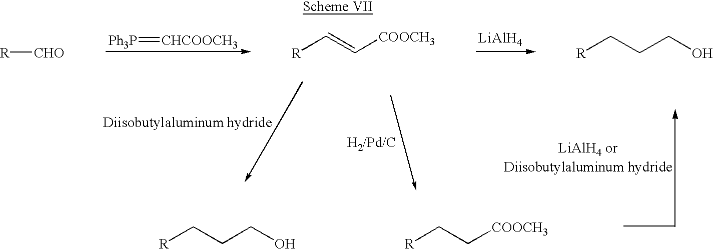

Longer chain alcohols may be prepared by homologation of benzylic and higher aldehydes. Alternatively, these aldehydes may be prepared by conversion of the corresponding phenylacetic and higher acids, and phenethyl and higher alcohols.

The general synthesis of the carboxylic acid isosteres of Formula LXV is outlined in Scheme VIII and IX:

N-glyoxylproline derivatives may be prepared by reacting L-proline methyl ester with methyl oxalyl chloride as shown in Scheme VIII. The resulting oxamates may be reacted with a variety of carbon nucleophiles to obtain compounds used in the present invention, as in Scheme IX.

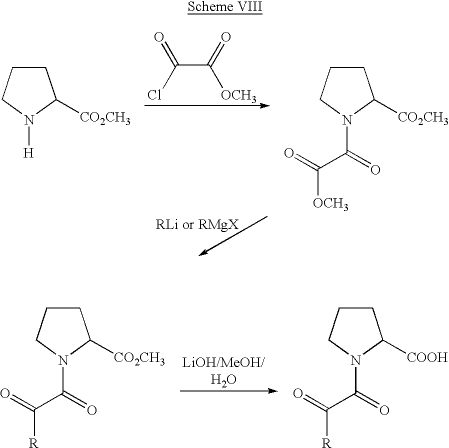

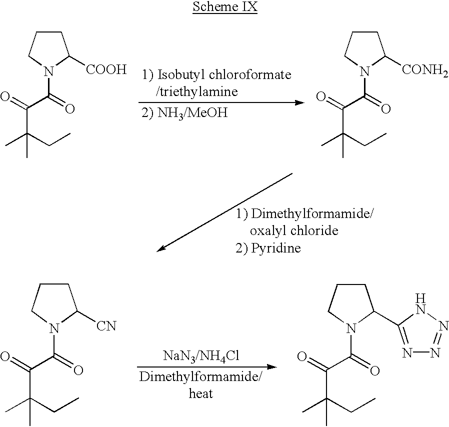

The compounds of formulae LXV may be readily prepared by standard techniques of organic chemistry, utilizing the general synthetic pathways depicted below for di-keto derivatives, sulfonamide derivatives, and urea or carbamate derivatives.

Cyclic amino acids 1 protected by suitable blocking groups P on the amino acid nitrogen may be reacted with thiols RSH to generate thioesters 2. After removal of the protecting group, the free amine 3 may be reacted with a variety of isocyanates or isothiocyanates to provide final ureas or thioureas, respectively.

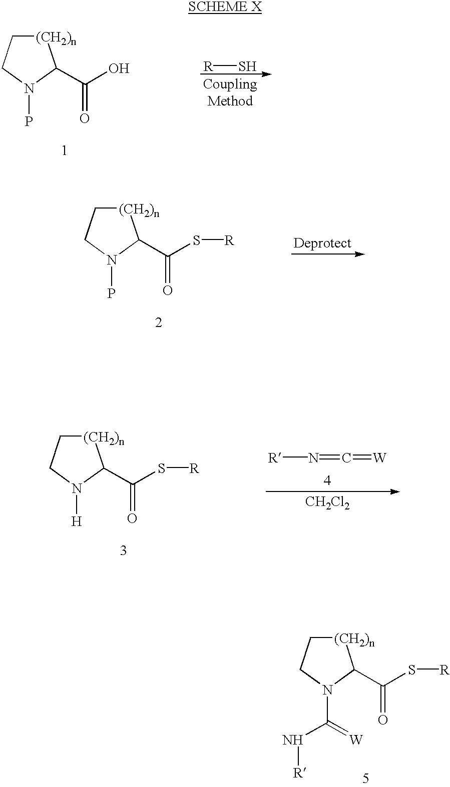

Another scheme for preparing ureas or carbamates is set forth below.

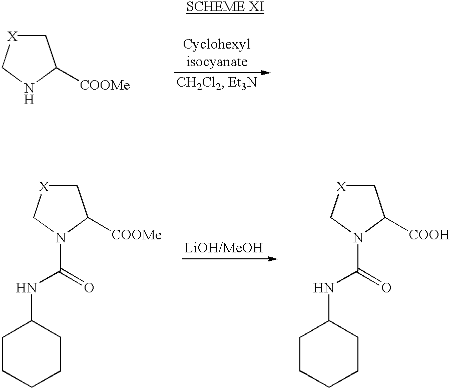

Isocyanates (R′NCO) or isothiocyanates (R′NCS) may be conveniently prepared from the corresponding readily available amines by reaction with phosgene or thiophosgene, as depicted below.

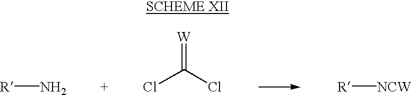

Thiols R—SH may be conveniently prepared from the corresponding readily available alcohols or halides via a two step replacement of halide by sulfur, as described below. Halides may be reacted with thiourea, and the corresponding alkyl thiouronium salts hydrolyzed to provide thiols RSH. If alcohols are used as the starting materials, they may be first converted to the corresponding halides by standard methods.

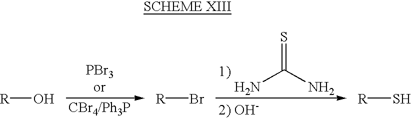

N-glyoxylproline derivatives may be prepared by reacting L-proline methyl ester with methyl oxalyl chloride as shown below. The resulting oxamates may be reacted with a variety of carbon nucleophiles to obtain compounds of the present invention or useful for preparing compounds of the present invention.

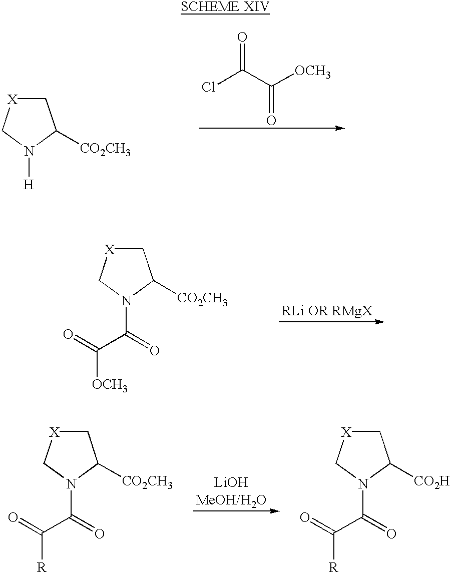

Synthetic schemes for preparing sulfonamide derivatives are known in the art and compounds of the present invention may be synthesized using schemes such as are set forth below.

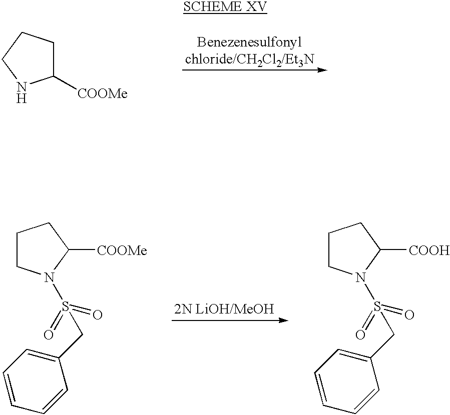

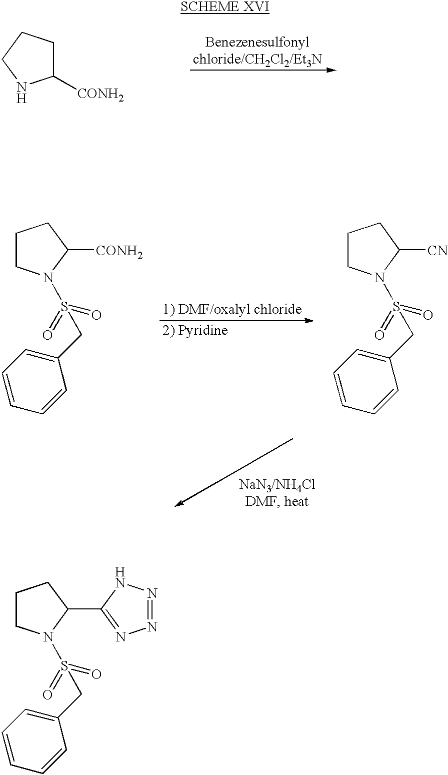

The general synthesis of the carboxylic acid isosteres of Formula LXVI may be prepared by a variety of synthetic sequences that utilize established chemical transformations. An exemplary general pathway to synthesize the present compounds is-described in Scheme XVII.

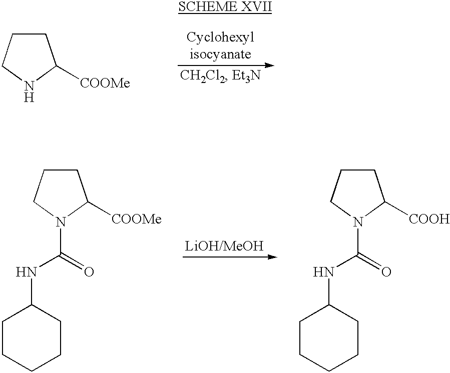

The compounds of formula LXVII may be prepared by a variety of synthetic sequences that utilize established chemical transformations. An exemplary general pathway to the present compounds is described in Schemes XVIII, XVI and XX.

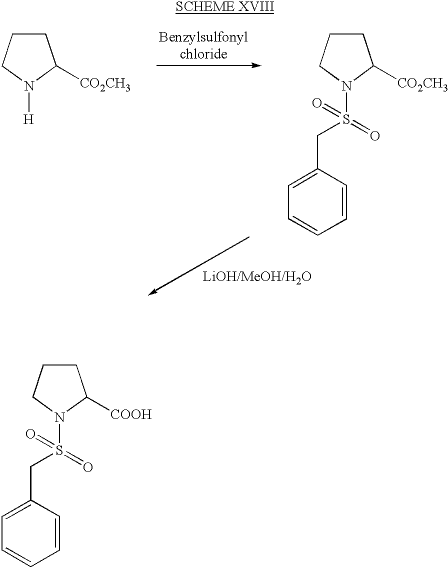

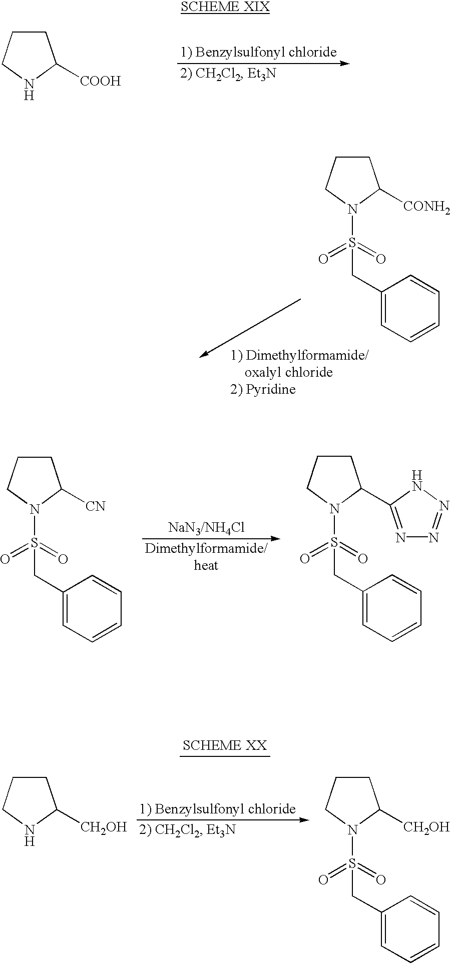

The compounds of formulae LXVIII-LXXIII can be readily prepared by standard techniques of organic chemistry, utilizing the general synthetic pathways depicted below in Schemes XXI and XXII.

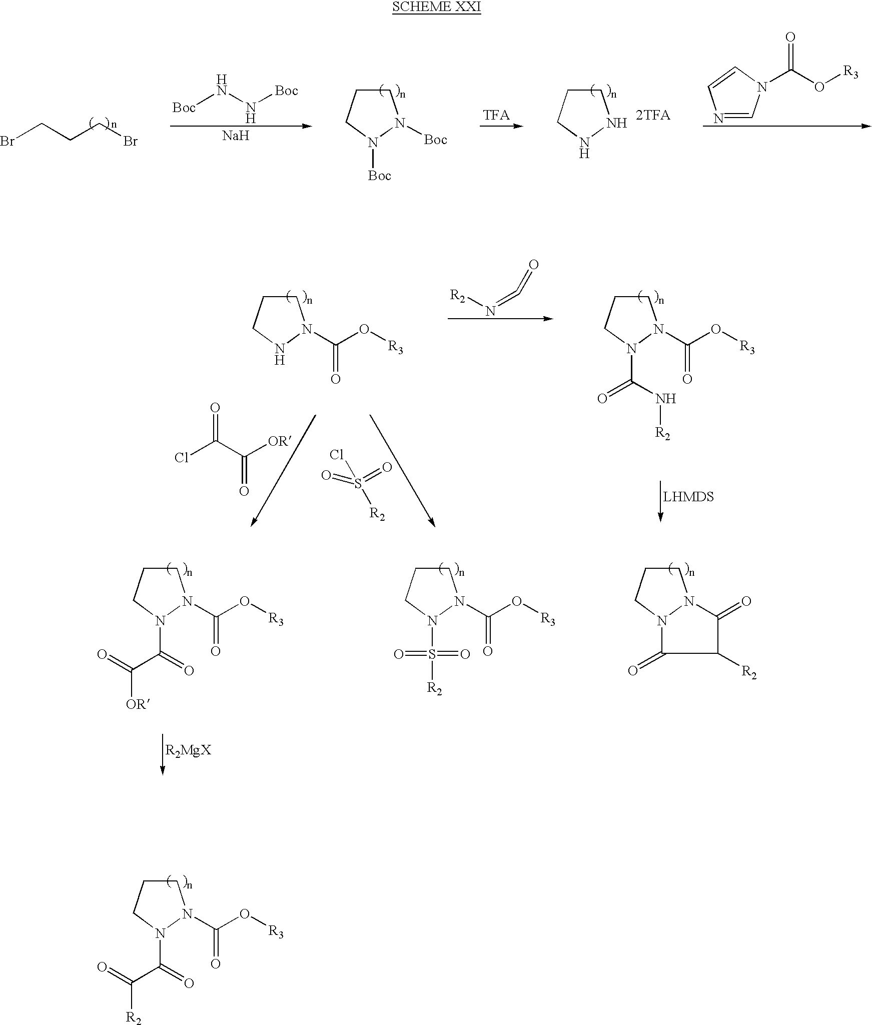

wherein, in Scheme XXI, n, R₃, and R₂ are as defined elsewhere throughout the specification; R′ is a straight or branched chain alkyl group which is optionally substituted in one or more positions; and X is a halogen, wherein any of these substituents are formed in any chemically reasonable substitution pattern. It is further contemplated as within the scope of the present invention that the chlorine atoms depicted in Scheme XXI above can be replaced with any other halogen atom.

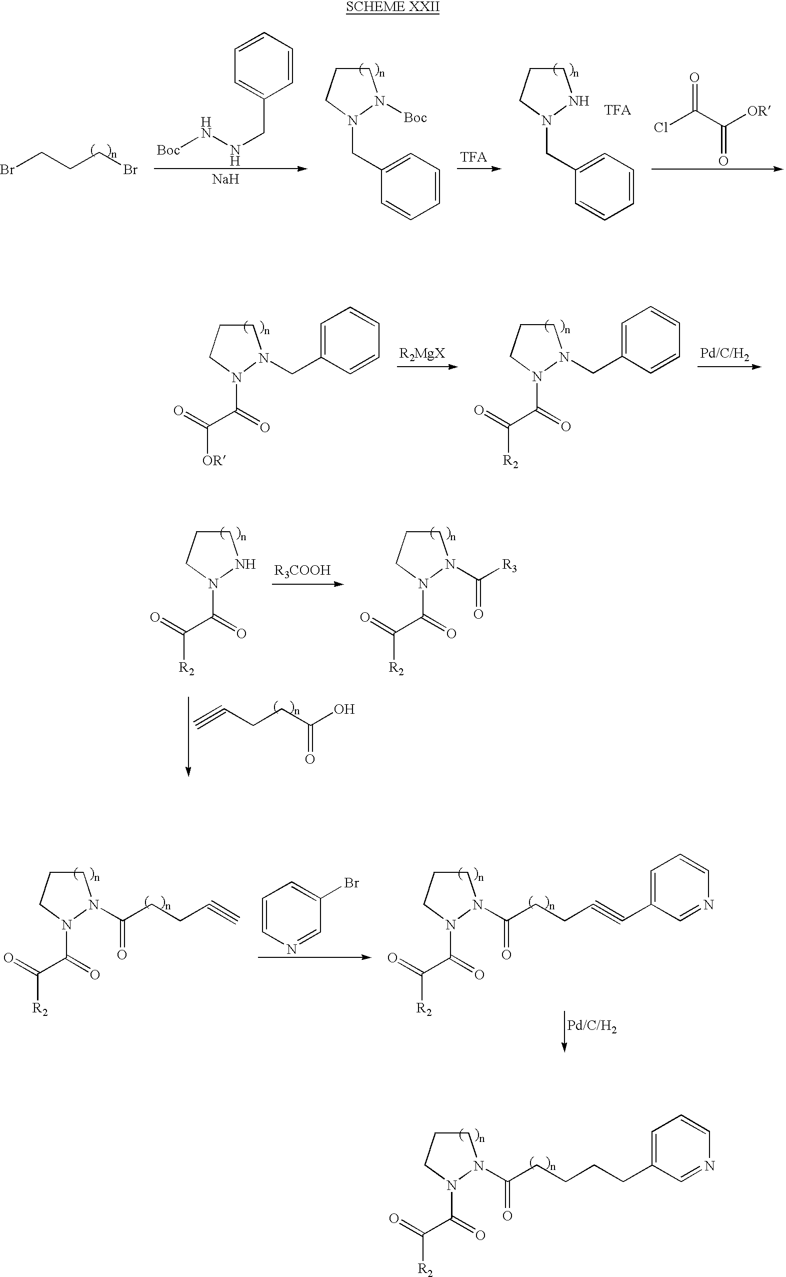

wherein, in Scheme XXII, n, R₁, and R₂ are as defined elsewhere throughout the specification; R′ is a straight or branched chain alkyl group which is optionally substituted in one or more positions; and X is a halogen, wherein any of these substituents are formed in any chemically reasonable substitution pattern. It is further contemplated as within the scope of the present invention that the benzyl groups depicted in Scheme XXII above can be replaced with any R₄ group, wherein R₄ is an alkyl chain substituted with an aryl group; and that the chlorine atoms depicted in Scheme XXII above can be replaced with any other halogen atom.

The compounds of formulae LXXII-LXXIII may be prepared by reacting amino acids with isocyanates and isothiocyanates, as shown in the general method of Scheme XXIII:

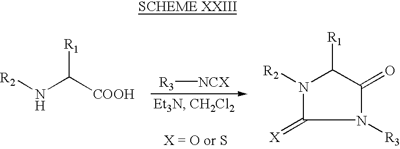

In the preparation of the compounds used in the methods of the present invention, one skilled in the art will understand that one may need to protect or block various reactive functionalities on the starting compounds or intermediates while a desired reaction is carried out on other portions of the molecule. After the desired reactions are complete, or at any desired time, normally such protecting groups will be removed under conditions which will not affect the remaining portion of the molecule, for example by hydrolytic or hydrogenolytic means and the like. Such protection and deprotection steps are conventional in organic chemistry. One skilled in the art is referred to “Protective Groups in Organic Chemistry,” McOmie, ed., Plenum Press, New York, New York; and “Protective Groups in Organic Synthesis,” Greene, ed., John Wiley & Sons, New York, N.Y. (1981) for the teaching of protective groups which may be useful in the preparation of compounds of the present invention. A preferred method involves' removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can be readily neutralized to yield the free amine. Carboxy protecting group, such as methyl, ethyl, benzyl, tert-butyl, 4-methoxyphenylmethyl and the like, can be removed under hydrolysis and hydrogenolysis conditions well known to those skilled in the art.

The product and intermediates may be isolated or purified using one or more standard purification techniques, including, for example, one or more of simple solvent evaporation, recrystallization, distillation, sublimation, filtration, chromatography, including thin-layer chromatography, HPLC (e.g. reverse phase HPLC), column chromatography, flash chromatography, radial chromatography, trituration, and the like.

### Affinity for FKBP12

The compounds used in the inventive methods and pharmaceutical compositions may have an affinity for the FK506 binding protein, particularly FKBP12. The inhibition of the prolyl peptidyl cis-trans isomerase activity of FKBP may be measured as an indicator of this affinity.

### K₁ Test Procedure

The binding to FBKP12 and inhibition of the peptidyl-prolyl isomerase (rotamase) activity of the compounds used in the inventive methods and pharmaceutical compositions can be evaluated by known methods described in the literature (Harding et al., Nature, 1989, 341:758-760; Holt et al. J. Am. Chem. Soc., 115:9923-9938). These values are obtained as apparent Ki's and are presented for representative compounds in TABLES IX to XVI.

The cis-trans isomerization of an alanine-proline bond in a model substrate, N-succinyl-Ala-Ala-Pro-Phe-p-nitroanilide, is monitored spectrophotometrically in a chymotrypsin-coupled assay, which releases paranitroanilide from the trans form of the substrate. The inhibition of this reaction caused by the addition of different concentrations of inhibitor is determined, and the data is analyzed as a change in first-order rate constant as a function of inhibitor concentration to yield the apparent K₁ values.

In a plastic cuvette are added 950 mL of ice cold assay buffer (25 mM HEPES, pH 7.8, 100 mM NaCl), 10 mL of FKBP (2.5 mM in 10 mM Tris-Cl pH 7.5, 100 mM NaCl, 1 mM dithiothreitol), 25 mL of chymotrypsin (50 mg/ml in 1 mM HCl) and 10 mL of test compound at various concentrations in dimethyl sulfoxide. The reaction is initiated by the addition of 5 mL of substrate (succinyl-Ala-Phe-Pro-Phe-para-nitroanilide, 5 mg/mL in 2.35 mM LiCl in trifluoroethanol).

The absorbance at 390 nm versus time is monitored for 90 seconds using a spectrophotometer and the rate constants are determined from the absorbance versus time data files.

TABLE XLI
In Vitro Test Results - Formulas I to XIV

| Compound | Kᵢ (nM) |
|---|---|
| 1 | 31 |
| 2 | 210 |
| 3 | 85 |
| 9 | 104 |
| 10 | 12 |
| 11 | 299 |
| 12 | 442 |
| 14 | 313 |
| 28 | 108 |
| 29 | 59 |
| 30 | 11 |
| 31 | 8.7 |
| 32 | 362 |
| 33 | 1698 |
| 34 | 34 |
| 35 | 62 |
| 36 | 7 |
| 37 | 68 |
| 38 | 8.9 |
| 39 | 347 |
| 40 | 1226 |
| 41 | 366 |
| 42 | 28 |
| 43 | 259 |
| 44 | 188 |
| 45 | 31 |
| 46 | 757 |
| 47 | 21 |
| 48 | 127 |
| 49 | 1334 |
| 50 | 55 |
| 51 | 33 |
| 52 | 6 |
| 53 | 261 |
| 54 | 37 |
| 55 | 30 |
| 56 | 880 |
| 57 | 57 |
| 58 | 79 |
| 59 | 962 |
| 60 | 90 |
| 61 | 139 |
| 62 | 196 |
| 63 | 82 |
| 64 | 163 |
| 65 | 68 |
| 66 | 306 |
| 67 | 177 |
| 68 | 284 |
| 69 | 49 |
| 70 | 457 |
| 71 | 788 |
| 80 | 215 |
| 81 | 638 |
| Parent | 7.5 |
| (unoxidized) | |
| compound of | |
| Example 6 | |
| 95 (Example 6) | 225 |

TABLE XLII
In Vitro Test Results - Formulas XV to XXIV

| Compound | Kᵢ (nM) |
|---|---|
| 101 | +++ |
| 102 | ++ |
| 103 | ++ |
| 104 | ++ |
| 105 | ++ |
| 106 | + |
| 107 | ++ |
| 108 | +++ |
| 109 | +++ |
| 110 | +++ |
| 111 | ++ |
| 112 | +++ |
| 113 | +++ |
| 114 | +++ |
| 115 | +++ |
| 116 | ++ |
| 117 | +++ |
| 118 | ++ |
| 119 | ++ |
| 120 | ++ |
| 121 | ++ |
| 122 | + |
| 123 | ++ |
| 124 | +++ |
| 125 | +++ |
| 126 | +++ |
| 127 | ++ |
| 128 | +++ |
| 129 | +++ |
| 130 | +++ |
| 131 | +++ |
| 132 | ++ |

Relative potencies of compounds are ranked according to the following scale: ++++ denotes K₁ or EDSO <1 nM; +++ denotes K₁ or ED50 of 1-50 nM; ++ denotes K₁ or ED 50 of nM; + denotes K₁ or ED of 201-500 nM.

TABLE XLIII
In Vitro Test Results - Formulas XXV to XXIX

| No. | Z | R′ | K₁ |
|---|---|---|---|
| 137 | 1,1-dimethylpropyl | 3-phenylpropyl | 42 |
| 138 | 1,1-dlmethylpropyl | 3-phenyl-prop-2-(E)-enyl | 125 |
| 139 | 1,1-dimethylpropyl | 3-(3,4,5- | 200 |
| | | trimethoxyphenyl) propyl | |
| 140 | 1,1-dimethylpropyl | 3-(3,4,5-trimethoxyphenyl)- | 65 |
| | | prop-2-(E)-enyl | |
| 141 | 1,1-dimethylpropyl | 3-(4,5-methylenedioxy)- | 170 |
| | | phenylpropyl | |
| 142 | 1,1-dimethylpropyl | 3-(4,5- | 160 |
| | | methylenedioxy)phenylprop-2- | |
| | | (E)-enyl | |
| 143 | 1,1-dimethylpropyl | 3-cyclohexylpropyl | 200 |
| 144 | 1,1-dimethylpropyl | 3-cyclohexylprop-2-(E)-enyl | 600 |
| 145 | 1,1-dimethylpropyl | (1R)-1,3-diphenyl-1-propyl | 52 |
| 146 | 2-furanyl | 3-phenylpropyl | 4000 |
| 147 | 2-thienyl | 3-phenylpropyl | 92 |
| 148 | 2-thiazolyl | 3-phenylpropyl | 100 |
| 149 | Phenyl | 3-phenylpropyl | 1970 |
| 150 | 1,1-dimethylpropyl | 3-(2,5- | 250 |
| | | dimethoxy)phenylpropyl | |
| 151 | 1, 1-dimethylpropyl | 3-(2,5-dimethoxy)phenylprop- | 450 |
| | | 2-(E)-enyl | |
| 152 | 1,1-dimethylpropyl | 2-(3,4,5- | 120 |
| | | trimethoxyphenyl)ethyl | |
| 153 | 1,1-dimethylpropyl | 3-(3-pyridyl)propyl | 5 |
| 154 | 1,1-dimethylpropyl | 3-(2-pyridyl)propyl | 195 |
| 155 | 1,1-dimethylpropyl | 3-(4-pyridyl)propyl | 23 |
| 156 | Cyclohexyl | 3-phenylpropyl | 82 |
| 157 | tert-butyl | 3-phenylpropyl | 95 |
| 158 | Cyclohexylethyl | 3-phenylpropyl | 1025 |
| 159 | Cyclohexylethyl | 3-(3-pyridyl)propyl | 1400 |
| 160 | tert-butyl | 3-(3-pyridyl)propyl | 3 |
| 161 | 1,1-dimethylpropyl | 3,3-diphenylpropyl | 5 |
| 162 | Cyclohexyl | 3-(3-pyridyl)propyl | 9 |
| 163 | 2-thienyl | 3-(3-pyridyl)propyl | 1000 |
| 164 | tert-butyl | 3,3-diphenylpropyl | 5 |
| 185 | Cyclohexyl | 3,3-diphenylpropyl | 20 |
| 186 | 2-thienyl | 3,3-diphenylpropyl | 150 |

TABLE XLIV
In Vitro Test Results

| Compound | Kᵢ (μM) |
|---|---|
| 172 | 140 |
| 175 | 13 |
| 177 | 170 |
| 178 | 250 |
| 179 | 25 |
| 181 | 17 |
| 185 | 12 |
| 202 | >10,000 |
| 207 | 1300 |
| 216 | >10,000 |
| 255 | 1800 |
| 256 | 28 |
| 257 | 39 |
| 258 | 75 |
| 259 | 70 |
| 260 | 165 |
| 261 | 740 |
| 262 | 725 |
| 263 | 130 |
| 264 | 30 |
| 265 | 60 |
| 266 | 15 |
| 267 | 12 |
| 268 | 120 |
| 269 | 20 |
| 270 | 103 |
| 271 | 760 |
| 272 | 210 |
| 273 | 32 |
| 274 | 2 |
| 275 | 24 |
| 276 | 5 |

### EXAMPLES

The following examples are illustrative of the present invention and are not intended to be limitations thereon. Unless otherwise indicated, all percentages are based upon 100% by weight of the final composition.

### Example 1

### Synthesis of (2S)-2-({1-oxo-5-phenyl}-pentyl-1-(3,3-dimethyl-1,2-dioxopentyl)pyrrolidine (1)

(2S)-2-(1-oxo-4-phenyl)butyl-N-benzylpyrrolidine

1-chloro-4-phenylbutane (1.78 g; 10.5 mmol) in 20 mL of THF was added to 0.24 g (10 mmol) of magnesium turnings in 50 mL of refluxing THF. After the addition was complete, the mixture was refluxed for an additional 5 hours, and then added slowly to a refluxing solution of N-benzyl-L-proline thyl ester (2.30 g (10 mmol) in 100 ML of THF. After 2 hours of further reflux, the mixture was cooled and treated with 5 mL of 2 N HCl. The reaction mixture was diluted with ether (100 mL) and washed with saturated NaHCO₃, water and brine. The organic phase was dried, concentrated and chromatographed, eluting with 5:1 CH₂Cl₂:EtOAc to obtain 2.05 g (64%) of the ketone as an oil. ¹H NMR (CDCl₃; 300 MHz): δ 1.49-2.18 (m, 8H); 2.32-2.46 (m, 1H); 2.56-2.65 (m, 2H); 2.97-3.06 (m, 1H); 3.17-3.34 (m, 1H); 3.44-3.62 (m, 1H); 4.02-4.23 (m, 2H); 7.01-7.44 (m, 10H).

(2S)-2-(1-oxo-4-phenyl)butylpyrrolidine

The ketone compound (500 mg) and palladium hydroxide (20% on carbon, 50 mg) was hydrogenated at 40 psi in a Paar shaker overnight. The catalyst was removed by filtration and the solvent was removed in vacuo. The free amine was obtained as a yellow oil (230 mg; 100%).

¹H NMR (CDCl₃; 300 MHz): δ 1.75-2.34 (m, 10H); 2.55 (m, 2H); 2.95 (dm, 1H); 3.45-3.95 (m, 1H); 4.05 (m, 1H); 7.37 (m, 5H).

(2S)-2-(1-oxo-4-phenyl)butyl-1-(1,2-dioxo-2-methoxyethyl)pyrrolidine

To a solution of (2S)-2-(1-oxo-4-phenyl) butylpyrrolidine (230 mg; 1.0 mmol) in CH₂Cl₂(20 mL) at 0° C. was added dropwise methyloxalyl chloride (135 mg; 1.1 mmol). After stirring at 0° C. for 3 hours, the reaction was quenched with saturated NH₄Cl and the organic phase was washed with water and brine and dried and concentrated. The crude residue was purified on a silica gel column, eluting with 20:1 CH₂Cl₂:EtOAc to obtain 300 mg of the oxamate as a clear oil (98%). ¹H NMR (CDCl₃; 300 MHz): δ 1.68 (m, 4H); 1.91-2.38 (m, 4H); 2.64 (t, 2H); 3.66-3.80 (m, 2H); 3.77, 3.85 (s, 3H total); 4.16 (m, 2H); 4.90 (m, 1H); 7.16 (m, 3H); 7.27 (m, 2H).

(2S)-2-({1-oxo-5-phenyl}-pentyl-1-(3,3-dimethyl-1,2-dioxopentyl)pyrrolidine (1)

To a solution of the oxamate above (250 mg; 0.79 mmol) in anhydrous ether (15 mL), cooled to −78° C., was added 1,1-dimethylpropyl-magnesium chloride (0.8 mL of a 1.0 M solution in ether; 0.8 mmol). After stirring the resulting mixture at −78° C. for 2 hours, the reaction was quenched by the addition of 2 mL of saturated NH₄Cl, followed by 100 mL of EtOAc. The organic phase was washed with brine, dried, concentrated, and purified on a silica gel column, eluting with 50:1 CH₂Cl₂:EtOAc. Compound 1 was obtained as a clear oil, 120 mg. ¹H NMR (CDCl₃, 300 MHz): δ 0.87 (t, 3H, J=7.5); 1.22 (s, 3H); 1.25 (s, 3H); 1.67 (m, 4H); 1.70-2.33 (m, 6H); 2.61 (t, 2H, J=7.1); 3.52 (m, 2H); 4.17 (t, 2H, J=6.2); 4.52 (m, 1H); 7.16-7.49 (m, 5H). Analysis calculated for C₂₂H₃₁NO₃—H₂O: C, 70.37; H, 8.86; N, 3.73. Found: 70.48; H, 8.35; N, 3.69.

### Example 2

### Synthesis of 2-phenyl-1-ethyl 1-(3,3-dimethyl-1,2-dioxopentyl)-2-piperidinecarbothioate (10)

Methyl(2S)-1(1,2-dioxo-2-methoxyethyl)-2-pyrrolidinecarboxylate

A solution of L-proline methyl ester hydrochloride (3.08 g; 18.60 mmol) in dry methylene chloride was cooled to 0° C. and treated with triethylamine (3.92 g; 38.74 mmol; 2.1 eq). After stirring the formed slurry under a nitrogen atmosphere for 15 min, a solution of methyl oxalyl chloride (3.20 g; 26.12 mmol) in methylene chloride (45 mL) was added dropwise. The resulting mixture was stirred at 0° C. for 1.5 hour. After filtering to remove solids, the organic phase was washed with water, dried over MgSO₄ and concentrated. The crude residue was purified on a silica gel column, eluting with 50% ethyl acetate in hexane, to obtain 3.52 g (88%) cf the product as a reddish oil. Mixture of cis-trans amide rotamers; data for trans rotamer given. ¹H NMR (CDCl₃) δ 1.93 (dm, 2H); 2.17 (m, 2H); 3.62 (m, 2H); 3.71 (s, 3H); 3.79, 3.84 (s, 3H total); 4.86 (dd, 1H, J=8.4, 3.3).

Methyl(2S)-1-(1,2-dioxo-3,3-dimethylpentyl)-2-pyrrolidinecarboxylate

A solution of methyl (2S)-1-(1,2-dioxo-2-methoxyethyl)-2-pyrrolidinecarboxylate (2.35 g; 10.90 mmol) in 30 mL of tetrahydrofuran (THF) was cooled to −78° C. and treated with 14.2 mL of a 1.0 M solution of 1,1-dimethylpropylmagnesium chloride in THF. After stirring the resulting homogeneous mixture at −78° C. for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 2.10 g (75%) of the oxamate as a colorless oil.

¹H NMR (CDCl₃): δ 0.88 (t, 3H); 1.22, 1.26 (s, 3H each); 1.75(dm, 2H); 1.87-2.10 (m, 3H); 2.23 (m, 1H); 3.54 (m, 2H); 3.76 (s, 3H); 4.52 (dm, 1H, J=8.4, 3.4).

(2S)-1-(1,2-dioxo-3,3-dimethylpentyl)-2-pyrrolidine-carboxylic Acid

A mixture of methyl (2S)-1-(1,2-dioxo-3,3-dimethylpentyl)-2-pyrrolidinecarboxylate (2.10 g; 8.23 mmol), 1 N LiOH (15 mL), and methanol (50 mL) was stirred at 0° C. for 30 minutes and at room temperature overnight. The mixture was acidified to pH 1 with 1 N HCl, diluted with water, and extracted into 100 mL of methylene chloride. The organic extract was washed with brine and concentrated to deliver 1.73 g (87%) of snow-whize solid which did not require further purification. ¹H NMR (CDC₃): δ 0.87 (t, 3H); 1.22, 1.25 (s, 3H each); 1.77 (dm, 2H); 2.02 (m, 2H); 2.17 (m, 1H); 2.25 (m, 1H); 3.53 (dd, 2H, J=10.4, 7.3); 4.55 (dd, 1H, J=8.6, 4.1).

2-phenyl-1-ethyl 1-(3,3-dimethyl-1,2-dioxopentyl)-2-piperidinecarbothioate (10)

To a solution of (2S)-1-(1,2-dioxo-3,3-dimethylpentyl)-2-pyrrolidinecarboxylic acid (241 mg; 1.0 mmol) in CH₂Cl₂ (10 mL) was added dicyclohexylcarbodiimide (226 mg; 1.1 mmol). After stirring the resulting mixture for 5 minutes, the solution was cooled to 0° C. and treated with a solution of phenyl mercaptan (138 mg; 1.0 mmol) and 4-dimethylaminopyridine (6 mg) in 5 ml of CH₂Cl₂. The mixture was allowed to warm to room temperature with stirring overnight. The solids were removed by filtration and the filtrate was concentrated in vacuo; the crude residue was purified by flash chromatography (10:1 hexane:EtOAc) to obtain 302 mg (84%) of compound 10 as an oil. ¹H NMR (CDCl₃, 300 MHz): 60.85 (t, 3H, J=7.5); 1.29 (s, 3H); 1.31 (s, 3H); 1.70-2.32 (m, 6H); 2.92 (t, 2H, J=7.4); 3.22(t, 2H, J=7.4); 3.58 (m, 2H); 4.72 (m, 1H); 7.23-7.34 (m, 5H). Analysis calculated for C₂₀H₂₇NO₃S —0.4H₂O: C, 65.15; H, 7.60; N, 3.80. Found: C, 65.41; H, 7.49; N, 3.72.

### Example 3

### Synthesis of 2-phenyl-1-ethyl (2S)-1-(3,3-dimethyl-1,2-dioxopentyl)-2-pyrrolidinecarbothioate (9)

Methyl 1-(1,2-dioxo-2-methoxyethyl)-2-piperidine-carboxylate

A solution of methyl pipecolate hydrochloride (8.50 g; 47.31 mmol) in dry methylene chloride (100 mL) was cooled to 0° C. and treated with triethylamine (10.5 g; 103 mmol; 2.1 eq). After stirring the formed slurry under a nitrogen atmosphere for 15 minutes, a solution of methyl oxalyl chloride (8.50 g; 69.4 mmol) in methylene chloride (75 mL) was added dropwise. The resulting mixture was stirred at 0° C. for 1,5 hours. After filtering to remove solids, the organic phase was washed with water, dried over MgSO₄ and concentrated. The crude residue was purified on a silica gel column, eluting with 50% ethyl acetate in hexane, to obtain 9.34 g (86%) of the product as a reddish oil. Mixture of cis-trans amide rotamers; data for trans rotamer given. ¹H NMR (CDCl₃): δ 1.22-1.45 (m, 2H); 1.67-1.78 (m, 3H); 2.29 (m, 1H); 3.33 (m, 1H); 3.55 (m, 1H); 3.76 (s, 3H); 3.85, 3.87 (s, 3H total); 4.52 (dd, 1H).

Methyl 1-(1,2-dioxo-3,3-dimethylpentyl)-2-piperidine-carboxylate

A solution of methyl 1-(1,2-dioxo-2-methoxyethyl)-2-piperidinecarboxylate (3.80 g; 16.57 mmol) in 75 mL of tetrahydrofuran (THF) was cooled to −78° C. and treated with 20.7 mL of a 1.0 M solution of 1,1-dimethyl-propylmagnesium chloride in THF. After stirring the resulting homogeneous mixture at −78° C. for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 3.32 g (74%) of the oxamate as a colorless oil. ¹H NMR (CDCl₃): δ 0.88 (t, 3H); 1.21, 1.25 (s, 3H each); 1.35-1.80 (m, 7H); 2.35 (m, 1H)/; 3.24 (m, 1H); 3.41 (m, 1H); 3.76 (s, 3H); 5.32 (d, 1H).

1-(1,2-dioxo-3,3-dimethylpentyl)-2-piperidine-carboxylic Acid

A mixture of methyl 1-(1,2-dioxo-3,3-dimethylpentyl)-2-piperidinecarboxylate (3.30 g; 12.25 mmol), 1 N LiOH (15 mL), and methanol (60 mL) was stirred at 0° C. for 30 minutes and at room temperature overnight. The mixture was acidified to pH 1 with 1 N HCl, diluted with water, and extracted into 100 mL of methylene chloride. The organic extract was washed with brine and concentrated to deliver 2.80 g (87%) of snow-white solid which did not require further purification. ¹H NMR (CDCl₃) δ 0.89 (t, 3H); 1.21, 1.24 (s, 3H each); 1.42-1.85 (m, 7H); 2.35 (m, 1H); 3.22 (d, 1H); 3.42(m, 1H); 5.31 (d, 1H).

2-phenyl-1-ethyl (2S)-1-(3,3-dimethyl-1,2-dioxopentyl)-2-pyrrolidinecarbothioate (9)

To a solution of 1-(1,2-dioxo-3,3-dimethylpentyl)-2-piperidine-carboxylic acid (255 mg; 1.0 mmol) in CH₂Cl₂ (10 mL) was added dicyclohexylcarbodiimide (226 mg; 1.1 mmol). After stirring the resulting mixture for 5 minutes, the solution was cooled to 0° C. and treated with a solution of phenyl mercaptan (138 mg; 1.0 mmol) and 4-dimethylaminopyridine (6 mg) in 5 ml of CH₂Cl₂. The mixture was allowed to warm to room temperature with stirring overnight. The solids were removed by filtration and the filtrate was concentrated in vacuo; the crude residue was purified by flash chromatography (10:1 hexane:EtOAc) to obtain 300 mg (80%) of compound 9 as an oil. ¹H NMR (CDCl₃, 300 MHz): δ 0.94 (t, 3H, J=7.5); 1.27 (s, 3H); 1.30 (s, 3H); 1.34-1.88 (m, 7H); 2.45 (m, 1H); 2.90 (t, 2H, J=7.7); 3.26 (t, 2H, J=7.7); 3.27 (m, 1H); 3.38 (m, 1H); 5.34 (m, 1H); 7.24-7.36 (m, 5H). Analysis calculated for C₂₁H₂₉NO₃S: C, 67.17; H, 7.78; N, 3.73. Found: C, 67.02; H, 7.83; N, 3.78.

### Example 4

### Synthesis of 3-phenyl-1-propyl(2S)-1-(3,3-dimethyl-1,2-dioxopentyl)-2-(4-thiazolidine)carboxylate (80)

1-(1,2-dioxo-2-methoxyethyl)2-(4-thiazolidine)-carboxylate

A solution of L-thioproline (1.51 g; 11.34 mmol)in 40 mL of dry methylene chloride was cooled to 0° C. and treated with 3.3 mL (2.41 g; 23,81 mmol) of triethylamine. After stirring this mixture for 30 minutes, a solution of methyl oxalyl chloride (1.81 g; 14.74 mmol) was added dropwise. The resulting mixture was stirred at 0° C. for 1.5 hours, filtered through Celite to remove solids, dried and concentrated. The crude material was purified on a silica gel column, eluting with 10% MeOH in methylene chloride, to obtain 2.0 g of the oxamate as an orange-yellow solid.

3-phenyl-1-propyl(2S)-1-(1,2-dioxo-2-methoxyethyl)2-(4-thiazolidine)carboxylate

1-(1,2-dioxo-2-methoxyethyl)2-(4-thiazolidine)-carboxylate (500 mg; 2.25 mmol), 3-phenyl-1-propanol (465 mg; 3.42 mmol), dicyclohexylcarbodiimide (750 mg; 3.65 mmol), 4-dimethylaminopyridine (95 mg; 0.75 mmol) and camphorsulfonic acid (175 mg; 0.75 mmol) in 30 mL of methylene chloride were stirred together overnight. The mixture was filtered through Celite to remove solids and chromatographed (25% ethyl acetate/hexane) to obtain 690 mg of material. ¹H NMR (CDCl₃, 300 MHz): δ 1.92-2.01 (m, 2H); 2.61-2.69 (m, 2H); 3.34 (m, 1H); 4.11-4.25 (m, 2H) 4.73 (m, 1H); 5.34 (m, 1H); 7.12 (m, 3H); 7.23 (m, 2H).

3-phenyl-1-propyl(2S)-1-(3,3-dimethyl-1,2-dioxopentyl)-2-(4-thiazolidine)carboxylate (80)

A solution of 3-phenyl-1-propyl(2S)-1-(1,2-dioxo-2-methoxyethyl)2-(4-thiazolidine)carboxylate (670 mg; 1.98 mmol) in tetrahydrofuran (10 mL) was cooled to −78° C. and treated with 2.3 mL of a 1.0 M solution of 1,1-dimethylpropylmagnesium chloride in ether. After stirring the mixture for 3 hours, it was poured into saturated ammonium chloride, extracted into ethyl acetate, and the organic phase was washed with water, dried and concentrated. The crude material was purified on a silica gel column, eluting with 25% ethyl acetate in hexane, to obtain 380 mg of the compound of Example 4 as a yellow oil. ¹H NMR (CDCl₃, 300 MHz): δ 0.86 (t, 3H); 1.21 (s, 3H); 1.26 (s, 3H); 1.62-1.91 (m, 3H); 2.01 (m, 2H); 2.71 (m, 2H); 3.26-3.33 (m, 2H); 4.19 (m, 2H); 4.58 (m, 1H); 7.19 (m, 3H); 7.30 (m, 2H). Analysis calculated for C₂₀H₂₇NO₄S: C, 63.63; H, 7.23; N, 3.71. Found: C, 64.29; H, 7.39; N, 3.46.

### Example 5

### Synthesis of 3-(3-pyridyl)-1-propyl(2S)-1-(3,3-dimethyl-1,2-dioxopentyl)-2-(4-thiazolidine) carboxylate (81)

The compound of Example 5 was prepared according to the procedure of Example 4, using 3-(3-pyridyl)-1-propanol in the final step, to yield 3-(3-pyridyl)-1-propyl(2S)-1-(3,3-dimethyl-1,2-dioxopentyl)-2-(4-thiazolidine)carboxylate. ¹H NMR (CDCl₃, 300 MHz): δ0.89 (t, 3H, J=7.3); 1.25 (s, 3H); 1.28 (s, 3H); 1.77 (q, 2H, J=7.3); 2.03 (tt, 2H, J=6.4, 7.5); 2.72 (t, 2H, J=7.5); 3.20 (dd, 1H, J=4.0, 11.8); 3.23 (dd, 1H, J=7.0, 11.8); 4.23 (t, 2H, J=6.4); 4.55 (d, 2H, T=8.9); 5.08 (dd, 1H, J=4.0, 7.0); 7.24 (m, 1H); 8.48 (m, 2H). Analysis calculated for C₁₉H₂₆N₂O₄S -0.5H₂O: C, 58.89; H, 7.02; N, 7.23. Found: C, 58.83; H, 7.05; N, 7.19.

### Example 6

### Synthesis of 3-(3-pyridyl)-1-propyl (2S)-1-(3,3-Dimethyl-1,2-dioxopentyl)-2-pyrrolidinecarboxylate, N-oxide (95)

Methyl (2S)-1-(1,2-dioxo-2-methoxyethyl)-2-pyrrolidinecarboxylate

A solution of L-proline methyl ester hydrochloride (3.08 g; 18.60 mmol) in dry methylene chloride was cooled to 0° C. and treated with triethylamine (3.92 g; 38.74 mmol; 2.1 eq). After stirring the formed slurry under a nitrogen atmosphere for 15 minutes, a solution of methyl oxalyl chloride (3.20 g; 26.12 mmol) in methylene chloride (45 mL) was added dropwise. The resulting mixture was stirred at 0° C. for 1.5 hour. After filtering to remove solids, the organic phase was washed with water, dried over MgSO₄ and concentrated. The crude residue was purified on a silica gel column, eluting with 50% ethyl acetate in hexane, to obtain 3.52 g (88%) of the product as a reddish oil. Mixture of cis-trans amide rotamers; data for trans rotamer given. ¹H NMR (CDCl₃): δ 1.93 (dm, 2H); 2.17 (m, 2H); 3.62 (m, 2H); 3.71 (s, 3H); 3.79, 3.84 (s, 3H total); 4.86 (dd, 1H, J=8.4, 3.3).

Methyl(2S)-1-(1,2-dioxo-3,3-dimethylcentyl)-2-pyrrolidinecarboxylate

A solution of methyl (2S)-1-(1,2-dioxo-2-methoxyethyl)-2-pyrrolidinecarboxilate (2.35 g; 10.90 mmol) in 30 mL of tetrahydrofuran (THE) was cooled to −78° C. and treated with 14.2 mL of a 1.0 M solution of 1,1-dimethylpropylmagnesium chloride in THF. After stirring the resulting homogeneous mixture at −78° C. for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 2.10 g (75%) of the oxamate as a colorless oil.

¹H NMR (CDCl₃): δ 0.88 (t, 3H); 1.22, 1.26 (s, 3H each); 1.75 (dm, 2H); 1.87-2.10 (m, 3H); 2.23 (m, 1H); 3.54 (m, 2H); 3.76 (s, 3H); 4.52 (dm, 1H, J=8.4, 3.4).

(2S)-1(1,2-dioxo-3,3-dimethylpentyl)-2-pyrrolidinecarboxylic Acid

A mixture of methyl (2S)-1-(1,2-dioxo-3,3-dimethylpentyl-2-pyrrolidine-carboxylate (2.10 g; 8.23 mmol), 1 N LiOH (15 mL), and methanol (50 mL) was stirred at 0° C. for 30 minutes and at room temperature overnight. The mixture was acidified to pH 1 with 1 N HCl, diluted with water, and extracted into 100 mL of methylene chloride. The organic extract was washed with brine and concentrated to deliver 1.73 g (87%) of snow-white solid which did not require further purification. ¹H NMR (CDCl₃): δ 0.87 (t, 3H); 1.22, 1.25 (s, 3H each); 1.77 (dm, 2H); 2 .02 (m, 2H); 2.17 (m, 1H); 2.25 (m, 1H); 3.53 (dd, 2H, J=10.4, 7.3); 4.55 (dd, 1H, J=8.6, 4.1).

3-(3-Pyridyl)-1-propyl(2S)-1-(3,3-dimethyl-1,2-dioxopentyl)-2-pyrrolidinecarboxylate

A mixture of (2S)-1-(1,2-dioxo-3,3-dimethylpentyl)-2-pyrrolidinecarboxylic acid (4.58 g; 19 mmol), 3-pyridinepropanol (3.91 g; 28.5 mmol), dicyclohexylcarbodiimide (6.27 g; 30.4 mmol), camphorsulfonic acid (1.47 g; 6.33 mmol) and 4-dimethyl aminopyridine (773 mg; 6.33 mmol) in methylene chloride (100 mL) was stirred overnight under a nitrogen atmosphere. The reaction mixture was filtered through Celite to remove solids and concentrated in vacuo. The crude material was triturated with several portions of ether, and the ether portions were filtered through Celite to remove solids and concentrated in vacuo. The concentrated filtrate was purified on a flash column (gradient elution, 25% ethyl acetate in hexane to pure ethyl acetate) to obtain 5.47 g (80%) of the captioned compound as a colorless oil (partial hydrate). ¹H NMR (CDCl₃, 300 MHz): δ 0.85 (t, 3H); 1.23, 1.26 (s, 3H each); 1.63-1.89 (m, 2H); 1.90-2.30 (m, 4H); 2.30-2.50 (m, 1H); 2.72 (t, 2H); 3.53 (m, 2H); 4.19 (m, 2H);-4.53 (m, 1H); 7.22 (m, 1H); 7.53 (dd, 1H); 8.45. Analysis calculated for C₂₀H₂₈NO₄-0.25H₂O: C, 65.82; H, 7.87; N, 7.68. Found: C, 66.01; H, 7.85; N, 7.64.

3-(3-Pyridyl)-1-propyl (2S)-1-(3,3-dimethyl-1,2-dioxopentyl)-2-pyrrolidinecarboxylate, N-oxide (95)

A solution of 3-(3-pyridyl)-1-propyl (2S)-1-(3,3-dimethyl-1,2-dioxopentyl)-2-pyrrolidinecarboxylate (190 mg; 0.52 mmol) and m-chloroperbenzoic acid (160 mg of 57%-86% material, 0.53 mmol) was stirred in methylene chloride (20 mL) at room temperature for 3 hours. The reaction mixture was diluted with methylene chloride and washed twice with 1 N NaOH. The organic extract was dried and concentrated, and the crude material was chromatographed, eluting with 10% methanol in ethyl acetate, to obtain 130 mg of the Compound 95 of Example 6. ¹H NMR (CDCl₃, 300 MHz): δ 0.83 (t, 3H); 1.21 (s, 3H) 1.25 (s, 3H); 1.75-2.23 (m, 8H); 2.69 (t, 2H, J=7.5); 3.52 (t, 2H, J=6.3); 4.17 (dd, 2H, J=6.3); 4.51 (m, 1H); 7.16-7.22 (m, 2H); 8.06-8.11 (m, 2H). Analysis calculated for C₂₀H₂₈N₂O₅-0.75H₂O: C, 61.60; H, 7.63; N, 7.18. Found: C, 61.79; H, 7.58; N, 7.23.

### Example 7

### Synthesis of 3-(3-Pyridyl)-1-propylmercaptyl 2S-1-[(2-methylbutyl)carbamoyl]pyrrolidine-2-carboxylate (101)

3-(3-Pyridyl)-1-propylchloride

To a solution of 3-(3-pyridyl)-1-propanol (10 g; 72.4 mmol) in chloroform (100 mL) was added dropwise a solution of thionyl chloride (12.9 g; 108.6 mmol) in chloroform (50 mL). The resulting mixture was refluxed for 1 hour, then poured into ice-cold 50% aqueous potassium hydroxide (150 mL). The layers were separated, and the organic phase was dried, concentrated, and purified on a silica gel column, eluting with 40% ethylacetate in hexane, to obtain 10 g (65%) of the chloride as a clear oil. ¹H NMR (300 MHz, CDCl₃): δ 2.02-2.11 (m, 2H); 2.77 (m, 2H); 3.51 (m, 2H); 7.20 (m, 1H); 7.49 (m, 1H); 8.45 (m, 2H).

3-(3-Pyridyl)-1-propylmercaptan

A mixture of 3-(3-pyridyl)-1-propylchloride (3 g; 19.4 mmol) and thiourea (1.48 g; 19.4 mmol) in ethanol (10 mL) was refluxed for 24 hours. Aqueous sodium hydroxide, 15 mL of a 0.75 N solution, was added, and the mixture was refluxed for an additional 2 hours. After cooling to room temperature, the solvent was removed in vacuo. Chromatographic purification of the crude thiol on a silica gel column eluting with 50% ethyl acetate in hexane delivered 1.2 g of 3-(3-Pyridyl)-1-propylmercaptan as a clear liquid. ¹H NMR (300 MHz, CDC₃): δ 1.34 (m, 1H); 1.90 (m, 2H); 2.52 (m, 2H); 2.71 (m, 2H); 7.81 (m, 1H); 7.47 (m, 1H); 8.42 (m, 2H).

3-(3-Pyridyl)-1-propylmercaptyl N-(tert-butyloxycarbonyl)pyrrolidine-2-carboxylate

A mixture of N-(tert-butyloxycarbonyl)-(S)-proline (3.0 g; 13.9 mmol); 3-(3-Pyridyl)-1-propylmercaptan (3.20 g; 20.9 mmol), dicyclohexylcarbodiimide (4.59 g; 22.24 mmol), camphorsulfonic acid (1.08 g; 4.63 mmol), and 4-dimethylaminopyridine (0.60 g; 4.63 mmol) in dry methylene chloride (100 mL) was stirred overnight. The reaction mixture was diluted with methylene chloride (50 mL) and water (100 mL), and the layers were separated. The organic phase was washed with water (3×100 mL), dried over magnesium sulfate, and concentrated, and the crude residue was purified on a silica gel column eluting with ethyl acetate to obtain 4.60 g (95%) of the thioester as a thick oil. ¹H NMR (300 MHz, CDCl₃): δ1.45 (s, 9H); 1.70-2.05 (m, 5H); 2.32 (m, 1H); 2.71 (t, 2H); 2.85 (m, 2H); 3.50 (m, 2H); 4.18 (m, 1H); 7.24 (m, 1H); 7.51 (m, 1H); 8.48 (m, 2H).

3-(3-Pyridyl)-1-propylmercaptyl pyrrolidine-2-carboxylate

A solution of 3-(3-Pyridyl)-1-mercaptyl N-(tert-butyloxycarbonyl)pyrrolidine-2-carboxylate (4.60 g; 13.1 mmol) in methylene chloride (60 mL) and trifluoroacetic acid (6 mL) was stirred at room temperature for three hours. Saturated potassium carbonate was added until the pH was basic, and the reaction mixture was extracted with methylene chloride (3×). The combined organic extracts were dried and concentrated to yIeld 2.36 g (75%) of the free amine as a thick oil. ¹H NMR (300 MHz, CDCl₃): δ 1.87-2.20 (m, 6H); 2.79 (m, 2H); 3.03-3.15 (m, 4H total); 3.84 (m, 1H); 7.32 (m, 1H); 7.60 (m, 1H); 8.57 (m, 2H).

3-(3-Pyridyl)-1-propylmercaptyl 2s⁻¹-[(2-methyl-butyl)carbamoyl]pyrrolidine-2-carboxylate (101)

A solution of 2-methylbutylamine (113 mg; 1.3 mmol) and triethylamine (132 mg; 1.3 mmol) in methylene chloride (5 mL) was added to a solution of triphosgene (128 mg; 0.43 mmol) in methylene chloride (5 mL). The resulting mixture was refluxed for 1 hour and then cooled to room temperature. 3-(3-Pyridyl)-1-propylmercaptyl pyrrolidine-2-carboxylate (300 mg; 1.3 mmol) in 5 mL of methylene chloride was added and the resulting mixture was stirred for 1 hour and then partitioned between water and a 1:1 mixture of ethyl acetate and hexane. The organic phase was dried, concentrated and purified by column chromatography (50% ethyl acetate/hexane) to obtain 250 mg (55%) of the compound of Example 7 (Compound 101, Table VII) as an oil. ¹H NMR (CDCl₃, 300 MHz): δ0.89-0.93 (m, 6H); 1.10-1.20 (m, 1H); 1.27 (s, 1H); 1.36-1.60 (m, 2H); 1.72 (s, 2H); 1.97-2.28 (m, 6H); 2.70-2.75 (m, 2H); 2.92-3.54 (m, 6H); 4.45-4.47 (m, 1H); 7.21-7.29 (m, 1H); 7.53-7.56 (dd, 1H); 8.46-8.48 (s, 2H).

### Example 8

### Synthesis of 3-(3-Pyridyl)-1-propyl 2S-1-[(1′,1′-Dimethylpropyl)carbamoyl]pyrrolidine-2-carboxylate (102)

Reaction of 3-(3-pyridyl)-1-proovlmercaptyl pyrrolidine-2-carboxylate with the isocyanate generated from tert-amylamine and triphosgene, as described for Example 7, provided the comoound of Example 8 (Compound 102, Table VII) in 62% yield. ¹H NMR (CDCl₃, 300 MHz): δ 0.83 (t, 3H); 1.27 (s, 6H); 1.64-1.71 (m, 2H); 1.91-2.02 (m, 7H); 2.66-2.71 (t, 2H); 2.85 (m, 2H); 3.29-3.42 (m, 2H); 4.11 (br, 1H); 4.37-4.41 (m, 1H).

### Example 9

### Synthesis of 3-(3-pyridyl)-1-propylmercaptyl 2S-1-[(cyclohexyl)thiocarbamoyl]-pyrrolidine-2-carboxylate (107)

A mixture of cyclohexylisothiocyanate (120 mg; 0.9 mmol), 3-(3-pyridyl)-1-propylmercaptyl pyrrolidine-2-carboxylate (200 mg; 0.9 mmol) and triethylamine (90 mg; 0.9 mmol) in 20 mL of methylene chloride was stirred for 1 hour and then partitioned between water and a 1:1 mixture of ethyl acetate and hexane. The organic phase was dried, concentrated and purified by column chromatography (50% ethyl acetate/hexane) to obtain-160 mg (47%) of the compound of Example 9 (Compound 107, Table VII). ¹H NMR (CDCl₃, 300 MHz): δ 1.16-1.40 (m, 6H) 1.50-1.71 (m, 4H); 1.95-2.08 (m, 7H); 2.70-2.75 (t, 2H); 3.03 (m, 2H); 3.40-3.60 (m, 2H); 4.95-4.98 (d, 1H); 5.26-5.29 (d, 1H); 7.17-7.25 (m, 1H).

### Example 10

### Synthesis of 3-(para-Methoxyphenyl)-1-propylmercaptyl(2S)-N-(benzenesulfonyl)pyrrolidine-2-carboxylate (120)

3-(p-Methoxyphenyl)-1-propylbromide

To a solution of 3-(p-methoxyphenyl)-1-propanol (16.6 g; 0.1 mol) in 250 mL of toluene, cooled to 0° C., was added dropwise 26 mL of phosphorus tribromide (0.27 mol). Following completion of the addition, the reaction was stirred at room temperature for 1 hour, then refluxed for an additional hour. The reaction was cooled and poured onto ice, the layers were separated, and the organic phase washed with saturated sodium bicarbonate (3×) and brine (3×). The crude material obtained upon drying and evaporation of the solvent was chromatographed, eluting with 10% EtOAc/hexane, to obtain 14 g (61%) of 3-(p-methoxyphenyl)-1-propylbromide.

3-(p-Methoxyphenyl)-1-propylmercaptan

A mixture of 3-(p-methoxyphenyl)-1-propylbromide (14 g; 61 mmol) and thiourea (5.1 g; 67 mmol) in ethanol (150 mL) was refluxed for 48 hours. Evaporation of the solvent provided a clear glassy compound, which was dissolved in 50 mL of water and treated with 100 mL of 40% aqueous sodium hydroxide. After stirring the resulting mixture for two hours, the product was extracted into ether (3×), and the combined organic extracts were washed with sodium bicarbonate and brine, dried, and concentrated. Chromatographic purification of the crude thiol on a silica gel column eluting with 2% either in hexane delivered 10.2 g of 3-(p-methoxyphenyl)-1-propylmercaptan as a clear liquid. ¹H NMR (300 MHz, CDCl₃): δ 1.34 (t, 1H); 1.88-1.92 (m, 2H); 2.49-2.53 (m, 2H); 2.64-2.69 (m, 2H); 3.77 (s, 3H); 6.80-6.84 (m, 2H); 7.06-7.24 (m, 2H)

3-(p-Methoxyphenyl)-1-mercaptyl N-(tert-butyloxycarbonyl)pyrrolidine-2-carboxylate

A mixture of N-(tert-butyloxycarbonyl)-(S)-proline (2.0 g; 9.29 mmol), 3-(p-methoxyphenyl)-1-propylmercaptan (1.86 g; 10.22 mmol), 1-(3-dimethylaminopropyl)-3-ethylcarbodiimide hydrochloride (1.96 g; 10.22 mmol), and 4-dimethylaminopyridine (catalytic) in dry methylene chloride (50 mL) was stirred overnight. The reaction mixture was diluted with methylene chloride (50 mL) and water 100 (mL), and the layers were separated. The organic phase was washed with water (3×100 mL), dried over magnesium sulfate, and concentrated to provide 3.05 g of the product (100%) as a thick oil. ¹H NMR (300 MHz, CDCl₃): δ 1.15 (s, 9H); 1.84-2.31 (m, 6H); 2.61 (m, 2H); 2.83 (m, 2H); 3.51 (m, 2H); 3.75 (s, 3H); 6.79 (d, 2H, J=8.04); 7.05 (m, 2H).

3-(p-Methoxyphenyl)-1-mercaptyl pyrrolidine-2-carboxylate

A solution of 3-(p-methoxyphenyl)-mercaptyl N-(tert-butyloxycarbonyl)pyrrolidine-2-carboxylate (3.0 g; 8.94 mmol) in methylene chloride (60 mL) and trifluoroacetic acid (6 mL) was stirred at room temperature for three hours. Saturated potassium carbonate was added until the pH was basic, and the reaction mixture was extracted with methylene chloride (3×). The combined organic extracts were dried and concentrated to yield 1.73 g (69%) of the free amine as a thick oil. ¹H NMR (300 MHz, CDCl₃): δ 1.80-2.23 (m, 6H); 2.62 (m, 2H); 2.81 (m, 2H); 3.01 (m, 2H); 3.75 (s, 3H); 3.89(m, 1H); 6.81 (m, 2H); 7.06 (m, 2H).

3-(para-Methoxyphenyl)-1-propylmercaptyl (2S)-N-(benzenesulfonyl)pyrrolidine-2-carboxylate (120)

A solution of 3-(p-methoxyphenyl)-1-mercaptyl pyrrolidine-2-carboxylate (567 mg; 2.03 mmol) and benzenesulfonyl chloride (358 mg; 2.03 mmol) in methylene chloride (5 mL) was treated with diisopropylethylamine (290 mg; 2.23 mmol) and stirred overnight at room temperature. The reaction mixture was filtered to remove solids and applied directly to a silica gel column, eluting with 25% ethyl acetate in hexane, to obtain 540 mg of Compound 120 (Table VIII) as a clear oil. ¹H NMR (300 MHz, CDCl₃): δ 1.65-1.89 (m, 6H); 2.61 (t, 2H, J=7.3); 2.87 (t, 2H, J=7.6); 3.26 (m, 1H); 3.54 (m, 1H); 3.76 (s, 3H); 4.34 (dd, 1H, J=2.7, 8.6); 6.79 (d, 2H, J=8.7); 7.06 (d, 2H, J=8.6); 7.49-7.59 (m, 3H); 7.86 (dd, 2H, J=1.5, 6.8).

### Example 11

### Synthesis of 3-(para-Methoxyphenyl)-1-propylmercaptyl(2S)-N-(a-toluenesulfonyl)pyrrolidine-2-carboxylate (121)

A solution of 3-(p-Methoxyphenyl)-1-mercaptyl pyrrolidine-2-carboxylate (645 mg; 2.30 mmol) and a-toluenesulfonyl chloride (440 mg; 2.30 mmol) in methylene chloride (5 mL) was treated with diisopropylethylamine (330 mg; 2.53 mmol) and stirred overnight at room temperature. Purification as described for Example 10 provided the compound of Example 11 (Compound 121, Table VIII) as a clear oil. ¹H NMR (300 MHz, CDCL₃): δ 1.65-2.25 (m, 8H); 2.65 (t, 2H); 2.89-2.96 (m, 2H); 3.55-3.73 (m, 2H); 3.80 (s, 3H); 4.32 (s, 2H); 4.70-4.81 (m, 1H); 6.83 (d, 2H); 7.09 (d, 2H); 7.14 (m, 3H); 7.26 (m, 2H).

### Example 12

### Synthesis of 3-(para-Methoxyphenyl)-1-propylmercaptyl(2S)-N-(a-toluenesulfonyl)pyrrolidine-2-carboxylate (122)

A solution of 3-(p-methoxyphenyl)-1-mercaptyl pyrrolidine-2-carboxylate (567 mg; 2.30 mmol) and p-toluenesulfonyl chloride (425 mg; 2.23 mmol) in methylene chloride (5 mL) was stirred overnight at room temperature. Purification as described for Example 10 provided the compound of Example 12 (Compound 122, Table VIII) as a clear oil. ¹H NMR (300 MHz, CDCl₃): δ 1.67-1.94 (m, 6H); 2.40 (s, 3H); 2.61 (t, 2H, J=7.3); 2.84 (m, 2H, J=7.2); 3.22 (m, 1H); 3.52 (m, 1H); 3.76 (s, 3H); 4.32 (dd, 1H, J-2.9, 8.5); 6.79 (d, 2H, J=6.5); 7.07 (d, 2H, J=6.5); 7.29 (d, 2H, J=6.5); 7.74 (d, 2H, J=6.5).

### EXAMPLE 13

### Synthesis of 1,5-Diphenyl-3-pentylmercaptyl N-(para-toluenesulfonyl)pipecolate (134)

3-Phenyl-1-propanal

Oxalyl chloride (2.90 g; 2.29 mm=1) in methylene chloride (50 mL), cooled to −78° C., was treated with dimethylsulfoxide (3.4 mL) in 10 mL of methylene chloride. After stirring for 5 min, 3-phenyl-1-propanol (2.72 g; 20 mmol) in 20 mL of methylene chloride was added, and the resulting mixture was stirred at −78° C. for 15 min, treated with 14 mL of triethylamine, stirred an additional 15 min, and poured into 100 mL of water. The layers were separated, the organic phase was dried and concentrated, and the crude residue was purified on a silica gel column, eluting with 10% ethyl acetate in hexane, to obtain 1.27 g (47%) of the aldehyde as a clear oil. ¹H NMR (300 MHz, CDCl₃): δ 2.80 (m, 2H); 2.98 (m, 2H); 7.27 (m, 5H); 9.81 (2, 1H).

1,5-Diphenyl-3-pentanol

A solution of 2-(bromoethyl)benzene (1.73 g; 9.33 mmol) in diethylether (10 mL) was added to a stirred slurry of magnesium turnings (250 mg; 10.18 mmol) in 5 mL of ether. The reaction was initiated with a heat gun, and after the addition was complete the mixture was heated on an oil bath for 30 min. 3-Phenyl-1-propanal (1.25 g; 9.33 mmol) was added in 10 mL of ether, and reflux was continued for 1 hour. The reaction was cooled and quenched with saturated ammonium chloride, extracted into 2× ethyl acetate, and the combined organic portions were dried and concentrated. Chromatographic purification on a silica gel column (10% ethyl acetate in hexane) delivered 1.42 g(63%) of the diphenyl alcohol.

¹H NMR (300 MHz, CDCl₃): δ 1.84 (m, 4H); 2.61-2.76(m, 4H) 3.65 (m, 1H); 7.19-7.29 (m, 10H).

1,5-Diphenyl-3-bromopentane

To a solution of 1,5-diphenyl-3-pentanol (1.20 g (5 mmol) and carbon tetrabromide (1.67 g; 5 mmol) in methylene chloride (20 mL) was added triphenylphosphine (1.31 g; 5 mmol) portionwise, at 0° C. After stirring at room temperature for 18 hours, the mixture was concentrated, triturated with ether, and the solids removed by filtration. The filtrate was passed through a plug of silica gel, eluting with hexane:methylene chloride, 10:1, to give 1.35 g (90%) of the bromide as an oil which was used without further purification. ¹H NMR (300 MHz, CDCl₃): δ 2.11-2.18 (m, 4H); 2.73 (m, 2H); 2.86 (m, 2H); 3.95 (m, 1H); 7.16-7.30 (m, 10H).

1,5-Diphenyl-3-pentylmercaptan

Using the procedure described in Example 10 for the conversion of bromides to thiols, 1,5-diphenyl-3-bromopentane was converted to 1,5-dlphenyl-3-pentylmercaptan in 35% overall yield. ¹H NMR (300 MHz, CDCl₃): δ 1.79 (m, 2H); 1.98 (m, 2H); 2.71 (m, 3H); 2.80 (m, 2H); 7.16-7.28 (m, 10H).

1,5-Diphenyl-3-pentylmercaptyl N-(tert-butyloxycarbonyl)pyrrolidine-2-carboxylate

A mixture of N-(tert-butyloxycarbonyl)-(S)-pipecolic acid (2.11 g; 9.29 mmol), 1,5-diphenyl-3-pentylmercaptan (2.58 g; 10.22 mmol), 1-(3-dimethylaminopropyl)-3-ethylcarbodiimide hydrochloride (1.96 g; 10.22 mmol) and 4-dimethylaminopyridine (catalytic) in dry methylene chloride (50 mL) was stirred overnight. the reaction mixture was diluted with methylene chloride (50 mL) and water (100 mL), and the layers were separated. The organic phase was washed with water (3×100 mL), dried over magnesium sulfate, and concentrated to provide 870 mg (20%) of the product as a thick oil, which was used without further purification.

1,5-Diphenyl-3-pentylmercaptyl Pyrrolidine-2-carboxylate

A solution of 1,5-diphenyl-3-pentylmercaptyl N-(tert-butyloxycarbonyl)pyrrolidine-2-carboxylate (850 mg; 1.8 mmol) in methylene chloride (10 mL) and trifluoroacetic acid (1 mL) was stirred at room temperature for three hours. Saturated potassium carbonate was added until the pH was basic, and the reaction mixture was extracted with methylene chloride. The combined organic extracts were dried and concentrated to yield 480 mg (72%) of the free amine as a thick oil, which was used without further purification.

1,5-Diohenyl-3-pentylmercaptyl N-(para-toluenesulfonyl)pipecolate (134)

1,5-Diphenyl-3-pentylmercaptyl N-(para-toluenesulfonyl)pipecolace(18) was prepared from 1,5-diphenyl-3-pentylmercaptyl pyrrolidine-2-carboxylate and para-toluenesulfonyl chloride as described for Example 12, in 65% vield. ¹H NMR (CDCl₃, 300 MHz): δ 0.80 (m, 4H); 1.23-1.97 (m, 5H); 2.15 (d, 1H); 2.61-2.69 (m, 4H); 3.23 (m, 1H); 3.44 (dm, 1H); 4.27 (s, 2H); 4.53 (d, 1H, J 4.5); 5.06 (m, 1H); 7.16-7.34 (m, 15H).

### Example 14

### Synthesis of 3-phenyl-1-propyl (2S)-1-(3,3-dimethyl-1,2-dioxopentyl)-2-pyrrolidinecarboxylate (137)

Methyl (2S)-1-(1,2-dioxo-2-methoxyethyl)-2-pyrrolidinecarboxylate

A solution of L-proline methyl ester hydrochloride (3.08 g; 18.60 mmol) in dry methylene chloride was cooled to 0° C. and treated with triethylamine (3.92 g; 38.74 mmol; 2.1 eq). After stirring the formed slurry under a nitrogen atmosphere for 15 min, a solution of methyl oxalyl chloride (3.20 g; 26.12 mmol) in methylene chloride (45 mL) was added dropwise. The resulting mixture was stirred at 0° C. for 1.5 hour. After filtering to remove solids, the organic phase was washed with water, dried over MgSO₄ and concentrated. The crude residue was purified on a silica gel column, eluting with 50% ethyl acetate in hexane, to obtain 3.52 g (88%) of the product as a reddish oil. Mixture of cis-trans amide rotamers; data for trans rotamer given. ¹H NMR (CDCl₃): δ 1.93 (dm, 2H); 2.17 (m, 2H); 3.62 (m, 2H); 3.71 (s, 3H); 3.79, 3.84 (s, 3H total); 4.86 (dd, 1H, J=8.4, 3.3).

Methyl (2S)-1-(1,2-dioxo-3,3-dlmethylpentyl)-2-pyrrolidinecarboxylate

A solution of methyl (25)-!-(1,2-dloxo-2-methoxyethyl)-2-pyrrolidinecarboxylate (2.35 g; 10.90 mmol) in 30 mL of tetrahydrofuran (THF) was cooled to −78° C. and treated with 14.2 mL of a 1.0 M solution of 1,1-dimethylpropylmagnesium chloride in THF. After stirring the resulting homogeneous mixture at −78° C. for three hours, the mixture was poured into saturated ammonium chloride (100 mL) and extracted into ethyl acetate. The organic phase was washed with water, cried, and concentrated, and the crude material obtained upon removal of the solvent was purified on a silica gel column, eluting with 25% ethyl acetate in hexane, to obtain 2.10 g (75%) of the oxamate as a colorless oil.

¹H NMR (CDCl₃): δ 0.88 (t, 3H); 1.22, 1.26 (s, 3H each); 1.75 (dm, 2H); 1.87-2.10 (m, 3H); 2.23 (m, 1H); 3.54 (m, 2H); 3.76 (s, 3H); 4.52 (dm, 1H, J=8.4, 3.4).

Synthesis of (2S)-1-(1,2-dioxo-3,3-dimethylpentyl)-2-pyrrolidinecarboxylic Acid

A mixture of methyl (2S)-1-(1,2-dioxo-3,3-dimethylpentyl)-2-pyrrolidinecarboxylate (2.10 g; 8.23 mmol), 1 N LiOH (15 mL), and methanol (50 mL) was stirred at 0° C. for 30 minutes and at room temperature overnight. The mixture was acidified to pH 1 with 1 N HCl, diluted with water, and extracted into 100 mL of methylene chloride. The organic extract was washed with brine and concentrated to deliver 1.73 g (87%) of snow-white solid which did not require further purification. ¹H NMR (CDC₃): δ 0.87 (t, 3H); 1.22, 1.25 (s, 3H each); 1.77 (dm, 2H); 2.02 (m, 2H); 2.17 (m, 1H); 2.25 (m, 1H); 3.53 (dd, 2H, J=10.4, 7.3); 4.55 (dd, 1H, J=8.6, 4.1).

3-Phenyl-1-propyl (2S)-1-(3,3-dimethyl-1,2-dioxopentyl)-2-pyrrolidinecarboxylate (137)

A mixture of (2S)-1-(1,2-dioxo-3,3-dimethylpentyl)-2-pyrrolidine-carboxylic acid (600 mg; 2.49 mmol), 3-phenyl-1-propanol (508 mg; 3.73 mmol), dicyclohexylcarbodiimide (822 mg; 3.98 mmol), camphorsulfonic acid (190 mg; 0.8 mmol) and 4-dimethylaminopyridine (100 mg; 0.8 mmol) in methylene chloride (20 mL) was stirred overnight under a nitrogen atmosphere. The reaction mixture was filtered through Celite to remove solids and concentrated in vacuo, and the crude material was purified on a flash column (25% ethyl acetate in hexane) to obtain 720 mg (80%) of Example 14 as a colorless oil. ¹H NMR (CDCl₃): δ 0.84 (t, 3H); 1.19 (s, 3H); 1.23 (s, 3H); 1.70 (dm, 2H); 1.98 (m, 5H); 2.22 (m, 1H); 2.64 (m, 2H); 3.47 (m, 2H); 4.14 (m, 2H); 4.51 (d, 1H); 7.16 (m, 3H); 7.26 (m, 2H).

### Example 15

The method of Example 14 was utilized to prepare the following illustrative compounds.

Compound 138: 3-phenyl-1-prop-2-(E)-enyl (2S)-1-(3,3-dimethyl-1,2-dioxopentyl)-2-pyrrolidinecarboxylate; 80%.

¹H NMR (360 MHz, CDCl₃): δ 0.86 (t, 3H); 1.21 (s, 3H); 1.25 (s, 3H); 1.54-2.10 (m, 5H); 2.10-2.37 (m, 1H); 3.52-3.55 (m, 2H); 4.56 (dd, 1H, J=3.8, 8.9); 4.78-4.83 (m, 2H); 6.27 (m, 1H); 6.67 (dd, 1H, J=15.9); 7.13-7.50 (m, 5H).

Compound 139: 3-(3,4,5-trimethoxyphenyl)-1-propyl (2S)-1-(3,3-dimethyl-1,2-dioxopentyl)-2-pyrrolidine-carboxylate, 61%. ¹H NMR (CDCl₃): δ 0.84 (t, 3H); 1.15 (s, 3H); 1.24 (s, 3H); 1.71 (dm, 2H); 1.98 (m, 5H); 2.24 (m, 1H); 2.63 (m, 2H); 3.51 (t, 2H); 3.79 (s, 3H); 3.83 (s, 3H); 4.14 (m, 2H); 4.52 (m, 1H); 6.36 (s, 2H).

Compound 140: 3-(3,4,5-trimethoxyphenyl)-1-prop-2-(E)-enyl (2S)-1-(3,3-dimethyl-1,2-dioxopentyl)-2-pyrrolidine carboxylate, 66%. ¹H NMR (CDCl₃): δ 0.85 (t, 3H); 1.22 (s, 3H); 1.25 (s, 3H); 1.50-2.11 (m, 5H); 2.11-2.40 (m, 1H); 3.55 (m, 2H); 3.85 (s, 3H); 3.88 (s, 6H); 4.56 (dd, 1H); 4.81 (m, 2H); 6.22 (m, 1H); 6.58 (d, 1H, J=16); 6.63 (s, 2H).

Compound 141: 3-(4,5-methylenedioxyphenyl)-1-propyl (2S)-1-(3,3-dimethyl-1,2-dioxopentyl)-2-pyrrolidine-carboxylate, 82%. ¹H NMR (360 MHz, CDCl₃): δ 0.86 (t, 3H); 1.22 (s, 3H); 1.25 (s, 3H); 1.60-2.10 (m, 5H); 3.36-3.79 (m, 2H); 4.53 (dd, 1H, J=3.8, 8.6); 4.61-4.89 (m, 2H); 5.96 (s, 2H); 6.10 (m, 1H); 6.57 (dd, 1H, J=6.2, 15.8); 6.75 (d, 1H, J=8.0); 6.83 (dd, 1H, J=1.3, 8.0); 6.93 (s, 1H).

Compound 142: 3-(4,5-methylenedioxyphenyl)-1-prop-2-(E)-enyl (2S)-1-(3,3-dimethyl-1,2-dioxopentyl)-2-pyrrolidinecarboxylate, 82%. ¹H NMR (360 MHz, CDCl₃): δ 0.86 (t, 3H); 1.22 (s, 3H); 1.25 (s, 3H); 1.60-2.10 (m, 5H); 2.10-2.39 (m, 1H); 3.36-3.79 (m, 2H); 4.53 (dd, 1H, J=3.8, 8.6); 4.61-4.89 (m, 2H); 5.96 (s, 2H); 6.10 (m, 1H); 6.57 (dd, 1H, J=6.2, 15.8); 6.75 (d, 1H, J=8.0); 6.83 (dd, 1H, J=1.3, 8.0); 6.93 (s, 1H).

Compound 144: 3-cyclohexyl-1-prop-2-(E)-enyl (2S)-1-(3,3-dimethyl-1,2-dioxopentyl)-2-pyrrolidine-carboxylate, 92%. ¹H NMR (360 MHz, CDCl₃): δ 0.86 (t, 3H); 1.13-1.40 (m+2 singlets, 9H total); 1.50-1.87 (m, 8H); 1.87-2.44 (m, 6H); 3.34-3.82 (m, 2H); 4.40-4.76 (m, 3H); 5.35-5.60 (m, 1H); 5.60-5.82 (dd, 1H, J=6.5, 16).

Compound 145: (1R)-1,3-Diphenyl-1-propyl (2S)-1-(3,3-dimethyl-1,2-dioxopentyl)-2-pyrrolidinecarboxylate, 90%.

¹H NMR (360 MHz, CDCl₃): δ 0.85 (t, 3H); 1.20 (s, 3H); 1.23 (s, 3H); 1.49-2.39 (m, 7H); 2.46-2.86 (m, 2H); 3.25-3.80 (m, 2H); 4.42-4.82 (m, 1H); 5.82 (td, 1H, J=1.8, 6.7); 7.05-7.21 (m, 3H); 7.21-7.46 (m, 7H).

Compound 146: 3-phenyl-1-propyl (2S)-1-(1,2-dioxo-2-[2-furanyl])ethyl-2-pyrrolidinecarboxylate, 99%. ¹H NMR (300 MHz, CDCl₃): δ 1.66-2.41 (m, 6H); 2.72 (t, 2H, J=7.5); 3.75 (m, 2H); 4.21 (m, 2H); 4.61 (m, 1H); 6.58 (m, 1H); 7.16-7.29 (m, 5H); 7.73 (m, 2H).

Compound 147: 3-phenyl-1-propyl (2S)-1-(1,2-dioxo-2-[2-thienyl])ethyl-2-pyrrolidinecarboxylate, 81%. ¹H NMR (300 MHz, CDCl₃): δ 1.88-2.41 (m, 6H); 2.72 (dm, 2H); 3.72 (m, 2H); 4.05 (m, 1H); 4.22 (m, 1H); 4.64 (m, 1H); 7.13-7.29 (m, 6H); 7.75 (dm, 1H); 8.05 (m, 1H).

Compound 149: 3-phenyl-1-propyl (2S)-1-(1,2-dioxo-2-phenyl)ethyl-2-pyrrolidinecarboxylate, 99%. ¹H NMR (300 MHz, CDCl₃): 1.97-2.32 (m, 6H); 2.74 (t, 2H, J=7.5); 3.57 (m, 2H); 4.24 (m, 2H); 4.67 (m, 1H); 6.95-7.28 (m, 5H); 7.51-7.64 (m, 3H); 8.03-8.09 (m, 2H).

Compound 150: 3-(2,5-dimethoxyphenyl)-1-propyl (2S)-1-(3,3-dimethyl-1,2-dioxopentyl)-2-pyrrolidine-carboxylate, 99%. ¹H NMR (300 MHz, CDCl₃): δ 0.87 (t, 3H); 1.22 (s, 3H); 1.26 (s, 3H); 1.69 (m, 2H); 1.96 (m, 5H); 2.24 (m, 1H); 2.68 (m, 2H); 3.55 (m, 2H); 3.75 (s, 3H); 3.77 (s, 3H); 4.17 (m, 2H); 4.53 (d, 1H); 6.72 (m, 3H)

Compound 151: 3-(2,5-dimethoxyphenyl)-1-prop-2-(E)-enyl (2S)-1-(3,3-dimethyl-1,2-dioxopentyl)-2-pyrrolidine-carboxylate, 99%. ¹H NMR (300 MHz, CDCl₃): δ 0.87 (t, 3H); 1.22 (s, 3H); 1.26 (s, 3H); 1.67 (m, 2H); 1.78 (m, 1H); 2.07 (m, 2H); 2.26 (m, 1H); 3.52 (m, 2H); 3.78 (s, 3H); 3.80 (s, 3H); 4.54 (m, 1H); 4.81 (m, 2H); 6.29 (dt, 1H, J=15.9); 6.98 (s, 1H).

Compound 152: 2-(3,4,5-trimethoxyphenyl)-1-ethyl (2S)-1-(3,3-dimethyl-1,2-dioxopentyl)-2-pyrrolidine-carboxylate, 97%. ¹H NMR (300 MHz, CDCl₃): δ 0.84 (t, 3H); 1.15 (s, 3H); 1.24 (s, 3H); 1.71 (dm, 2H); 1.98 (m, 5H); 2.24 (m, 1H); 2.63 (m, 2H); 3.51 (t, 2H); 3.79 (s, 3H); 3.83 (s, 3H); 4.14 (m, 2H); 4.52 (m, 1H); 6.36 (s, 2H).

Compound 153: 3-(3-Pyridyl)-1-propyl (2S)-1-(3,3-dimethyl-1,2-dioxopentyl)-2-pyrrolidinecarboxylate, 80%.

¹H NMR (CDCl₃, 300 MHz): δ 0.85 (t, 3H); 1.23, 1.26 (s, 3H each); 1.63-1.89 (m, 2H); 1.90-2.30 (m, 4H); 2.30-2.50 (m, 1H); 2.72 (t, 2H); 3.53 (m, 2H); 4.19 (m, 2H); 4.53 (m, 1H); 7.22 (m, 1H); 7.53 (dd, 1H); 8.45.

Compound 154: 3-(2-Pyridyl)-1-propyl (2S)-1-(3,3-dimethyl-1,2-dioxopentyl)-2-pyrrolidinecarboxylate, 88%.

¹H NMR (CDCl₃, 300 MHz): δ 0.84 (t, 3H); 1.22, 1.27 (s, 3H each); 1.68-2.32 (m, 8H); 2.88 (t, 2H, J=7.5); 3.52 (m, 2H); 4.20 (m, 2H); 4.51 (m, 1H); 7.09-7.19 (m, 2H); 7.59 (m, 1H); 8.53 (d, 1H, J=4.9).

Compound 155: 3-(4-Pyridyl)-1-propyl (2S)-1-(3,3-dimethyl-1,2-dioxopentyl)-2-pyrrolidinecarboxylate, 91%.

¹H NMR (CDCl₃, 300 MHz): δ 6.92-6.80 (m, 4H); 6.28 (m, 1H); 5.25 (d, 1H, J=5.7); 4.12 (m, 1H); 4.08 (s, 3H); 3.79 (s, 3H); 3.30 (m, 2H); 2.33 (m, 1H); 1.85-1.22 (m, 7H); 1.25 (s, 3H); 1.23 (s, 3H); 0.89 (t, 3H, J=7.5).

Compound 156: 3-phenyl-1-propyl (2S)-1-(2-cyclohexyl-1,2-dioxoethyl)-2-pyrrolidinecarboxylate, 91%. ¹H NMR (CDCl₃, 300 MHz): δ 1.09-1.33 (m, 5H); 1.62-2.33 (m, 12H); 2.69 (t, 2H, J=7.5); 3.15 (dm, 1H); 3.68 (m, 2H); 4.16 (m, 2H); 4.53, 4.84 (d, 1H total); 7.19 (m, 3H); 7.29 (m, 2H).

Compound 157: 3-phenyl-1-propyl (2S)-1-(2-tert-butyl-1,2-dioxoethyl)-2-pyrrolidinecarboxylate, 92%. ¹H NMR (CDCl₃, 300 MHz): δ 1.29 (s, 9H); 1.94-2.03 (m, 5H); 2.21 (m, 1H); 2.69 (m, 2H); 3.50-3.52 (m, 2H); 4.16 (m, 2H); 4.53 (m, 1H); 7.19 (m, 3H); 7.30 (m, 2H).

Compound 158: 3-phenyl-1-propyl (2S)-1-(2-cyclohexylethyl-1,2-dioxoethyl)-2-pyrrolidinecarboxylate, 97%. ¹H NMR (CDCl₃, 300 MHz): δ 0.88 (m, 2H); 1.16 (m, 4H); 1.43-1.51 (m, 2H); 1.67 (m, 5H); 1.94-2.01 (m, 6H); 2.66-2.87 (m, 4H); 3.62-3.77 (m, 2H); 4.15 (m, 2H); 4.86 (m, 1H); 7.17-7.32 (m, 5H).

Compound 159: 3-(3-pyridyl)-1-propyl (2S)-1-(2-cyclohexylethyl-1,2-dioxoethyl)-2-pyrrolidinecarboxylate, 70%.

¹H NMR (CDCl₃, 300 MHz): 60.87 (m, 2H); 1.16 (m, 4H) 1.49 (m, 2H); 1.68 (m, 4H); 1.95-2.32 (m, 7H); 2.71 (m, 2H); 2.85 (m, 2H); 3.63-3.78 (m, 2H); 4.19 (m, 2H); 5.30 (m, 1H); 7.23 (m, 1H); 7.53 (m, 1H); 8.46 (m, 2H).

Compound 160: 3-(3-pyridyl)-1-propyl (25)-1-(2-tert-butyl-1,2-dioxoethyl)-2-pyrrolidinecarboxylate, 83%. ¹H NMR (CDCl₃, 300 MHz): δ 1.29 (s, 9H); 1.95-2.04 (m, 5H); 2.31 (m, 1H); 2.72 (t, 2H, J=7.5); 3.52 (m, 2H); 4.18 (m, 2H); 4.52 (m, 1H); 7.19-7.25 (m, 1H); 7.53 (m, 1H); 8.46 (m, 2H).

Compound 161: 3,3-diphenyl-1-propyl (2S)-1-(3,3-dimethyl-1,2-dioxopentyl)-2-pyrrolidinecarboxylate, 99%.

¹H NMR (CDCl₃, 300 MHz): δ 0.85 (t, 3H); 1.21, 1.26 (s, 3H each); 1.68-2.04 (m, 5H); 2.31 (m, 1H); 2.40 (m, 2H); 3.51 (m, 2H); 4.08 (m, 3H); 4.52 (m, 1H); 7.18-7.31 (m, 10H).

Compound 162: 3-(3-pyridyl)-1-propyl (2S)-1-(2-cyclohexyl-1,2-dioxoethyl)-2-pyrrolidinecarboxylate, 88%. ¹H NMR (CDCl₃, 300 MHz): 61.24-1.28 (m, 5H); 1.88-2.35 (m, 11H); 2.72 (t, 2H, J=7.5); 3.00-3.33 (dm, 1H); 3.69 (m, 2H); 4.19 (m, 2H); 4.55 (m, 1H); 7.20-7.24 (m, 1H); 7.53 (m, 1H); 8.47 (m, 2H).

Compound 163: 3-(3-Pyridyl)-1-propyl (2S)-N-([2-thienyl]glyoxyl)pyrrolidinecarboxylate, 49%. ¹H NMR (CDCl₃, 300 MHz): 61.81-2.39 (m, 6H); 2.72 (dm, 2H); 3.73 (m, 2H); 4.21 (m, 2H); 4.95 (m, 1H); 7.19 (m, 2H); 7.61 (m, 1H); 7.80 (d, 1H); 8.04 (d, 1H); 8.46 (m, 2H).

Compound 164: 3,3-Diphenyl-1-propyl (2S)-1-(3,3-dimethyl-1,2-dioxobutyl)-2-pyrrolidinecarboxylate, 99%.

¹H NMR (CDCl₃, 300 MHz): 61.27 (s, 9H); 1.96 (m, 2H) 2.44 (m, 4H); 3.49 (m, 1H); 3.64 (m, 1H); 4.08 (m, 4H) 4.53 (dd, 1H); 7.24 (m, 10H).

Compound 165: 3,3-Diphenyl-1-propyl (2S)-1-cyclohexyl glyoxyl-2-pyrrolidinecarboxylate, 91%. ¹H NMR (CDCl₃, 300 MHz): 61.32 (m, 6H); 1.54-2.41 (m, 10H); 3.20 (dm, 1H); 3.69 (m, 2H); 4.12 (m, 4H); 4.52 (d, 1H); 7.28 (m, 10H).

Compound 166: 3,3-Diphenyl-1-propyl (2S)-1-(2-thienyl) glyoxyl-2-pyrrolidinecarboxylate, 75%. ¹H NMR (CDCl₃, 300 MHz): δ 2.04 (m, 3H); 2.26 (m, 2H); 2.48 (m, 1H); 3.70 (m, 2H); 3.82-4.18 (m, 3H total); 4.64 (m, 1H); 7.25 (m, 1H); 7.76 (dd, 1H); 8.03 (m, 1H).

### Example 16

General procedure for the synthesis of acrylic esters, exemplified for methyl (3,3,5-trimethoxy)-trans-cinnamate.

A solution of 3,4,5-trimethoxybenzaldehyde (5.0 g; 25.48 mmol) and methyl (triphenyl-phosphoranylidene)acetate (10.0 g; 29.91 mmol) in tetrahydrofuran (250 mL) was refluxed overnight. After cooling, the reaction mixture was diluted with 200 mL of ethyl acetate and washed with 2×200 mL of water, dried, and concentrated in vacuo. The crude residue was chromatographed on a silica gel column, eluting with 25% ethyl acetate in hexane, to obtain 5.63 g (88%) of the cinnamate as a white crystalline solid. ¹H NMR (300 MHz; CDCl₃): δ 3.78 (s, 3H); 3.85 (s, 6H); 6.32 (d, 1H, J 16); 6.72 (s, 2H); 7.59 (d, 1H, J=16).

### Example 17

General procedure for the synthesis of saturated alcohols from acrylic esters, exemplified for (3,4,5-trimethoxy) phenylpropanol.

A solution of methyl (3,3,5-trimethoxy)-trans-cinnamate (1.81 g; 7.17 mmol) in tetrahydrofuran (30 mL) was added in a dropwise manner to a solution of lithium aluminum hydride (14 mmol) in THF (35 mL), with stirring and under an argon atmosphere. After the addition was complete, the mixture was heated to 75° C. for 4 hours. After cooling, it was quenched by the careful addition of 15 mL of 2 N NaOH followed by 50 mL of water. The resulting mixture was filtered through Celite to remove solids, and the filter cake was washed with ethyl acetate. The combined organic fractions were washed with water, dried, concentrated in vacuo, and purified on a silica gel column, eluting with ethyl acetate to obtain 0.86 g (53%) of the alcohol as a clear oil. ¹H NMR (300 MHz; CDCl₃): δ 1.23 (br, 1H); 1.87 (m, 2H); 2.61 (t, 2H, J=7.1); 3.66 (t, 2H); 3.80 (s, 3H); 3.83 (s, 6H); 6.40 (s, 2H).

### Example 18

General procedure for the synthesis of trans-allylic alcohols from acrylic esters, exemplified for (3,4,5-trimethoxy)phenylprop-2-(E)-enol.

A solution of methyl (3,3,5-trimethoxy)-trans-cinnamate (1.35 g; 5.35 mmol) in toluene (25 mL) was cooled to −10° C. and treated with a solution of diisobutylaluminum hydride in toluene (11.25 mL of a 1.0 M solution; 11.25 mmol). The reaction mixture was stirred for 3 hours at 0° C. and then quenched with 3 mL of methanol followed by 1 N HCl until the pH was 1. The reaction mixture was extracted into ethyl acetate and the organic phase was washed with water, dried and concentrated. Purification on a silica gel column eluting with 25% ethyl acetate in hexane furnished 0.96 g (80%) of a thick oil. ¹H NMR (360 MHz; CDCl₃): δ3.85 (s, 3H); 3.87 (s, 6H); 4.32 (d, 2H, J=5.6); 6.29 (dt, 1H, J=15.8, 5.7), 6.54 (d, 1H, J=15.8); 6.61 (s, 2H).

### Example 19

### Synthesis of (2S)-1-(3,3-dimethyl-1,2-dioxopentyl)-2-pyrrolidinecarboxylate (421)

Synthesis of (2S)-1-(1,2-dioxo-2-methoxyethyl)-2-pyrrolidinecarboxylate.

A solution of L-proline methyl ester hydrochloride (3.08 g; 18.60 mmol) in dry methylene chloride was cooled to 0° C. and treated with triethylamine (3.92 g; 38.74 mmol; 2.1 eq). After stirring the formed slurry under a nitrogen atmosphere for 15 min, a solution of methyl oxalyl chloride (3.20 g; 26.12 mmol) in methylene chloride (45 mL) was added dropwise. The resulting mixture was stirred at 0° C. for 1.5 hr. After filtering to remove solids, the organic phase was washed with water, dried over MgSO₄ and concentrated. The crude residue was purified on a silica gel column, eluting with 50% ethyl acetate in hexane, to obtain 3.52 g (88%) of the product as a reddish oil. Mixture of cis-trans amide rotamers; data for trans rotamer given. ¹H NMR (CDCl₃): δ 1.93 (dm, 2H); 2.17 (m, 2H); 3.62 (m, 2H); 3.71 (s, 3H); 3.79, 3.84 (s, 3H total); 4.86 (dd, 1H, J=8.4, 3.3).

Synthesis of methyl (2S)-1-(1,2-dioxo-3,3-dimethylpentyl)-2-pyrrolidinecarboxylate.

A solution of methyl (2S)-1-(1,2-dioxo-2-methoxyethyl)-2-pyrrolidinecarboxylate (2.35 g; 10.90 mmol) in 30 mL of tetrahydrofuran (THF) was cooled to −78° C. and treated with 14.2 mL of a 1.0 M solution of 1,1-dimethylpropylmagnesium chloride in THF. After stirring the resulting homogeneous mixture at −78° C. for three hours, the mixture was poured into saturated ammonium chloride (100 mL) and extracted into ethyl acetate. The organic phase was washed with water, dried, and concentrated, and the crude material obtained upon removal of the solvent was purified on a silica gel column, eluting with 25% ethyl acetate in hexane, to obtain 2.10 g (75%) of the oxamate as a colorless oil. ¹H NMR (CDCl₃): δ 0.88 (t, 3H); 1.22, 1.26 (s, 3H each); 1.75 (dm, 2H); 1.87-2.10 (m, 3H); 2.23 (m, 1H); 3.54 (m, 2H); 3.76 (s, 3H); 4.52 (dm, 1H, J=8.4, 3.4).

Synthesis of (2S)-1-(1,2-dioxo-3,3-dimethylpentyl)-2-pyrrolidinecarboxylic Acid

A mixture of methyl (2S)-1-(1,2-dioxo-3,3-dimethylpentyl)-2-pyrrolidinecarboxylate (2.10 g; 8.23 mmol), 1 N LiOH (15 mL), and methanol (50 mL) was stirred at 0° C. for 30 min and at room temperature overnight. The mixture was acidified to pH 1 with 1 N HCl, diluted with water, and extracted into 100 mL of methylene chloride. The organic extract was washed with brine and concentrated to deliver 1.73 g (87%) of snow-white solid which did not require further purification. ¹H NMR (CDCl₃): δ 0.87 (t, 3H); 1.22, 1.25 (s, 3H each); 1.77 (dm, 2H); 2.02 (m, 2H); 2.17 (m, 1H); 2.25 (m, 1H); 3.53 (dd, 2H, J=10.4, 7.3); 4.55 (dd, 1H, J=8.6, 4.1).

### Example 20

### Synthesis of (2S)-1-(1,2-dioxo-3,3-dimethylpentyl)-2-pyrrolidinecarboxamide (318)

Isobutyl chloroformate (20 mmol, 2.7 mL) was added to a solution containing (2S)-1-(1,2-dioxo-3,3-dimethylpentyl)-2-pyrrolidinecarboxylic acid (4.89 g, 20 mmol)(from Example 19) in 50 mL methylene chloride at −10° C. with stirring. After 5 minutes, ammonia was added dropwise (20 mmol, 10 mL of 2 M ethyl alcohol solution). The reaction was warmed up to room temperature after stirring at −10° C. for 30 minutes. The mixture was diluted with water, and extracted into 200 mL methylene chloride. The organic extract was concentrated and further purified by silica gel to give 4.0 g of product as a white solid (81.8% yield). ¹H NMR (CDCl₃): δ 0.91 (t, 3H, J=7.5); 1.28 (s, 6H, each); 1.63-1.84 (m, 2H); 1.95-2.22 (m, 3H); 2.46 (m, 1H); 3.55-3.67 (m, 2H); 4.67 (t, 1H, J=7.8); 5.51-5.53 (br, 1H, NH); 6.80 (br, 1H, NH).

### Example 21

### Synthesis of (2S)-1-(1,2-dioxo-3,3-dimethylpentyl)-2-pyrrolidinecarbonitrile (313)

To a solution of 0.465 mL DMF (6 mmol) in 10 mL acetonitrile at 0° C. was added 0.48 mL (5.5 mmol) of oxalyl chloride. A white precipitate formed immediately and was accompanied by gas evolution. When complete, a solution of 1.2 g (5 mmol) of (2S)-1-(1,2-dioxo-3,3-dimethylpentyl)-2-pyrrolidinecarboxamide (from Example 20) in 2.5 mL acetonitrile was added. When the mixture became homogeneous, 0.9 mL (11 mmol) pyridine was added. After 5 min., the mixture was diluted into water and extracted by 200 mL ethyl acetate. The organic layer was concentrated and further purified by silica gel to give 0.8 g product as a white solid (72% yield). ¹H NMR (CDCl₃): δ 0.87 (t, 3H, J=7.5); 1.22 (s, 3H); 1.24 (s, 3H); 1.80 (m, 2H); 2.03-2.23 (m, 4H); 3.55 (m, 2H); 4.73 (m, 1H)

### Example 22

### Synthesis of (2S)-1-(1,2-dioxo-3,3-dimethylpentyl)-2-pyrrolidinetetrazole (314)

A mixture of (2S)-1-(1,2-dioxo-3,3-dimethylpentyl)-2-pyrrolidinecarbonitrile (222 mg, 1 mmol)(from Example 21), NaN₃ (81 mg, 1.3 mmol) and NH₄Cl (70 mg, 1.3 mmol) in 3 mL DME was stirred at 130° C. for 16 hours. The mixture was concentrated and purified by silica gel to afford 200 mg product as white solid (75.5% yield). ¹H NMR (CDCl₃): δ 0.88 (t, 3H, J=7.5); 1.22 (s, 6H); 1.68 (m, 2H); 2.05-2.36 (m, 3H); 2.85 (m, 1H); 3.54 (m, 1H); 3.75 (m, 1H); 5.40 (m, 1H).

### Example 23

### Synthesis of 3-(3,3-dimethyl-2-oxopentanoyl)-1,3-oxazolidine-4-carboxylic Acid (612)

Methyl 1,3-oxazolidine-4-carboxylate

This compound was synthesized according to the procedure found in J. Med. Chem. (1990) 33:1459-1469.

Methyl 2-[4-(methoxycarbonyl)(1,3-oxazolidin-3-yl)]-2-oxoacetate

To an ice cooled solution of methyl 1,3-oxazolidine-4-carboxylate (0.65 g, 4.98 mM) were added triethylamine (0.76 ml, 5.45 mM) and methyl oxalyl chloride (0.5 ml, 5.45 mM). This mixture was stirred at 0° C. for 2 hours. After this time the mixture was washed with water, then brine, dried with anhydrous magnesium sulfate, filtered and evaporated. The resulting pale yellow oil was flash chromatographed eluting with 30% EtOAc/hexane, 50% EtOAc/hexane, and finally 75% EtOAc/hexane. A clear oil of product (0.52 g, 48%) was obtained. Anal. (C₈H₁₁NO₆)C,H,N; ¹H NMR (CDCl₃, 400 MHz): δ (2 rotamers 1:1) 3.78 (s, 1.5H); 3.79 (s, 1.5H); 3.87 (s, 1.5H); 3.91 (s, 1.5H); 4.14-4.36 (m, 2H); 4.70 (dd, 0.5H, J=4.1, 6.8); 5.08 (dd,0.5H, J=3.1,6.7); 5.10 (d, 0.5H, J=5.9); 5.27 (d, 0.5H, J=5.8); 5.36 (dd, 1H, J=5.3, 17.8).

Methyl 3-(3,3-dimethyl-2-oxopentanoyl)-1,3-oxazolidine-4-carboxylate

To a solution of methyl 2-[4-(methoxycarbonyl)-(1,3-oxazolidin-3-yl)]-2-oxoacetate (0.84 g, 3.87 mM) in THF (50 ml) cooled to −78° C. was added 1,1-dimethylpropyl-magnesium chloride (1M in THF, 8 ml, 8 mM). After 3 hrs. at −78° C. the mixture was quenched with saturated NH₄Cl (50 ml) and extracted with ethyl acetate (100 ml). The organic layer separated, washed with brine (100 ml), dried with anhydrous magnesium sulfate, filtered and evaporated. The resulting pale yellow oil was flash chromatographed eluting with 20% EtOAc/hexane. A clear oil (3) (0.61 g, 61%) was obtained. ¹H NMR (CDCl₃, 400 MHz): δ 0.85 (t, 3H, J=7.5); 1.25 (s, 3H); 1.26 (s, 3H); 1.67-1.94 (m, 2H); 3.79 (s, 3H); 4.12-4.31 (m, 2H); 4.64 (dd, 1H, J=4.1, 6.8); 5.04 (dd, 2H, J=4.9, 9.4).

3-(3,3-dimethyl-2-oxopentanoyl)-1,3-oxazolidine-4-carboxylic Acid (612)

Methyl 3-(3,3-dimethyl-2-oxopentanoyl)-1,3-oxazolidine-4-carboxylate (3) (0.6 g, 2.33 mM) was dissolved in MeOH (25 ml) and added LiOH (1M in water, 10 ml, 10 mM). This mixture was stirred overnight at room temperature. The residues were evaporated and partitioned between EtOAc (50 ml) and 2N HCl (50 mL). The aqueous layer was extracted twice more with EtOAc (2×25 ml). The extracts were washed with brine (50 ml), dried with anhydrous magnesium sulfate, filtered and evaporated. A clear oil product (0.49 g, 86%) was obtained. Anal. (C₁₁H₁₇NO₅) C, H, N; ¹H NMR (CDCl₃, 400 MHz): δ 0.84 (t, 3H, J=7.5); 1.25 (s, 6H); 1.70-1.95 (m, 2H); 4.22-4.29 (m, 2H); 4.66 (dd, 1H, J=4.6, 6.5); 5.04 (dd, 2H, J=5.0, 8.9); 7.67 (bs, 1H).

### Example 24

### Synthesis of (2S)-1-(N-cyclohexylcarbamoyl) pyrrolidine-2-carboxylic Acid (619)

Methyl (2S)-1-(N-cyclohexylcarbamoyl)pyrrolidine-2-carboxylate.

A mixture of cyclohexyl isocyanate (3.88 g; 31 mmol), L-proline ester hydrochloride (5.0 g; 30.19 mmol), and triethylamine (9 mL) in methylene chloride (150 ml) was stirred overnight at room temperature. The reaction mixture was washed with 2×100 ml of 1 N HCL and 1×100 ml of water. The organic phase was dried, concentrated and purified on a silica gel column (50% EtOAc/hexane) to yield the urea as a thick oil, ¹H NMR (CDCL₃, 400 MHz): δ 1.09-1.15 (m, 3H); 1.33 (m, 2H); 1.68 (m, 3H); 1.93-2.05 (m, 6H); 3.33 (m, 1H); 3.43 (m, 1H); 3.46 (m, 1H); 3.73 (s, 3H); 4.39 (m, 1H); 4.41 (m, 1H).

(2S)-1-(N-cyclohexylcarbamoyl)pyrrolidine-2-carboxylic Acid (619)

Methyl (2S)-1-(N-cyclohexylcarbamoyl)pyrrolidine-2-carboxylate (3.50 g) was dissolved in methanol (60 ml), cooled to 0° C., and treated with 2N LiOH (20 ml). After stirring overnight, the mixture was partitioned between ether and water. The ether layer was discarded and the aqueous layer was made acidic (pH 1) with 1N HCl and extracted with methylene chloride. Drying and removal of the solvent provided 2.20 g of the product as a white solid, ¹H NMR (CDCl₃, 400 MHz): δ 1.14-1.18 (m, 3H); 1.36-1.38 (m, 2H); 1.71-1.75 (m, 3H); 1.95-2.04 (m, 5H); 2.62 (m, 1H); 3.16 (m, 1H); 3.30-3.33 (m, 1H); 3.67 (m, 1H); 4.38 (br, 1H); 4.46 (m, 1H).

### Example 25

### Synthesis of (2S)-N-(benzylsulfonyl)-2-pyrrolidinecarboxylic Acid (719)

To a cooled (0° C.) solution of proline methyl ester hydrochloride salt (5.0 g; 30.19 mmol) in 200 mL of methylene chloride was added triethylamine (35 mL) and benzenesulfonyl chloride (5.75 g; 30.19 mmol). The mixture was stirred for one hour at 0° C. and then washed with 2×100 mL of water. The organic phase was dried and concentrated. Chromatography eluting with 50% EtOAc/hexane delivered 8.14 g (5%) of the N-sulfonamide methyl ester, which was dissolved in 120 mL of methanol, cooled to 0° C., and treated with 40 mL of 1 N lithium hydroxide. The mixture was stirred for 1 hour at 0° C. and then overnight at room temperature. After making the reaction mixture acidic (pH 1) with 1 N HCl, the product was extracted into methylene chloride and dried and concentrated to yield 4.25 g of (2S)-N-(benzylsulfonyl)-2-pyrrolidinecarboxylic acid (A) as a white solid, ¹H NMR (CDCl₃, 400 MHz): δ 1.85-1.90 (m, 2H); 2.08 (m, 1H); 2.18 (m, 1H); 3.04 (m, 1H); 3.27 (m, 1H); 4.32-4.35 (m, 2H); 4.45 (m, 1H); 4.45 (m, 2H); 7.36 (m, 3H); 7.48 (m, 2H); 10.98 (br, 1H).

### Example 26

### Synthesis of (2S)-1-(phenylmethylsulfonyl)-2-hydroxymethylpyrrolidine (813)

To a solution of (S)-(+)-2-pyrrolidinemethanol (1.01 g, 10 mmol) and triethylamine (1.5 ml, 11 mmol) in 30 ml methylene chloride was added 1.9 g (10 mmol) α-toluenesulfonyl chloride at 0° C. with stirring. The reaction was gradually warmed up to room temperature and stirred overnight. The mixture was diluted with water, and extracted into 200 ml methylene chloride. The organic extract was concentrated and further purified by silica gel to give 1.5 g product as a white solid (58.9% yield). ¹H NMR (CDCl₃): δ 01.71-1.88 (m, 4H); 2.05 (br, 1H, OH); 3.22 (m, 2H); 3.47 (m, 2H); 3.67 (m, 1H); 4.35 (s, 2H); 7.26-7.44 (m, 5H, aromatic).

### Example 27

### Synthesis of (2S)-1-(phenylmethyl)sulfonyl-2-pyrrolidinecarboxamide (814)

To a solution of L-prolinamide (2.28 g, 20 mmol) and triethylamine (5.76 ml, 42 mmol) in 40 ml methylene chloride was added 3.92 g (20 mmol) α-toluenesulfonyl chloride at 0° C. with stirring. The reaction was gradually warmed up to room temperature and stirred overnight. The mixture was diluted with water, and extracted into 200 ml methylene chloride. The organic extract was concentrated and further purified by silica gel to give 3.0 g product as a white solid (55.7% yield).

¹H NMR (CDC₃): δ 01.89 (m, 3H); 2.25 (m, 1H); 3.40 (m, 1H); 3.50 (m, 1H); 3.96 (m, 1H); 4.35 (s, 2H); 7.39-7.45 (m, 5H, aromatic).

### Example 28

### Synthesis of (2S)-1-(phenylmethyl)sulfonyl-2-pyrrolidinecarbonitrile (815)

To a solution of 0.67 ml DMF (8.7 mmol)in 10 ml acetonitrile at 0° C. was added 0.70 ml (8.0 mmol) oxalyl chloride. A white precipitate was formed immediately and was accompanied by gas evolution. When complete, a solution of 2.0 g (7.5 mmol) of (2S)-1-(phenylmethyl)sulfonyl-2-pyrrolidine-carboxamide in 5.0 ml acetonitrile was added. When the mixture became homogeneous, 1.35 ml (16.5 mmol) pyridine was added. After 5 min., the mixture was diluted with water, and extracted by 200 ml ethyl acetate. The organic layer was concentrated and further purified by silica gel to give 1.5 g product as a white solid (80% yield). ¹H NMR (CDCl₃): δ 1.92 (m, 2H); 2.01 (m, 1H); 2.11 (m, 1H); 3.45 (m, 2H); 4.35 (s, 2H); 4.65 (m, 1H); 7.26-7.45 (m, 5H, aromatic).

### Example 29

### Synthesis of (2S)-1-(phenylmethyl)sulfonyl-2-pyrrolidinetetrazole (722).

A mixture of (2S)-1-(phenylmethyl)sulfonyl-2-pyrrolidinecarbonitrile (250 mg, 1 mmol), NaN₃ (81 Mg, 1.3 mmol) and NH₄Cl (70 Mg, 1.3 mmol) in 3 ml DMF was stirred at 130° C. for 16 hours. The mixture was concentrated and purified by silica gel to give 120 mg product as a white solid (41.1% yield). ¹H NMR (CDCl₃) δ 01.95 (m, 2H); 2.21 (m, 1H); 2.90 (m, 1H); 3.40 (m, 2H); 4.27 (s, 2H); 5.04 (m, 1H); 7.36-7.41 (m, 5H, aromatic); 8.05 (s, 1H, NH).

The following neurotrophic compounds (referenced by Compound No.) were used in the following non-limiting examples to demonstrate the efficacy of the compounds of the invention in the treatment of nerve injury caused as a consequence of prostate surgery:

| Compound No. | Structure |
|---|---|
| I | 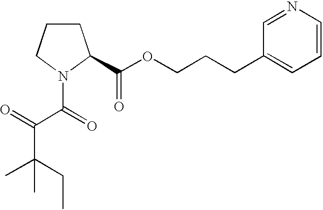 |
| II | 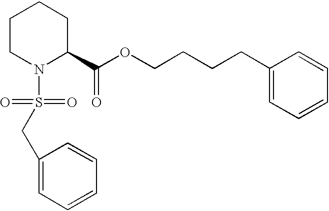 |
| III | 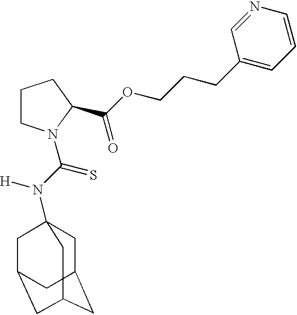 |
| IV | 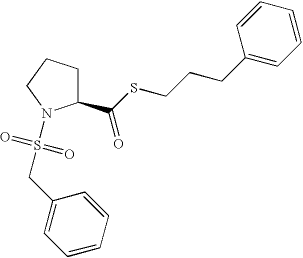 |
| V | 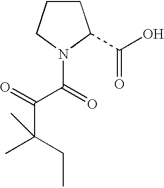 |
| VI | 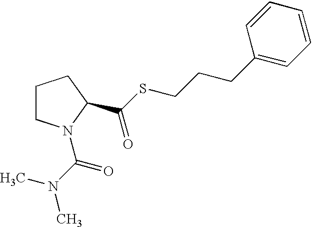 |
| VII | 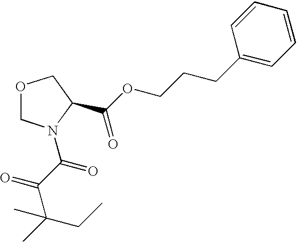 |
| VIII | 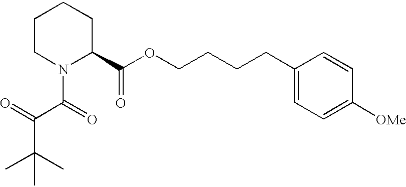 |
| IX | 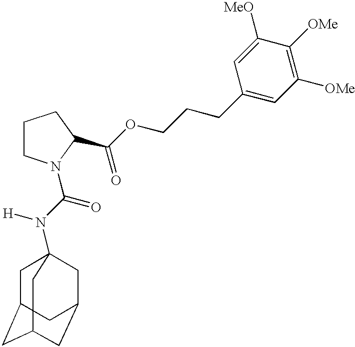 |
| X | 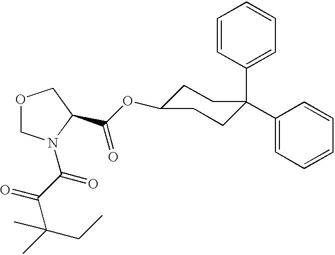 |
| XI | 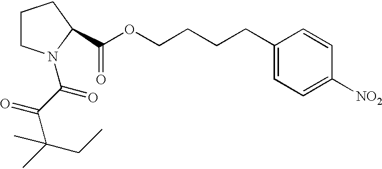 |
| XII | 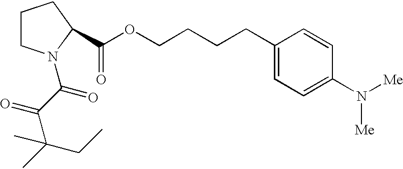 |
| XIII | 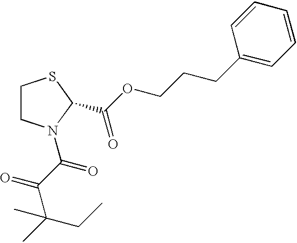 |
| XIV | 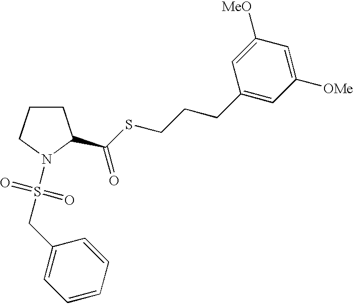 |
| XV | 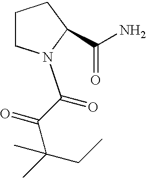 |
| XVI | 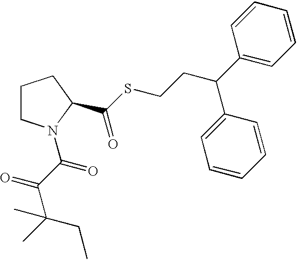 |
| XVII | 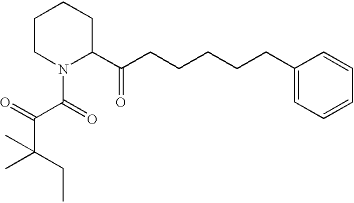 |
| XVIII | 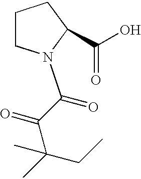 |
| XIX | 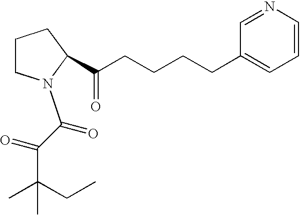 |
| XX | 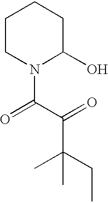 |
| XXI | 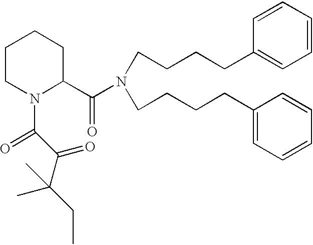 |
| XXII | 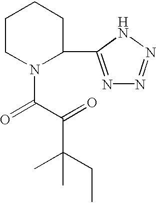 |
| XXIII | 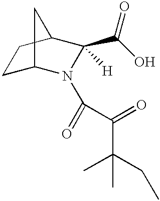 |
| XXIV | 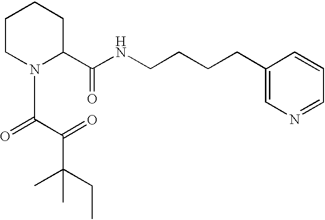 |
| XXV | 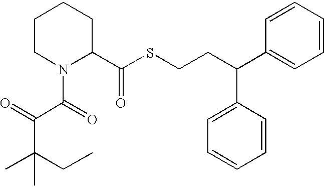 |

Example 30 addresses the effect of Compound 153 administration on crushed cavernous nerves. This example clearly demonstrates that the neurotrophic compound regenerate the penile cavernous nerve and are useful in the treatment of nerve injury caused as a consequence of prostate surgery.

### Example 30

Cavernous nerve injury was performed in 12 week old Sprague-Dawley rats by crushing the right cavernous nerve for 3×15 seconds with a fine tip forceps. The rats were treated with saline or Compound 153 (15 mg/kg i.p.) just prior to nerve crush. The right and left major pelvic ganglia were processed for nNOS immunoreactivity. Intracavernosal pressure (ICP) responses to electrostimulation of the right (injured) and left (intact) cavernous nerves were recorded for each animal at 24 hours or 7 days post injury.

TABLE XLV
Maximal Effects Of Compound 153 and FK506 (i.p.) on
ICP Response 1 Day Following R-Cavernous Nerve Crush
Injury (+/− sem)

| | | | Significance |
|---|---|---|---|
| Treatment | Control | Crush | (p value)* |
| Vehicle (1 ml/kg) | 49.4 +/− 6.0 | 23.6 +/− 5.9 | .01 |
| FK506 (1 mg/kg) | 36.9 +/− 7.7 | 32.0 +/− 6.7 | .6 |
| Compound 153 (15 mg/kg) | 42.8 +/− 1.9 | 42.7 +/− 2.2 | 1.0 |

(n = 5-6 animals/group)
*Comparison of the cavernous pressure on the control side versus the crush side for each treatment
(The animals treated with FK506 or Compound 153 are well protected and the intracavernous pressure is maintained with drug treatment)

The invention being thus described, it will be obvious that the same may be varied in many ways. Such variations are not to be regarded as a departure from the spirit and scope of the invention and all such modifications are intended to be included within the scope of the following claims.

We claim:

1. A method for the treatment, prophylactic treatment or prevention of nerve injury caused as a consequence of prostate surgery which comprises administering to a mammal in need of such treatment a compound of formula I 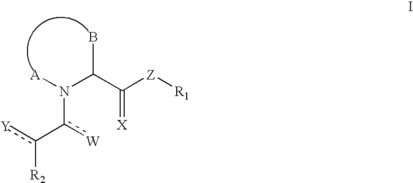
or a pharmaceutically acceptable salt, ester or solvate thereof, wherein:
A and B, together with the nitrogen and carbon atoms to which they are respectively attached, form a 5-7 membered saturated or unsaturated heterocyclic ring optionally containing in addition to the nitrogen atom one or more heteroatom(s) independently selected from the group consisting of O, S, SO, SO₂, N, NH and NR₂;
X is O or S;
Z is S, CH₂, CHR₃ or CR₁R₃;
W and Y are independently O, S, CH₂ or H₂;
R₁ and R₃ are independently C₁-C₆ straight or branched chain alkyl or C₂-C₆ straight or branched chain alkenyl, wherein said alkyl or alkenyl is substituted with one or more substituent(s) independently selected from the group consisting of (Ar₁)ₙ, C₁-C₆ straight or branched chain alkyl or C₂-C₆ straight or branched chain alkenyl substituted with (Ar₁)ₙ, C₃-C₈ cycloalkyl, C₁-C₆ straight or branched chain alkyl or C₂-C₆ straight or branched chain alkenyl substituted with C₃-C₈ cycloalkyl, and Ar₂;
n is 1 or 2;
R₂ is C₁-C₉ straight or branched chain alkyl, C₂-C₉ straight or branched chain alkenyl, C₃-C₈ cycloalkyl, C₅-C₇ cycloalkenyl or Ar₁, wherein said alkyl, alkenyl, cycloalkyl or cycloalkenyl is unsubstituted or substituted with one or more substituent(s) independently selected from the group consisting of C₁-C₄ straight or branched chain alkyl, C₂-C₄ straight or branched chain alkenyl and hydroxy; and
Ar₁ and Ar₂ are independently an alicyclic or aromatic, mono-, bi- or tricyclic, carbo- or heterocyclic ring, wherein said ring is unsubstituted or substituted with one or more substituent(s) independently selected from the group consisting of halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy and amino; wherein the individual ring size is 5-8 members; and wherein the heterocyclic ring contains 1-6 heteroatom(s) independently selected from the group consisting of O, N and S.

2. The method of claim 1, wherein the nerve injury is injury to a penile cavernous nerve of the mammal.

3. The method of claim 1, wherein the nerve injury results in erectile dysfunction of the mammal.

4. A method for the treatment, prophylactic treatment or prevention of nerve injury caused as a consequence of prostate surgery which comprises administering to a mammal in need of such treatment a compound of formula II 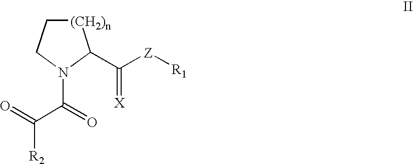
or a pharmaceutically acceptable salt, ester or solvate thereof, wherein:
n is 1 or 2;
X is O or S;
Z is S, CH₂, CHR₃ or CR₁R₃;
R₁ and R₃ are independently C₁-C₅ straight or branched chain alkyl, C₂-C₅ straight or branched chain alkenyl, or Ar₁, wherein said alkyl, alkenyl or Ar₁ is unsubstituted or substituted with one or more substituent(s) independently selected from the group consisting of halo, nitro, C₁-C₆ straight or branched chain alkyl, C₂-C₆ straight or branched chain alkenyl, hydroxy, C₁-C₄ alkoxy, C₂-C₄ alkenyloxy, phenoxy, benzyloxy, amino and Ar₁;
R₂ is C₁-C₉ straight or branched chain alkyl, C₂-C₉ straight or branched chain alkenyl, C₃-C₈ cycloalkyl, C₅-C₇ cycloalkenyl or Ar₁; and
Ar₁ is phenyl, benzyl, pyridyl, fluorenyl, thioindolyl or naphthyl, wherein said Ar₁ is unsubstituted or substituted with one or more substituent(s) independently selected from the group consisting of halo, trifluoromethyl, hydroxy, nitro, C₁-C₆ straight or branched chain alkyl, C₂-C₆ straight or branched chain alkenyl, C₁-C₄ alkoxy, C₂-C₄ alkenyloxy, phenoxy, benzyloxy and amino.

5. The method of claim 4, wherein the nerve injury is injury to a penile cavernous nerve of the mammal.

6. The method of claim 4, wherein the nerve injury results in erectile dysfunction of the mammal.

7. A method for the treatment, prophylactic treatment or prevention of nerve injury caused as a consequence of prostate surgery which comprises administering to a mammal in need of such treatment a therapeutically effective non-immunosuppressive amount of a neurotrophic compound having an affinity for an FKBP-type immunophilin, wherein the immunophilin exhibits rotamase activity and the neurotrophic compound inhibits the rotamase activity of the immunophilin.

8. The method of claim 7, wherein the nerve injury is injury to a penile cavernous nerve of the mammal.

9. The method of claim 7, wherein the nerve injury results in erectile dysfunction of the mammal.

10. A method for the treatment, prophylactic treatment or prevention of nerve injury caused as a consequence of prostate surgery which comprises administering to a mammal in need of such treatment a compound of formula XXVI 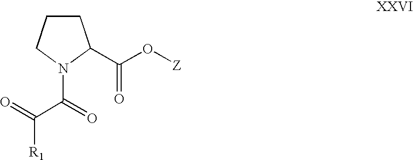
or a pharmaceutically acceptable salt, ester or solvate thereof, wherein:
R₁ is C₁-C₉ straight or branched chain alkyl, C₂-C₉ straight or branched chain alkenyl, C₃-C₈ cycloalkyl, C₅-C₇ cycloalkenyl or Ar₁, wherein said R₁ is unsubstituted or substituted with one or more substituent(s) independently selected from the group consisting of C₁-C₆ alkyl, C₂-C₆ alkenyl, C₃-C₈ cycloalkyl, C₃-C₇ cycloalkenyl, hydroxy and Ar₂;
Ar₁ and Ar₂ are independently 1-napthyl, 2-napthyl, 2-indolyl, 3-indolyl, 2-furyl, 3-furyl, 2-thienyl, 3-thienyl, 2-pyridyl, 3-pyridyl, 4-pyridyl or phenyl, wherein said Ar₁ is unsubstituted or substituted with one or more substituent(s) independently selected from the group consisting of halo, hydroxy, nitro, trifluoromethyl, C₁-C₆ straight or branched chain alkyl, C₂-C₆ straight or branched chain alkenyl, C₁-C₄ alkoxy, C₂-C₄ alkenyloxy, phenoxy, benzyloxy and amino;
Z is C₁-C₆ straight or branched chain alkyl or C₂-C₆ straight or branched chain alkenyl, wherein said Z is substituted with one or more substituent(s) independently selected from the group consisting of Ar₁, C₃-C₈ cycloalkyl, and C₁-C₆ straight or branched chain alkyl or C₂-C₆ straight or branched chain alkenyl substituted with C₃-C₈ cycloalkyl; or Z is a fragment 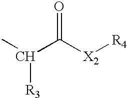
wherein:
R₃ is C₁-C₉ straight or branched chain alkyl which is unsubstituted or substituted with C₃-C₈ cycloalkyl or Ar₁;
X₂ is O or NR₅;
R₅ is hydrogen, C₁-C₆ straight or branched chain alkyl or C₂-C₆ straight or branched chain alkenyl; and
R₄ is phenyl, benzyl, C₁-C₅ straight or branched chain alkyl, C₂-C₅ straight or branched chain alkenyl, C₁-C₅ straight or branched chain alkyl substituted with phenyl, or C₂-C₅ straight or branched chain alkenyl substituted with phenyl.

11. The method of claim 10, wherein the nerve injury is injury to a penile cavernous nerve of the mammal.

12. The method of claim 10, wherein the nerve injury results in erectile dysfunction of the mammal.

13. The method of claim 10, wherein R₁ is C₁-C₉ straight or branched chain alkyl, 2-cyclohexyl, 4-cyclohexyl, 2-furanyl, 2-thienyl, 2-thiazolyl or 4-hydroxybutyl.

14. The method of claim 10, wherein Z and R₁ are lipophilic.

15. A method for the treatment, prophylactic treatment or prevention of nerve injury caused as a consequence of prostate surgery which comprises administering to a mammal in need of such treatment a compound of formula XXVIII 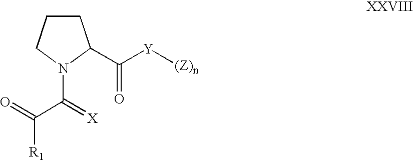
or a pharmaceutically acceptable salt, ester or solvate thereof, wherein:
R₁ is C₁-C₆ straight or branched chain alkyl, C₂-C₆ straight or branched chain alkenyl, C₃-C₆ cycloalkyl or Ar₁, wherein said alkyl or alkenyl is unsubstituted or substituted with C₃-C₆ cycloalkyl or Ar₂;
Ar₁ and Ar₂ are independently 2-furyl, 2-thienyl or phenyl;
X is oxygen or sulfur;
Y is oxygen or NR₂, wherein R₂ is a direct bond, hydrogen or C₁-C₆ alkyl;
Z is hydrogen, C₁-C₆ straight or branched chain alkyl, or C₂-C₆ straight or branched chain alkenyl, wherein said Z is substituted with one or more substituent(s) independently selected from the group consisting of 2-furyl, 2-thienyl, C₃-C₆ cycloalkyl, pyridyl and phenyl, each having one or more substituent(s) independently selected from the group consisting of hydrogen and C₁-C₄ alkoxy; and
n is 1 or 2.

16. The method of claim 15, wherein the nerve injury is injury to a penile cavernous nerve of the mammal.

17. The method of claim 15, wherein the nerve injury results in erectile dysfunction of the mammal.

18. The method of claim 15, wherein the neurotrophic compound is selected from the group consisting of:
3-(2,5-dimethoxyphenyl)-1-propyl (2S)-1-(3,3-dimethyl-1,2-dioxopentyl)-2-pyrrolidinecarboxylate;
3-(2,5-dimethoxyphenyl)-1-prop-2-(E)-enyl (2S)-1-(3,3-dimethyl-1,2-dioxopentyl)-2-pyrrolidinecarboxylate;
2-(3,4,5-trimethoxyphenyl)-1-ethyl (2S)-1-(3,3-dimethyl-1,2-dioxopentyl)-2-pyrrolidinecarboxylate;
3-(3-pyridyl)-1-propyl (2S)-1-(3,3-dimethyl-1,2-dioxopentyl)-2-pyrrolidinecarboxylate;
3-(2-pyridyl)-1-propyl (2S)-1-(3,3-dimethyl-1,2-dioxopentyl)-2-pyrrolidinecarboxylate;
3-(4-pyridyl)-1-propyl (2S)-1-(3,3-dimethyl-1,2-dioxopentyl)-2-pyrrolidinecarboxylate;
3-phenyl-1-propyl (2S)-1-(2-tert-butyl-1,2-dioxoethyl)-2-pyrrolidinecarboxylate;
3-phenyl-1-propyl (2S)-1-(2-cyclohexylethyl-1,2-dioxo-ethyl)-2-pyrrolidinecarboxylate;
3-(3-pyridyl)-1-propyl (2S)-1-(2-cyclohexylethyl-1,2-dioxoethyl)-2-pyrrolidinecarboxylate;
3-(3-pyridyl)-1-propyl (2S)-1-(2-tert-butyl-1,2-dioxo-ethyl)-2-pyrrolidinecarboxylate;
3,3-diphenyl-1-propyl (2S)-1-(3,3-dimethyl-1,2-dioxo-pentyl)-2-pyrrolidinecarboxylate;
3-(3-pyridyl)-1-propyl (2S)-1-(2-cyclohexyl-1,2-dioxoethyl)-2-pyrrolidinecarboxylate;
3-(3-pyridyl)-1-propyl (2S)-N-([2-thienyl]glyoxyl)-pyrrolidinecarboxylate;
3,3-diphenyl-1-propyl (2S)-1-(3,3-dimethyl-1,2-dioxobutyl)-2-pyrrolidinecarboxylate;
3,3-diphenyl-1-propyl (2S)-1-cyclohexylglyoxyl-2-pyrrolidinecarboxylate;
3,3-diphenyl-1-propyl (2S)-1-(2-thienyl)glyoxyl-2-pyrrolidinecarboxylate; and
pharmaceutically acceptable salts, esters and solvates thereof.

19. A method for the treatment, prophylactic treatment or prevention of nerve injury caused as a consequence of prostate surgery which comprises administering to a mammal in need of such treatment a compound of formula LXIV 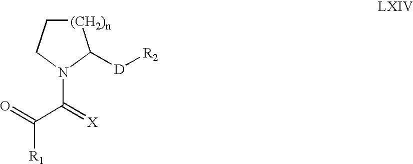
or a pharmaceutically acceptable salt, ester or solvate thereof, wherein:
n is 1-3;
X is O or S;
R₁ is C₁-C₉ straight or branched chain alkyl, C₂-C₉ straight or branched chain alkenyl, aryl, heteroaryl, carbocycle or heterocycle;
D is a bond, C₁-C₁₀ straight or branched chain alkyl, C₂-C₁₀straight or branched chain alkenyl or C₂-C₁₀ straight or branched chain alkynyl; and
R₂ is a carboxylic acid or a carboxylic acid isostere.

20. The method of claim 19, wherein the nerve injury is injury to a penile cavernous nerve of the mammal.

21. The method of claim 19, wherein the nerve injury results in erectile dysfunction of the mammal.

22. The method of claim 19, wherein R₂ is 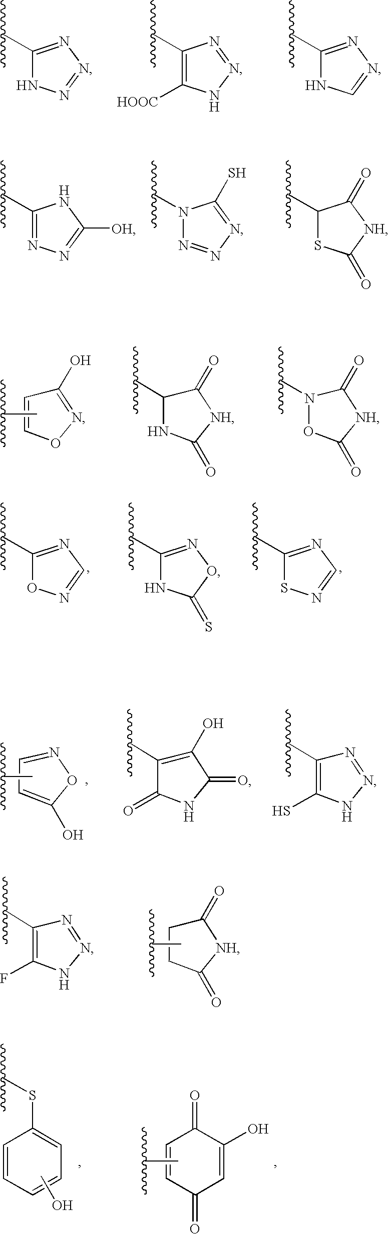
—COOH, —SO₃H, —SO₂HNR, —PO₂(R³)₂, —CN, —PO₃(R³)₂, —OR³ , —SR³, —NHCOR³, —N(R³)₂, —CON(R³)₂, CONH(O)R³, —CONHNHSO₂R³, —COHNSO₂R³ or —CONR³CN;
R³ is hydrogen, hydroxy, halo, halo-C₁-C₆ alkyl, thiocarbonyl, C₁-C₆ alkoxy, C₂-C₆ alkenoxy, C₁-C₆ alkylaryloxy, aryloxy, aryl-C₁-C₆ alkyloxy, cyano, nitro, imino, C₁-C₆ alkylamino, amino-C₁-C₆ alkyl, sulfhydryl, thio-C₁-C₆-alkyl, C₁-C₆-alkylthio, sulfonyl, C₁-C₆ straight or branched chain alkyl, C₂-C₆ straight or branched chain alkenyl or alkynyl, aryl, heteroaryl, carbocycle, heterocycle or CO₂R⁴; and
R⁴ is hydrogen, C₁-C₉ straight or branched chain alkyl or C₂-C₉ straight or branched chain alkenyl.

23. A method for the treatment, prophylactic treatment or prevention of nerve injury caused as a consequence of prostate surgery which comprises administering to a mammal in need of such treatment a compound of formula LXVIII 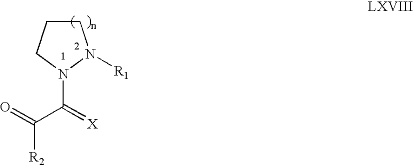
or a pharmaceutically acceptable salt, ester or solvate thereof, wherein:
n is 1-3;
R₁ is —CR₃, —COOR₃, —COR₃, —COOH, —SO₃H, —SO₂HNR₃, —PO₂(R³)₂, —CN, —PO₃(R₃)₂, —OR₃, —SR₃, —NHCOR₃, —N(R₃)₂, —CON(R₃)₂, —CONH(O)R₃, —CONHNHSO₂R₃, —COHNSO₂R₃, —CONR₃CN, 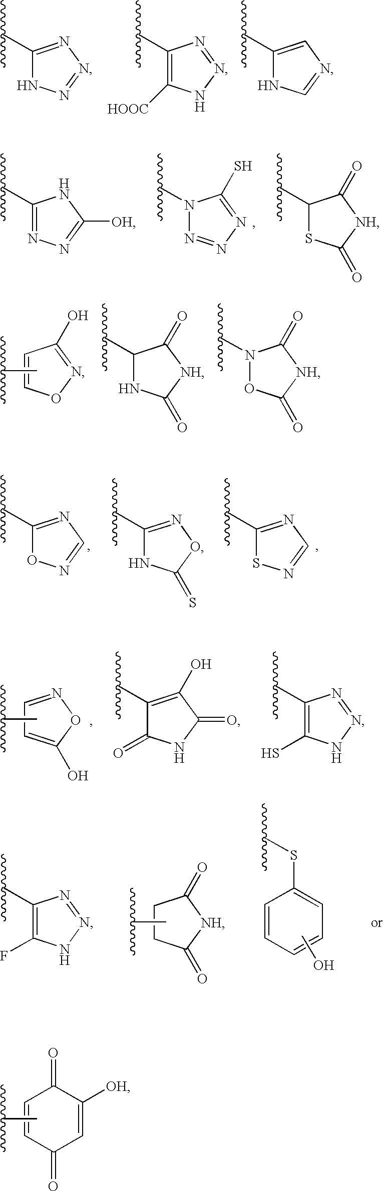
wherein said R₁ is unsubstituted or substituted with R₃;
R₂ is hydrogen, C₁-C₉ straight or branched chain alkyl, C₂-C₉ straight or branched chain alkenyl, C₂-C₉ straight or branched chain alkynyl, aryl, heteroaryl, carbocycle or heterocycle, wherein said alkyl, alkenyl, alkynyl, aryl, heteroaryl, carbocycle or heterocycle is unsubstituted or substituted with one or more substituent(s) selected from R₃;
R₃ is hydrogen, C₁,C₉ straight or branched chain alkyl, C₂-C₉ straight or branched chain alkenyl, C₂-C₉ straight or branched chain alkynyl, C₁-C₉ alkoxy, C₂-C₉ alkenyloxy, aryloxy, phenoxy, benzyloxy, hydroxy, carboxy, C₁-C₉ thioalkyl, C₂-C₉ thioalkenyl, C₁-C₉ alkylamino, C₂ C₉ alkenylamino, cyano, nitro, imino, sulfonyl, thiocarbonyl, sulfhydryl, halo, haloalkyl, trifluoromethyl, aryl, heteroaryl, carbocycle or heterocycle, wherein said alkyl, alkenyl, alkynyl, alkoxy, alkenyloxy, aryloxy, thioalkyl, thioalkenyl, alkylamino, alkenylamino, aryl, heteroaryl, carbocycle or heterocycle is unsubstituted or substituted with hydroxy, carboxy, carbonyl, cyano, nitro, imino, sulfonyl, thiocarbonyl, sulfhydryl, halo, haloalkyl, trifluoromethyl, aryl, heteroaryl, carbocycle or heterocycle; and
X is O or S.

24. A method for the treatment, prophylactic treatment or prevention of nerve injury caused as a consequence of prostate surgery which comprises administering to a mammal in need of such treatment a compound of formula LXXII 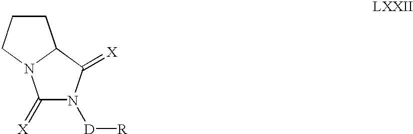
or a pharmaceutically acceptable salt, ester or solvate thereof, wherein:
each X is independently O, S or NR₂;
R₂ is cyano, nitro, hydrogen, C₁-C₄ alkyl, hydroxy or C₁-C₄ alkoxy;
D is a direct bond, C₁-C₈ alkyl or C₂-C₈ alkenyl; and
R is hydrogen or an alicyclic or aromatic, mono-, bi- or tricyclic, carbo- or heterocyclic ring, wherein R is unsubstituted or substituted with halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenyl, phenoxy, benzyloxy or amino.

25. A method for the treatment, prophylactic treatment or prevention of nerve injury caused as a consequence of prostate surgery which comprises administering to a mammal in need of such treatment a compound of formula LXXIII 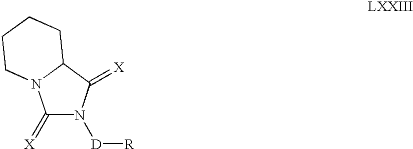
or a pharmaceutically acceptable salt, ester or solvate thereof, wherein:
each X is independently O, S or NR₂;
R₂ is cyano, nitro, hydrogen, C₁-C₄ alkyl, hydroxy or C₁-C₄ alkoxy;
D is a direct bond, C₁-C₈ alkyl or C₂-C₈ alkenyl; and
R is hydrogen or an alicyclic or aromatic, mono-, bi- or tricyclic, carbo- or heterocyclic ring, wherein R is unsubstituted or substituted with halo, hydroxyl, nitro, trifluoromethyl, C₁-C₆ straight or branched chain alkyl, C₂-C₆ straight or branched chain alkenyl, C₁-C₄ alkoxy, C₂-C₄ alkenyloxy, phenyl, phenoxy, benzyloxy or amino.
